(19) United States
(12) Patent Application Publication
(10) Pub. No.: US 2012/0040951 A1
(43) Pub. Date: Feb. 16, 2012

(54) HETEROARYL COMPOUNDS USEFUL AS RAF KINASE INHIBITORS

(76) Inventors: Claudio Chuaqui, Arlington, MA (US); Jennifer Cossrow, San Diego, CA (US); James Dowling, Somerville, MA (US); Bing Guan, Needham, MA (US); Michael Hoemann, Marlborough, MA (US); Alexey Ishchenko, Somerville, MA (US); John Howard Jones, Framingham, MA (US); Lori Kabigting, South San Francisco, CA (US); Gnanasambandam Kumaravel, Westford, MA (US); Hairuo Peng, Needham, MA (US); Noel Powell, Westford, MA (US); Brian Raimundo, San Francisco, CA (US); Hiroko Tanaka, Foster City, CA (US); Kurt van Vloten, Bellingham, MA (US); Jeffrey Vessels, Marlborough, MA (US); Zhili Xin, Lexington, MA (US)

(21) Appl. No.: 13/142,603

(22) PCT Filed: Dec. 30, 2009
(86) PCT No.: PCT/US09/69795

§ 371 Date: Nov. 2, 2011

Related U.S. Application Data
(60) Provisional application No. 61/141,561, filed on Dec. 30, 2008.

(51) Int. Cl. A61K 31/437; A61K 31/5377; C07D 417/14; A61P 35/00; A61P 9/00; A61P 25/28; A61P 37/00; A61P 37/06; A61P 29/00; A61P 31/12; A61P 19/08; A61P 1/16; A61P 7/00; A61P 3/10; C12N 9/99; C07D 471/04

(52) U.S. Cl. 514/210.18; 546/118; 514/303; 544/127; 514/234.2; 544/105; 540/579; 540/568; 435/184

(57) ABSTRACT

The present invention provides compounds of formula (I) useful as inhibitors of Raf protein kinase. The present invention also provides compositions thereof, and methods of treating Raf-mediated diseases.

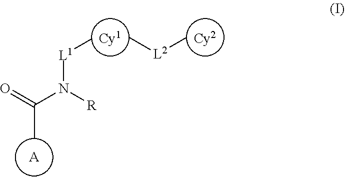

### CROSS-REFERENCE TO RELATED APPLICATIONS

The present application claims priority to U.S. provisional application Ser. No. 61/141,561, filed Dec. 30, 2008, the entirety of which is hereby incorporated by reference.

### TECHNICAL FIELD OF THE INVENTION

The present invention relates to compounds useful as inhibitors of protein kinases. The invention also provides pharmaceutically acceptable compositions comprising compounds of the present invention and methods of using said compositions in the treatment of various disorders.

### BACKGROUND OF THE INVENTION

Cancer results from the deregulation of the normal processes that control cell division, differentiation and apoptotic cell death. Protein kinases play a critical role in this regulatory process. A partial non-limiting list of such kinases includes abl, ATK, bcr-abl, Blk, Brk, Btk, c-kit, c-met, c-src, CDK1, CDK2, CDK4, CDK6, cRaf1, CSF1R, CSK, EGFR, ErbB2, ErbB3, ErbB4, ERK, Fak, fes, FGFR1, FGFR2, FGFR3, FGFR4, FGFR5, Fgr, FLK4, flt-1, Fps, Frk, Fyn, Hck, IGF-1R, INS-R, Jak, KDR, Lck, Lyn, MEK, p38, PDGFR, PIK, PKC, PYK2, ros, tie₁, tie₂, TRK, Yes and Zap70. In mammalian biology, such protein kinases comprise mitogen activated protein kinase (MAPK) signalling pathways. MAPK signalling pathways are inappropriately activated by a variety of common disease-associated mechanisms such as mutation of ras genes and deregulation of growth factor receptors (Magnuson et al., Seminars in Cancer Biology; 1994 (5), 247-252).

Additionally, protein kinases have been implicated as targets in central nervous system disorders (such as Alzheimer's), inflammatory disorders (such as psoriasis, arthritis), bone diseases (such as osteoporosis), atherosclerosis, restenosis, thrombosis, metabolic disorders (such as diabetes) and infectious diseases (such as viral and fungal infections).

One of the most commonly studied pathways involving kinase regulation is intracellular signalling from cell surface receptors to the nucleus. One example of this pathway includes a cascade of kinases in which members of the Growth Factor receptor Tyrosine Kinases (such as EGF-R, PDGF-R, VEGF-R, IGF1-R, the Insulin receptor) deliver signals through phosphorylation to other kinases such as Src Tyrosine kinase, and the Raf, Mek and Erk serine/threonine kinase families. Each of these kinases is represented by several family members, which play related, but functionally distinct roles. The loss of regulation of the growth factor signalling pathway is a frequent occurrence in cancer as well as other disease states.

The signals mediated by kinases have also been shown to control growth, death and differentiation in the cell by regulating the processes of the cell cycle. Progression through the eukaryotic cell cycle is controlled by a family of kinases called cyclin dependent kinases (CDKs). The regulation of CDK activation is complex, but requires the association of the CDK with a member of the cyclin family of regulatory subunits. A further level of regulation occurs through both activating and inactivating phosphorylations of the CDK subunit. The coordinate activation and inactivation of different cyclin/CDK complexes is necessary for normal progression through the cell cycle. Both the critical G1-S and G2-M transitions are controlled by the activation of different cyclin/CDK activities. In G1, both cyclin D/CDK4 and cyclin E/CDK2 are thought to mediate the onset of S-phase. Progression through S-phase requires the activity of cyclin A/CDK2 whereas the activation of cyclin A/cdc2 (CDK1) and cyclin B/cdc2 are required for the onset of metaphase. It is not surprising, therefore, that the loss of control of CDK regulation is a frequent event in hyperproliferative diseases and cancer.

Raf protein kinases are key components of signal transduction pathways by which specific extracellular stimuli elicit precise cellular responses in mammalian cells. Activated cell surface receptors activate ras/rap proteins at the inner aspect of the plasma membrane which in turn recruit and activate Raf proteins. Activated Raf proteins phosphorylate and activate the intracellular protein kinases MEK1 and MEK2. In turn, activated MEKs catalyze phosphorylation and activation of p42/p44 mitogen-activated protein kinase (MAPK). Various cytoplasmic and nuclear substrates of activated MAPK are known which directly or indirectly contribute to the cellular response to environmental change. Three distinct genes have been identified in mammals that encode Raf proteins; A-Raf, B-Raf and C-Raf (also known as Raf-1) and isoformic variants that result from differential splicing of mRNA are known.

Inhibitors of Raf kinases have been suggested for use in disruption of tumor cell growth and hence in the treatment of cancers, e.g., histiocytic lymphoma, lung adenocarcinoma, small cell lung cancer, and pancreatic and breast carcinoma; and also in the treatment and/or prophylaxis of disorders associated with neuronal degeneration resulting from ischemic events, including cerebral ischemia after cardiac arrest, stroke and multi-infarct dementia and also after cerebral ischemic events such as those resulting from head injury, surgery, and/or during childbirth.

Accordingly, there is a great need to develop compounds useful as inhibitors of protein kinases. In particular, it would be desirable to develop compounds that are useful as Raf inhibitors.

### SUMMARY OF THE INVENTION

It has now been found that compounds of this invention, and pharmaceutically acceptable compositions thereof, are effective as inhibitors of one or more protein kinases. Such compounds are of formula I:

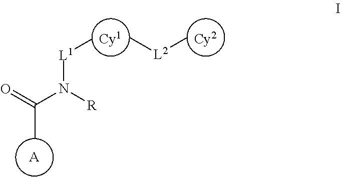

or a pharmaceutically acceptable salt thereof, wherein each of Ring A, R, L¹, L², Cy¹, and Cy² are as defined and described in classes and subclasses herein. Provided compounds are useful as inhibitors of one or more protein kinases (e.g., Raf), and thus are useful, for example, for the treatment of Raf-mediated diseases.

In certain other embodiments, the invention provides pharmaceutical compositions comprising a compound of the invention, wherein the compound is present in an amount effective to inhibit Raf activity. In certain other embodiments, the invention provides pharmaceutical compositions comprising a compound of the invention and optionally further comprising an additional therapeutic agent. In yet other embodiments, the additional therapeutic agent is an agent for the treatment of cancer.

In yet another aspect, the present invention provides methods for inhibiting kinase (e.g., Raf) activity in a patient or a biological sample, comprising administering to said patient, or contacting said biological sample with, an effective inhibitory amount of a compound of the invention. In still another aspect, the present invention provides methods for treating any disorder involving Raf activity, comprising administering to a subject in need thereof a therapeutically effective amount of a compound of the invention.

### DETAILED DESCRIPTION OF CERTAIN EMBODIMENTS OF THE INVENTION

### 1. General Description of Compounds of the Invention

In certain embodiments, the present invention provides a compound of formula I:

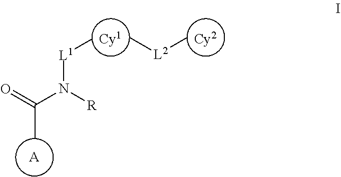

or a pharmaceutically acceptable salt thereof, wherein:
Cy¹ is phenylene, 5-6 membered saturated or partially unsaturated carbocyclylene, 7-10 membered saturated or partially unsaturated bicyclic carbocyclylene, a 5-6 membered saturated or partially unsaturated heterocyclylene ring having 1-2 heteroatoms independently selected from nitrogen, oxygen, and sulfur, a 7-10 membered saturated or partially unsaturated bicyclic heterocyclylene ring having 1-3 heteroatoms independently selected from nitrogen, oxygen, and sulfur, 8-10 membered bicyclic arylene, a 5-6 membered heteroarylene ring having 1-3 heteroatoms independently selected from nitrogen, oxygen, and sulfur, or an 8-10 membered bicyclic heteroarylene ring having 1-4 heteroatoms independently selected from nitrogen, oxygen, and sulfur, wherein:
   Cy¹ is optionally substituted with one or two groups independently selected from halogen, —R^{c}, —CN, —NO₂, —OR^{c}, —N(R^{c})₂, and —SR^{c}, wherein each R^{c} is independently hydrogen or a C₁₋₂ alkyl group optionally substituted with 1-3 groups independently selected from halogen, —OH, —NH₂, —SH, and —CN;
Cy² is an optionally substituted group selected from phenyl, a 5-8 membered saturated or partially unsaturated carbocyclic ring, a 7-10 membered saturated or partially unsaturated bicyclic carbocyclic ring, a 5-8 membered saturated or partially unsaturated heterocyclic ring having 1-2 heteroatoms independently selected from nitrogen, oxygen, and sulfur, a 7-10 membered saturated or partially unsaturated bicyclic heterocyclic ring having 1-3 heteroatoms independently selected from nitrogen, oxygen, and sulfur, an 8-10 membered bicyclic aryl ring, a 5-6 membered heteroaryl ring having 1-3 heteroatoms independently selected from nitrogen, oxygen, and sulfur, or an 8-10 membered bicyclic heteroaryl ring having 1-4 heteroatoms independently selected from nitrogen, oxygen, and sulfur;
L¹ is an optionally substituted, straight or branched bivalent C₁₋₆ alkylene chain;
L² is —NR¹— or —C(O)NR¹—;
R and R¹ are independently hydrogen or an optionally substituted C₁₋₆ aliphatic group; and
Ring A is an aromatic ring selected from the group consisting of Ring A¹, Ring A², Ring A³, Ring A⁴, and Ring A⁵, wherein:
(a) Ring A¹ is:

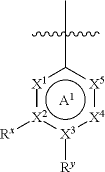

wherein:
X¹, X⁴ and X⁵ are independently CR⁴ or N;
X² is C or N, provided that when X² is N, R^{x} and R^{y} are taken together with their intervening atoms to form a fused heteroaromatic ring;
X³ is C;
R^{x} and R^{y} are independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂; or
R^{x} and R^{y} are taken together with their intervening atoms to form a 5-7 membered partially unsaturated or aromatic fused ring having 0-3 ring heteroatoms independently selected from nitrogen, oxygen, and sulfur; wherein:
   any substitutable carbon on the ring formed by R^{x} and R^{y} is optionally substituted with —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —C═NN(R³)₂, —C═NOR², —N(R³)C(O)NR³)₂, —N(R³)SO₂N(R³)₂, —N(R³)SO₂R², or —OC(O)N(R³)₂, and
   any substitutable nitrogen on the ring formed by R^{x} and R^{y} is optionally substituted with —R², —C(O)R², —CO₂R², —C(O)C(O)R², —C(O)CH₂—C(O)R², —S(O)R², —S(O)₂R², —C(O)N(R³)₂, —SO₂N(R³)₂, —OC(O)R², or —OC(O)N(R³)₂;
each R² is independently hydrogen or an optionally substituted group selected from C₁₋₆ aliphatic, phenyl, a 3-8 membered saturated or partially unsaturated carbocyclic ring, a 4-8 membered saturated or partially unsaturated heterocyclic ring having 1-2 heteroatoms independently selected from nitrogen, oxygen, and sulfur, a 7-10 membered saturated or partially unsaturated bicyclic heterocyclic ring having 1-4 heteroatoms independently selected from nitrogen, oxygen, and sulfur, an 8-10 membered bicyclic aryl ring, a 5-6 membered heteroaryl ring having 1-3 heteroatoms independently selected from nitrogen, oxygen, and sulfur, or an 8-10 membered bicyclic heteroaryl ring having 1-4 heteroatoms independently selected from nitrogen, oxygen, and sulfur;
each R³ is independently —R², or two R³ on the same nitrogen are taken together with the nitrogen to form an optionally substituted 5-8 membered saturated or partially unsaturated ring having 1-4 heteroatoms independently selected from nitrogen, oxygen, and sulfur; and
each R⁴ is independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂;
(b) Ring A² is:

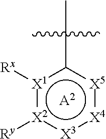

wherein:
X¹ and X² are independently C or N, provided that when X¹ or X² is N, R^{x} and R^{y} are taken together with their intervening atoms to form a fused heteroaromatic ring;
X³, X⁴, and X⁵ are independently CR⁴ or N;
R^{x} and R^{y} are independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂; or
R^{x} and R^{y} are taken together with their intervening atoms to form a 5-7 membered partially unsaturated or aromatic fused ring having 0-3 ring heteroatoms independently selected from nitrogen, oxygen, and sulfur; wherein:
   any substitutable carbon on the ring formed by R^{x} and R^{y} is optionally substituted with —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —C═NN(R³)₂, —C═NOR², —N(R³)C(O)NR³)₂, —N(R³)SO₂N(R³)₂, —N(R³)SO₂R², or —OC(O)N(R³)₂, and
   any substitutable nitrogen on the ring formed by R^{x} and R^{y} is optionally substituted with —R², —C(O)R², —CO₂R², —C(O)C(O)R², —C(O)CH₂—C(O)R², —S(O)R², —S(O)₂R², —C(O)N(R³)₂, —SO₂N(R³)₂, —OC(O)R², or —OC(O)N(R³)₂;
each R² is independently hydrogen or an optionally substituted group selected from C₁₋₆ aliphatic, phenyl, a 3-8 membered saturated or partially unsaturated carbocyclic ring, a 4-8 membered saturated or partially unsaturated heterocyclic ring having 1-2 heteroatoms independently selected from nitrogen, oxygen, and sulfur, a 7-10 membered saturated or partially unsaturated bicyclic heterocyclic ring having 1-4 heteroatoms independently selected from nitrogen, oxygen, and sulfur, an 8-10 membered bicyclic aryl ring, a 5-6 membered heteroaryl ring having 1-3 heteroatoms independently selected from nitrogen, oxygen, and sulfur, or an 8-10 membered bicyclic heteroaryl ring having 1-4 heteroatoms independently selected from nitrogen, oxygen, and sulfur;
each R³ is independently —R², or two R³ on the same nitrogen are taken together with the nitrogen to form an optionally substituted 5-8 membered saturated or partially unsaturated having 1-4 heteroatoms independently selected from nitrogen, oxygen, and sulfur; and each R⁴ is independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂;
(c) Ring A³ is:

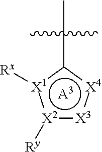

wherein:
X¹ and X² are independently C or N;
X³ and X⁴ are independently CR⁴, NR⁵, N, O, or S, as valency permits;
R^{x} and R^{y} are independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂; or
R^{x} and R^{y} are taken together with their intervening atoms to form a 5-7 membered partially unsaturated or aromatic fused ring having 0-3 ring heteroatoms independently selected from nitrogen, oxygen, and sulfur; wherein:
   any substitutable carbon on the ring formed by R^{x} and R^{y} is optionally substituted with —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —C═NN(R³)₂, —C═NOR², —N(R³)C(O)N(R³)₂, —N(R³)SO₂N(R³)₂, —N(R³)SO₂R², or —OC(O)N(R³)₂, and
   any substitutable nitrogen on the ring formed by R^{x} and R^{y} is optionally substituted with —R², —C(O)R², —CO₂R², —C(O)C(O)R², —C(O)CH₂C(O)R², —S(O)R², —S(O)₂R², C(O)N(R³)₂, —SO₂N(R³)₂, —OC(O)R², or —OC(O)N(R³)₂;
each R² is independently hydrogen or an optionally substituted group selected from C₁₋₆ aliphatic, phenyl, a 3-8 membered saturated or partially unsaturated carbocyclic ring, a 4-8 membered saturated or partially unsaturated heterocyclic ring having 1-2 heteroatoms independently selected from nitrogen, oxygen, and sulfur, a 7-10 membered saturated or partially unsaturated bicyclic heterocyclic ring having 1-4 heteroatoms independently selected from nitrogen, oxygen, and sulfur, an 8-10 membered bicyclic aryl ring, a 5-6 membered heteroaryl ring having 1-3 heteroatoms independently selected from nitrogen, oxygen, and sulfur, or an 8-10 membered bicyclic heteroaryl ring having 1-4 heteroatoms independently selected from nitrogen, oxygen, and sulfur;
each R³ is independently —R², or two R³ on the same nitrogen are taken together with the nitrogen to form an optionally substituted 5-8 membered saturated or partially unsaturated ring having 1-4 heteroatoms independently selected from nitrogen, oxygen, or sulfur;
each R⁴ is independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂; and
each R⁵ is independently —R², halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂;
(d) Ring A⁴ is:

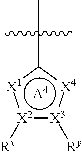

wherein:
X¹ and X⁴ are independently CR⁴, NR⁵, N, O, or S, as valency permits;
X² and X³ are independently C or N;
R^{x} and R^{y} are independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂; or
R^{x} and R^{y} are taken together with their intervening atoms to form a 5-7 membered partially unsaturated or aromatic fused ring having 0-3 ring heteroatoms independently selected from nitrogen, oxygen, and sulfur; wherein:
   any substitutable carbon on the ring formed by R^{x} and R^{y} is optionally substituted with —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —C═NN(R³)₂, —C═NOR², —N(R³)C(O)N(R³)₂, —N(R³)SO₂N(R³)₂, —N(R³)SO₂R², or —OC(O)N(R³)₂, and
   any substitutable nitrogen on the ring formed by R^{x} and R^{y} is optionally substituted with —R², —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², or —OC(O)N(R³)₂;
each R² is independently hydrogen or an optionally substituted group selected from C₁₋₆ aliphatic, phenyl, a 3-8 membered saturated or partially unsaturated carbocyclic ring, a 4-8 membered saturated or partially unsaturated heterocyclic ring having 1-2 heteroatoms independently selected from nitrogen, oxygen, and sulfur, a 7-10 membered saturated or partially unsaturated bicyclic heterocyclic ring having 1-4 heteroatoms independently selected from nitrogen, oxygen, and sulfur, an 8-10 membered bicyclic aryl ring, a 5-6 membered heteroaryl ring having 1-3 heteroatoms independently selected from nitrogen, oxygen, and sulfur, or an 8-10 membered bicyclic heteroaryl ring having 1-4 heteroatoms independently selected from nitrogen, oxygen, and sulfur;
each R³ is independently —R², or two R³ on the same nitrogen are taken together with the nitrogen to form an optionally substituted 5-8 membered saturated or partially unsaturated ring having 1-4 heteroatoms independently selected from nitrogen, oxygen, and sulfur;
each R⁴ is independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂; and
each R⁵ is independently —R², halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂;
(e) Ring A⁵ is:

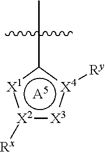

wherein:
X¹ and X³ are independently CR⁴, NR⁵, N, O, or S, as valency permits;
X² and X⁴ are independently C or N;
R^{x} and R^{y} are independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂;
each R² is independently hydrogen or an optionally substituted group selected from C₁₋₆ aliphatic, phenyl, a 3-8 membered saturated or partially unsaturated carbocyclic ring, a 4-8 membered saturated or partially unsaturated heterocyclic ring having 1-2 heteroatoms independently selected from nitrogen, oxygen, and sulfur, a 7-10 membered saturated or partially unsaturated bicyclic heterocyclic ring having 1-4 heteroatoms independently selected from nitrogen, oxygen, and sulfur, an 8-10 membered bicyclic aryl ring, a 5-6 membered heteroaryl ring having 1-3 heteroatoms independently selected from nitrogen, oxygen, and sulfur, or an 8-10 membered bicyclic heteroaryl ring having 1-4 heteroatoms independently selected from nitrogen, oxygen, and sulfur;
each R³ is independently —R², or two R³ on the same nitrogen are taken together with the nitrogen to form an optionally substituted 5-8 membered saturated or partially unsaturated ring having 1-4 heteroatoms independently selected from nitrogen, oxygen, and sulfur;
each R⁴ is independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂; and
each R⁵ is independently —R², halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂.

### 2. Compounds and Definitions

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972).

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

As used herein a “direct bond” or “covalent bond” refers to a single, double or triple bond. In certain embodiments, a “direct bond” refers to a single bond.

The terms “halo” and “halogen” as used herein refer to an atom selected from fluorine (fluoro, —F), chlorine (chloro, —Cl), bromine (bromo, —Br), and iodine (iodo, —I).

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-6 carbon atoms. In some embodiments, aliphatic groups contain 1-4 carbon atoms, and in yet other embodiments aliphatic groups contain 1-3 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “unsaturated”, as used herein, means that a moiety has one or more units of unsaturation.

The terms “cycloaliphatic”, “carbocycle”, “carbocyclyl”, “carbocyclo”, or “carbocyclic”, used alone or as part of a larger moiety, refer to a saturated or partially unsaturated cyclic aliphatic monocyclic or bicyclic ring systems, as described herein, having from 3 to 10 members, wherein the aliphatic ring system is optionally substituted as defined above and described herein. Cycloaliphatic (i.e. carbocyclic) groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloalkyl has 3-6 carbons. The terms “cycloaliphatic”, “carbocycle”, “carbocyclyl”, “carbocyclo”, or “carbocyclic” also include aliphatic rings that are fused to one or more aromatic or nonaromatic rings, such as decahydronaphthyl, tetrahydronaphthyl, decalin, or bicyclo[2.2.2]octane, where the radical or point of attachment is on an aliphatic ring.

As used herein, the term “cycloalkylene” refers to a bivalent cycloalkyl group. In certain embodiments, a cycloalkylene group is a 1,1-cycloalkylene group (i.e., a spiro-fused ring). Exemplary 1,1-cycloalkylene groups include

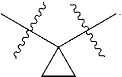

In other embodiments, a cycloalkylene group is a 1,2-cycloalkylene group or a 1,3-cycloalkylene group. Exemplary 1,2-cycloalkylene groups include

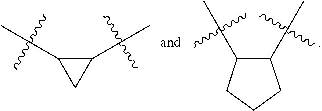

Similarly, the term “carbocyclylene” refers to a bivalent carbocyclic group.

The term “alkyl,” as used herein, refers to saturated, straight- or branched-chain hydrocarbon radicals derived from an aliphatic moiety containing between one and six carbon atoms by removal of a single hydrogen atom. In some embodiments, the alkyl group employed in the invention contains 1-5 carbon atoms. In another embodiment, the alkyl group employed contains 1-4 carbon atoms. In still other embodiments, the alkyl group contains 1-3 carbon atoms. In yet another embodiment, the alkyl group contains 1-2 carbons. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl,” as used herein, denotes a monovalent group derived from a straight- or branched-chain aliphatic moiety having at least one carbon-carbon double bond by the removal of a single hydrogen atom. In certain embodiments, the alkenyl group employed in the invention contains 2-6 carbon atoms. In certain embodiments, the alkenyl group employed in the invention contains 2-5 carbon atoms. In some embodiments, the alkenyl group employed in the invention contains 2-4 carbon atoms. In another embodiment, the alkenyl group employed contains 2-3 carbon atoms. Alkenyl groups include, for example, ethenyl, propenyl, butenyl, 1-methyl-2-buten-1-yl, and the like.

The term “alkynyl,” as used herein, refers to a monovalent group derived from a straight- or branched-chain aliphatic moiety having at least one carbon-carbon triple bond by the removal of a single hydrogen atom. In certain embodiments, the alkynyl group employed in the invention contains 2-6 carbon atoms. In certain embodiments, the alkynyl group employed in the invention contains 2-5 carbon atoms. In some embodiments, the alkynyl group employed in the invention contains 2-4 carbon atoms. In another embodiment, the alkynyl group employed contains 2-3 carbon atoms. Representative alkynyl groups include, but are not limited to, ethynyl, 2-propynyl (propargyl), 1-propynyl, and the like.

The term “aryl” used alone or as part of a larger moiety as in “aralkyl”, “aralkoxy”, or “aryloxyalkyl”, refers to monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenantriidinyl, or tetrahydronaphthyl, and the like. The term “arylene” refers to a bivalent aryl group.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted. The term “heteroarylene” refers to a bivalent heteroaryl group.

As used herein, the terms “heterocycle”, “heterocyclyl”, “heterocyclic radical”, and “heterocyclic ring” are used interchangeably and refer to a stable 4- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuryl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, 2-azabicyclo [2.2.1]heptanyl, octahydroindolyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted. The term “heterocyclylene” refers to a bivalent heterocyclic group.

As used herein, the term “partially unsaturated” refers to a ring moiety that includes at least one double or triple bond between ring atoms. The term “partially unsaturated” is intended to encompass rings having multiple sites of unsaturation, but is not intended to include aryl or heteroaryl moieties, as herein defined.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

Generally, the suffix “-ene” is used to describe a bivalent group. Thus, any of the terms above can be modified with the suffix “-ene” to describe a bivalent version of that moiety. For example, a bivalent carbocycle is “carbocyclylene”, a bivalent aryl ring is “arylene”, a bivalent benzene ring is “phenylene”, a bivalent heterocycle is “heterocyclylene”, a bivalent heteroaryl ring is “heteroarylene”, a bivalent alkyl chain is “alkylene”, a bivalent alkenyl chain is “alkenylene”, a bivalent alkynyl chain is “alkynylene”, and so forth.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned under this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘})₂; —OP(O)R^{∘})₂; —OP(O)(OR^{∘)}₂; —SiR^{∘}₃; —(C₁₋₄ straight or branched)alkylene)O—N(R^{∘})₂; or —(C₁₋₄ straight or branched)alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{}, -(haloR^{}), —OH, —OR^{}, —O(haloR^{}), —CN, —C(O)OH, —C(O)OR_{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

### 3. Description of Exemplary Compounds

As defined above, Ring A is selected from the group consisting of Ring A¹, A², A³, A⁴, and A⁵:

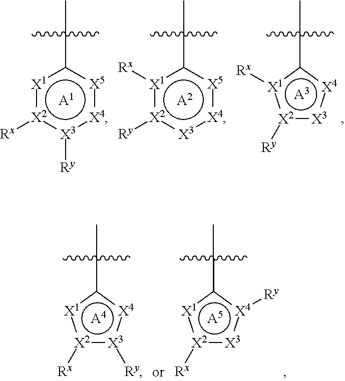

wherein each variable is as defined above and described herein.

In some embodiments, Ring A is Ring A¹:

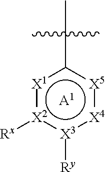

wherein X¹, X⁴ and X⁵ are independently CR⁴ or N; X² is C or N; X³ is C; and R^{x}, R^{y}, and R⁴ are as defined above and described herein. In some embodiments, when X² is N, R^{x} and R^{y} are taken together to form a fused aromatic ring. In certain embodiments, Ring A¹ is:

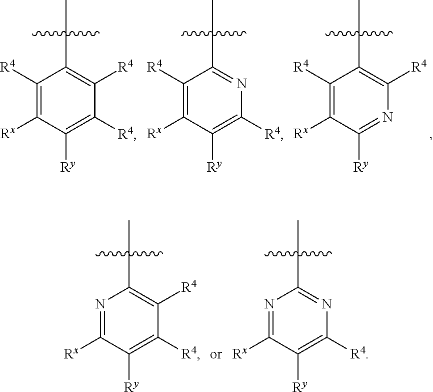

In other embodiments, Ring A¹ is:

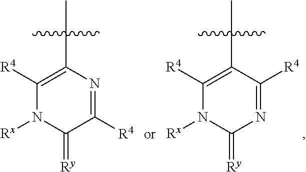

wherein R^{x} and R^{y} are taken together to form a fused heteroaromatic ring.

In some embodiments, Ring A is Ring A²:

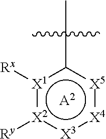

wherein X¹ and X² are independently C or N; X³, X⁴, and X⁵ are independently CR⁴ or N; and R^{x}, R^{y}, and R⁴ are as defined above and described herein. In some embodiments, X¹ is nitrogen, and R^{x} and R^{y} are taken together with their intervening atoms to form a fused heteroaromatic ring. In other embodiments, X² is nitrogen, and R^{x} and R^{y} are taken together with their intervening atoms to form a fused heteroaromatic ring. In certain embodiments, X³ and X⁵ are not simultaneously nitrogen. In certain embodiments, X³ and X⁵ are simultaneously nitrogen. In certain embodiments, Ring A² is:

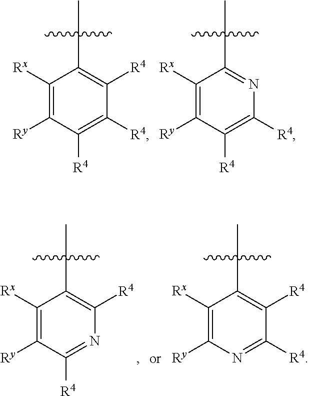

In other embodiments, Ring A² is:

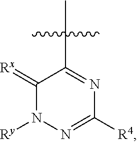

wherein R^{x} and R^{y} are taken together to form a fused heteroaromatic ring.

In some embodiments, Ring A is Ring A³:

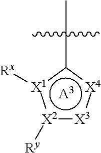

wherein X¹ and X² are independently C or N; X³ and X⁴ are independently CR⁴, NR⁵, N, O, or S, as valency permits; and R^{x}, R^{y}, R⁴ and R⁵ are as defined above and described herein. In certain embodiments, Ring A³ is:

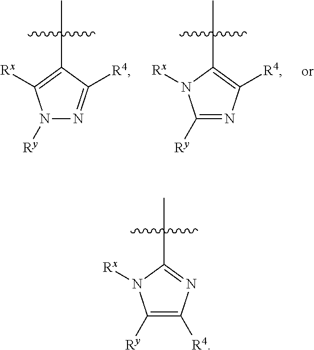

In some embodiments, Ring A is Ring A⁴:

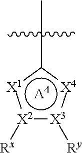

wherein X¹ and X⁴ are independently CR⁴, NR⁵, N, O, or S, as valency permits; X² and X³ are independently C or N; and R^{x}, R^{y}, R⁴ and R⁵ are as defined above and described herein. In certain embodiments, Ring A⁴ is:

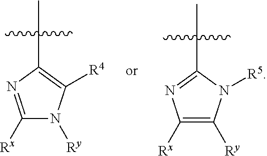

In some embodiments, Ring A is Ring A⁵:

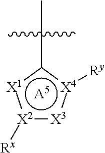

wherein X¹ and X³ are independently CR⁴, NR⁵, N, O, or S, as valency permits; X² and X⁴ are independently C or N; and R^{x}, R^{y}, R⁴ and R⁵ are as defined above and described herein. In certain embodiments, Ring A⁵ is:

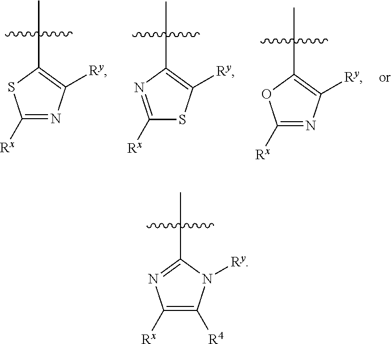

In some embodiments, R^{x} and R^{y} are independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂, wherein R² and R³ are as defined above and described herein.

In some embodiments, R^{x} is —R², oxo, halo, —CN, —OR², —N(R³)₂, or —N(R³)C(O)R², wherein R² and R³ are as defined above and described herein. In certain embodiments, R^{x} is —R² or halo. In some embodiments, R^{x} is hydrogen, —CN, an optionally substituted C₁₋₆ aliphatic group, or halo. In certain embodiments, R^{x} is hydrogen. In some embodiments, R^{x} is fluoro, chloro or bromo. In some embodiments, R^{x} is −OR². In certain embodiments, R^{x} is —OCH₃. In other embodiments, R^{x} is —N(R³)₂. In some embodiments, R^{x} is —NH(R³). In certain embodiments, R^{x} is —NH(C₁₋₆ alkyl). In certain other embodiments, R^{x} is —N(R³)C(O)R². In yet other embodiments, R^{x} is —NHC(O)CH₃.

In some embodiments, R^{x} is an optionally substituted C₁₋₆ aliphatic group. In certain embodiments, R^{x} is an optionally substituted C₁₋₆ alkyl group. In other embodiments, R^{x} is an optionally substituted C₁₋₃ alkyl group. In certain embodiments, R^{x} is an optionally substituted methyl, ethyl, n-propyl or isopropyl group. In certain embodiments, R^{x} is an optionally substituted methyl group. In certain embodiments, one or more substituents present on the C₁₋₆ aliphatic, C₁₋₆ alkyl, C₁₋₃ alkyl, n-propyl, isopropyl, ethyl or methyl group include —OR^{∘} and —N(R^{∘})₂, wherein R^{∘} is as described herein. In certain embodiments, a substituent on the methyl group is selected from morpholinyl, —OCH₃, piperidinyl, methylamino, pyrrolidinyl, cyclopropylamino, difluoropyrrolidinyl, or fluoroethylamino.

In certain embodiments, R^{x} is an optionally substituted C₈₋₁₀ bicyclic aryl ring. In some embodiments, R^{x} is an optionally substituted phenyl ring.

In some embodiments, R^{x} is an optionally substituted 4-8 membered saturated or partially unsaturated heterocyclic ring having 1-2 heteroatoms independently selected from nitrogen, oxygen, and sulfur. In some embodiments, R^{x} is an optionally substituted 7-10 membered saturated or partially unsaturated bicyclic heterocyclic ring having 1-4 heteroatoms independently selected from nitrogen, oxygen, and sulfur. In certain embodiments, R^{x} is an optionally substituted 5,6- or 6,6-fused saturated or partially unsaturated bicyclic ring having 1-4 heteroatoms independently selected from nitrogen, oxygen, and sulfur. In other embodiments, R^{x} is an optionally substituted 5-6 membered saturated or partially unsaturated heterocyclic ring having 1-2 heteroatoms independently selected from nitrogen, oxygen, and sulfur.

In certain embodiments, R^{x} is an optionally substituted 5-membered saturated heterocyclic ring having 1-2 heteroatoms independently selected from nitrogen, oxygen, and sulfur. In certain embodiments, R^{x} is an optionally substituted 6-membered saturated heterocyclic ring having 1-2 heteroatoms independently selected from nitrogen, oxygen, and sulfur. Exemplary R^{x} groups include optionally substituted octahydroazocinyl, thiocyclopentanyl, thiocyclohexanyl, pyrrolidinyl, piperidinyl, piperazinyl, tetrahydrothiopyranyl, tetrahydrothienyl, dithiolanyl, tetrahydrofuryl, tetrahydropyranyl, dioxanyl, thioxanyl, morpholinyl, oxathiolanyl, imidazolidinyl, oxathiolanyl, oxazolidinyl, and thiazolidinyl. In certain embodiments, R^{x} is optionally substituted imidazolidinyl, oxathiolanyl, oxazolidinyl, or thiazolidinyl. In some embodiments, R^{x} is optionally substituted piperidinyl, piperazinyl, morpholinyl, or pyrrolidinyl. In certain embodiments, R^{x} is optionally substituted morpholinyl. In certain embodiments, R^{x} is optionally substituted tetrahydropyridyl.

In certain embodiments, R^{x} is an optionally substituted 5-6 membered heteroaryl ring having 1-3 heteroatoms selected from nitrogen, oxygen, and sulfur. In some embodiments, R^{x} is an optionally substituted 5-6 membered heteroaryl ring having 1-2 heteroatoms selected from nitrogen, oxygen, and sulfur. In other embodiments, R^{x} is an optionally substituted 5-6 membered heteroaryl ring having 2 heteroatoms selected from nitrogen, oxygen, and sulfur. In certain embodiments, R^{x} is an optionally substituted 5-6 membered heteroaryl ring having 1 heteroatom selected from nitrogen, oxygen, and sulfur. Exemplary R^{x} groups include optionally substituted pyrrolyl, pyrazolyl, imidazolyl, triazolyl, tetrazolyl, thienyl, furyl, thiazolyl, isothiazolyl, thiadiazolyl, oxazolyl, isoxazolyl, oxadiaziolyl, pyridyl, pyrimidinyl, pyrazolyl, pyrazinyl, pyridazinyl, triazinyl, and tetrazinyl. In certain embodiments, R^{x} is optionally substituted pyridyl.

In certain embodiments, R^{x} is an optionally substituted 8-10 membered bicyclic heteroaryl ring having 1-4 heteroatoms independently selected from nitrogen, oxygen, and sulfur. In some embodiments, R^{x} is an optionally substituted 5,6-fused or 6,6-fused heteroaryl ring having 1-4 heteroatoms independently selected from nitrogen, oxygen, and sulfur. In other embodiments, R^{x} is an optionally substituted 5,6-fused or 6,6-fused heteroaryl ring having 1-2 heteroatoms independently selected from nitrogen, oxygen, and sulfur. In certain embodiments, R^{x} is an optionally substituted 5,6-fused or 6,6-fused heteroaryl ring having 1 heteroatom independently selected from nitrogen, oxygen, and sulfur.

Exemplary R^{x} groups include those set forth in Examples 1-357, inclusive, in the Examples section, infra.

In some embodiments, R^{y} is —R², oxo, halo, —CN, —OR², —N(R³)₂, or —N(R³)C(O)R², wherein R² and R³ are as defined above and described herein. In certain embodiments, R^{y} is —R² or halo. In some embodiments, R^{y} is hydrogen, —CN, an optionally substituted C₁₋₆ aliphatic group, or halo. In certain embodiments, R^{y} is hydrogen. In some embodiments, R^{x} is fluoro, chloro or bromo. In some embodiments, R^{y} is —OR². In certain embodiments, R^{y} is —OCH₃. In other embodiments, R^{y} is —N(R³)₂. In certain embodiments, R^{y} is —NH(R³). In certain other embodiments, R^{y} is —NH(C₁₋₆ alkyl). In some embodiments, R^{y} is —N(R³)C(O)R². In certain embodiments, R^{y} is —NHC(O)CH₃.

In some embodiments, R^{y} is an optionally substituted C₁₋₆ aliphatic group. In certain embodiments, R^{y} is an optionally substituted C₁₋₆ alkyl group. In other embodiments, R^{y} is an optionally substituted C₁₋₃ alkyl group. In certain embodiments, R^{y} is an optionally substituted methyl, ethyl, n-propyl or isopropyl group. In certain embodiments, R^{y} is an optionally substituted methyl group. In certain embodiments, one or more substituents present on the C₁₋₆ aliphatic, C₁₋₆ alkyl, C₁₋₃ alkyl, n-propyl, isopropyl, ethyl or methyl group include —OR^{∘} and —N(R^{∘})₂, wherein R^{∘} is as described herein. In certain embodiments, a substituent on the methyl group is morpholinyl, —OCH₃, piperidinyl, methylamino, pyrrolidinyl, cyclopropylamino, difluoropyrrolidinyl, or fluoroethylamino.

In certain embodiments, R^{y} is an optionally substituted C₈₋₁₀ bicyclic aryl ring. In some embodiments, R^{y} is an optionally substituted phenyl ring.

In some embodiments, R^{y} is an optionally substituted 4-8 membered saturated or partially unsaturated heterocyclic ring having 1-2 heteroatoms independently selected from nitrogen, oxygen, and sulfur. In some embodiments, R^{y} is an optionally substituted 7-10 membered saturated or partially unsaturated bicyclic heterocyclic ring having 1-4 heteroatoms independently selected from nitrogen, oxygen, and sulfur. In certain embodiments, R^{y} is an optionally substituted 5,6- or 6,6-fused saturated or partially unsaturated bicyclic ring having 1-4 heteroatoms independently selected from nitrogen, oxygen, and sulfur. In other embodiments, R^{y} is an optionally substituted 5-6 membered saturated or partially unsaturated heterocyclic ring having 1-2 heteroatoms independently selected from nitrogen, oxygen, and sulfur.

In certain embodiments, R^{y} is an optionally substituted 5-membered saturated heterocyclic ring having 1-2 heteroatoms independently selected from nitrogen, oxygen, and sulfur. In certain embodiments, R^{y} is an optionally substituted 6-membered saturated heterocyclic ring having 1-2 heteroatoms independently selected from nitrogen, oxygen, and sulfur. Exemplary R^{y} groups include optionally substituted octahydroazocinyl, thiocyclopentanyl, thiocyclohexanyl, pyrrolidinyl, piperidinyl, piperazinyl, tetrahydrothiopyranyl, tetrahydrothienyl, dithiolanyl, tetrahydrofuryl, tetrahydropyranyl, dioxanyl, thioxanyl, morpholinyl, oxathiolanyl, imidazolidinyl, oxathiolanyl, oxazolidinyl, and thiazolidinyl. In certain embodiments, R^{y} is optionally substituted imidazolidinyl, oxathiolanyl, oxazolidinyl, or thiazolidinyl. In some embodiments, R^{y} is optionally substituted piperidinyl, piperazinyl, morpholinyl, or pyrrolidinyl. In certain embodiments, R^{y} is optionally substituted morpholinyl. In certain embodiments, R^{y} is optionally substituted tetrahydropyridyl.

In certain embodiments, R^{y} is an optionally substituted 5-6 membered heteroaryl ring having 1-3 heteroatoms selected from nitrogen, oxygen, and sulfur. In some embodiments, R^{y} is an optionally substituted 5-6 membered heteroaryl ring having 1-2 heteroatoms selected from nitrogen, oxygen, and sulfur. In other embodiments, R^{y} is an optionally substituted 5-6 membered heteroaryl ring having 2 heteroatoms selected from nitrogen, oxygen, and sulfur. In certain embodiments, R^{y} is an optionally substituted 5-membered heteroaryl ring having 1 heteroatom selected from nitrogen, oxygen, and sulfur. In certain embodiments, R^{y} is an optionally substituted 5-6 membered heteroaryl ring having 1 nitrogen, and an additional heteroatom selected from sulfur and oxygen. Exemplary R^{y} groups include optionally substituted pyrrolyl, pyrazolyl, imidazolyl, triazolyl, tetrazolyl, thienyl, furyl, thiazolyl, isothiazolyl, thiadiazolyl, oxazolyl, isoxazolyl, oxadiaziolyl, pyridyl, pyrimidinyl, pyrazolyl, pyrazinyl, pyridazinyl, triazinyl, and tetrazinyl. In certain embodiments, R^{y} is optionally substituted pyridyl.

In certain embodiments, R^{y} is an optionally substituted 8-10 membered bicyclic heteroaryl ring having 1-4 heteroatoms independently selected from nitrogen, oxygen, and sulfur. In some embodiments, R^{y} is an optionally substituted 5,6-fused or 6,6-fused heteroaryl ring having 1-4 heteroatoms independently selected from nitrogen, oxygen, and sulfur. In other embodiments, R^{y} is an optionally substituted 5,6-fused or 6,6-fused heteroaryl ring having 1-2 heteroatoms independently selected from nitrogen, oxygen, and sulfur. In certain embodiments, R^{y} is an optionally substituted 5,6-fused or 6,6-fused heteroaryl ring having 1 heteroatom independently selected from nitrogen, oxygen, and sulfur.

Exemplary R^{y} groups include those set forth in Examples 1-357, inclusive, in the Examples section, infra.

In some embodiments, R^{x} and R^{y} are taken together with their intervening atoms to form a 5-membered partially unsaturated or aromatic fused ring having 0-3 heteroatoms independently selected from nitrogen, oxygen, and sulfur, wherein said ring is optionally substituted as defined above and described herein.

In some embodiments, R^{x} and R^{y} are taken together with their intervening atoms to form a 5-membered partially unsaturated or aromatic fused carbocyclic ring, wherein said ring is optionally substituted as defined above and described herein. In certain embodiments, R^{x} and R^{y} are taken together to form a cyclopentenyl or cyclopentadienyl ring, wherein said ring is optionally substituted as defined above and described herein.

In certain embodiments, R^{x} and R^{y} are taken together with their intervening atoms to form a 5-membered partially unsaturated fused ring having 1-3 heteroatoms independently selected from nitrogen, oxygen, and sulfur, wherein said ring is optionally substituted as defined above and described herein. In some embodiments, R^{x} and R^{y} are taken together with their intervening atoms to form a 5-membered partially unsaturated fused ring having 1-3 nitrogens, wherein said ring is optionally substituted as defined above and described herein. In other embodiments, R^{x} and R^{y} are taken together with their intervening atoms to form a 5-membered partially unsaturated fused ring having 1-2 nitrogens, wherein said ring is optionally substituted as defined above and described herein. In some embodiments, R^{x} and R^{y} are taken together to form an imidazolidinono-, oxazolidinono-, or pyrrolidinono-fused ring, wherein said ring is optionally substituted as defined above and described herein. In other embodiments, R^{x} and R^{y} are taken together to form an imidazolidino- or pyrrolidino-fused ring, wherein said ring is optionally substituted as defined above and described herein.

In certain embodiments, R^{x} and R^{y} are taken together with their intervening atoms to form a 5-membered aromatic fused ring having 1-3 heteroatoms independently selected from nitrogen, oxygen, and sulfur, wherein said ring is optionally substituted as defined above and described herein. In some embodiments, R^{x} and R^{y} are taken together with their intervening atoms to form a 5-membered aromatic fused ring having 1 or 2 heteroatoms independently selected from nitrogen, oxygen, and sulfur, wherein said ring is optionally substituted as defined above and described herein. In certain embodiments, R^{x} and R^{y} are taken together with their intervening atoms to form a 5-membered aromatic fused ring having 2 or 3 nitrogens, wherein said ring is optionally substituted as defined above and described herein. In certain embodiments, R^{x} and R^{y} are taken together to form a pyrrolo-, pyrazolo-, imidazolo-, triazolo-, thieno-, furo-, thiazolo-, isothiazolo-, thiadiazolo-, oxazolo-, isoxazolo-, or oxadiaziolo-fused ring, wherein said ring is optionally substituted as defined above and described herein. In certain embodiments, R^{x} and R^{y} are taken together to form a pyrazolo-, imidazolo-, or thiazolo-fused ring, wherein said ring is optionally substituted as defined above and described herein. In certain embodiments, R^{x} and R^{y} are taken together to form an imidazolo-fused ring, wherein said ring is optionally substituted as defined above and described herein.

In certain embodiments, R^{x} and R^{y} are taken together with their intervening atoms to form a 6-membered partially unsaturated or aromatic fused ring having 0-3 heteroatoms independently selected from nitrogen, oxygen, and sulfur, wherein said ring is optionally substituted as defined above and described herein.

In certain embodiments, R^{x} and R^{y} are taken together with their intervening atoms to form a 6-membered partially unsaturated or aromatic fused carbocyclic ring, wherein said ring is optionally substituted as defined above and described herein. In some embodiments, R^{x} and R^{y} are taken together with their intervening atoms to form a 6-membered partially unsaturated fused carbocyclic ring, wherein said ring is optionally substituted as defined above and described herein. In certain embodiments, R^{x} and R^{y} are taken together with their intervening atoms to form a benzo-fused ring, wherein said ring is optionally substituted as defined above and described herein.

In certain embodiments, R^{x} and R^{y} are taken together with their intervening atoms to form a 6-membered partially unsaturated fused ring having 1-3 heteroatoms independently selected from nitrogen, oxygen, and sulfur, wherein said ring is optionally substituted as defined above and described herein. In some embodiments, R^{x} and R^{y} are taken together with their intervening atoms to form a 6-membered partially unsaturated fused ring having 1 or 2 heteroatoms independently selected from nitrogen, oxygen, and sulfur, wherein said ring is optionally substituted as defined above and described herein. In certain embodiments, R^{x} and R^{y} are taken together to form a dioxano-, morpholino-, morpholinono-, tetrahydropyrimidino-, piperazino-, or piperidino-fused ring, wherein said ring is optionally substituted as defined above and described herein. In certain embodiments, R^{x} and R^{y} are taken together to form a morpholinono-, piperidino-, or tetrahydropyrimidino-fused ring, wherein said ring is optionally substituted as defined above and described herein.

In certain embodiments, R^{x} and R^{y} are taken together with their intervening atoms to form a 6-membered aromatic fused ring having 1-3 heteroatoms independently selected from nitrogen, oxygen, and sulfur, wherein said ring is optionally substituted as defined above and described herein. In some embodiments, R^{x} and R^{y} are taken together with their intervening atoms to form a 6-membered aromatic fused ring having 1-3 nitrogens, wherein said ring is optionally substituted as defined above and described herein. In certain embodiments, R^{x} and R^{y} are taken together to form a pyrazino-, pyrido-, pyrimidino-, pyridazino-, or triazino-fused ring, wherein said ring is optionally substituted as defined above and described herein. In certain embodiments, R^{x} and R^{y} are taken together to form a pyrazino- or pyrido-fused ring, wherein said ring is optionally substituted as defined above and described herein.

In certain embodiments, R^{x} and R^{y} are taken together with their intervening atoms to form a 7-membered partially unsaturated fused ring having 0-3 heteroatoms independently selected from nitrogen, oxygen, and sulfur, wherein said ring is optionally substituted as defined above and described herein. In some embodiments, R^{x} and R^{y} are taken together with their intervening atoms to form a 7-membered partially unsaturated carbocyclic fused ring, wherein said ring is optionally substituted as defined above and described herein. In certain embodiments, R^{x} and R^{y} are taken together to form a cyclohepteno-, cycloheptadieno-, or cycloheptatrieno-fused ring, wherein said ring is optionally substituted as defined above and described herein.

In certain embodiments, R^{x} and R^{y} are taken together with their intervening atoms to form a 7-membered partially unsaturated fused ring having 1-3 heteroatoms independently selected from nitrogen, oxygen, and sulfur, wherein said ring is optionally substituted as defined above and described herein. In other embodiments, R^{x} and R^{y} are taken together with their intervening atoms to form a 7-membered partially unsaturated fused ring having 1 or 2 heteroatoms independently selected from nitrogen, oxygen, and sulfur, wherein said ring is optionally substituted as defined above and described herein. In certain embodiments, R^{x} and R^{y} are taken together to form a oxepino-, oxepinono-, thiepino-, thiepinono, azepino-, diazapino-, azepinono-, or diazepinono-fused ring, wherein said ring is optionally substituted as defined above and described herein. In certain embodiments, R^{x} and R^{y} are taken together to form an azepino- or diazepino-fused ring, wherein said ring is optionally substituted as defined above and described herein.

In some embodiments, any substitutable carbon on the ring formed by R^{x} and R^{y} is optionally substituted with —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —C═NN(R³)₂, —C═NOR², —N(R³)C(O)NR³)₂, —N(R³)SO₂N(R³)₂, —N(R³)SO₂R², or —OC(O)N(R³)₂, wherein R² and R³ are as defined above and described herein. In certain embodiments, any substitutable carbon on the ring formed by R^{x} and R^{y} is optionally substituted with hydrogen, halo, or oxo. In certain embodiments, any substitutable carbon on the ring formed by R^{x} and R^{y} is optionally substituted with —R². In some embodiments, any substitutable carbon on the ring formed by R^{x} and R^{y} is optionally substituted with hydrogen, oxo or an optionally substituted C₁₋₆ aliphatic group. In some embodiments, any substitutable carbon on the ring formed by R^{x} and R^{y} is optionally substituted with an optionally substituted 5-6 membered heteroaryl ring having 1-3 heteroatoms independently selected from nitrogen, oxygen, and sulfur. In certain embodiments, any substitutable carbon on the ring formed by R^{x} and R^{y} is optionally substituted with optionally substituted pyrimidinyl or pyridyl. In other embodiments, any substitutable carbon on the ring formed by R^{x} and R^{y} is optionally substituted with hydrogen, oxo or methyl. In certain embodiments, any substitutable carbon on the ring formed by R^{x} and R^{y} is optionally substituted with a halogen. In certain embodiments, any substitutable carbon on the ring formed by R^{x} and R^{y} is optionally substituted with bromo. In some embodiments, any substitutable carbon on the ring formed by R^{x} and R^{y} is optionally substituted with —N(R³)₂, wherein R³ is as defined above and described herein. In certain embodiments, any substitutable carbon on the ring formed by R^{x} and R^{y} is optionally substituted with —NH₂.

In some embodiments, any substitutable nitrogen on the ring formed by R^{x} and R^{y} is optionally substituted with —R², —C(O)R², —CO₂R², —C(O)C(O)R², —C(O)CH₂—C(O)R², —S(O)R², —S(O)₂R², —C(O)N(R³)₂, —SO₂N(R³)₂, —OC(O)R², or —OC(O)N(R³)₂, wherein R² and R³ are as defined above and described herein. In certain embodiments, any substitutable nitrogen on the ring formed by R^{x} and R^{y} is optionally substituted with hydrogen, —C(O)R², or —CO₂R². In certain embodiments, any substitutable nitrogen on the ring formed by R^{x} and R^{y} is optionally substituted with —R². In some embodiments, any substitutable nitrogen on the ring formed by R^{x} and R^{y} is optionally substituted with hydrogen or an optionally substituted C₁₋₆ aliphatic group. In some embodiments, any substitutable nitrogen on the ring formed by R^{x} and R^{y} is optionally substituted with an optionally substituted 4-7 membered saturated ring having 1-2 heteroatoms independently selected from nitrogen, oxygen, and sulfur. In certain embodiments, any substitutable nitrogen on the ring formed by R^{x} and R^{y} is optionally substituted with optionally substituted cyclobutyl. In certain embodiments, any substitutable nitrogen on the ring formed by R^{x} and R^{y} is optionally substituted with optionally substituted azetidinyl or pyrrolidinyl. In other embodiments, any substitutable nitrogen on the ring formed by R^{x} and R^{y} is optionally substituted with hydrogen, methyl, ethyl, or isobutyl. In certain embodiments, any substitutable nitrogen on the ring formed by R^{x} and R^{y} is optionally substituted with a methyl group.

As defined generally above, each R² is independently hydrogen or an optionally substituted group selected from C₁₋₆ aliphatic, phenyl, a 3-8 membered saturated or partially unsaturated carbocyclic ring, a 4-8 membered saturated or partially unsaturated heterocyclic ring having 1-2 heteroatoms independently selected from nitrogen, oxygen, and sulfur, a 7-10 membered saturated or partially unsaturated bicyclic heterocyclic ring having 1-4 heteroatoms independently selected from nitrogen, oxygen, and sulfur, an 8-10 membered bicyclic aryl ring, a 5-6 membered heteroaryl ring having 1-3 heteroatoms independently selected from nitrogen, oxygen, and sulfur, or an 8-10 membered bicyclic heteroaryl ring having 1-4 heteroatoms independently selected from nitrogen, oxygen, and sulfur.

In certain embodiments, R² is hydrogen. In some embodiments, R² is an optionally substituted C₁₋₆ aliphatic group. In certain embodiments, R² is an optionally substituted C₁₋₆ alkyl group. In other embodiments, R² is an optionally substituted C₁₋₃ alkyl group. In certain embodiments, R² is an optionally substituted methyl, ethyl, n-propyl or isopropyl group. In certain embodiments, R² is an optionally substituted methyl group.

In certain embodiments, R² is an optionally substituted C₈₋₁₀ bicyclic aryl ring. In some embodiments, R² is an optionally substituted phenyl ring.

In some embodiments, R² is an optionally substituted 4-8 membered saturated or partially unsaturated heterocyclic ring having 1-2 heteroatoms independently selected from nitrogen, oxygen, and sulfur. In some embodiments, R² is an optionally substituted 7-10 membered saturated or partially unsaturated bicyclic heterocyclic ring having 1-4 heteroatoms independently selected from nitrogen, oxygen, and sulfur. In certain embodiments, R² is an optionally substituted 5,6- or 6,6-fused saturated bicyclic ring having 1-4 heteroatoms independently selected from nitrogen, oxygen, and sulfur. In other embodiments, R² is an optionally substituted 5-6 membered saturated heterocyclic ring having 1-2 heteroatoms independently selected from nitrogen, oxygen, and sulfur.

In certain embodiments, R² is an optionally substituted 5-6 membered saturated heterocyclic ring having 1-2 heteroatoms independently selected from nitrogen, oxygen, and sulfur. In some embodiments, R² is an optionally substituted 5-6 membered saturated heterocyclic ring having 2 heteroatoms independently selected from nitrogen, oxygen, and sulfur. Exemplary R² groups include optionally substituted octahydroazocinyl, thiocyclopentanyl, thiocyclohexanyl, pyrrolidinyl, piperidinyl, piperazinyl, tetrahydrothiopyranyl, tetrahydrothienyl, dithiolanyl, tetrahydrofuryl, tetrahydropyranyl, dioxanyl, thioxanyl, morpholinyl, oxathiolanyl, imidazolidinyl, oxathiolanyl, oxazolidinyl, and thiazolidinyl. In certain embodiments, R² is optionally substituted imidazolidinyl, oxathiolanyl, oxazolidinyl, or thiazolidinyl. In some embodiments, R² is optionally substituted piperidinyl, piperazinyl, morpholinyl, or pyrrolidinyl. In certain embodiments, R² is optionally substituted morpholinyl.

In certain embodiments, R² is an optionally substituted 5-6 membered heteroaryl ring having 1-3 heteroatoms selected from nitrogen, oxygen, and sulfur. In some embodiments, R² is an optionally substituted 5-6 membered heteroaryl ring having 1-2 heteroatoms selected from nitrogen, oxygen, and sulfur. In other embodiments, R² is an optionally substituted 5-6 membered heteroaryl ring having 2 heteroatoms selected from nitrogen, oxygen, and sulfur. In certain embodiments, R² is an optionally substituted 5-6 membered heteroaryl ring having 1 heteroatom selected from nitrogen, oxygen, and sulfur. Exemplary R² groups include optionally substituted pyrrolyl, pyrazolyl, imidazolyl, triazolyl, tetrazolyl, thienyl, furyl, thiazolyl, isothiazolyl, thiadiazolyl, oxazolyl, isoxazolyl, oxadiazolyl, pyridyl, pyrimidinyl, pyrazolyl, pyrazinyl, pyridazinyl, triazinyl, and tetrazinyl. In certain embodiments, R² is optionally substituted pyridyl.

In certain embodiments, R² is an optionally substituted 8-10 membered bicyclic heteroaryl ring having 1-4 heteroatoms independently selected from nitrogen, oxygen, and sulfur. In some embodiments, R² is an optionally substituted 5,6-fused or 6,6-fused heteroaryl ring having 1-4 heteroatoms independently selected from nitrogen, oxygen, and sulfur. In other embodiments, R² is an optionally substituted 5,6-fused or 6,6-fused heteroaryl ring having 1-2 heteroatoms independently selected from nitrogen, oxygen, and sulfur. In certain embodiments, R² is an optionally substituted 5,6-fused or 6,6-fused heteroaryl ring having 1 heteroatom independently selected from nitrogen, oxygen, and sulfur.

As defined above, each R³ is independently —R², or two R³ on the same nitrogen are taken together with the nitrogen to form an optionally substituted 5-8 membered saturated or partially unsaturated ring having 1-4 heteroatoms independently selected from nitrogen, oxygen, and sulfur. In certain embodiments, R³ is —R² as described in classes and subclasses herein.

In some embodiments, two R³ on the same nitrogen are taken together with the nitrogen to form an optionally substituted 5-8 membered saturated, partially unsaturated, or aromatic ring having 1-4 heteroatoms independently selected from nitrogen, oxygen, and sulfur. In certain embodiments, two R³ on the same nitrogen are taken together with the nitrogen to form an optionally substituted 5-8 membered saturated ring having 1-4 heteroatoms independently selected from nitrogen, oxygen, and sulfur. In certain embodiments, two R³ on the same nitrogen are taken together with the nitrogen to form an optionally substituted 5-8 membered partially unsaturated ring having 1-4 heteroatoms independently selected from nitrogen, oxygen, and sulfur. In certain embodiments, two R³ on the same nitrogen are taken together with the nitrogen to form an optionally substituted pyrrolidine, piperidine, homopiperidine, or morpholine ring.

As defined generally above, each R⁴ is independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂, wherein groups R² and R³ are as defined above and described herein.

In some embodiments, R⁴ is —R², oxo, halo, —CN, —OR², —N(R³)₂, or —N(R³)C(O)R², wherein R² and R³ are as defined above and described herein. In certain embodiments, R⁴ is —R² or halo. In some embodiments, R⁴ is hydrogen, —CN, an optionally substituted C₁₋₆ aliphatic group, or halo. In certain embodiments, R⁴ is hydrogen. In some embodiments, R⁴ is fluoro, chloro or bromo. In some embodiments, R⁴ is —OR². In certain embodiments, R⁴ is —OCH₃. In other embodiments, R⁴ is —N(R³)₂. In some embodiments, R⁴ is —NH(R³). In certain embodiments, R⁴ is —NH(C₁₋₆ alkyl). In certain other embodiments, R⁴ is —N(R³)C(O)R². In yet other embodiments, R⁴ is —NHC(O)CH₃.

In some embodiments, R⁴ is an optionally substituted C₁₋₆ aliphatic group. In certain embodiments, R⁴ is an optionally substituted C₁₋₆ alkyl group. In other embodiments, R⁴ is an optionally substituted C₁₋₃ alkyl group. In certain embodiments, R⁴ is an optionally substituted methyl, ethyl, n-propyl or isopropyl group. In certain embodiments, R⁴ is an optionally substituted methyl group. In certain embodiments, one or more substituents present on the C₁₋₆ aliphatic, C₁₋₆ alkyl, C₁₋₃ alkyl, n-propyl, isopropyl, ethyl or methyl group include —OR^{∘} and —N(R^{∘})₂, wherein R^{∘} is as described herein. In certain embodiments, a substituent on the methyl group is selected from morpholinyl, —OCH₃, piperidinyl, methylamino, pyrrolidinyl, cyclopropylamino, difluoropyrrolidinyl, or fluoroethylamino.

In certain embodiments, R⁴ is —R² as defined and described in classes and subclasses herein.

Exemplary R⁴ groups include those set forth in Examples 1-357, inclusive, in the Examples section, infra.

As defined generally above, each R⁵ is independently —R², halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂, wherein groups R² and R³ are as defined above and described herein.

In some embodiments, R⁵ is —R², halo, —CN, —OR², —N(R³)₂, or —N(R³)C(O)R², wherein R² and R³ are as defined above and described herein. In certain embodiments, R⁵ is —R² or halo. In some embodiments, R⁵ is hydrogen, —CN, an optionally substituted C₁₋₆ aliphatic group, or halo. In certain embodiments, R⁵ is hydrogen. In some embodiments, R⁵ is fluoro, chloro or bromo. In some embodiments, R⁵ is —OR². In certain embodiments, R⁵ is —OCH₃. In other embodiments, R⁵ is —N(R³)₂. In some embodiments, R⁵ is —NH(R³). In certain embodiments, R⁵ is —NH(C₁₋₆ alkyl). In certain other embodiments, R⁵ is —N(R³)C(O)R². In yet other embodiments, R⁵ is —NHC(O)CH₃.

In some embodiments, R⁵ is an optionally substituted C₁₋₆ aliphatic group. In certain embodiments, R⁵ is an optionally substituted C₁₋₆ alkyl group. In other embodiments, R⁵ is an optionally substituted C₁₋₃ alkyl group. In certain embodiments, R⁵ is an optionally substituted methyl, ethyl, n-propyl or isopropyl group. In certain embodiments, R⁵ is an optionally substituted methyl group. In certain embodiments, one or more substituents present on the C₁₋₆ aliphatic, C₁₋₆ alkyl, C₁₋₃ alkyl, n-propyl, isopropyl, ethyl or methyl group include —OR^{∘} and —N(R^{∘})₂, wherein R^{∘} is as described herein. In certain embodiments, a substituent on the methyl group is selected from morpholinyl, —OCH₃, piperidinyl, methylamino, pyrrolidinyl, cyclopropylamino, difluoropyrrolidinyl, or fluoroethylamino.

In certain embodiments, R⁵ is —R² as defined in classes and subclasses herein.

Exemplary R⁵ groups include those set forth in Examples 1-357, inclusive, in the Examples section, infra.

In some embodiments, Ring A is a monocyclic aromatic ring. In certain embodiments, Ring A is a phenyl ring. In other embodiments, Ring A is a pyridyl, pyrimidinyl, piperazinyl, pyridazinyl, or triazinyl ring. In yet other embodiments, Ring A is a pyrrolyl, pyrazolyl, imidazolyl, triazolyl, tetrazolyl, thienyl, furyl, thiazolyl, isothiazolyl, thiadiazolyl, oxazolyl, isoxazolyl, or oxadiaziolyl ring.

In one aspect, Ring A is

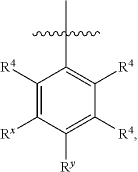

and at least one of R^{x}, R^{y}, and R⁴ is —OH, —OCH₃, or —NH₂.

A person of ordinary skill in the art will appreciate that when R^{x}, R^{y}, or R⁴ is oxo, it means that R^{x}, R^{y}, or R⁴ is a divalent ═O moiety, such that Ring A retains its aromaticity. Exemplary Ring A moieties in which one of R^{x}, R^{y}, or R⁴ is oxo include pyridone, pyrimidone, pyrazinone, imidazolone, oxazolidone, isoxazolidone, thiazolidone, pyrrolidone, and pyrazolone.

In some embodiments, Ring A is a bicyclic aromatic ring. In certain embodiments, Ring A is a quinolinyl, quinoxalinyl, quinazolinyl, pyridopyrazinyl, or pyridopyrimidinyl ring. In certain other embodiments, Ring A is an indolyl, benzimidazolyl, benzothiazolyl, benzofuranyl, benzotriazolyl, benzoxazolyl, benzothienyl, indazolyl, imidazopyridyl, imidazopyrimidinyl, imidazopyrazinyl, imidazopyridazinyl, pyrazolopyridyl, pyrazolopyrimidinyl, pyrazolopyrazinyl, pyrazolopyridazinyl, pyrrolothiazolyl, imidazothiazolyl, thiazolopyridyl, thiazolopyrimidinyl, thiazolopyrazinyl, thiazolopyrimidinyl, oxazolopyridyl, oxazolopyrimidinyl, oxazolopyrazinyl, or oxazolopyridazinyl ring.

In some embodiments, Ring A is a bicyclic ring comprising a partially unsaturated ring fused to an aromatic ring as described herein.

Exemplary Ring A groups are set forth in Table 1.

TABLE 1
Ring A Groups

| | |
|---|---|
| 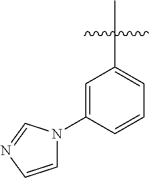 | i |
| 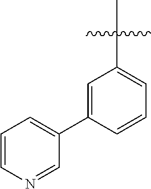 | ii |
| 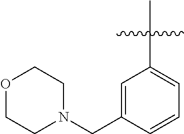 | iii |
| 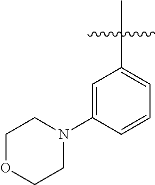 | iv |
| 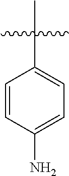 | v |
| 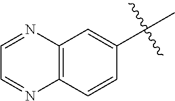 | vi |
| 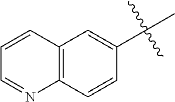 | vii |
| 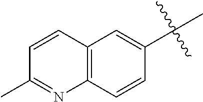 | viii |
| 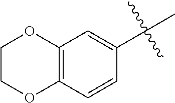 | ix |
| 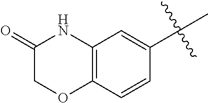 | x |
| 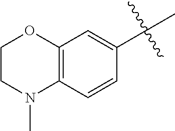 | xi |
| 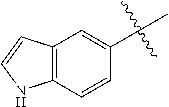 | xii |
| 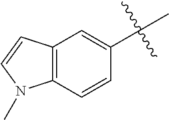 | xiii |
| 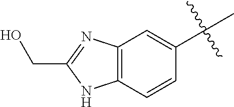 | xiv |
| 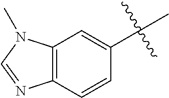 | xv |
| 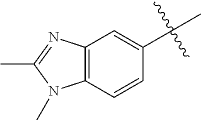 | xvi |
| 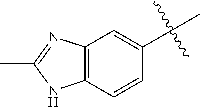 | xvii |
| 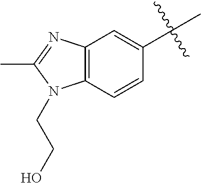 | xviii |
| 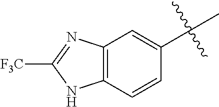 | xix |
| 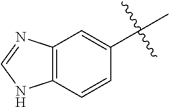 | xx |
| 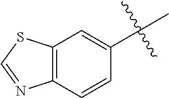 | xxi |
| 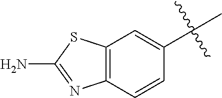 | xxii |
| 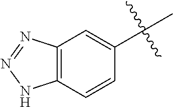 | xxiii |
| 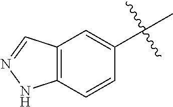 | xxiv |
| 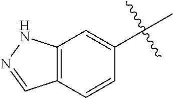 | xxv |
| 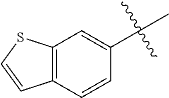 | xxvi |
| 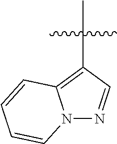 | xxvii |
| 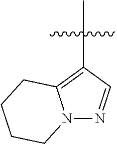 | xxviii |
| 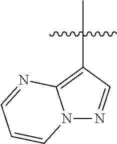 | xxix |
| 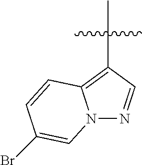 | xxx |
| 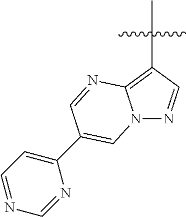 | xxxi |
| 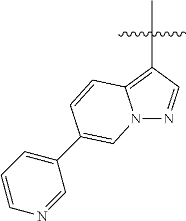 | xxxii |
| 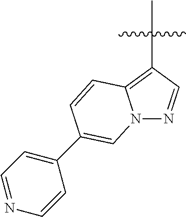 | xxxiii |
| 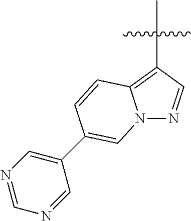 | xxxiv |
| 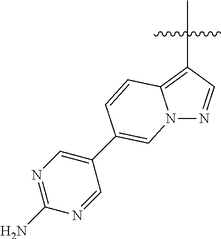 | xxxv |
| 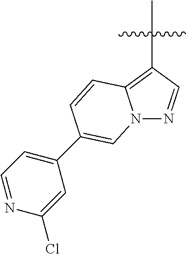 | xxxvi |
| 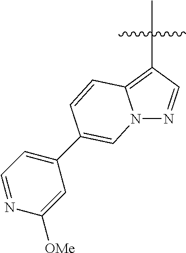 | xxxvii |
| 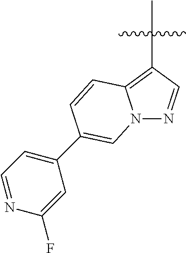 | xxxviii |
| 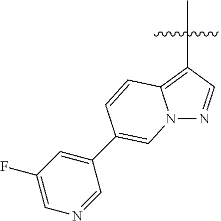 | xxxix |
| 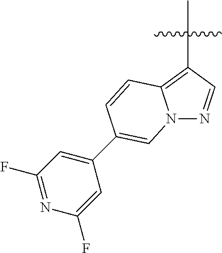 | xl |
| 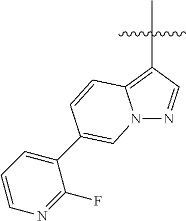 | xli |
| 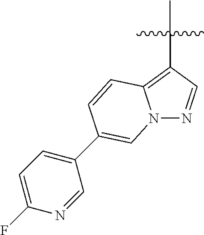 | xlii |
| 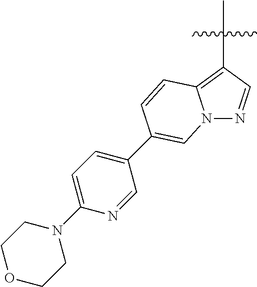 | xliii |
| 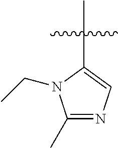 | xliv |
| 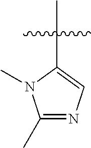 | xlv |
| 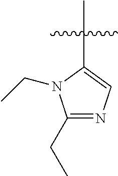 | xlvi |
| 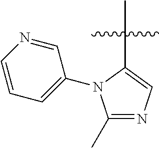 | xlvii |
| 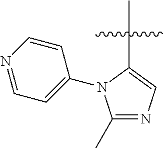 | xlviii |
| 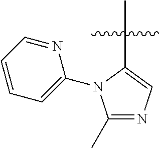 | xlix |
| 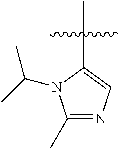 | l |
| 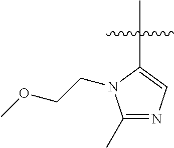 | li |
| 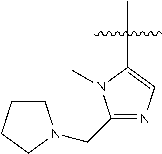 | lii |
| 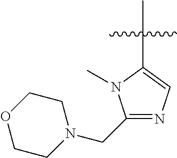 | liii |
| 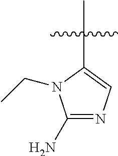 | liv |
| 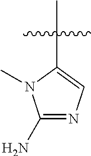 | lv |
| 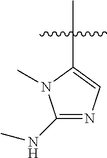 | lvi |
| 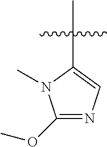 | lvii |
| 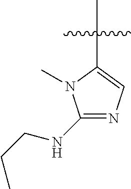 | lviii |
| 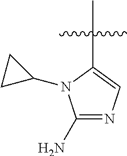 | lix |
| 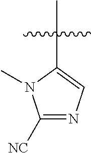 | lx |
| 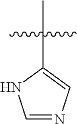 | lxi |
| 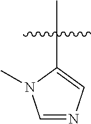 | lxii |
| 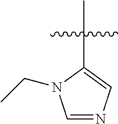 | lxiii |
| 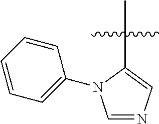 | lxiv |
| 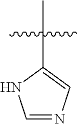 | lxv |
| 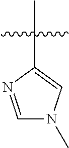 | lxvi |
| 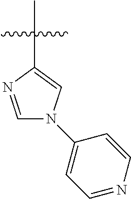 | lxvii |
| 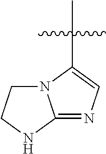 | lxviii |
| 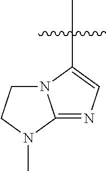 | lxix |
| 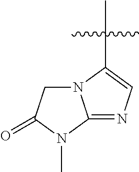 | lxx |
| 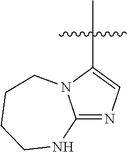 | lxxi |
| 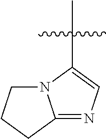 | lxxii |
| 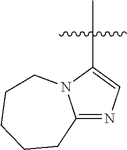 | lxxiii |
| 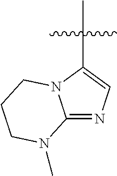 | lxxiv |
| 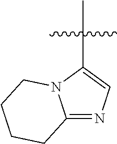 | lxxv |
| 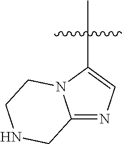 | lxxvi |
| 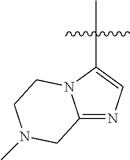 | lxxvii |
| 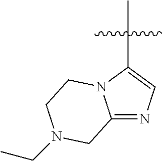 | lxxviii |
| 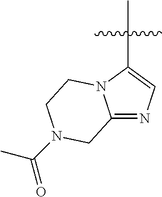 | lxxix |
| 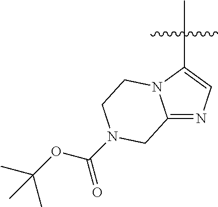 | lxxx |
| 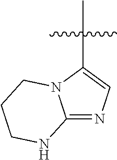 | lxxxi |
| 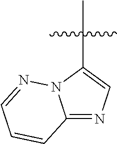 | lxxxii |
| 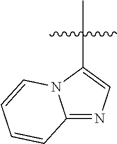 | lxxxiii |
| 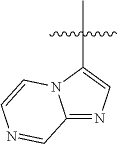 | lxxxiv |
| 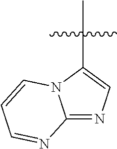 | lxxxv |
| 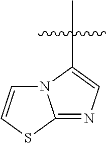 | lxxxvi |
| 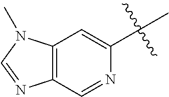 | lxxxvii |
| 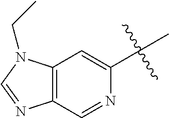 | lxxxviii |
| 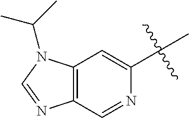 | lxxxix |
| 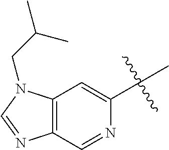 | xc |
| 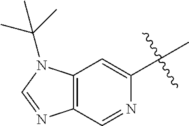 | xci |
| 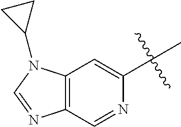 | xcii |
| 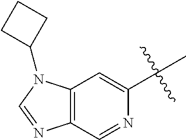 | xciii |
| 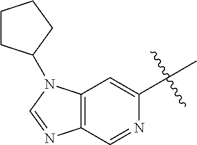 | xciv |
| 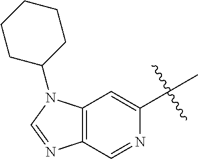 | xcv |
| 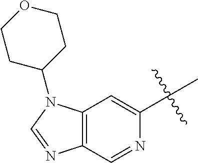 | xcvi |
| 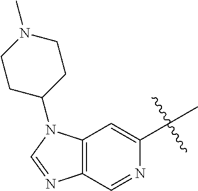 | xcvii |
| 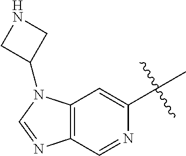 | xcviii |
| 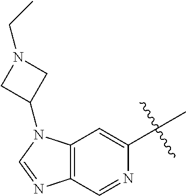 | xcix |
| 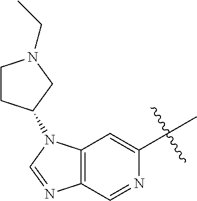 | c |
| 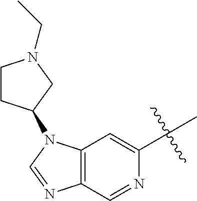 | ci |
| 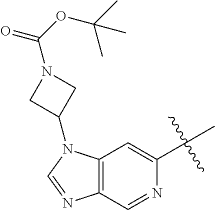 | cii |
| 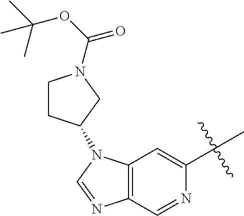 | ciii |
| 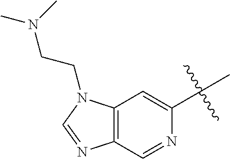 | civ |
| 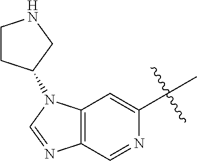 | cv |
| 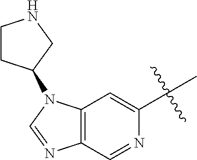 | cvi |
| 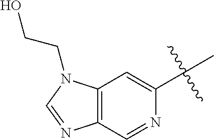 | cvii |
| 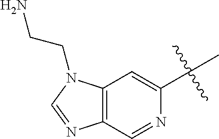 | cviii |
| 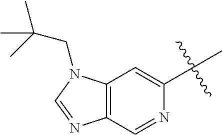 | cix |
| 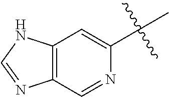 | cx |
| 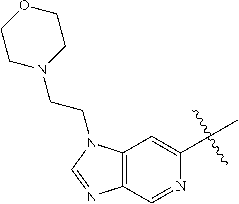 | cxi |
| 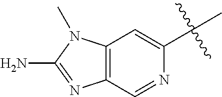 | cxii |
| 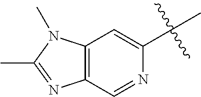 | cxiii |
| 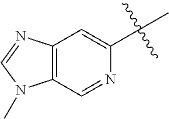 | cxiv |
| 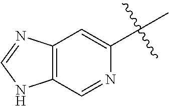 | cxv |
| 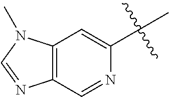 | cxvi |
| 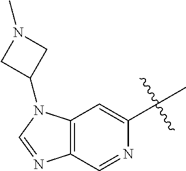 | cxvii |
| 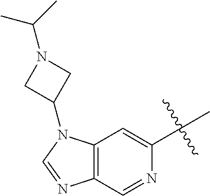 | cxviii |
| 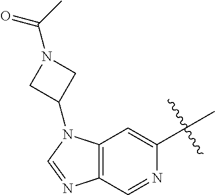 | cxix |
| 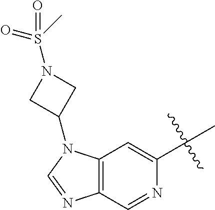 | cxx |
| 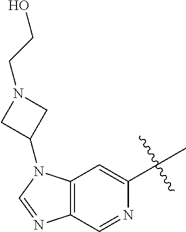 | cxxi |
| 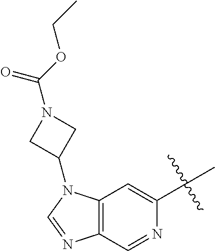 | cxxii |
| 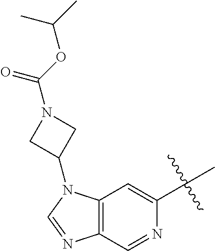 | cxxiii |
| 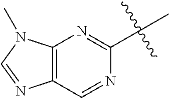 | cxxiv |
| 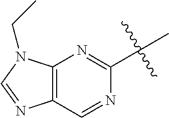 | cxxv |
| 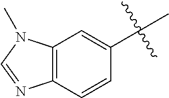 | cxxvi |
| 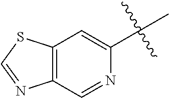 | cxxvii |
| 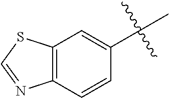 | cxxviii |
| 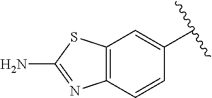 | cxxix |
| 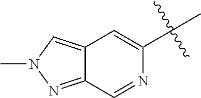 | cxxx |
| 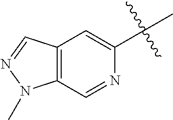 | cxxxi |
| 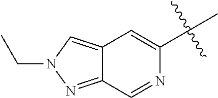 | cxxxii |
| 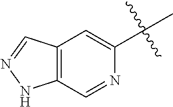 | cxxxiii |
| 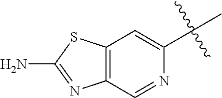 | cxxxiv |
| 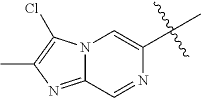 | cxxxv |
| 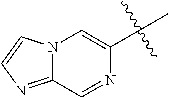 | cxxxvi |
| 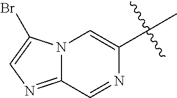 | cxxxvii |
| 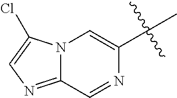 | cxxxviii |
| 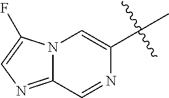 | cxxxix |
| 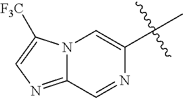 | cxl |
| 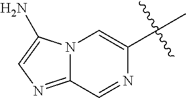 | cxli |
| 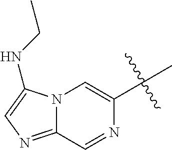 | cxlii |
| 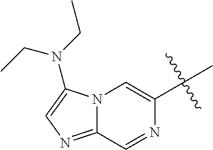 | cxliii |
| 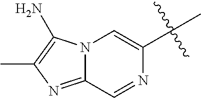 | cxliv |
| 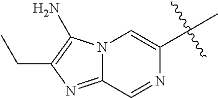 | cxlv |
| 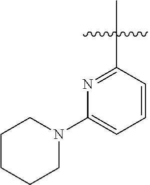 | cxlvi |
| 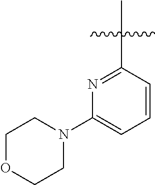 | cxlvii |
| 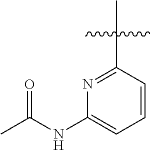 | cxlviii |
| 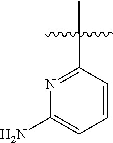 | cxlix |
| 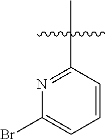 | cl |
| 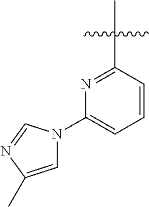 | cli |
| 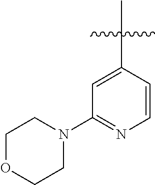 | clii |
| 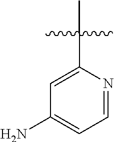 | cliii |
| 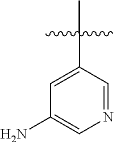 | cliv |
| 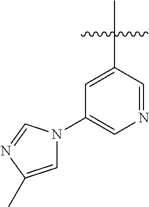 | clv |
| 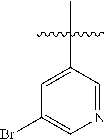 | clvi |
| 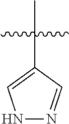 | clvii |
| 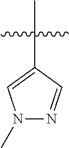 | clviii |
| 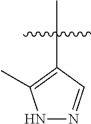 | clix |
| 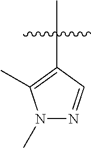 | clx |
| 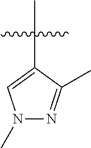 | clxi |
| 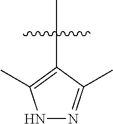 | clxii |
| 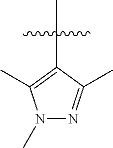 | clxiii |
| 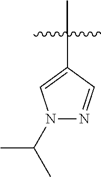 | clxiv |
| 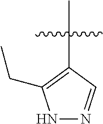 | clxv |
| 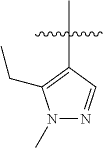 | clxvi |
| 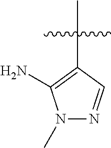 | clxvii |
| 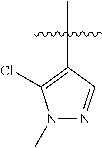 | clxviii |
| 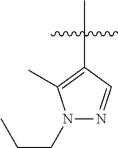 | clxixi |
| 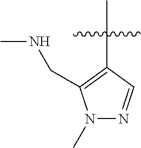 | clxx |
| 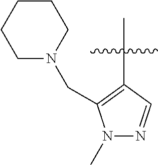 | clxxi |
| 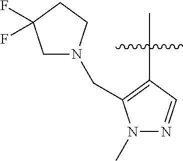 | clxxii |
| 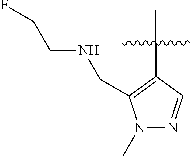 | clxxiii |
| 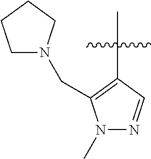 | clxxiv |
| 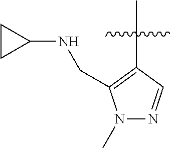 | clxxv |
| 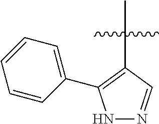 | clxxvi |
| 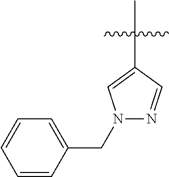 | clxxvii |
| 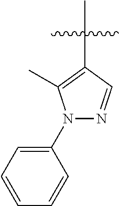 | clxxviii |
| 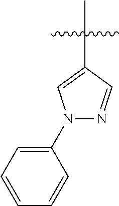 | clxxix |
| 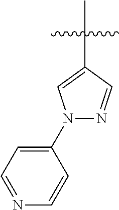 | clxxx |
| 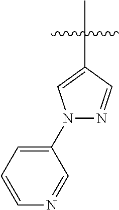 | clxxxi |
| 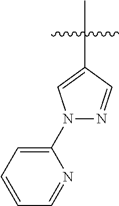 | clxxxii |
| 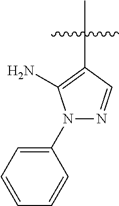 | clxxxiii |
| 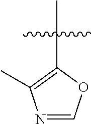 | clxxxiv |
| 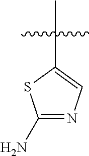 | clxxxv |
| 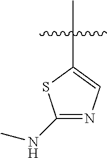 | clxxxvi |
| 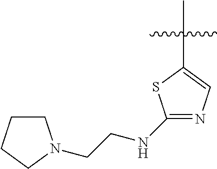 | clxxxvii |
| 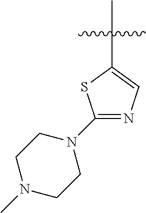 | clxxxviii |
| 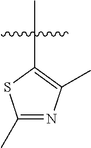 | clxxxix |
| 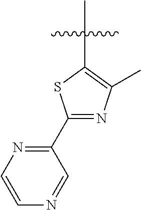 | cxc |
| 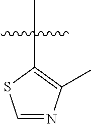 | cxci |
| 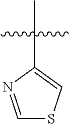 | cxcii |
| 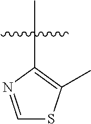 | cxciii |
| 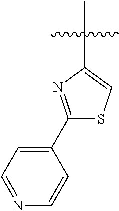 | cxciv |
| 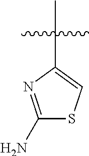 | cxcv |
| 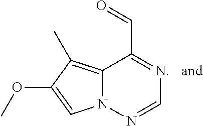 | cxcvi |

In certain embodiments, Ring A is selected from vi, vii, x, xxi, xxii, xxvii, xxviii, xxxii, xxxiii, xxxiv, xxxv, xliii, xliv, xlv, xlvii, xlviii, l, li, liv, lv, lxviii, lxxi, lxxii, lxiii, lxxv, lxxxi, lxxxiii, lxxxiv, lxxxvii, lxxxviii, xc, xciii, xcix, c, cxii, cxvi, cxxv, cxxvii, cxxx, cxxxvii, clx, clxvii, clxviii, and clxxxv.

As defined above, R is hydrogen or an optionally substituted C₁₋₆ aliphatic group. In certain embodiments, R is hydrogen. In other embodiments, R is an optionally substituted C₁₋₆ aliphatic group. In certain embodiments, R is an optionally substituted C₁₋₆ alkyl group. In some embodiments, R is an optionally substituted C₁₋₃ alkyl group. In certain embodiments, R is an optionally substituted methyl or ethyl group. In certain embodiments, R is an optionally substituted methyl group. In certain embodiments, R is methyl.

As defined above, L¹ is an optionally substituted, straight or branched bivalent C₁₋₆ alkylene chain. In certain embodiments, L¹ is an optionally substituted, straight or branched C₁₋₅ alkylene chain. In some embodiments, L¹ is an optionally substituted, straight or branched C₁₋₄ alkylene chain. In other embodiments, L¹ is an optionally substituted, straight or branched C₁₋₃ alkylene chain. According to some embodiments, L¹ is an optionally substituted, straight or branched C₁₋₂ alkylene chain.

In certain embodiments, L¹ is an optionally substituted C₁ alkylene chain. In some embodiments, L¹ is an optionally substituted, straight or branched C₂ alkylene chain. In other embodiments, L¹ is an optionally substituted, straight or branched C₃ alkylene chain. According to some embodiments, L¹ is an optionally substituted, straight or branched C₄ alkylene chain. In certain aspects, L¹ is an optionally substituted, straight or branched C₅ alkylene chain. In other aspects, L¹ is an optionally substituted, straight or branched C₆ alkylene chain.

In certain embodiments, L¹ is an optionally substituted, straight C₁₋₆ alkylene chain. In some embodiments, L¹ is a straight C₁₋₆ alkylene chain. In other embodiments, L¹ is an optionally substituted, branched C₁₋₆ alkylene chain. In certain aspects, L¹ is a branched C₁₋₆ alkylene chain. In certain embodiments, L¹ is —CH(C₁₋₆alkyl)—, —CH(C₁₋₅alkyl)—, —CH(C₁₋₄alkyl)—, —CH(C₁₋₃alkyl)—, or —CH(C₁₋₂alkyl)—. In certain embodiments, L¹ is —CH(CH₃)—.

As defined generally above, Cy¹ is phenylene, 5-6 membered saturated or partially unsaturated carbocyclylene, a 7-10 membered saturated or partially unsaturated bicyclic carbocyclylene, a 5-6 membered saturated or partially unsaturated heterocyclylene having 1-2 heteroatoms independently selected from nitrogen, oxygen, and sulfur, a 7-10 membered saturated or partially unsaturated bicyclic heterocyclylene having 1-3 heteroatoms independently selected from nitrogen, oxygen, and sulfur, 8-10 membered bicyclic arylene, a 5-6 membered heteroarylene having 1-3 heteroatoms independently selected from nitrogen, oxygen, and sulfur, or an 8-10 membered bicyclic heteroarylene having 1-4 heteroatoms independently selected from nitrogen, oxygen, and sulfur, wherein Cy¹ is optionally substituted with one or two groups independently selected from halogen, —R^{c}, —CN, —NO₂, —OR^{c}, —N(R^{c})₂, and —SR^{C}, wherein each R^{c} is independently hydrogen or a C₁₋₂ alkyl group, wherein R^{c} is optionally substituted with 1-3 groups independently selected from halogen, —OH, —NH₂, —SH, and —CN.

In some embodiments, Cy¹ is optionally substituted 5-membered saturated carbocyclylene. In other embodiments, Cy¹ is optionally substituted 6-membered saturated carbocyclylene. In certain embodiments, Cy¹ is optionally substituted 5-membered partially unsaturated carbocyclylene. In certain other embodiments, Cy¹ is optionally substituted 6-membered partially unsaturated carbocyclylene. In some embodiments, Cy¹ is optionally substituted 7-10 membered bicyclic carbocyclylene. In other embodiments, Cy¹ is an optionally substituted 7-10 membered bicyclic heterocyclylene having 1-3 heteroatoms independently selected from nitrogen, oxygen, and sulfur.

In some embodiments, Cy¹ is optionally substituted phenylene. In other embodiments, Cy¹ is optionally substituted 8-10 membered bicyclic arylene. In certain embodiments, Cy¹ is optionally substituted naphthylene. In certain embodiments, Cy¹ is an optionally substituted 6-membered saturated or partially unsaturated heterocyclylene having 1-2 heteroatoms independently selected from nitrogen, oxygen, and sulfur. In certain embodiments, Cy¹ is an optionally substituted 6-membered heteroarylene having 1-3 heteroatoms independently selected from nitrogen, oxygen, and sulfur. In other embodiments, Cy¹ is an optionally substituted 6-membered heteroarylene having 1 nitrogen. In certain other embodiments, Cy¹ is an optionally substituted 6-membered heteroarylene having 2 nitrogens. In yet other embodiments, Cy¹ is an optionally substituted 6-membered heteroarylene having 3 nitrogens. In other embodiments, Cy¹ is an optionally substituted 5-membered saturated or partially unsaturated heterocyclylene having 1-2 heteroatoms independently selected from nitrogen, oxygen, and sulfur. In certain embodiments, Cy¹ is an optionally substituted 5-membered heteroarylene having 1-3 heteroatoms independently selected from nitrogen, oxygen, and sulfur. In certain embodiments, Cy¹ is an optionally substituted 5-membered heteroarylene having 1 heteroatom independently selected from nitrogen, oxygen, and sulfur. In certain embodiments, Cy¹ is an optionally substituted 5-membered heteroarylene having 2 heteroatoms independently selected from nitrogen, oxygen, and sulfur. In other embodiments, Cy¹ is an optionally substituted 5-membered heteroarylene having 2 heteroatoms independently selected from nitrogen and oxygen. In some embodiments, Cy¹ is an optionally substituted 5-membered heteroarylene having 2 heteroatoms independently selected from nitrogen and sulfur. In some embodiments, Cy¹ is an optionally substituted 8-10 membered bicyclic heteroarylene having 1-4 heteroatoms independently selected from nitrogen, oxygen, and sulfur. In other embodiments, Cy¹ is an optionally substituted 10-membered bicyclic heteroarylene having 1-3 nitrogens. In certain embodiments, Cy¹ is an optionally substituted 10-membered bicyclic heteroarylene having one nitrogen.

Exemplary Cy¹ groups include optionally substituted phenylene, naphthylene, pyridylene, pyrimidinylene, pyrazinylene, pyridazinylene, triazinylene, pyrrolylene, pyrazolylene, imidazolylene, triazolylene, tetrazolylene, thienylene, furylene, thiazolylene, isothiazolylene, thiadiazolylene, oxazolylene, isoxazolylene, oxadiaziolylene, quinolinylene, quinazolinylene, and quinoxalinylene. In certain embodiments, Cy¹ is optionally substituted phenylene. In some embodiments, Cy¹ is unsubstituted phenylene. In certain embodiments, Cy¹ is optionally substituted quinolinylene. In certain embodiments, Cy¹ is optionally substituted thiazolylene, isoxazolylene, or thienylene. In other embodiments, Cy¹ is optionally substituted thiazolylene. In some embodiments, Cy¹ is unsubstituted thiazolylene. In certain embodiments, Cy¹ is optionally substituted pyrazinylene, pyrimidinylene, or pyridylene. In certain embodiments, Cy¹ is unsubstituted pyrazinyl.

As defined generally above, L² is —NR¹— or —C(O)NR¹—, wherein R¹ is hydrogen or an optionally substituted C₁₋₆ aliphatic group. In some embodiments, L² is —NR¹—. In certain embodiments, L² is —NH—. In other embodiments, L² is —C(O)NR¹—. In certain other embodiments, L² is —C(O)NH—.

As defined above, R¹ is hydrogen or an optionally substituted C₁₋₆ aliphatic group. In certain embodiments, R¹ is hydrogen. In other embodiments, R¹ is optionally substituted C₁₋₆ aliphatic. In certain embodiments, R¹ is optionally substituted C₁₋₆ alkyl. In some embodiments, R¹ is optionally substituted C₁₋₃ alkyl. In certain aspects, R¹ is optionally substituted methyl or ethyl. In certain embodiments, R¹ is optionally substituted methyl. In certain embodiments, R¹ is methyl.

As defined generally above, Cy² is an optionally substituted group selected from phenyl, a 5-8 membered saturated or partially unsaturated carbocyclic ring, a 7-10 membered saturated or partially unsaturated bicyclic carbocyclic ring, a 5-8 membered saturated or partially unsaturated heterocyclic ring having 1-2 heteroatoms independently selected from nitrogen, oxygen, and sulfur, a 7-10 membered saturated or partially unsaturated bicyclic heterocyclic ring having 1-3 heteroatoms independently selected from nitrogen, oxygen, and sulfur, an 8-10 membered bicyclic aryl ring, a 5-6 membered heteroaryl ring having 1-3 heteroatoms independently selected from nitrogen, oxygen, and sulfur, or an 8-10 membered bicyclic heteroaryl ring having 1-4 heteroatoms independently selected from nitrogen, oxygen, and sulfur.

In some embodiments, Cy² is an optionally substituted 5-8 membered saturated or partially unsaturated carbocyclic ring. In certain embodiments, Cy² is an optionally substituted 7-10 membered saturated or partially unsaturated bicyclic carbocyclic ring. In other embodiments, Cy² is an optionally substituted 5-8 membered saturated or partially unsaturated heterocyclic ring having 1-2 heteroatoms independently selected from nitrogen, oxygen, and sulfur. In certain embodiments, Cy² is optionally substituted phenyl. In other embodiments, Cy^{2 is an optionally substituted} 5-6 membered heteroaryl ring having 1-3 heteroatoms independently selected from nitrogen, oxygen, and sulfur. In some embodiments, Cy² is an optionally substituted 8-10 membered bicyclic aryl ring. In other embodiments, Cy² is an optionally substituted 8-10 membered bicyclic heteroaryl ring having 1-4 heteroatoms independently selected from nitrogen, oxygen, and sulfur.

In certain embodiments, Cy² is an optionally substituted 5-membered saturated or partially unsaturated heterocyclic ring having 1-2 heteroatoms independently selected from nitrogen, oxygen, and sulfur. In certain embodiments, Cy² is an optionally substituted 5-membered heteroaryl ring having 1-3 heteroatoms independently selected from nitrogen, oxygen, and sulfur. In some embodiments, Cy² is an optionally substituted 5-membered heteroaryl ring having 1-2 heteroatoms independently selected from nitrogen, oxygen, or sulfur. In other embodiments, Cy² is an optionally substituted 5-membered heteroaryl ring having 1-2 nitrogens. Exemplary Cy² groups include optionally substituted pyrrolyl, pyrazolyl, imidazolyl, triazolyl, tetrazolyl, thienyl, furyl, thiazolyl, isothiazolyl, thiadiazolyl, oxazolyl, isoxazolyl, and oxadiaziolyl.

In some embodiments, Cy² is an optionally substituted 6-membered saturated or partially unsaturated heterocyclic ring having 1-2 heteroatoms independently selected from nitrogen, oxygen, and sulfur. In other embodiments, Cy² is an optionally substituted 6-membered heteroaryl ring having 1-3 heteroatoms independently selected from nitrogen, oxygen, and sulfur. In some embodiments, Cy² is an optionally substituted 6-membered heteroaryl ring having 1-2 heteratoms independently selected from nitrogen, oxygen, and sulfur. In other embodiments, Cy² is an optionally substituted 6-membered heteroaryl ring having 1-3 nitrogens. In some embodiments, Cy² is an optionally substituted 6-membered heteroaryl ring having 1-2 nitrogens. In certain embodiments, Cy² is optionally substituted pyridyl, pyrimidinyl, pyrazinyl, pyridazinyl, triazinyl, or tetrazinyl. In some embodiments, Cy² is optionally substituted pyridyl, pyrimidinyl or pyridazinyl.

In certain embodiments, Cy² is an optionally substituted 7-10 membered saturated or partially unsaturated bicyclic heterocyclic ring having 1-3 heteroatoms independently selected from nitrogen, oxygen, and sulfur. In certain embodiments, Cy² is an optionally substituted 8-10 membered bicyclic heteroaryl ring having 1-4 heteroatoms independently selected from nitrogen, oxygen, and sulfur. In some embodiments, Cy² is an optionally substituted 5,5-fused, 5,6-fused, or 6,6-fused saturated or partially unsaturated bicyclic heterocyclic ring having 1-3 heteroatoms independently selected from nitrogen, oxygen, and sulfur. In other embodiments, Cy² is an optionally substituted 5,5-fused, 5,6-fused, or 6,6-fused heteroaryl ring having 1-4 heteroatoms independently selected from nitrogen, oxygen, and sulfur. In certain embodiments, Cy² is an optionally substituted 5,5-fused, 5,6-fused, or 6,6-fused heteroaryl ring having 1-4 nitrogens. In other embodiments, Cy² is an optionally substituted 5,6-fused heteroaryl ring having 1-4 nitrogens. In certain embodiments, Cy² is optionally substituted pyyrolizinyl, indolyl, quinolinyl, isoquinolinyl, benzimidazolyl, imidazopyridyl, indazolyl, purinyl, cinnolinyl, quinazolinyl, phthalazinyl, naphthridinyl, quinoxalinyl, thianaphtheneyl, or benzofuranyl. In certain embodiments, Cy² is optionally substituted benzimidazolyl, imidazopyridyl or purinyl.

In some embodiments, Cy² is an optionally substituted 5-8 membered saturated or partially unsaturated carbocyclic ring. In certain embodiments, Cy² is optionally substituted phenyl. In other embodiments, Cy² is an optionally substituted 5-6 membered saturated or partially unsaturated carbocyclic ring. In certain embodiments, Cy² is an optionally substituted 5-membered saturated or partially unsaturated carbocyclic ring. In certain embodiments, Cy² is an optionally substituted 6-membered saturated or partially unsaturated carbocyclic ring.

In certain embodiments, Cy² is an optionally substituted 8-10 membered saturated, partially unsaturated, or aromatic monocyclic or bicyclic carbocyclic ring. In certain embodiments, Cy² is an optionally substituted 5,5-fused, 5,6-fused, or 6,6-fused saturated, partially unsaturated, or aromatic bicyclic ring. In some embodiments, Cy² is an optionally substituted 5,5-fused, 5,6-fused, or 6,6-fused aromatic bicyclic ring. In other embodiments, Cy² is optionally substituted naphthalenyl, indanyl or indenyl group.

In certain embodiments, Cy², as described above and herein, is optionally substituted with one or more groups selected from —R^{∘}, halo, —NO₂, —CN, —OR^{∘}, —SR^{∘}, —N(R^{∘})₂, —C(O)R^{∘}, —CO₂R^{∘}, —C(O)C(O)R^{∘}, —C(O)CH₂C(O)R^{∘}, —S(O)R^{∘}, —S(O)₂R^{∘}, —C(O)N(R^{∘})₂, —SO₂N(R^{∘})₂, —OC(O)R^{∘}, —N(R^{∘})C(O)R^{∘}, —N(R^{∘})N(R^{∘})₂, —C═NN(R^{∘})₂, —C═NOR^{∘}, —N(R^{∘})C(O)N(R)₂, —N(R^{∘})SO₂N(R^{∘})₂, —N(R^{∘})SO₂R^{∘}, or —OC(O)N(R^{∘})₂; wherein R^{∘} is as defined above and described herein. In other embodiments, Cy² is optionally substituted with C₁₋₆ aliphatic or halogen. In some embodiments, Cy² is optionally substituted with —Cl, —F, —CF₃, or —C₁₋₄ alkyl. In certain embodiments, Cy² is optionally substituted with —CF₃. Exemplary substituents on Cy² include methyl, tert-butyl, 1-methylcyclopropyl, and trifluoromethyl. Other exemplary substituents on Cy² include hydrogen, fluoro, bromo, chloro, —OCH₃, —N(CH₃)₂, —OCH₂CH₃, —CH₂OH, —OCH₂CH₂OCH₃, —OCF₃, oxetanyl, —C(CF₃)(CH₃)₂, —C(CN)(CH₃)₂, —CO₂H, —CONH₂, —CONHCH₃, —CN, —SO₂CF₃, —NH₂, —NHCH₃,

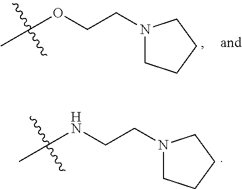

In other embodiments, Cy² is mono- or di-substituted. In certain embodiments, Cy² is optionally substituted at the meta or the para position with any one of the above-mentioned substituents.

Exemplary Cy² groups are shown in Table 2.

TABLE 2
Cy² Groups

| | |
|---|---|
| 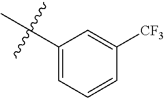 | i |
| 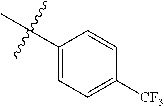 | ii |
| 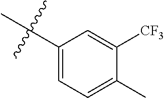 | iii |
| 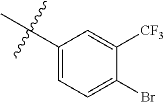 | iv |
| 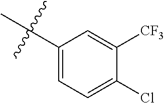 | v |
| 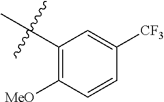 | vi |
| 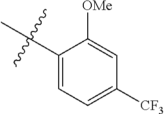 | vii |
| 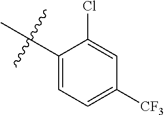 | viii |
| 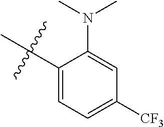 | ix |
| 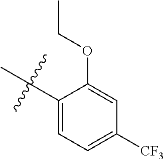 | x |
| 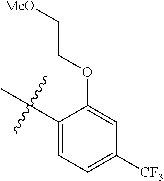 | xi |
| 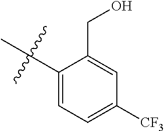 | xii |
| 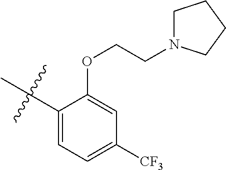 | xiii |
| 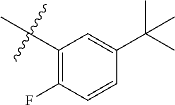 | xiv |
| 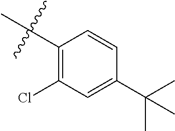 | xv |
| 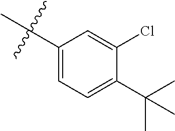 | xvi |
| 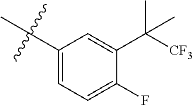 | xvii |
| 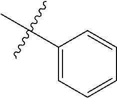 | xviii |
| 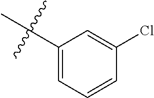 | xix |
| 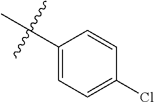 | xx |
| 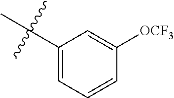 | xxi |
| 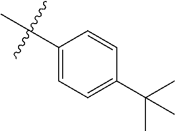 | xxii |
| 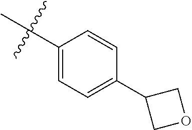 | xxiii |
| 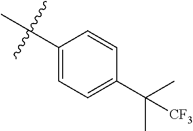 | xxiv |
| 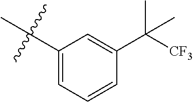 | xxv |
| 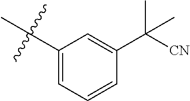 | xxvi |
| 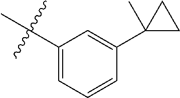 | xxvii |
| 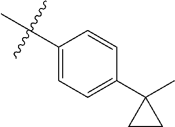 | xxviii |
| 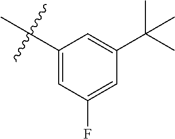 | xxix |
| 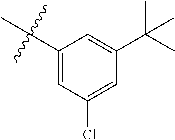 | xxx |
| 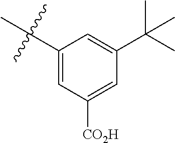 | xxxi |
| 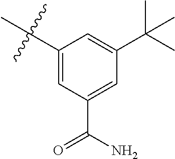 | xxxii |
| 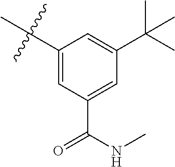 | xxxiii |
| 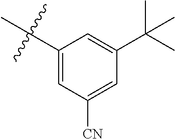 | xxxiv |
| 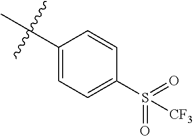 | xxxv |
| 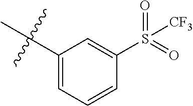 | xxxvi |
| 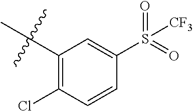 | xxxvii |
| 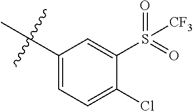 | xxxviii |
| 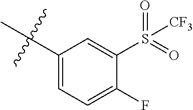 | xxxix |
| 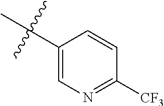 | xl |
| 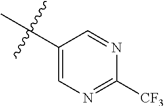 | xli |
| 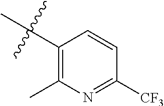 | xlii |
| 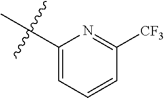 | xliii |
| 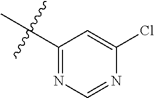 | xliv |
| 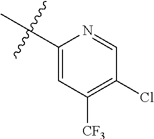 | xlv |
| 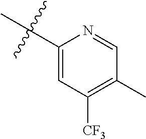 | xlvi |
| 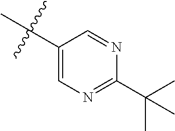 | xlvii |
| 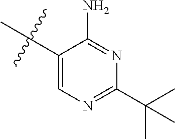 | xlviii |
| 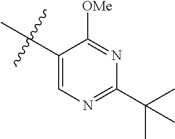 | xlix |
| 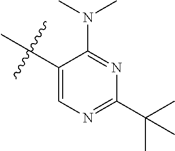 | l |
| 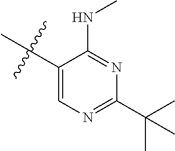 | li |
| 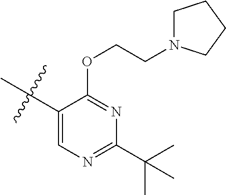 | lii |
| 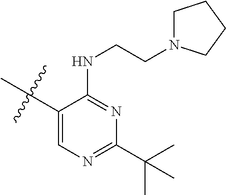 | liii |

According to one aspect, the present invention provides a compound of formula II:

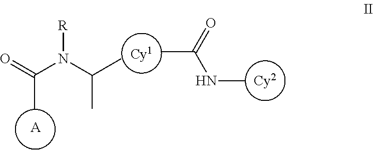

or a pharmaceutically acceptable salt thereof, wherein:
R¹, R^{x}, and R^{y} are as defined above and described herein;
Cy¹ is phenylene or a 5-6 membered heteroarylene having 1-3 heteroatoms independently selected from nitrogen, oxygen, and sulfur, wherein Cy¹ is optionally substituted with 1-2 groups independently selected from halogen, C₁₋₂ alkyl, C₁₋₂ haloalkyl, —CN, —NO₂, —OH, —O(C₁₋₂ alkyl), —NH₂, —NH(C₁₋₂ alkyl), —N(C₁₋₂ alkyl)₂, —SH, and —S(C₁₋₂ alkyl); and
Cy² is optionally substituted phenyl or an optionally substituted 6-membered heteroaryl ring having 1-3 nitrogens.

Another aspect of the present invention provides a compound of one of formulae II-a and II-b:

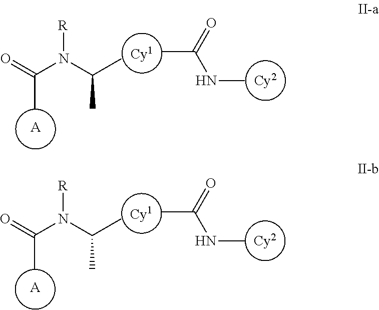

or a pharmaceutically acceptable salt thereof, wherein:
Ring A and R are as defined above and described herein;
Cy¹ is phenylene or a 5-6 membered heteroarylene having 1-3 heteroatoms independently selected from nitrogen, oxygen, and sulfur, wherein Cy¹ is optionally substituted with 1-2 groups independently selected from halogen, C₁₋₂ alkyl, C₁₋₂ haloalkyl, —CN, —NO₂, —OH, —O(C₁₋₂ alkyl), —NH₂, —NH(C₁₋₂ alkyl), —N(C₁₋₂ alkyl)₂, —SH, and —S(C₁₋₂ alkyl); and
Cy² is optionally substituted phenyl or an optionally substituted 6-membered aromatic ring having 1-3 nitrogens.

In certain embodiments, Cy¹ of formula II, II-a, or II-b is a 5-membered heteroarylene having 1-3 heteroatoms independently selected from nitrogen, oxygen, and sulfur. In other embodiments, Cy¹ of formula II, II-a, or II-b is a 6-membered heteroarylene having 1-3 nitrogens. In yet other embodiments, Cy¹ of formula II, II-a, or II-b is phenylene.

In certain embodiments, the present invention provides a compound of one of the following formulae:

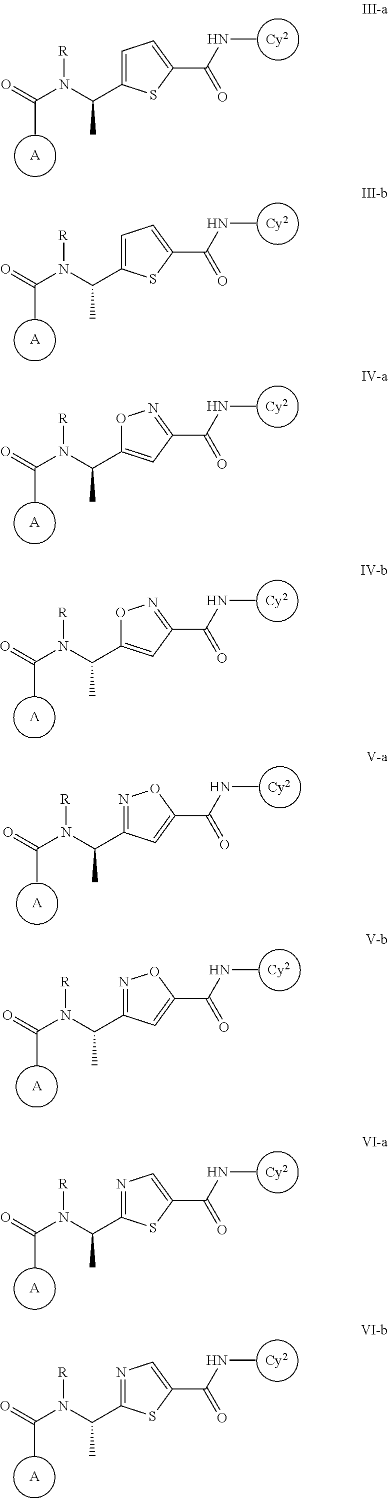

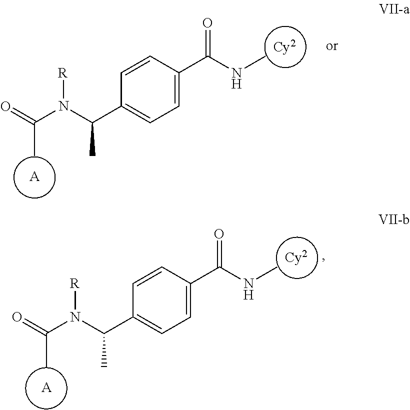

wherein Ring A, R, and Cy² are as defined above and described herein.

Yet another aspect of the present invention provides a compound of formula VIII:

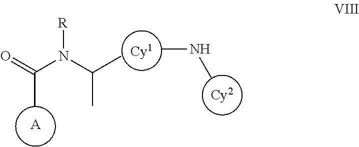

or a pharmaceutically acceptable salt thereof, wherein:
Ring A and R are as defined above and described herein;
Cy¹ is phenylene, a 5-6 membered saturated or partially unsaturated heterocyclylene having 1-2 heteroatoms independently selected from nitrogen, oxygen, and sulfur, or a 5-6 membered heteroarylene having 1-3 heteroatoms independently selected from nitrogen, oxygen, or sulfur, wherein Cy¹ is optionally substituted with 1-2 groups selected from halogen, C₁₋₂ alkyl, C₁₋₂ haloalkyl, —CN, —NO₂, —OH, —O(C₁₋₂ alkyl), —NH₂, —NH(C₁₋₂ alkyl), —N(C₁₋₂ alkyl)₂, —SH, or —S(C₁₋₂ alkyl); and
Cy² is optionally substituted phenyl or an optionally substituted 6-membered heteroaryl ring having 1-3 nitrogens.

In certain embodiments, the present invention provides a compound of one of formulae VIII-a and VIII-b:

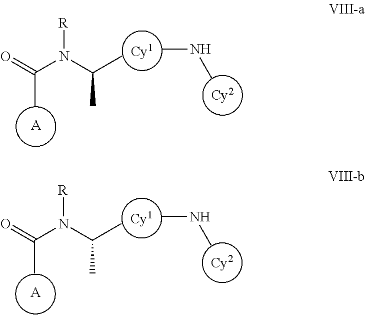

or a pharmaceutically acceptable salt thereof, wherein:
Ring A and R are as defined above and described herein;
Cy¹ is phenylene, a 5-6 membered saturated or partially unsaturated heterocyclylene having 1-2 heteroatoms independently selected from nitrogen, oxygen, and sulfur, or a 5-6 membered heteroarylene having 1-3 heteroatoms independently selected from nitrogen, oxygen, and sulfur, wherein Cy¹ is optionally substituted with 1-2 groups selected from halogen, C₁₋₂ alkyl, C₁₋₂ haloalkyl, —CN, —NO₂, —OH, —O(C₁₋₂ alkyl), —NH₂, —NH(C₁₋₂ alkyl), —N(C₁₋₂ alkyl)₂, —SH, or —S(C₁₋₂ alkyl); and
Cy² is optionally substituted phenyl or an optionally substituted 6-membered heteroaryl ring having 1-3 nitrogens.

In certain embodiments, the present invention provides a compound of formula VIII, VIII-a, or VIII-b wherein Cy¹ is a 5-membered heteroarylene having 1-3 heteroatoms independently selected from nitrogen, oxygen, and sulfur. In certain embodiments, the present invention provides a compound of formula VIII, VIII-a, or VIII-b wherein Cy¹ is thiazolylene.

In certain embodiments, the present invention provides a compound of formula VIII, VIII-a, or VIII-b wherein Cy¹ is a 6-membered heteroarylene having 1-3 nitrogens. In certain embodiments, the present invention provides a compound of formula VIII, VIII-a, or VIII-b

wherein Cy¹ is pyrazinylene.

In another aspect, the present invention provides a compound of formula IX-a or IX-b:

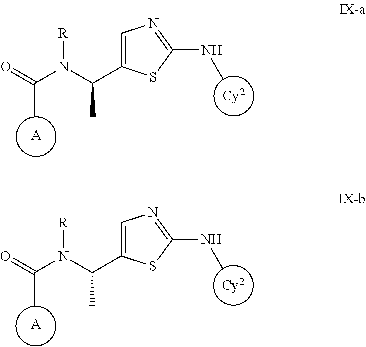

wherein Ring A, R, and Cy² are as defined above and described herein.

In yet another aspect, the present invention provides a compound of formula X-a or X-b:

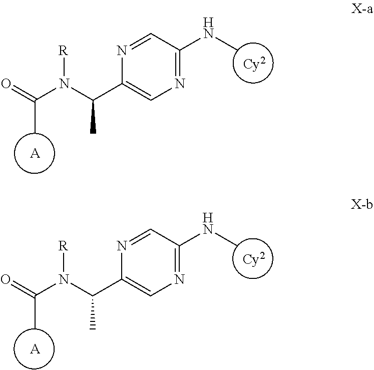

wherein Ring A, R, and Cy² are as defined above and described herein.

In certain embodiments, each of R, Ring A, L¹, L², Cy¹, and Cy² is selected from those groups depicted in the Schemes and in Examples 1-357, inclusive, found in the Examples section, infra.

In some embodiments, the present invention provides any compound shown in Table 3, below.

TABLE 3
Exemplary compounds

| | |
|---|---|
| 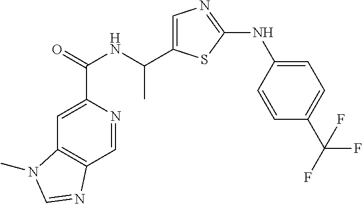 | 2 |
| 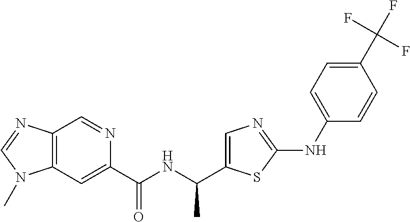 | 4 |
| 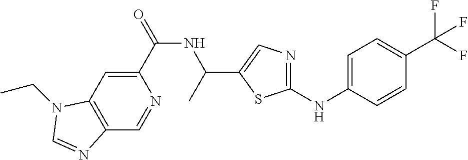 | 6 |
| 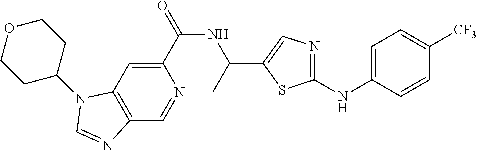 | 9 |
| 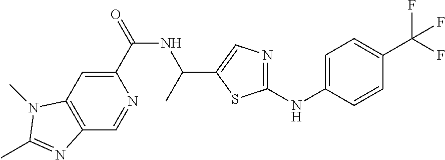 | 10 |
| 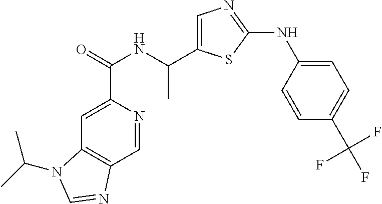 | 12 |
| 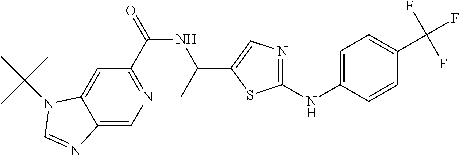 | 13 |
| 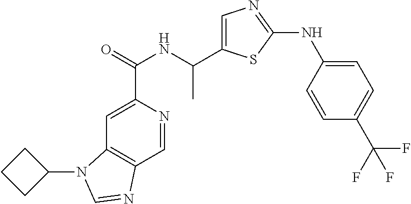 | 14 |
| 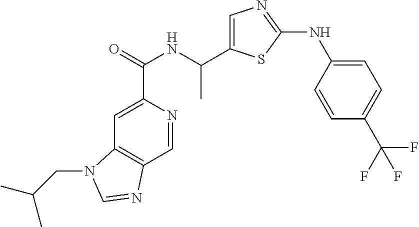 | 15 |
| 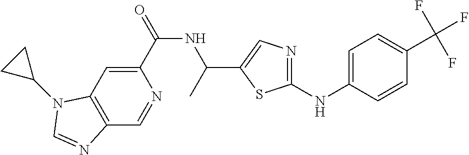 | 19 |
| 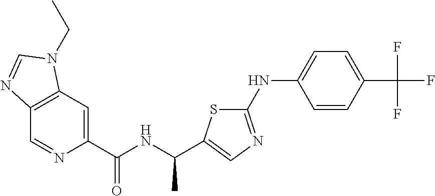 | 20 |
| 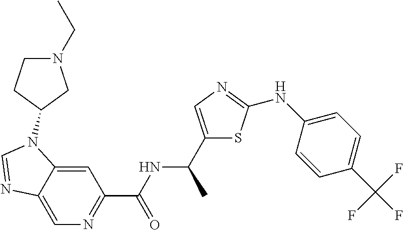 | 28 |
| 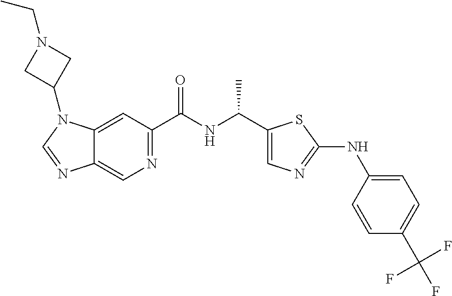 | 30 |
| 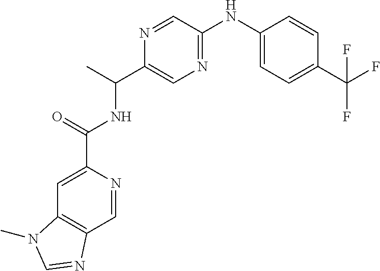 | 35 |
| 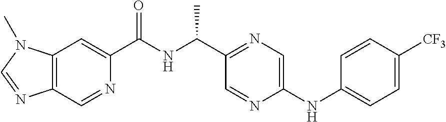 | 37 |
| 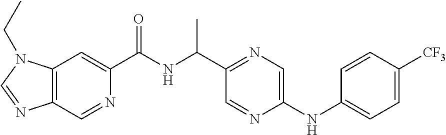 | 38 |
| 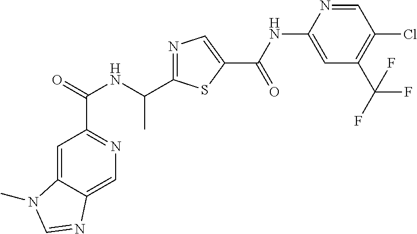 | 40 |
| 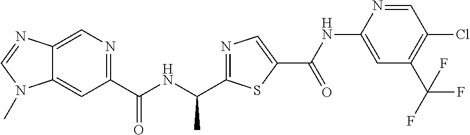 | 42 |
| 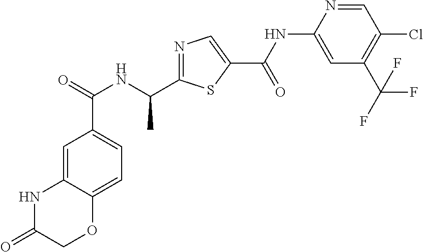 | 66 |
| 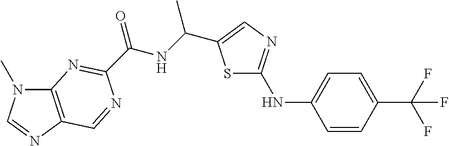 | 190 |
| 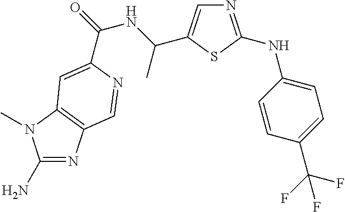 | 199 |
| 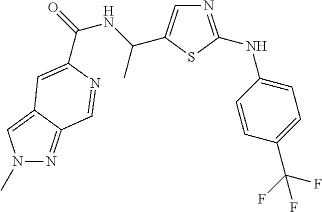 | 203 |
| 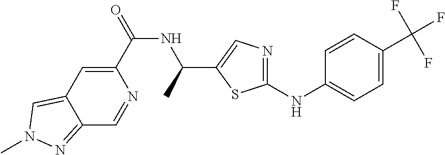 | 205 |
| 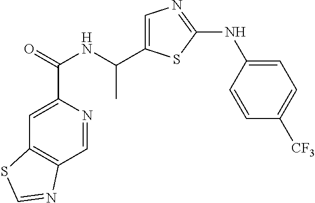 | 208 |
| 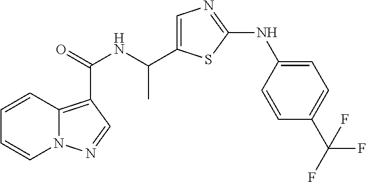 | 224 |
| 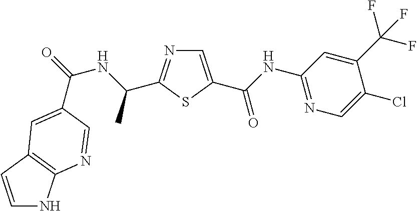 | 81 |
| 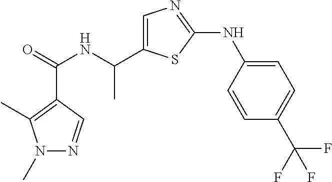 | 82 |
| 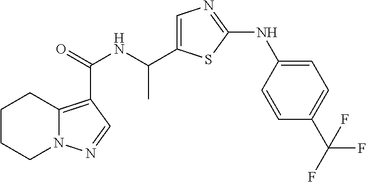 | 86 |
| 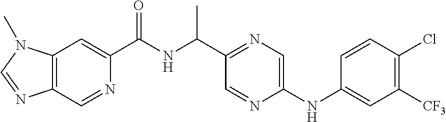 | 134 |
| 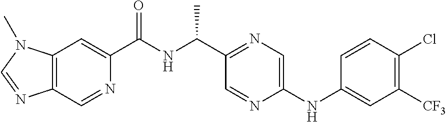 | 236 |
| 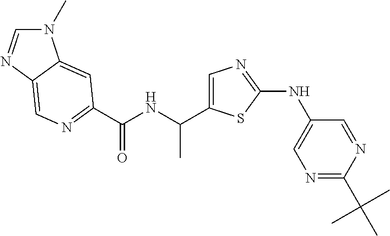 | 240 |
| 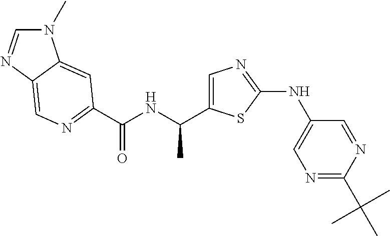 | 241 |
| 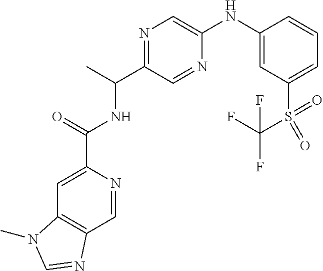 | 243 |
| 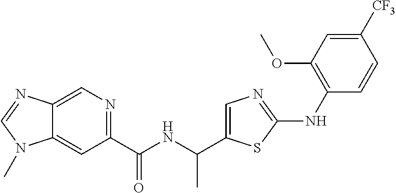 | 244 |
| 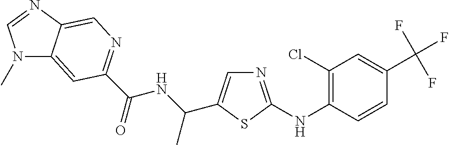 | 245 |
| 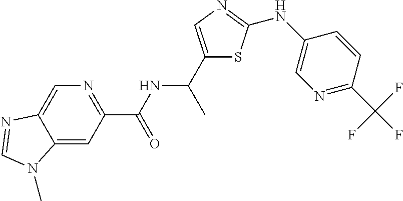 | 246 |
| 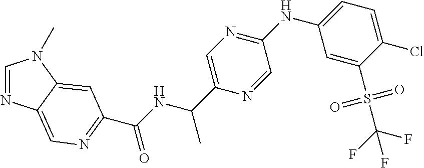 | 269 |
| 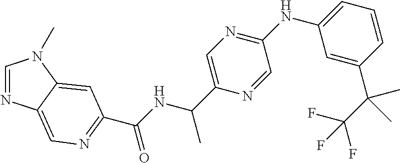 | 273 |
| 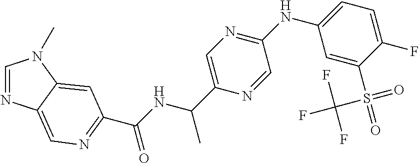 | 268 |
| 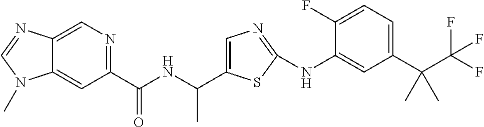 | 274 |
| 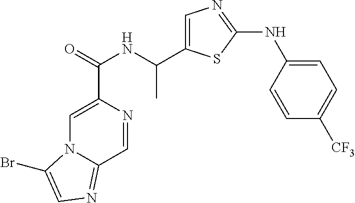 | 297 |
| 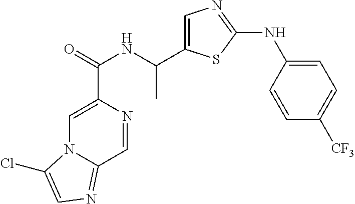 | 299 |
| 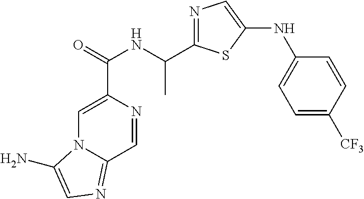 | 302 |
| 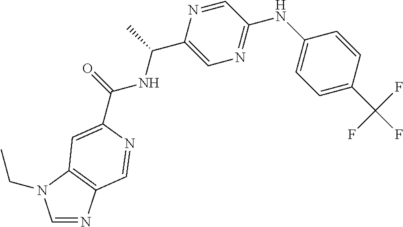 | 174 |
| 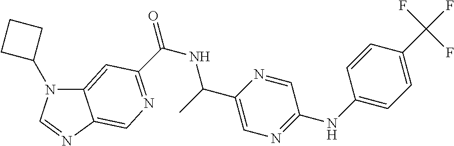 | 175 |
| 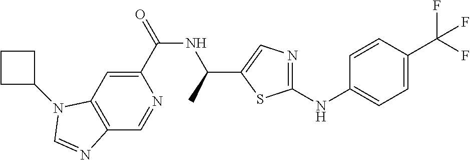 | 176 |
| 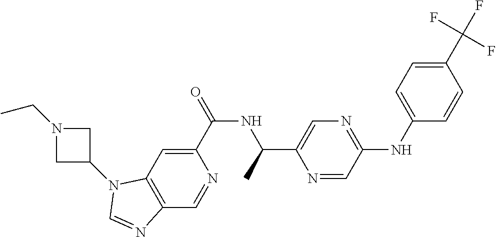 | 180 |
| 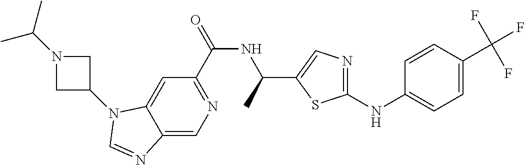 | 183 |
| 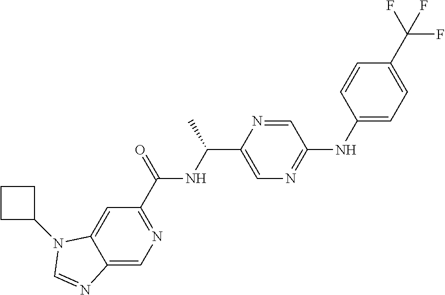 | 188 |
| 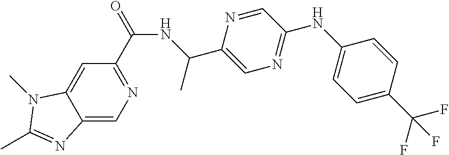 | 201 |
| 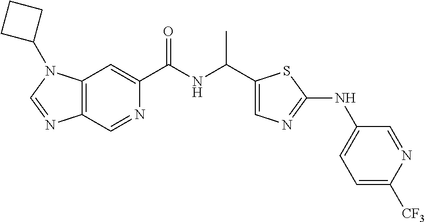 | 292 |
| 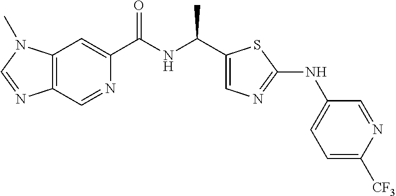 | 267 |
| 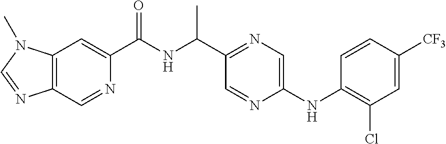 | 265a |
| 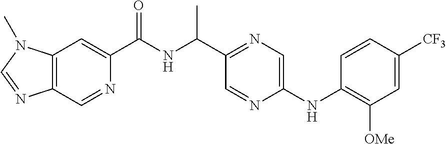 | 265b |
| 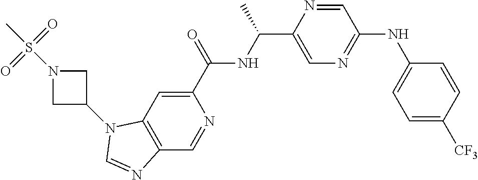 | 345 |
| 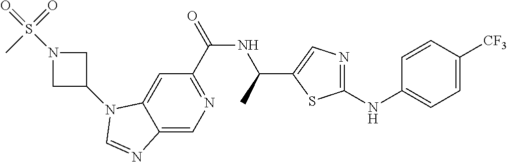 | 346 |
| 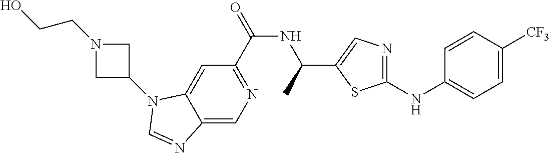 | 348 |
| 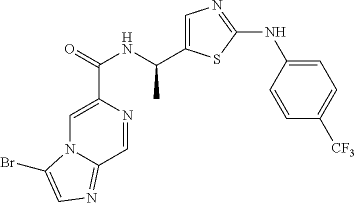 | 298 |
| 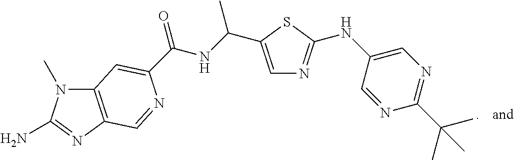 | 287 |

In some embodiments, the present invention provides one of the following compounds shown in Table 2: 2, 4, 6, 9, 12, 13, 14, 15, 19, 20, 28, 30, 35, 37, 38, 40, 42, 199, 203, 205, 208, 224, 232, 236, 240, 241, 243, 244, 245, 269, 274, 297, 268, 274, 297, 174, 176, 180, 183, 188, 201, 292, 267, 265a, 265b, 345, 346, 348, 298, or 287.

### 4. Uses, Formulation and Administration

Pharmaceutically Acceptable Compositions

As discussed above, the present invention provides compounds that are inhibitors of protein kinases (e.g., Raf kinase), and thus the present compounds are useful for the treatment of diseases, disorders, and conditions mediated by Raf kinase. In certain embodiments, the present invention provides a method for treating a Raf-mediated disorder. As used herein, the term “Raf-mediated disorder” includes diseases, disorders, and conditions mediated by Raf kinase. Such Raf-mediated disorders include melanoma, leukemia, or cancers such as colon, breast, gastric, ovarian, lung, brain, larynx, cervical, renal, lymphatic system, genitourinary tract (including bladder and prostate), stomach, bone, lymphoma, melanoma, glioma, papillary thyroid, neuroblastoma, and pancreatic cancer.

Raf-mediated disorders further include diseases afflicting mammals which are characterized by cellular proliferation. Such diseases include, for example, blood vessel proliferative disorders, fibrotic disorders, mesangial cell proliferative disorders, and metabolic diseases. Blood vessel proliferative disorders include, for example, arthritis and restenosis. Fibrotic disorders include, for example, hepatic cirrhosis and atherosclerosis. Mesangial cell proliferative disorders include, for example, glomerulonephritis, diabetic nephropathy, malignant nephrosclerosis, thrombotic microangiopathy syndromes, organ transplant rejection, and glomerulopathies. Metabolic disorders include, for example, psoriasis, diabetes mellitus, chronic wound healing, inflammation, and neurodegenerative diseases.

In another aspect of the present invention, pharmaceutically acceptable compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

It will also be appreciated that certain of the compounds of present invention can exist in free form for treatment, or where appropriate, as a pharmaceutically acceptable derivative thereof. According to the present invention, pharmaceutically acceptable derivatives include, but are not limited to, pharmaceutically acceptable salts, esters, salts of such esters, or any other adducts or derivatives that, upon administration to a patient in need, are capable of providing, directly or indirectly, a compound as otherwise described herein, or a metabolite or residue thereof.

As used herein, the term “pharmaceutically acceptable salt” refers to those salts that are, within the scope of sound medical judgement, suitable for use in contact with the tissues of humans or animals without undue toxicity, irritation, allergic response, or the like, and are offer with a reasonable benefit/risk ratio. A “pharmaceutically acceptable salt” means any at least substantially non-toxic salt or salt of an ester of a compound of this invention that, upon administration to a recipient, is capable of providing, either directly or indirectly, a compound of this invention or an inhibitorily active metabolite or residue thereof. As used herein, the term “inhibitory metabolite or residue thereof” means that a metabolite or residue thereof is also an inhibitor of a Raf kinase.

Pharmaceutically acceptable salts are well known in the art. For example, S. M. Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, use of such a conventional carrier medium is within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Uses of Compounds and Pharmaceutically Acceptable Compositions

According to the present invention, provided compounds may be assayed in any of the available assays known in the art for identifying compounds having kinase inhibitory activity. For example, the assay may be cellular or non-cellular, in vivo or in vitro, high- or low-throughput format, etc.

In certain exemplary embodiments, compounds of this invention were assayed for their ability to inhibit protein kinases, more specifically Raf.

Thus, in one aspect, compounds of this invention which are of particular interest include those which:

are inhibitors of protein kinases;

exhibit the ability to inhibit Raf kinase;

are useful for treating mammals (e.g., humans) or animals suffering from an Raf-mediated disease or condition, and for helping to prevent or delay the onset of such a disease or condition;

exhibit a favorable therapeutic profile (e.g., safety, efficacy, and stability).

In certain embodiments, compounds of the invention are Raf kinase inhibitors. In certain exemplary embodiments, compounds of the invention are Raf inhibitors. In certain exemplary embodiments, compounds of the invention have ^{Cell}IC₅₀ values ≦100 μM. In certain other embodiments, compounds of the invention have ^{Cell}IC₅₀ values ≦75 μM. In certain other embodiments, compounds of the invention have ^{Cell}IC₅₀ values ≦50 μM. In certain other embodiments, compounds of the invention have ^{Cell}IC₅₀ values ≦25 μM. In certain other embodiments, compounds of the invention have ^{Cell}IC₅₀ values ≦10 μM. In certain other embodiments, compounds of the invention have ^{Cell}IC₅₀ values ≦7.5 μM. In certain other embodiments, of the invention compounds have ^{Cell}IC₅₀ values ≦5 μM. In certain other embodiments, of the invention compounds have ^{Cell}IC values ≦2.5 μM. In certain other embodiments, of the invention compounds have ^{Cell}IC₅₀ values ≦1 μM. In certain other embodiments, of the invention compounds have ^{Cell}IC₅₀ values ≦800 nM. In certain other embodiments, of the invention compounds have ^{Cell}IC₅₀ values ≦600 nM. In certain other embodiments, inventive compounds have ^{Cell}IC₅₀ values ≦500 nM. In certain other embodiments, compounds of the invention have ^{Cell}C₅₀ values ≦300 nM. In certain other embodiments, compounds of the invention have ^{Cell}IC₅₀ values ≦200 nM. In certain other embodiments, of the invention compounds have ^{Cell}IC₅₀ values ≦100 nM.

In yet another aspect, a method for the treatment or lessening the severity of an Raf-mediated disease or condition is provided comprising administering an effective amount of a compound, or a pharmaceutically acceptable composition comprising a compound to a subject in need thereof. In certain embodiments of the present invention an “effective amount” of the compound or pharmaceutically acceptable composition is that amount effective for treating or lessening the severity of a Raf-mediated disease or condition. The compounds and compositions, according to the method of the present invention, may be administered using any amount and any route of administration effective for treating or lessening the severity of a Raf-mediated disease or condition. The exact amount required will vary from subject to subject, depending on the species, age, and general condition of the subject, the severity of the infection, the particular agent, its mode of administration, and the like. In certain embodiments, compounds of the invention are formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and eye drops comprising a provided compound are also within the scope of this invention. Additionally, the present invention includes use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As described generally above, the compounds of the invention are useful as inhibitors of protein kinases. In one embodiment, the compounds of the invention are Raf kinase inhibitors, and thus, without wishing to be bound by any particular theory, the compounds and compositions are particularly useful for treating or lessening the severity of a disease, condition, or disorder where activation of Raf kinase is implicated in the disease, condition, or disorder. When activation of Raf kinase is implicated in a particular disease, condition, or disorder, the disease, condition, or disorder may also be referred to as a “Raf-mediated disease”. Accordingly, in another aspect, the present invention provides a method for treating or lessening the severity of a disease, condition, or disorder where activation of Raf kinase is implicated in the disease state.

The activity of a compound utilized in this invention as an Raf kinase inhibitor, may be assayed in vitro, in vivo, ex vivo, or in a cell line. In vitro assays include assays that determine inhibition of either the phosphorylation activity or ATPase activity of activated Raf. Alternate in vitro assays quantitate the ability of the inhibitor to bind to Raf. Inhibitor binding may be measured by radiolabelling the inhibitor (e.g., synthesizing the inhibitor to include a radioisotope) prior to binding, isolating the inhibitor/Raf, complex and determining the amount of radiolabel bound. Alternatively, inhibitor binding may be determined by running a competition experiment where new inhibitors are incubated with Raf bound to known radioligands.

The term “measurably inhibit”, as used herein means a measurable change in Raf activity between a sample comprising said composition and a Raf kinase and an equivalent sample comprising Raf kinase in the absence of said composition.

It will also be appreciated that the compounds and pharmaceutically acceptable compositions of the present invention can be employed in combination therapies, that is, the compounds and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, compound of the invention may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”.

For example, other therapies, chemotherapeutic agents, or other anti-proliferative agents may be combined with the compounds of this invention to treat proliferative diseases and cancer. Examples of therapies or anticancer agents that may be used in combination with the inventive anticancer agents of the present invention include surgery, radiotherapy (e.g., gamma-radiation, neutron beam radiotherapy, electron beam radiotherapy, proton therapy, brachytherapy, and systemic radioactive isotopes), endocrine therapy, biologic response modifiers (e.g., interferons, interleukins, and tumor necrosis factor (TNF), hyperthermia and cryotherapy, agents to attenuate any adverse effects (e.g., antiemetics), and other approved chemotherapeutic drugs.

Examples of chemotherapeutic anticancer agents that may be used as second active agents in combination with compounds of the invention include, but are not limited to, alkylating agents (e.g. mechlorethamine, chlorambucil, cyclophosphamide, melphalan, ifosfamide), antimetabolites (e.g., methotrexate), purine antagonists and pyrimidine antagonists (e.g. 6-mercaptopurine, 5-fluorouracil, cytarabine, gemcitabine), spindle poisons (e.g., vinblastine, vincristine, vinorelbine, paclitaxel), podophyllotoxins (e.g., etoposide, irinotecan, topotecan), antibiotics (e.g., doxorubicin, daunorubicin, bleomycin, mitomycin), nitrosoureas (e.g., carmustine, lomustine), inorganic ions (e.g., platinum complexes such as cisplatin, carboplatin), enzymes (e.g., asparaginase), hormones (e.g., tamoxifen, leuprolide, flutamide, and megestrol), topoisomerase II inhibitors or poisons, EGFR (Herl, ErbB-1) inhibitors (e.g., gefitinib), antibodies (e.g., rituximab), IMIDs (e.g., thalidomide, lenalidomide), various targeted agents (e.g., HDAC inhibitors such as vorinostat , Bcl-2 inhibitors, VEGF inhibitors); proteasome inhibitors (e.g., bortezomib), cyclin-dependent kinase inhibitors, and dexamethasone.

For a more comprehensive discussion of updated cancer therapies see, The Merck Manual, Seventeenth Ed. 1999, the entire contents of which are hereby incorporated by reference. See also the National Cancer Institute (NCI) website (www.nci.nih.gov) and the Food and Drug Administration (FDA) website for a list of the FDA approved oncology drugs (www.fda.gov/cder/cancer/i See Appendix).

Other examples of agents the inhibitors of this invention may also be combined with include, without limitation: treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory agents, including immunosuppressive agents, such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, and anti-Parkinson's agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

Those additional agents may be administered separately from composition containing a compound of the invention, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention or pharmaceutically acceptable compositions thereof may also be incorporated into compositions for coating implantable medical devices, such as prostheses, artificial valves, vascular grafts, stents and catheters. Accordingly, the present invention, in another aspect, includes a composition for coating an implantable device comprising a compound of the present invention as described generally above, and in classes and subclasses herein, and a carrier suitable for coating said implantable device. In still another aspect, the present invention includes an implantable device coated with a composition comprising a compound of the present invention as described generally above, and in classes and subclasses herein, and a carrier suitable for coating said implantable device.

Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

Another aspect of the invention relates to inhibiting Raf activity in a biological sample or a patient, which method comprises administering to the patient, or contacting said biological sample with a compound of the present invention or a composition comprising said compound. The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of Raf kinase activity in a biological sample is useful for a variety of purposes that are known to one of skill in the art. Examples of such purposes include, but are not limited to, blood transfusion, organ-transplantation, biological specimen storage, and biological assays.

### Treatment Kit

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### EXAMPLES

As depicted in the Examples below, in certain exemplary embodiments, compounds are prepared according to the following general procedures. It will be appreciated that, although the synthetic methods and Schemes depict the synthesis of certain compounds of the present invention, the following methods and other methods known to one of ordinary skill in the art can be applied to all compounds and subclasses and species of each of these compounds, as described herein.

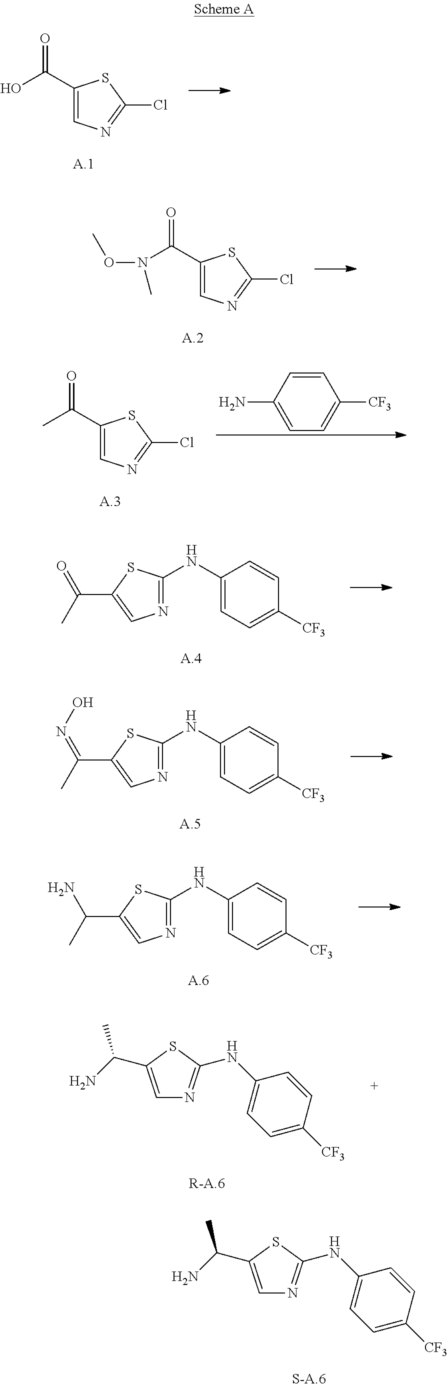

Synthesis of 2-chloro-N-methoxy-N-methylthiazole-5-carboxamide A.2. A 4-neck 5 L round bottom flask equipped with a nitrogen inlet, mechanical stirrer and thermowell was charged with 2-chlorothiazole-5-carboxylic acid A.1 (147 g, 0.9 mol), N,O-dimethylhydroxyamine hydrochloride (104.8 g, 1.08 mol), N-(3-dimethylaminopropyl)-N′-ethylcarbodiimide hydrochloride (189.8 g, 0.99 mol), HOBT (24.3 g, 0.18 mol) and CH₂Cl₂ (2.2 L). To the resulting mixture was slowly added diisopropylethyl amine (376 mL, 2.16 mol). The reaction was stirred at room temperature overnight and water (2 L) was added. The layers were separated and the organic layer was washed with saturated sodium bicarbonate solution (2 L), 1 N HCl (2 L), saturated sodium bicarbonate solution again (2 L) and brine (1 L). The organic layer was dried over sodium sulfate and the solvent was evaporated in vacuo to afford 2-chloro-N-methoxy-N-methylthiazole-5-carboxamide A.2 as a light brown solid (167 g, 90% yield), which was used for the next step without further purification.

Synthesis of 1-(2-chlorothiazol-5-yl)ethanone A.3. A 4-neck 12 L round bottom flask equipped with a nitrogen inlet, mechanical stirrer and thermowell was charged with 2-chloro-N-methoxy-N-methylthiazole-5-carboxamide A.2 (157 g, 0.762 mol) and anhydrous THF (3.14 L). The resulting mixture was cooled to −10° C. by ice/salt bath and methyl magnesium chloride (3 M solution in THF, 305 mol, 0.914 mol) was added dropwise to maintain the temperature below 0° C. After addition, the cooling bath was removed and the reaction mixture was stirred at room temperature overnight. The reaction was quenched by the slow addition of saturated ammonium chloride solution and extracted with MTBE (2×4 L). The organic layers were combined, washed with brine (2 L) and dried over sodium sulfate. The solvent was evaporated in vacuo to afford a crude solid, which was further purified by flash chromatography on silica gel (MTBE/hexanes as elute) to give 1-(2-chlorothiazol-5-yl)ethanone A.3 as a white solid (135 g, 80% yield).

Synthesis of 1-(2-(4-(trifluoromethyl)phenylamino)thiazol-5-yl)ethanone A.4. To a 5 L round bottom flask equipped with a reflux condenser was added 1-(2-chlorothiazol-5-yl)ethanone A.3 (196 g, 1.217 mol), 4-(trifluoromethyl)aniline (152.7 mL, 1.217 mol), 1-butanol (3.9 L) and catalytic amount (48 mL) of HCl in dioxane (4 M). The resulting mixture was heated to reflux for 2 hours and monitored by TLC. After cooling to room temperature, the solvent was evaporated in vacuo and ethyl acetate (4 L) was added to the residue. The organic suspension was washed with saturated sodium bicarbonate solution (2×3 L). The organic layer was dried over sodium sulfate, filtered and evaporated to dryness to afford a brown solid, which was triturated with MTBE/heptane (20%) to give 1-(2-(4-(trifluoromethyl)phenylamino)thiazol-5-yl)ethanone A.4 as yellow solid. The mother liquor was concentrated to dryness and triturated with minimum amount of MTBE to afford a 2nd crop (total 266 g, 76% yield).

Synthesis of 1-(2-(4-(trifluoromethyl)phenylamino)thiazol-5-yl)ethanone oxime A.5. A 4-neck 22 L round bottom flask equipped with a nitrogen inlet, mechanical stirrer and thermowell was charged with 1-(2-(4-(trifluoromethyl)phenylamino)thiazol-5-yl)ethanone A.4 (336 g, 1.17 mol), methanol (6.7 L) and hydroxylamine hydrochloride (161 g, 2.34 mol). The resulting mixture was cooled to 0° C. and pyridine (392 mL, 4.68 mol) was added dropwise. The reaction was stirred at room temperature overnight and the solvent was evaporated in vacuo to afford a brown residue, which was then suspended in water (4 L). The solid was collected by vacuum filtration, washed with water (3×0.5 L) and dried in the vacuum oven at 40° C. overnight to give 1-(2-(4-(trifluoromethyl)phenylamino)thiazol-5-yl)ethanone oxime A.5 as brown solid (339 g, 96% yield).

Synthesis of 5-(1-aminoethyl)-N-(4-(trifluoromethyl)phenyl)thiazol-2-amine A.6. A 4-neck 12 L round bottom flask equipped with a nitrogen inlet, mechanical stirrer and thermowell was charged with 1-(2-(4-(trifluoromethyl)phenylamino)thiazol-5-yl)ethanone oxime A.5 (212 g, 0.702 mol), methanol (3.18 L) and acetic acid (3.18 L). Zinc powder (274 g, 4.212 mol) was added and the resulting mixture was heated to 50° C. for 4 hours. The excess zinc was removed by filtering through Celite and the filter cake was washed with methanol (3×1 L). The filtrate was concentrated to dryness. The residue was suspended in water, basified with aqueous ammonium hydroxide and extracted with ethyl acetate (2×6 L). The organic layers were combined, washed with brine (2 L), dried over sodium sulfate and filtered. The solvent was evaporated in vacuo to afford a crude oil, which was purified by flash chromatography (CH₂Cl₂/methanol as elute) to give 5-(1-aminoethyl)-N-(4-(trifluoromethyl)phenyl)thiazol-2-amine A.6 as a light yellow solid (99 g, 50% yield).

Synthesis of (R)-5-(1-aminoethyl)-N-(4-(trifluoromethyl)phenyl)thiazol-2-amine R-A.6 and (S)-5-(1-aminoethyl)-N-(4-(trifluoromethyl)phenyl)thiazol-2-amine S-A.6. 5-(1-Aminoethyl)-N-(4-(trifluoromethyl)phenyl)thiazol-2-amine A.6 (160 g) was purified by preparative super-critical fluid chromatography on a Chiralpak AS-H (2×25 cm, #07-8620) with an isocratic eluant of 20% MeOH(0.1% Et₂NH)/CO₂ at 100 bar, a flow rate of 80 mL/min, an injection vol of 1 mL of a 50 mg/mL MeOH/CH₂Cl₂ solution, and monitoring by UV detection at 220 nM to yield 63 g (39% yield, >99% ee) of (S)-5-(1-aminoethyl)-N-(4-(trifluoromethyl)phenyl)thiazol-2-amine S-A.6 as the first eluting peak and 61 g (38% yield, >99% ee) of (R)-5-(1-aminoethyl)-N-(4-(trifluoromethyl)phenyl)thiazol-2-amine R-A.6 as the second eluting peak. Enantiomeric purity was determined by analytical SCF chromatography Chiralpak AS-H (25×0.46 cm) with an isocratic eluant of 30% MeOH(0.1% Et₂NH)/CO₂ at 100 bar, a flow rate of 3 mL/min, and monitoring by UV detection at 220 nM.

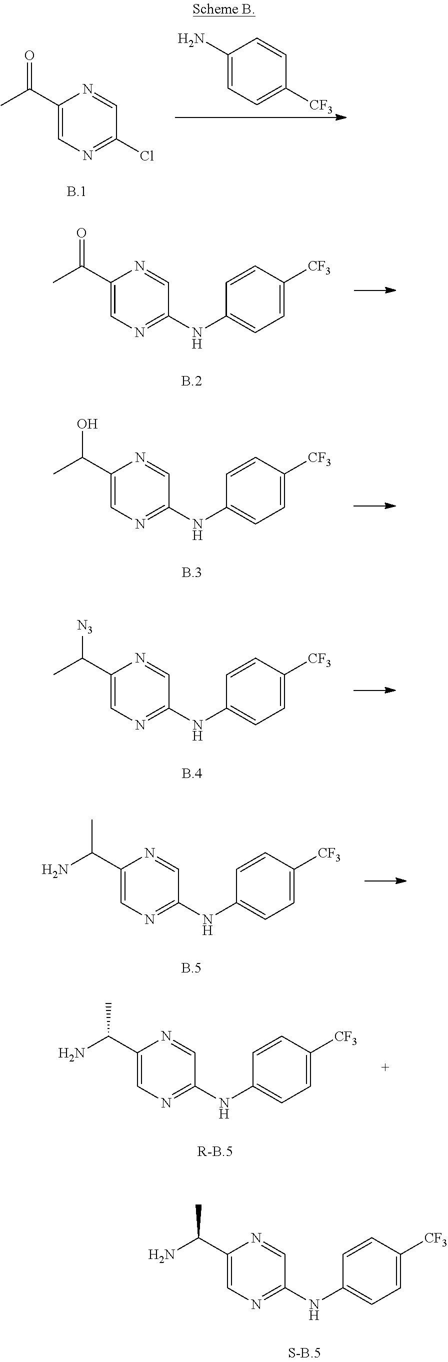

Synthesis of 1-(5-(4-(trifluoromethyl)phenylamino)pyrazin-2-yl)ethanone B.2. A stirred solution of 2-chloro-4-acetylpyrazine B.1 (500 mg, 3.2 mmol) in EtOH (3 ml) was treated with 4-trifluoromethylaniline (619 mg, 3.8 mmol) at room temperature, followed by the addition of 4N HCl in Dioxane (0.32 ml). The resulting reaction mixture was stirred at 100° C. for 16 hr in a sealed tube. After consumption of the starting material (by TLC), the reaction mixture was concentrated under reduced pressure, and the resulting crude was purified by column chromatography (20% ethyl acetate/hexane) using silica gel (60-120 mesh) to afford 430 mg (47%) of 1-(5-(4-(trifluoromethyl)phenylamino)pyrazin-2-yl)ethanone B.2. ¹H-NMR (DMSO-D₆, 200 MHz) δ 10.51 (s, 1NH), 8.73 (d, J=2 Hz, 1H), 8.31 (d, J=2 Hz, 1H), 7.99 (d, J=10 Hz, 2H), 7.72 (d, J=8 Hz, 2H), 2.49 (s, 3H). LCMS m/z=281.9 [M+1].

Synthesis of 1-(5-(4-(trifluoromethyl)phenylamino)pyrazin-2-yl)ethanol B.3. A solution of 1-(5-(4-(trifluoromethyl)phenylamino)pyrazin-2-yl)ethanone B.2 (100 mg, 0.35 mmol) in EtOH (3.5 ml) in an ice bath was treated with NaBH₄ (27 mg, 0.71 mmol) portion wise. The reaction mixture was allowed to stir at room temperature for 1 hr. After consumption of the starting material (by TLC) the reaction mixture was quenched with cold water, and concentrated under reduced pressure to remove the volatiles. The aqueous layer was extracted with EtOAc (2×15 ml). The combined organic layers was dried over Na₂SO₄ and concentrated under reduced pressure to afford 90 mg (90%) of 1-(5-(4-(trifluoromethyl)phenylamino)pyrazin-2-yl)ethanol B.3 as a white solid. ¹H-NMR (CDCl₃+DMSO-D₆, 200 MHz) δ 9.13 (s, 1NH), 8.26 (d, J=2 Hz, 1H), 7.83 (d, J=8 Hz, 2H), 7.53 (d, J=10 Hz, 2H), 4.91-4.85 (m, 1H), 4.47 (d, J=4 Hz, 1H), 1.53 (d, J=6 Hz, 3H). LCMS m/z=284.0 [M+1].

Synthesis of 5-(1-azidoethyl)-N-(4-(trifluoromethyl)phenyl)pyrazin-2-amine B.4. A mixture of 150 mg (0.35 mmol) of 1-(5-(4-(trifluoromethyl)phenylamino)pyrazin-2-yl)ethanol B.3 in 2.4 mL CH₂Cl₂ was cooled in an ice bath and treated with 0.11 ml (0.52 mmol) of diphenylphosphonic azide at 0° C. for 10 min, followed by the drop wise addition of 0.070 ml (0.52 mmol) of DBU at 0° C. The reaction mixture was allowed to stir at room temperature for 1 hr. After consumption of the starting material (by TLC), the reaction mixture was quenched with cold water and extracted with CH₂Cl₂ (3×20 ml). The combined organic layers was dried over Na₂SO₄ and concentrated under reduced pressure. Purification by column chromatography afford 86 mg (80%) of 5-(1-azidoethyl)-N-(4-(trifluoromethyl)phenyl)pyrazin-2-amine B.4. 1H-NMR (DMSO-D6, 200 MHz) δ 10.05 (s, 1NH), 8.31 (d, J=10 Hz, 2H), 7.93 (d, J=10 Hz, 2H), 7.67 (d, J=8 Hz, 2H), 4.77-4.74 (m, 1H), 1.54 (d, J=6 Hz, 3H). LCMS m/z=308.9 [M+1].

Synthesis of 5-(1-aminoethyl)-N-(4-(trifluoromethyl)phenyl)pyrazin-2-amine B.5. A solution of 80 mg (0.25 mmol) of 5-(1-azidoethyl)-N-(4-(trifluoromethyl)phenyl)pyrazin-2-amine B.4 in 2.5 mL of 4:1 THF/H₂O was treated with 102 mg (0.38 mmol) of triphenylphosphine. The reaction mixture was heated at 60° C. for 16 hr. After consumption of the starting material (by TLC), volatiles were removed by concentration under reduced pressure. The aqueous layer was extracted with ethyl acetate (3×20 ml). The combined organic layers was dried over Na₂SO₄ and concentrated under reduced pressure to afford 100 mg (73% as crude) of 5-(1-aminoethyl)-N-(4-(trifluoromethyl)phenyl)pyrazin-2-amine B.5. This material was used for the next step without any future purification. LCMS m/z=283.6 [M+1].

Synthesis of (R)-5-(1-aminoethyl)-N-(4-(trifluoromethyl)phenyl)pyrazin-2-amine R-B.5 and (S)-5-(1-aminoethyl)-N-(4-(trifluoromethyl)phenyl)pyrazin-2-amine S-B.5. 5-(1-Aminoethyl)-N-(4-(trifluoromethyl)phenyl)pyrazine-2-amine B.5 (50.08 g) was purified by preparative chiral chromatography on a Chiralpak AS-H column with an isocratic eluant of 75/25/0.05 Hexane/Ethanol/diethylamine, and monitoring by UV detection at 370 nM to yield 21.9 g (86% yield, 99.8% ee) of (R)-5-(1-aminoethyl)-N-(4-(trifluoromethyl)phenyl)pyrazine-2-amine R-B.5 as the first eluting peak and 22.3 g (88.3% yield, 99.6% ee) of (S)-5-(1-aminoethyl)-N-(4-(trifluoromethyl)phenyl)pyrazine-2-amine S-B.6 as the second eluting peak. Enantiomeric purity was determined by analytical chromatography on a Chiralpak ASHSADI006-401291 (4.6×250 mm) with an isocratic eluant of 75/25/0.1 Hexane/Ethanol/diethylamine, a flow rate of 1 mL/min, and monitoring by UV detection at 220 nm.

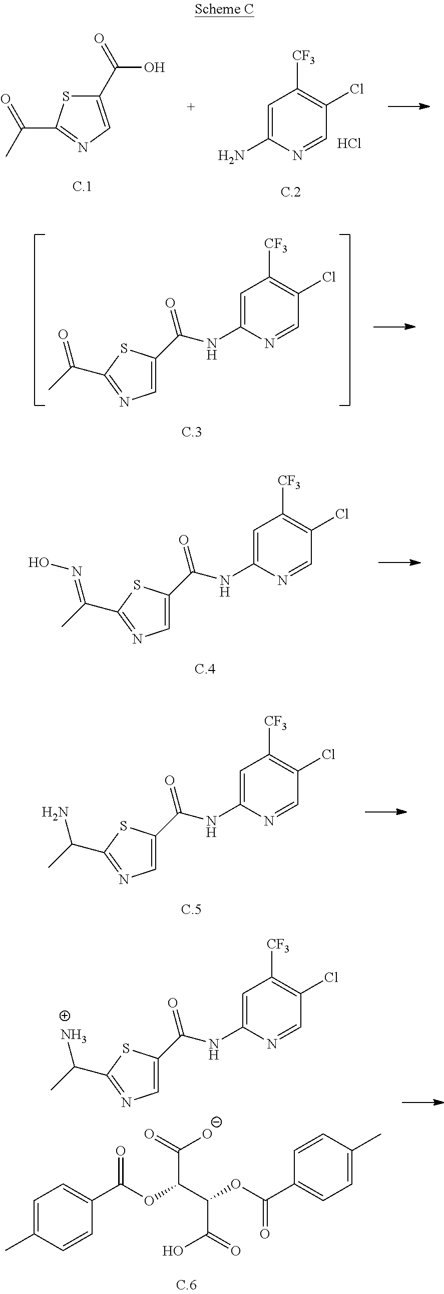

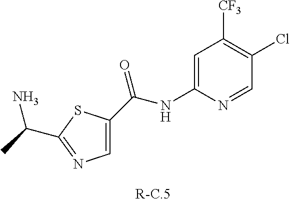

Synthesis of Compound C.3 To a clean dry flask was charged 21.83 g (127.5 mmols, 1.06 eq) of 2-acetylthiazole-5-carboxylic acid (ComPound C.1), 40.5 mL of 1,2-dimethoxyethane, and 42.8 mg (5 mol %) of N,N-dimethylformamide under a nitrogen atmosphere. The resulting mixture was allowed to stir at 20-30° C. while 15.85 g (123.8 mmoles, 1.03 eq) of oxalyl chloride was charged dropwise over 30 minutes. The resulting reaction solution was allowed to stir for at least 3 hr at 25° C. In a separate flask was charged 28.07 g (120.5 mmoles, 1 eq) of 5-chloro-4-(trifluoromethyl)pyridine-2-amine hydrochloride (Compound C.2), 87 mL of acetonitrile, and 29.1 mL of (360.3 mmoles, 2.99 eq) pyridine under a nitrogen atmosphere. The resulting solution was cooled to 10° C. with stirring. To the cooled C.2 solution was added the activated C.1 solution dropwise over 30 minutes. The final combined solution was allowed to warm to room temperature, and the stirring was continued for an additional 2 hours. This solution may be used in the next step without isolation. However, Compound C.3 can be isolated from the solution at this point by adding water dropwise until a thick slurry is obtained.

Synthesis of Compound C.4. The solution of C.3, from the procedure described above, was heated to 45° C. while maintaining stirring under a nitrogen atmosphere. To the heated solution was added 9.30 g of NH₂OH dropwise over 5 minutes. After the addition was complete, stirring was continued at 45° C. for an additional 4 hr. The reaction solution was then heated to 60° C. and 215 mL of water was added over the course of 1 hr. The resulting slurry was cooled to room temperature and filtered to collect the solids. The filter cake was washed with 25% v/v acetonitrile/water, then water, and dried to constant weight at room temperature. A total of 44.26 g of compound C.4 was produced in 98% yield. Mass spectra showed a molecular ion [M+1] of 365.01.

Synthesis of Compound C.5. To a clean dry flask was charged 11.5 g (31.5 mmoles, 1 eq) of compound C.4, 4.6 g (70.3 mmoles, 2.23 eq) of zinc dust, 35 mL of water, and 57 mL of 1-butanol under a nitrogen atmosphere. While stirring vigorously, the resulting mixture was cooled to 0-5° C. To the cold mixture was charged 10.8 mL (188.7 mmoles, 6 eq) of acetic acid dropwise, while maintaining the internal reaction temperature of <10° C. Once the addition is complete, the reaction was allowed to warm to 30° C., and the stirring was continued for an additional 3-4 hr. After aging the reaction solution, the contents of the flask were cooled to ˜5° C., and 56 mL of NH₄OH was added dropwise while maintaining an internal temperature <10° C. The biphasic mixture was warmed to 35° C. and the aqueous phase was removed. The organic layer was washed once more with a mixture of 24 mL of NH₄OH and 24 mL of water at 35° C. The aqueous phase was removed and the 16 mL of heptane was added to the organic layer. The organic solution was then washed with a solution of 1.15 g of EDTA in 50 mL of water at 35° C. The aqueous phase was removed, and the organic phase, at 35° C., was filtered through a 4-5.5 micron filter funnel into a separate clean dry flask. To the filtered solution was added 215 mL of heptane at ambient temperature with stirring over the course of 1 hr. The slurry was cooled to 0-5° C. and held with stirring for an additional 3 hr. The solids were collected by filtration and washed with 35 mL of heptane in 2 portions. The wet solids were dried at 50° C. under high vacuum for 30 hr. Compound C.5, 8.52 g, was isolated as a pale pink solid in a 77% yield. The mass spectrum showed a molecular ion [M+1] of 351.35.

Synthesis of Compound C.6. To a clean dry flask was charged 80 g (228 mmoles, 1 eq) of Compound C.5, 263 g of 2-propanol, and 263 mL of water under a nitrogen atmosphere. The resulting mixture was heated to 53° C. and stirred until all the solids dissolved. In a separate clean dry flask was charged 59.2 g (153 mmoles, 0.67 eq) of D-ditoluoyl tartaric acid, 481 g of 2-propanol, and 206 g of water under a nitrogen atmosphere. The tartaric acid solution was stirred until all the solids dissolved at room temperature, and then added to the Compound C.5 solution through a coarse filter funnel at such a rate to maintain the internal temperature of the Compound C.5 solution at 45-53° C. The coarse filter funnel was washed with an additional 40 mL of a 3:1 2-propanol:water solution. Immediately following the funnel wash, the stirring of combined solutions was stopped, and the contents of the flask were held at 45° C. for 9 hr. After aging, the reaction mixture was cooled to 20° C., and the stirring was resumed. The contents of the flask were held at 20° C. with stirring for approximately 12 hr. The solids were then collected by filtration, and the wet solids were washed with 80 mL of a cold 2-propanol:water (3:1) solution in 2 portions. The wet solids were then dried at 50° C. under vacuum to constant weight. A total of 74.2 g of Compound C.6 was obtained in 88% yield.

The stereochemical purity of Compound C.6 was further enhanced by the following procedure. To a clean dry flask was charged 66.5 g (90 mmoles, 1 eq) of Compound C.6, 335 g of water, and 1330 g of 2-propanol under a nitrogen atmosphere. With stirring, the contents of the flask were heated to 60° C., and held at that temperature for 1 hr. After aging, the stirring was stopped, and the contents of the flask were cooled to 0° C. over 4 hr. During this cooling period, the stirring was started and stopped after approximately 20 seconds 5 times over evenly spaced intervals. The contents of the flask were held at 0° C. for 2 hr without stirring. After aging, the solids were collected by filtration. The wet solids were dried at 50° C. under vacuum to constant weight. A total of 53.8 g of Compound C.6 was obtained in a 81% yield. Mass spectral analysis (positive mode) showed a molecular ion of 351.43 [M+1].

Synthesis of Compound R-C.5. To a clean dry flask was charged 156 g (217 mmoles, 1 eq) of Compound C.6, 1560 mL of methyl tert-butyl ether, and 780 mL of methanol under a nitrogen atmosphere. The contents of the flask were then stirred at room temperature, and a solution of 250 g (1110 mmoles, 5.26 eq) of sodium bicarbonate in 2340 mL of water was added slowly to maintain the internal temperature of ≦30° C. The resulting mixture was stirred for an additional hour at 30° C. After aging, the stirring was stopped and the organic and aqueous layers were allowed to separate. The aqueous layer was removed, and the organic layer was concentrated under vacuum to obtain a thick slurry. To the slurry was added 1000 mL of heptane, and the resulting mixture was cooled to 0-5° C. The solids were collected from the cold solution by filtration. The wet solids were then dried at 50° C. under vacuum to constant weight. A total of 68.7 g of Compound R-C.5 was obtained in a 92% yield. Mass spectral analysis showed a molecular ion [M+1] of 351.35.

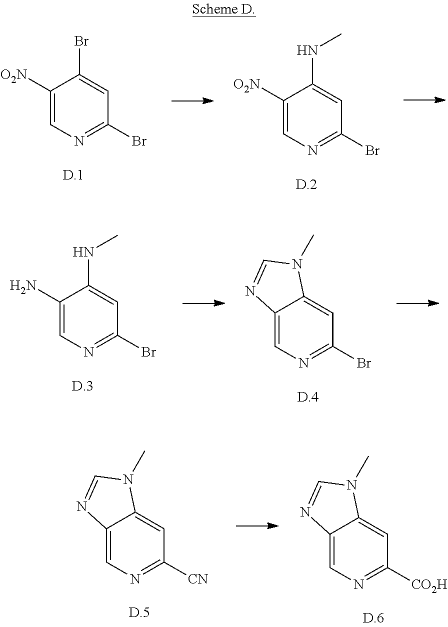

Synthesis of 2-bromo-N-methyl-5-nitropyridin-4-amine D.2. A 2.0 M solution of methyl amine in THF (480 mL, 958 mmol) was added to a solution of 2,4-dibromo-5-nitropyridine D.1 (135 g, 479 mmol) in 2800 mL of anhydrous THF over a 1 hr period. The reaction mixture was stirred at room temperature for an additional 1 hr. The reaction mixture was poured into saturated aqueous sodium chloride and extracted with ethyl acetate (2×4 L). The combined organics were concentrated under reduced pressure, dissolved in dichloromethane (1.2 L), and absorbed onto silica gel (200 g). The material was then purified on s silica gel column (1.0 Kg) and eluted with a 40% solution of ethyl acetate in heptane (20 L) to give 103.4 g (93%) of 2-bromo-N-methyl-5-nitropyridin-4-amine D.2.

Synthesis of 6-bromo-N⁴-methylpyridine-3,4-diamine D.3. A solution of 103.4 g (444 mmol) of 2-bromo-N-methyl-5-nitropyridin-4-amine D.2 in 1.5 L of glacial acetic acid was added to a 70° C. solution of 99 g (1.78 mol) of iron fillings in 1.5 L of glacial acetic acid over 1 hr (slight exotherm). The resulting grey suspension was stirred at 70° C. for an additional 1 hr. The reaction mixture was filtered trough a bed of celite and washed with acetic acid (250 mL). The reaction was concentrated under reduced pressure and carefully added to a solution of potassium carbonate (500 g) in water (1 L). The mixture is extracted with ethyl acetate (2×2 L), dried over Na₂SO₄, and absorbed onto silica gel (200 g). The mixture was loaded onto a silica gel column (I Kg) and eluted with ethyl acetate (20 L) to 74 g (82%) of 6-bromo-N⁴-methylpyridine-3,4-diamine D.3.

Synthesis of 6-bromo-1-methyl-1H-imidazo[4,5-c]pyridine D.4. A mixture of 60 g (295.5 mmol) of 6-bromo-N⁴-methylpyridine-3,4-diamine D.3 in 1.5 L of triethyl orthoformate was heated at 120-125° C. for 48 hr. The reaction mixture was concentrated under reduced pressure and the resulting solid was triturated with MTBE (100 mL) to give the 38.2 g (61%) of 6-bromo-1-methyl-1H-imidazo[4,5-c]pyridine D.4.

Synthesis of 1-methyl-1H-imidazo[4,5-c]pyridine-6-carbonitrile D.5. A suspension of 38 g (180 mmol) of 6-bromo-1-methyl-1H-imidazo[4,5-c]pyridine D.4, 12.7 g (108 mmol) of zinc cyanide, 2.4 g (36 mmol) of zinc dust, and 7.4 g (9 mmol) of PdCl₂(dppf)-CH₂Cl₂ were suspended in a solution of dimethyl acetamide (450 mL) and stirred for 30 min while a stream of nitrogen was bubbled through the suspension. The reaction was heated at 95-100° C. for 2.5 hr. The majority of the dimethyl acetamide was removed under reduced pressure. The resulting mixture was diluted with saturated ammonium chloride (250 mL), concentrated ammonium hydroxide (200 mL), water (200 mL) and dichloromethane (500 mL). Ethyl acetate (1.5 L) was added and the mixture was filtered to remove residual solids. The layers were then separated and the aqueous layer was washed with ethyl acetate (8×500 mL). The combined organics were dried over sodium sulfate, concentrated under reduced pressure and absorbed onto silica gel (100 g). This material was loaded on a silica gel column (600 g) and eluted with dichloromethane (4 L), 2.5% methanol/dichloromethane (6 L), and finally with 5% methanol/dichloromethane (6 L) to give 9.4 g of 1-methyl-1H-imidazo[4,5-c]pyridine-6-carbonitrile D.5. The solids (13 g) from the initial filtration were found to be mostly product. This material was purified as described above to give an additional 9.2 g of 1-methyl-1H-imidazo[4,5-c]pyridine-6-carbonitrile D.5 for an overall combined yield of 65%.

Synthesis of 1-methyl-1H-imidazo[4,5-c]pyridine-6-carboxylic acid D.6. A mixture of 11.3 g (71.5 mmol) of 1-methyl-1H-imidazo[4,5-c]pyridine-6-carbonitrile was heated at 90-95° C. for 5 hr in 6 N HCl (200 mL). The solvent was removed under reduced pressure and the solid was triturated in MTBE (100 mL). The solid was dried at 50° C. in a vacuum oven for 4 hr to give the 17.3 g (quant yield) of 1-methyl-1H-imidazo[4,5-c]pyridine-6-carboxylic acid D.6 as the diHCl salt. LCMS m/z=178 [M+1].

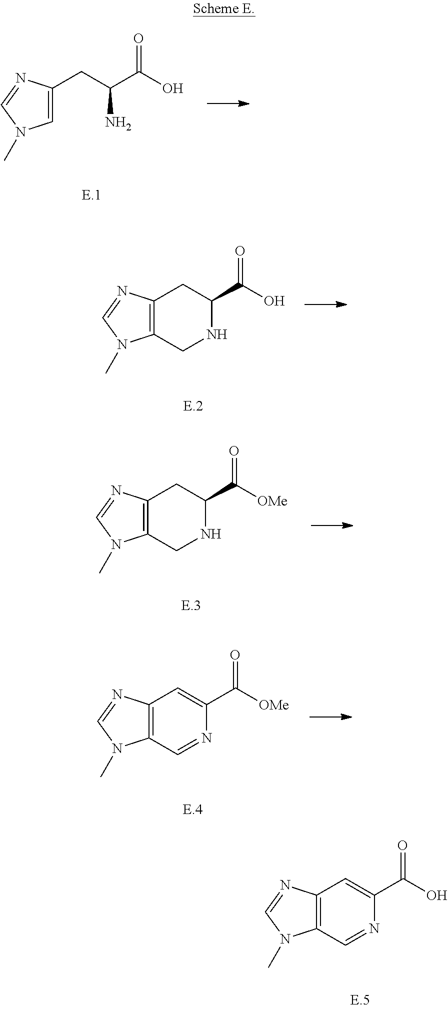

Synthesis of (S)-3-methyl-4,5,6,7-tetrahydro-3H-imidazo[4,5-c]pyridine-6-carboxylic acid E.2. A solution of 0.2 g (1.18 mmol) of (S)-2-amino-3-(1-methyl-1H-imidazol-4-yl)propanoic acid E.1 in 5 mL of water was treated with 0.07 mL (2.3 mmol) of conc. HCl and (0.66 mL, 2.3 mmol) of formaldehyde slowly at 0° C. After being stirred for 30 min at 0° C., the reaction mixture was slowly heated to reflux temperature and continued stirring for 12 hr. After completion of starting material (by TLC), the volatiles were evaporated under reduced pressure to give crude compound. The crude material was suspended in isopropanol (4 mL) and HCl (1 mL of 4M solution in 1,4-dioxane) and stirred for 30 min. The precipitated solid was filtered, washed with diethyl ether and dried under vacuum to afford 0.2 g (80%) of (S)-3-methyl-4,5,6,7-tetrahydro-3H-imidazo[4,5-c]pyridine-6-carboxylic acid E.2 as off-white solid. ¹H-NMR (200 MHz, DMSO-d₆) δ 11.4-10.8 (brs, 2H, D₂O exchangeable), 9.00 (s, 1H), 4.61-4.40 (m, 2H), 4.38-4.21 (m, 1H), 3.81 (s, 3H), 3.42-3.20 (m, 1H), 3.20-3.01 (m, 1H). LCMS m/z=182.0 [M+1].

Synthesis of (S)-methyl 3-methyl-4,5,6,7-tetrahydro-3H-imidazo[4,5-c]pyridine-6-carboxylate E.3. Thionyl chloride (0.24 mL, 3.3 mmol) was added in a drop-wise fashion to 10 mL of anhydrous MeOH at 0° C. under inert atmosphere. After being stirred for 10 min, 0.2 g (1.1 mmol) of (S)-3-methyl-4,5,6,7-tetrahydro-3H-imidazo[4,5-c]pyridine-6-carboxylic acid E.2 was added to the reaction mixture slowly at 0° C. After complete addition, the reaction mixture was stirred at reflux temperature for 10 hr. After completion of starting material (by TLC), the volatiles were evaporated under vacuum to give crude compound. The crude material was washed with diethyl ether to afford 0.2 g (85%) of (S)-methyl 3-methyl-4,5,6,7-tetrahydro-3H-imidazo[4,5-c]pyridine-6-carboxylate E.3 as a white solid. ¹H-NMR (200 MHz, DMSO-d₆) δ 11.4-10.8 (brs, 1H, D₂O exchangeable), 9.00 (s, 1H), 4.71-4.60 (m, 1H), 4.58-4.24 (m, 2H), 3.81 (s, 6H), 3.42-3.15 (m, 2H). LCMS m/z=195.9 [M+1].

Synthesis of methyl 3-methyl-3H-imidazo[4,5-c]pyridine-6-carboxylate E.4. To a solution of 0.2 g (1.0 mmol) of (S)-methyl 3-methyl-4,5,6,7-tetrahydro-3H-imidazo[4,5-c]pyridine-6-carboxylate E.3 in 10 mL of CCl₄ were added 1.0 mL (7.2 mmol) of triethylamine and 0.28 g (2.5 mmol) selenium dioxide, followed by a catalytic amount of PPSE (˜5 mg) at room temperature under inert atmosphere. The reaction mixture was stirred at reflux temperature for 12 hr. After completion of starting precursor (by TLC), the volatiles were evaporated under reduced pressure to get crude compound. The crude material was purified over silica gel column chromatography eluting with EtOAc/NH₄OH/MeOH (8:1:1) to afford 0.12 g (61%) of methyl 3-methyl-3H-imidazo[4,5-c]pyridine-6-carboxylate E.4 as a light yellow solid. ¹H-NMR (200 MHz, DMSO-d₆) δ 9.02 (s, 1H), 8.59 (s, 1H), 8.39 (s, 1H), 4.01 (s, 3H), 3.85 (s, 3H). LCMS m/z=191.9 [M+1].

Synthesis of 3-methyl-3H-imidazo[4,5-c]pyridine-6-carboxylic acid E.5. To a stirred solution of 0.12 g (0.62 mmol) of methyl 3-methyl-3H-imidazo[4,5-c]pyridine-6-carboxylate E.4 in 2 mL of THF and 2 mL of water was added 52 mg (1.2 mmol) of lithium hydroxide at room temperature and the reaction mixture was stirred for 16 hr at room temperature. After completion of starting precursor (by TLC), volatiles were evaporated under reduced pressure. Resulting residue was diluted with water and washed with 10 mL of EtOAc. Aqueous layer was acidified using conc. HCl and evaporated under vacuum. The resulting residue was dried by co-distillation with toluene to afford 0.1 g (90%) of 3-methyl-3H-imidazo[4,5-c]pyridine-6-carboxylic acid E.5 as light brown solid. ¹H-NMR (200 MHz, DMSO-d₆) δ 9.42 (s, 1H), 8.99 (s, 1H), 8.46 (s, 1H), 4.11 (s, 3H).

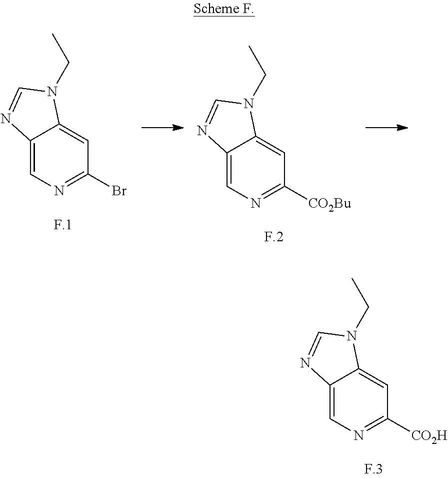

Synthesis of Compound F.1. The compound F.1 was prepared as described previously in Scheme D using ethylamine in place of methylamine. ¹H NMR (CDCl₃, 200 MHz) δ 8.89 (s, 1H), 8.02 (s, 1H), 7.59 (s, 1H), 4.25 (q, J=7.6 Hz, 3H), 1.59 (t, J=6.6 Hz, 3H); LCMS m/z=226 [M+1].

Synthesis of Compound F.2. To a stirred solution of F.1 (8 g, 0.037 mol) in acetonitrile:n-Butanol (80 ml of 1:1) was added BINAP (4.4 g, 0.008 mol), DIPEA (8 ml), Pd(CH₃CN)₂C12 (1.8 g, 0.006 mol) at room temperature. The reaction mixture was heated under CO pressure at 100° C. in a steel bomb. After consumption of the starting material (by TLC), volatiles were removed under reduced pressure. The crude material was purified by column chromatography [silica gel (60-120 mesh, 40 g) 40 mm, gradient 5% MeOH/CH₂Cl_{2]} to afford compound F.2 (5.5 g, 60%) as brown color liquid. ¹H NMR (CDCl₃, 200 MHz) δ 9.25 (s, 1H), 8.37 (s, 1H), 8.15 (s, 1H), 4.52 (t, J=7.2 Hz, 2H), 4.35 (q, J=7.6 Hz, 2H), 1.92-1.83 (m, 2H), 1.64 (t, J=7.2 Hz, 3H), 1.50-1.42 (m, 2H), 0.97 (d, J=6.6 Hz, 3H); LCMS m/z=248.1 [M+1].

Synthesis of Compound F.3. Compound F.2 (5 g, 0.020 mol) was dissolved in TEA (25 ml) and water (50 ml), and was stirred at room temperature for 48 hr. After consumption of the starting material (by TLC), volatiles were removed under reduced pressure. The crude material was dried with co-distillation with toluene to afford 3.5 g of compound F.3 as off-white solid that was used without further purification. ¹H NMR (CD₃OD, 200 MHz) δ 9.06 (s, 1H), 8.70 (s, 1H), 8.57 (s, 1H), 4.53 (q, J=7.5 Hz, 2H), 1.60 (t, J=6.5 Hz, 3H); LCMS m/z=192 [M+1].

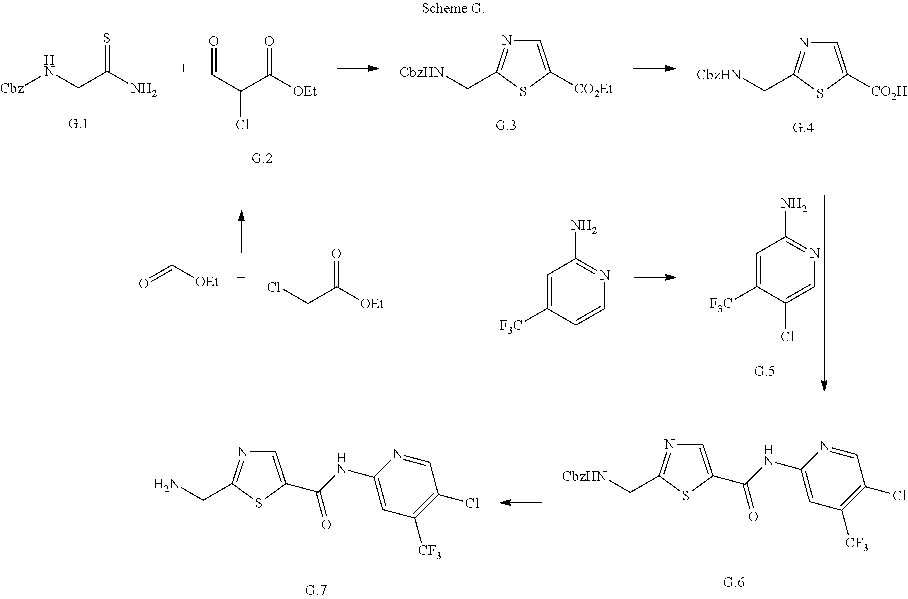

Synthesis of Compound G.2. Ethyl chloroacetate (50 g, 0.409 mol) and ethyl formate (30.3 g, 0.409 mol) were taken in anhydrous toluene (500 mL) and cooled to 0° C. NaOEt (33 g, 0.485 mol) was added portion wise. The reaction mixture was stirred at 0° C. for 5 hr and then at room temperature for 12 hr. The reaction mixture was quenched with water (250 mL) and washed with Et₂O (2×250 mL). The aqueous layer was cooled to 0° C. and acidified to pH 4 using 5N HCl. The aqueous layer was extracted with Et₂O (3×300 mL). The combined organic layers were dried (Na₂SO₄) and concentrated under reduced pressure to obtain compound G.2 as light brown oil (54 g, 88%), which was used without further purification.

Synthesis of Compound G.3. To a solution of aldehyde G.2 (54 g, 0.36 mol) in anhydrous DMF (42 mL), was added a solution of compound G.1 (40.3 g, 0.18 mol) in anhydrous DMF (320 mL). The reaction was heated at 50° C. for 3 days. The mixture was cooled to 0° C., and Et₂O (390 mL) followed by sat. NaHCO₃ solution (200 mL) were added slowly. After separation of the phases, the aqueous layer was extracted with Et₂O (2×300 mL). The combined organic extracts were washed with sat. NaHCO₃ (3×500 mL), dried (Na₂SO₄) and concentrated under reduced pressure to give crude material as thick brown oil, which was purified by column chromatography (EtOAc/hexanes) to give compound G.3 as a brown solid (22 g, 19%). ¹H NMR: (CDCl₃, 200 MHz) δ 8.3 (s, 1H), 7.4 (s, 5H), 5.6 (brs, 1H), 5.2 (s, 2H), 4.7 (d, 2H, J=5 Hz), 4.4 (m, 2H), 1.4 (m, 3H); LCMS: m/z 320.9 [M+1].

Synthesis of Compound G.4. To an ice-cold solution of compound G.3 (10 g, 0.0311 mol) in THF/H₂O (80 mL, 1:1) was added LiOH (2.6 g, 0.062 mol). The reaction was stirred for 3 hr, whereupon THF was removed under reduced pressure and the aqueous layer was extracted with Et₂O (2×50 mL). The aqueous layer was cooled to 0° C. and acidified with 3N HCl (20 mL) during which solid precipitated out. The solid was filtered, washed with water (2×100 mL) and dried to give compound G.4 as a white solid (7 g, 77%). ¹H NMR: (CDCl₃-DMSO-d₆) δ 8.2 (s, 1H), 7.4 (s, 5H), (brs, 1H), 5.2 (s, 2H), 4.8 (d, 2H, J=4 Hz); ¹³C NMR: (DMSO-d₆, 60 MHz): 176.33, 162.04, 156.39, 147.62, 136.78, 130.25, 128.3, 127.7, 65.9, 42.71, 40.34; LCMS: m/z 292.8 [M+1].

Synthesis of Compound G.5. To a solution of 2-amino-4-trifluoromethyl-pyridine (2.00 g, 0.0123 mol) in DMF (4 mL, 0.05 mol) was added a solution of 1,3-dichloro-5,5-dimethylhydantoin (1.4 g, 0.0074 mol) in DMF (4 mL) dropwise. The reaction was stirred at room temperature for 2 hr, whereupon the reaction mixture was diluted with ether (80 mL) and washed with water (10 mL). The organic phase was dried and concentrated to give the crude product, which was purified on combiflash (0-20% EtOAc/Hexanes) to give compound G.5 as light yellow oil. (65% yield); ¹H NMR: (DMSO-d₆) δ 8.16 (s, 1H), 6.87 (s, 1H), 6.76 (brs, 1H); LCMS: m/z 197 [M+1].

Synthesis of Compound G.6. A 20 mL vial was charged with compound G.4 (191.8 mg, 0.65 mmol), CH₂Cl₂ (3.0 mL), a 2.0 M solution of oxalyl chloride in CH₂Cl₂ (390 μL), and DMF (10.0 μL, 0.129 mmol). The reaction mixture was stirred for 15 minutes at room temperature, then concentrated in vacuo and the resultant residue was taken up in acetonitrile (3.0 mL). To this solution was added a solution of compound G.5 (129 mg, 0.65 mmol) and pyridine (0.5 mL, 0.006 mol) in acetonitrile (1.5 mL). The reaction mixture was stirred at room temperature overnight. The solvent was removed under reduced pressure, and the residue was purified by flash column chromatography (SiO₂, 0-30% EtOAc/CH₂Cl₂) to give compound G.6 in 49% yield. LCMS: m/z=471 [M+1].

Synthesis of Compound G.7. A vial was charged with compound G.6 (1.0E2 mg, 0.21 mmol), acetic acid (1.0 mL, 0.018 mol) and hydrogen bromide (300 μL, 4 M/acetic acid). The reaction mixture was stirred at room temperature for 2 hr. The reaction mixture was diluted with methanol and concentrated under reduced pressure. The residue was diluted with aqueous NaHCO₃ and ethyl acetate. After separation of the phases, the organic layer was washed with aqueous NaHCO₃ and brine, dried over sodium sulfate, and concentrated to give compound G.7 as a light brown solid (73% yield), which was used without further purification. ¹H NMR (300 MHz, DMSO-d₆) δ 8.85 (s, 1H), 8.79 (s, 1H), 8.57 (s, 1H), 4.48 (brs, 2H). LCMS: m/z 337 [M+1].

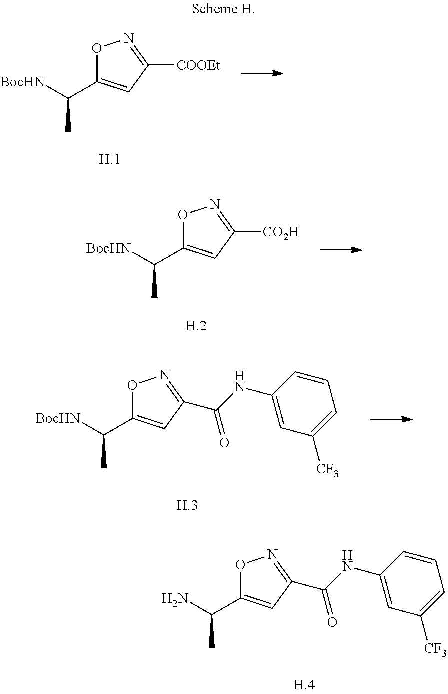

Synthesis of Compound H.2. To a solution of (R)-ethyl 5-(1-(tert-butoxycarbonylamino)ethyl)isoxazole-3-carboxylate H.1 (WO2006065703) in THF (2 L) was added aqueous 2.5 N LiOH (1 L) at room temperature. The mixture was stirred for 1 hr, and then evaporated under reduced pressure to remove THF. The residue was partitioned between water (1 L) and ethyl acetate (0.5 L). The organic layer was separated and the aqueous layer was extracted with ethyl acetate twice. The aqueous layer was adjusted to pH 2 with 10% HCl and extracted with ethyl acetate (2×1 L). All the organic layers were combined, washed with water, dried over Na₂SO₄, filtered and concentrated under reduced pressure. The residue was dried under vacuum to give the crude product (R)-5-(1-(tert-butoxycarbonylamino)ethyl)isoxazole-3-carboxylic acid H.2 (55.2 g, 44.8%), which was used without further purification. ¹H NMR (CDCl₃) δ6.57 (s, 1H), 4.12 (q, 1H), 1.56 (d, 3H), 1.37 (s, 9H).

Synthesis of Compound H.4. Compound H.4 was prepared as described previously in Scheme F replacing the 2-amino-5-chloro-4-trifluoromethyl-pyridine with 3-trifluoromethylaniline.

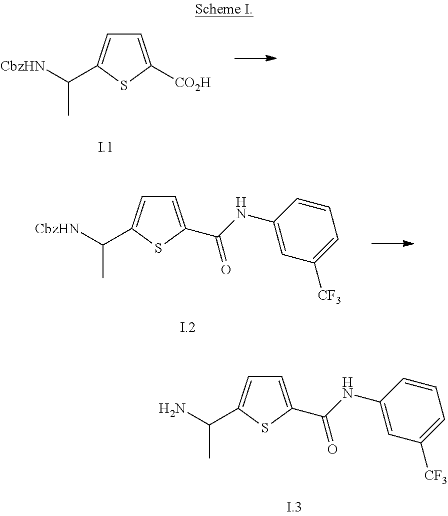

Synthesis of Compound 1.3. Compound 1.3 was prepared a described previously in Scheme G starting with 5-(1-(benzyloxycarbonylamino)ethyl)thiophene-2-carboxylic acid 1.1 (JP2003073357).

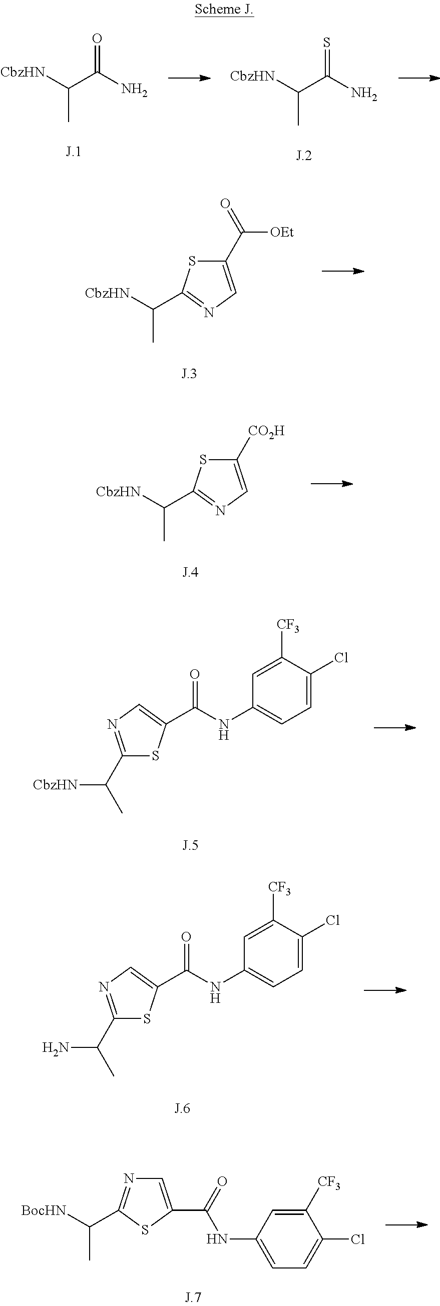

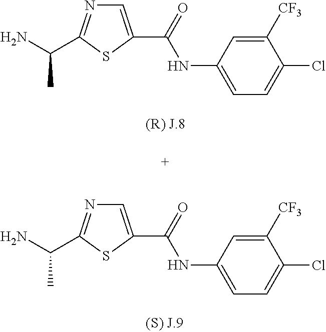

Synthesis of Compound J.2. To a solution of Z-alanine-NH₂ J.1 (5 g, 22.5 mmol) in dioxane (100 mL) was added Lawesson's reagent (5.4 g, 13.5 mmol). The reaction was heated at 60° C. overnight. The solvent was removed under reduced pressure, the resulting residue was diluted with a 1:1 mixture of saturated aqueous NaHCO₃:H₂O (100 mL), and extracted with ethyl acetate (3×100 mL). The combined extracts were washed with brine (100 mL), dried over anhydrous sodium sulfate, and concentrated in vacuo. Purification by flash column chromatography (10-60% EtOAc/hexanes) afforded compound J.2 (4.7 g, 90%) as a white solid. LCMS: m/z=239 [M+1].

Synthesis of Compound J.3. Compound J.2 was condensed with compound G.2 according to the procedure as described previously in Scheme G to afford compound J.3 (50% yield) as a light yellow solid. ¹H NMR (CDCl₃, 200 MHz): δ 8.3 (s, 1H), 7.3-7.5 (m, 5H), 5.4-5.5 (m, 1H), 5.1 (m, 2H), 4.3-4.4 (m, 2H), 1.6-1.7 (d, 2H), 1.3-1.4 (t, 3H); LCMS: m/z 335 [M+1].

Synthesis of Compound J.4. Hydrolysis of compound J.3 according to the procedure described previously in Scheme G to afford compound J.4 (83.5% yield) as a white solid. ¹H NMR (CDCl₃, 200 MHz): δ 8.2 (s, 1H), 7.2-7.4 (m, 5H), 5.1 (m, 2H), 4.8-4.9 (m, 1H), 1.3-1.5 (d, 2H); ¹³C NMR (75 MHz, DMSO-d₆): δ 181.12, 162.22, 155.81, 147.85, 136.89, 130.05, 128.46, 128.0, 127.89, 65.86, 20.47; LCMS: m/z 307 [M+1].

Synthesis of Compound J.6. Compound J.4 was coupled to 4-chloro-3-trifluoromethyl-phenylamine and deprotected according to procedures described in Scheme G to afford compound J.6. ¹H NMR (400 MHz, DMSO-d₆): δ 11.54 (s, 1H), 9.06 (s, 1H), 8.92 (br. s, 3H), 8.30 (d, J=Hz, 1H), 8.05 (dd, J=8.8, 2 Hz, 1H), 7.86 (d, J=8.8 Hz, 1H), 4.91 (quintet, J=6 Hz, 1H), 1.65 (d, J=6.8 Hz, 3H). LCMS: m/z 350 [M+1].

Synthesis of Compound J.7. To a flask containing compound J.6 (10.3 mg, 0.0294 mmol) was added a solution of carbonic acid di-tert-butyl ester (17.6 mg, 0.0799 mmol) in CH₂Cl₂ (0.6 mL) at room temperature. Triethylamine (8 μL) was added and the reaction was stirred at room temperature overnight. Water and ethyl acetate were added to the reaction mixtures and the layers were separated. The aqueous layer was extracted once more with ethyl acetate. The combined organic layers were dried over anhydrous sodium sulfate and concentrated in vacuo. Purification by column chromatography (EtOAc/Hexanes) afforded compound J.7 as a white solid (8.2 mg, 62%). Rf=0.1 (100% EtOAc); LCMS: m/z=450 [M+1].

Synthesis of Compound J.8 and J.9. Compound J.7 was separated by preparative chiral HPLC, using CHIRALPAK AD column and hexanes/EtOH (85:15) as the mobile phase. The compounds were deprotected by treatment with 4M-hydrochloric acid in dioxane at room temperature to afford compound J.8 and compound J.9. LCMS: m/z=350 [M+1].

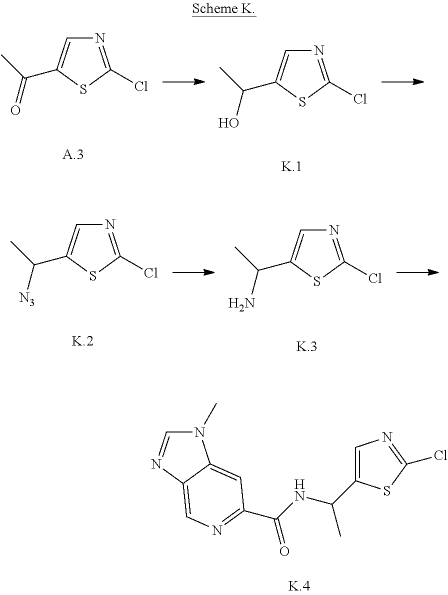

Synthesis of Compound K.1. To a stirred solution of compound A.3 (500 mg, 0.0031 mol) in EtOH (10 ml) was added NaBH₄ (234 mg, 0.0062 mol) portion wise at 0° C. The resulting reaction mixture was stirred at room temperature for 2 hr. After consumption of the starting material (by TLC), the reaction mixture was quenched with cold water (10 ml), and the volatiles were evaporated under reduced pressure. The crude material was extracted with CH₂Cl₂ (2×15 ml). The combined organic layers was dried over Na₂SO₄ and the solvent was evaporated under reduced pressure to afford compound K.1 (450 mg, 88.9%) as a colorless liquid. ¹H-NMR (CDCl₃, 200 MHz) δ 7.38 (s, 1H), 5.12 (q, J=5.8 Hz, 1H), 1.90 (bs, 1H), 1.61 (d, J=6.6 Hz, 3H). LCMS m/z=164 [M+1].

Synthesis of Compound K.2. To a stirred solution of compound K.1 (450 mg, 0.0027 mol) in CH₂Cl₂ (9 ml) was added diphenyl phosphoryl azide (1.1 g, 0.0041 mol) at 0° C. and stirred for 10 min then DBU (630 mg, 0.0041 mol) was added at 0° C. The resulting reaction mixture was stirred at 0° C. for 2 h. After consumption of the starting material (by TLC), the reaction mixture was quenched with cold water and extracted with CH₂Cl₂ (3×20 ml). The combined organic layers was dried over Na₂SO₄ and evaporated under reduced pressure. The resulting crude material was purified by column chromatography [silica gel (60-120 mesh, 20 g), gradient (5-15% EtOAc/Hexane)] to afford compound K.2 (400 mg, 78.4%) as a colorless oil. ¹H-NMR (CDCl₃, 200 MHz) δ 7.45 (s, 1H), 4.85 (q, J=6.6 Hz, 1H), 1.63 (d, J=6.6 Hz, 3H).

Synthesis of Compound K.3. To a stirred solution of compound K.2 (400 mg, 0.0021 mol) in THF:H₂O (8.4 ml of 20:1) was added triphenylphosphine (585 mg, 0.0022 mol) at room temperature. The resulting reaction mixture was stirred under reflux for 2 hr. After consumption of the starting material (by TLC), volatiles were evaporated under reduced pressure. The crude material was extracted with EtOAc (3×20 ml). The combined organic layers was dried over Na₂SO₄ and evaporated under reduced pressure to afford 200 mg of compound K.3 as a light yellow solid that was used without further purification. LCMS m/z=163 [M+1].

Synthesis of Compound K.4. A mixture of compound D.6 (4.7 g, 26.4 mmol), EDCI.HCl (11 g, 60.2 mmol), HOBT (1.6 g, 11.9 mmol) and compound K.3 (3.9 g, 24.1 mmol) in pyridine (40 ml) was stirred at room temperature for 5 hr. After consumption of the starting material (by TLC), the reaction mixture was diluted with water (100 ml) and extracted with EtOAc (2×100 ml). The combined organic layers was dried over Na₂SO₄ and evaporated under reduced pressure. The resulting crude material was purified by column chromatography [silica gel (60-120 mesh, 200 g), gradient (70% EtOAc/Hexane-Neat EtOAc)] to afford compound K.4 (3 g, 40%) as a light brown solid. ¹H-NMR (CD₃OD, 500 MHz) δ 9.06 (s, 1H), 8.62 (s, 1H), 8.59 (s, 1H), 556.-5.54 (q, J=6.5 Hz, 1H), 4.13 (s, 3H), 1.75 (d, J=7.5 Hz, 3H); LCMS m/z=322 [M+1].

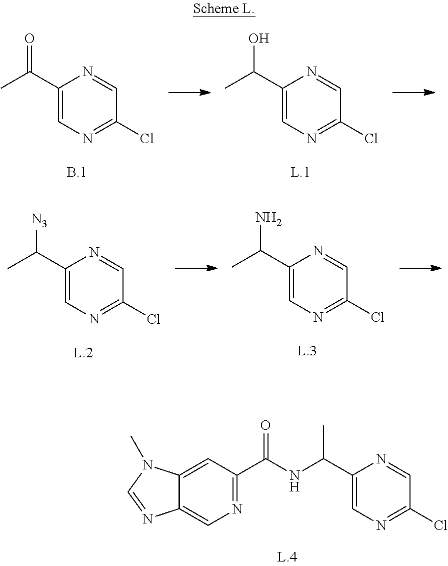

Synthesis of Compound L.1. Compound L.1 was prepared as described previously in Scheme K using compound B.1. ¹H-NMR (CDCl₃, 200 MHz) δ 8.53 (s, 1H), 8.47 (s, 1H), 5.05-4.95 (m, 1H), 1.58 (d, J=6.5 Hz, 3H); LCMS m/z=157.8 [M+1].

Synthesis of Compound L.2. Compound L.2 was prepared as described previously in Scheme K. ¹H-NMR (CDCl₃, 200 MHz) δ 8.57 (s, 1H), 8.43 (s, 1H), 4.76-4.65 (m, 1H), 1.67 (d, J=6.5 Hz, 3H); LCMS m/z=184.2 [M+1].

Synthesis of Compound L.3. Compound L.3 was prepared as described previously in Scheme K. LCMS m/z=158 [M+1].

Synthesis of Compound L.4. Compound L.4 was prepared as described previously in Scheme K. ¹H-NMR (CDCl₃, 200 MHz) δ 9.0 (s, 1H), 8.63 (s, 1H), 8.58 (s, 1H), 8.40 (s, 1H), 8.39 (s, 1H), 5.41-5.40 (m, 1H), 4.0 (s, 3H), 1.67 (d, J=7 Hz, 3H); LCMS m/z=317.1 [M+1].

### General Coupling of the Carboxylic Acid and NH₂-L¹-Cy¹-L²-Cy² Moieties

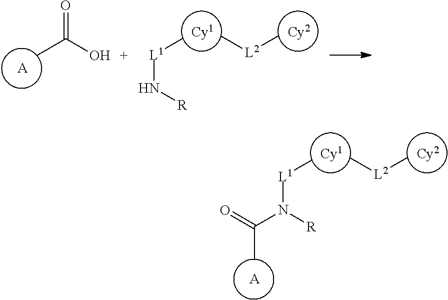

To a solution of the acid (1.1-1.6 equiv), the amine (1 equiv), and HOBT (1.3 equiv) in DMF (50 equiv) was added N-(3-dimethylaminopropyl)-N′-ethylcarbodiimide hydrochloride (1.5 eq.) and 4-methylmorpholine (1.0 equiv). If the amine was used as a salt at least one additional equivalent of 4-methylmorpholine was added. The reaction mixture was stirred at room temperature for 3-16 hr, and monitored by LCMS. After the reaction was complete, the solution was diluted with EtOAc, washed with water and brine. The solvent was removed from the organic phase, and the residue purified on flash column chromatography (EtOAc/Hexanes or MeOH/CH₂Cl₂ as eluents) or reverse phase preparative HPLC (mobile phase: acetonitrile/water, buffered with 0.1% TFA or 0.1% formic acid) to give the desired product. In the case of a chiral final product, the chiral purity was monitored by chiral HPLC using a Chiralcel OC or OJ-H column (mobile phase: ethanol/hexane buffered with 0.1% diethylamine).

In some instances an additional chemical transformation(s) was performed after amide bond formation. In these instances the following procedures were utilized.

General t-butyl carbamate deprotection conditions. To a room temperature or 0° C. solution of the t-butyl carbamate protected amine in dichloromethane was added trifluoroacetic acid. The reaction mixture was stirred until TLC or LCMS indicated complete consumption of the carbamate. Volatiles were removed under vacuum and the crude residue was purified by reverse-phase HPLC to afford the desired amine as a TFA or formic acid salt. The free base could be obtained by dissolving the salt in dichloromethane, washing with aqueous NaHCO₃, and evaporation under vacuum.

General reductive amination conditions. A room temperature solution of amine in MeOH was treated with 1-2 equiv of the corresponding aldehyde or ketone, 0.1 equiv of glacial AcOH, and 1.2 equiv of Na(CN)BH₃. The reaction mixture was stirred until TLC or LCMS indicated complete consumption of the amine. Purification by reverse-phase HPLC afforded the desired product as a TFA or formic acid salt. The free base could be obtained by dissolving the salt in dichloromethane, washing with aqueous NaHCO₃, and evaporation under vacuum.

TABLE 4
The following compounds of the present invention, set forth in Table 4,
below, were prepared by the general amide bond coupling method described above using the
appropriate amine from Schemes A-J and the appropriate carboxylic acids that are either
commercially available or prepared as described in Schemes A-J. Compounds containing an
additional amino functionality were prepared by coupling the appropriate t-butyl carbamate
protected carboxylic acid by the general amide bond coupling procedure. The t-butyl carbamate
group was removed under the general t-butyl carbamate deprotection conditions described above.
The resulting amine could be substituted using the general reductive amination conditions
described above.

| Example | Structure | Characterization Data |
|---|---|---|
| 1 | 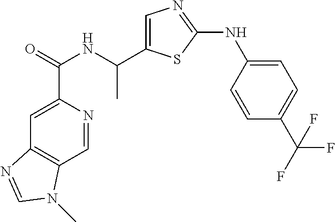 | ¹H-NMR (DMSO-D6, 500 MHz) δ 10.45 (s, 1H), 9.03 (d, J = 9 Hz, 1H), 8.99 (s, 1H), 8.51 (s, 1H), 8.28 (s, 1H), 7.77 (d, J = 8.5 Hz, 2H), 7.62 (d, J = 8.5 Hz, 2H), 7.20 (s, 1H), 5.37-5.34 (m, 1H), 3.99 (s, 3H), 1.64 (d, J = 7 Hz, 3H); LCMS m/z = 446.6 [M + 1]. |
| 2 | 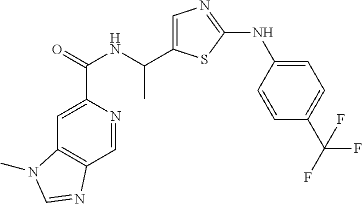 | ¹H-NMR (DMSO-D6, 500 MHz) δ 10.45 (s, 1H), 9.07 (d, J = 8.5 Hz, 1H), 8.96 (s, 1H), 8.47 (s, 1H), 8.34 (s, 1H), 7.77 (d, J = 8.5 Hz, 2H), 7.62 (d, J = 8.5 Hz, 2H), 7.20 (s, 1H), 5.37-5.34 (m, 1H), 3.94 (s, 3H), 1.63 (d, J = 7 Hz, 3H); LCMS m/z = 447 [M + 1]. |
| 3 | 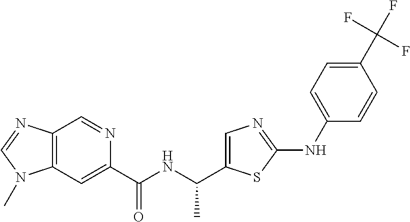 | 1H-NMR (DMSO-D6, 500 MHz) δ 10.45 (s, 1H), 9.07 (d, J = 8.5 Hz, 1H), 8.96 (s, 1H), 8.47 (s, 1H), 8.34 (s, 1H), 7.77 (d, J = 8.5 Hz, 2H), 7.62 (d, J = 8.5 Hz, 2H), 7.20 (s, 1H), 5.37-5.34 (m, 1H), 3.94 (s, 3H), 1.63 (d, J = 7 Hz, 3H); LCMS m/z = 447 [M + 1] |
| 4 | 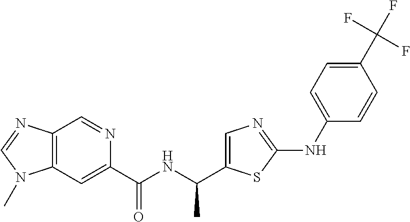 | 1H-NMR (DMSO-D6, 500 MHz) δ 10.45 (s, 1H), 9.07 (d, J = 8.5 Hz, 1H), 8.96 (s, 1H), 8.47 (s, 1H), 8.34 (s, 1H), 7.77 (d, J = 8.5 Hz, 2H), 7.62 (d, J = 8.5 Hz, 2H), 7.20 (s, 1H), 5.37-5.34 (m, 1H), 3.94 (s, 3H), 1.63 (d, J = 7 Hz, 3H); LCMS m/z = 447 [M + 1] |
| 5 | 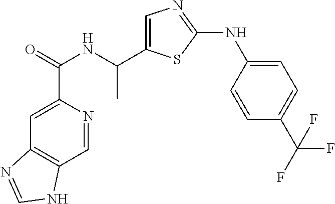 | ¹H-NMR (DMSO-D6, 500 MHz) δ 13.19 (s, 1H), 10.52 (s, 1H), 9.20-9.10 (m, 2H), 8.50 (s, 1H), 8.26 (s, 1H), 7.75 (d, J = 8 Hz, 2H), 7.68 (d, J = 8 Hz, 2H), 7.21 (s, 1H), 5.38-5.28 (m, 1H), 1.68 (d, J = 6.5 Hz, 3H); LCMS m/z = 433 [M + 1]. |
| 6 | 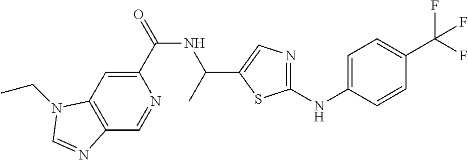 | ¹H-NMR (DMSO-D6, 500 MHz) δ 10.46 (s, 1H), 9.09 (d, J = 8.0 Hz, 1H), 8.99 (s, 1H), 8.57 (s, 1H), 8.37 (s, 1H), 7.77 (d, J = 8.0 Hz, 2H), 7.63 (d, J = 8.0 Hz, 2H), 7.21 (s, 1H), 5.39-5.36 (m, 1H), 4.42 (q, J = 8.0 Hz, 2H), 1.65 (d, J = 6.5 Hz, 3H), 1.43 (t, J = 7.5 Hz, 3H); LCMS m/z = 461 [M + 1]. |
| 7 | 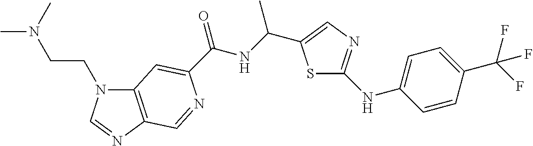 | ¹H-NMR (DMSO-D6, 500 MHz) δ 10.47 (s, 1H), 9.10 (d, J = 9.0 Hz, 1H), 8.95 (s, 1H), 8.50 (s, 1H), 8.38 (s, 1H), 7.76 (d, J = 8.5 Hz, 2H), 7.62 (d, J = 8.5 Hz, 2H), 7.20 (s, 1H), 5.39-5.36 (m, 1H), 4.46-4.45 (m, 1H), 2.62-2.60 (m, 2H), 2.15 (s, 6H), 1.63 (d, J = 7.0 Hz, 3H); LCMS m/z = 504 [M + 1]. |
| 8 | 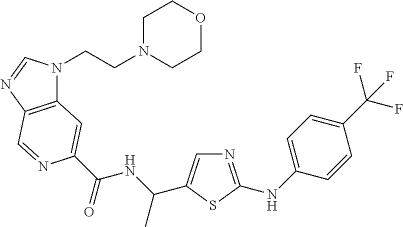 | ¹H-NMR (DMSO-D6, 500 MHz) δ 10.44 (s, 1H), 9.05 (d, J = 8.5 Hz, 1H), 8.94 (s, 1H), 8.50 (s, 1H), 8.41 (s, 1H), 7.76 (d, J = 8.5 Hz, 2H), 7.61 (d, J = 8.5 Hz, 2H), 7.20 (s, 1H), 5.39-5.36 (m, 1H), 4.49-4.46 (m, 2H), 3.50-3.49 (m, 4H), 2.68-2.66 (m, 2H), 2.41-2.40 (m, 4H), 1.63 (d, J = 7.0 Hz, 3H); LCMS m/z = 545.9 [M + 1]. |
| 9 | 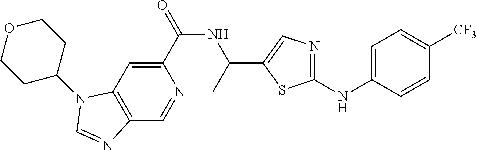 | ¹H-NMR (CDCl₃, 500 MHz) δ 9.01 (s, 1H), 8.67 (d, J = 6.0 Hz, 1H), 8.45 (s, 1H), 8.19 (s, 1H), 7.77 (d, J = 9.0 Hz, 2H), 7.63 (d, J = 9.0 Hz, 2H), 7.20 (d, J = 8.0 Hz, 1H), 5.55-5.45 (m, 1H), 4.60-4.42 (m, 1H), 4.25- 4.15 (m, 2H), 3.63-3.58 (m, 2H), 2.32-2.15 (m, 4H), 1.79 (d, J = 7 Hz, 3H); LCMS m/z = 517.1 [M + 1]. |
| 10 | 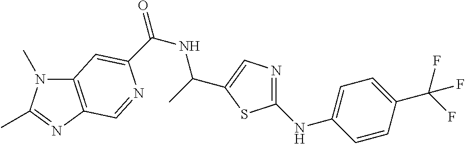 | ¹H-NMR (DMSO-D6, 500 MHz) δ 10.45 (s, 1H), 9. 0 (d, J = 9.0 Hz, 1H), 8.80 (s, 1H), 8.15 (s, 1H), 7.76 (d, J = 8.5 Hz, 2H), 7.62 (d, J = 8.5 Hz, 2H), 7.10 (s, 1H), 5.39-5.36 (m, 1H), 3.91 (s, 3H), 2.65 (s, 3H), 1.63 (d, J = 7.0 Hz, 3H); LCMS m/z = 461.1 [M + 1]. |
| 11 | 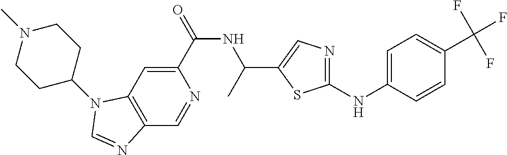 | ¹H-NMR (DMSO-D6, 500 MHz) δ 10.45 (s, 1H), 9.08 (d, J = 6.0 Hz, 1H), 8.98 (s, 1H), 8.69 (s, 1H), 8.41 (s, 1H), 7.77 (d, J = 9.0 Hz, 2H), 7.63 (d, J = 9.0 Hz, 2H), 7.21 (s, 1H), 5.39-5.36 (m, 1H), 4.59-4.53 (m, 1H), 2.93-2.91 (m, 2H), 2.25 (s, 3H), 2.17-2.16 (m, 4H), 2.02-198 (m, 2H), 1.65 (d, J = 7 Hz, 3H); LCMS m/z = 530.1 [M + 1]. |
| 12 | 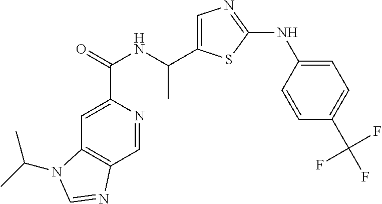 | ¹H-NMR (DMSO-D6, 500 MHz) δ 10.42 (s, 1H), 9.15 (d, J = 8.5 Hz, 1H), 8.98 (s, 1H), 8.63 (s, 1H), 8.29 (s, 1H), 7.78 (d, J = 8.5 Hz, 2H), 7.60 (d, J = 8.0 Hz, 2H), 7.20 (s, 1H), 5.40-5.35 (m, 1H), 4.95-4.85 (m, 1H), 1.65 (d, J = 7 Hz, 3H), 1.55 (d, J = 7 Hz, 6H); LCMS m/z = 475.2 [M + 1]. |
| 13 | 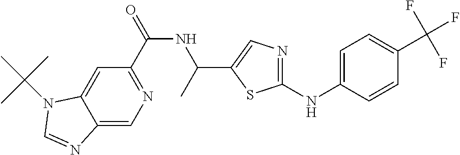 | ¹H-NMR (DMSO-D6, 500 MHz) δ 10.44 (s, 1H), 9.10 (d, J = 8.5 Hz, 1H), 8.98 (s, 1H), 8.57 (s, 1H), 8.37 (s, 1H), ), 7.76 (d, J = 9 Hz, 2H), 7.61 (d, J = 8.5 Hz, 2H), 7.20 (s, 1H), 5.37-5.34 (m, 1H), 1.72 (s, 9H), 1.63 (d, J = 6.5 Hz, 3H); LCMS m/z = 489.2 [M + 1]. |
| 14 | 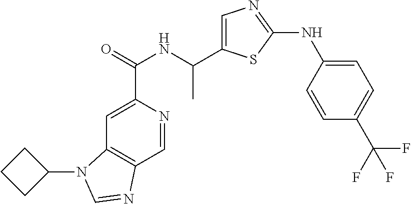 | ¹H-NMR (CD₃OD, 500 MHz) δ 8.98 (s, 1H), 8.59 (s, 1H), 8.39 (s, 1H), 7.71 (d, J = 8.5 Hz, 2H), 7.56 (d, J = 8.5 Hz, 2H), 7.23 (s, 1H), 5.50-5.46 (m, 1H), 5.14-5.11 (m, 1H), 2.72-2.65 (m, 4H), 2.12-2.05 (m, 2H), 1.74 (d, J = 7 Hz, 3H); LCMS m/z = 487.3 [M + 1]. |
| 15 | 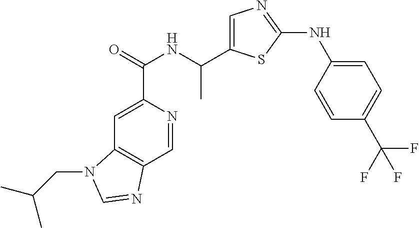 | ¹H-NMR (DMSO-D6, 500 MHz) δ 10.45 (s, 1H), 9.15 (d, J = 8.5 Hz, 1H), 8.99 (s, 1H), 8.52 (s, 1H), 8.41 (s, 1H), 7.79 (d, J = 9 Hz, 2H), 7.62 (d, J = 8.5 Hz, 2H), 7.20 (s, 1H), 5.42-5.36 (m, 1H), 4.24 (d, J = 8.5 Hz, 2H), 2.20-2.15 (m, 1H), 1.63 (d, J = 7 Hz, 3H), 0.85 (d, J = 7 Hz, 6H); LCMS m/z = 489.3 [M + 1]. |
| 16 | 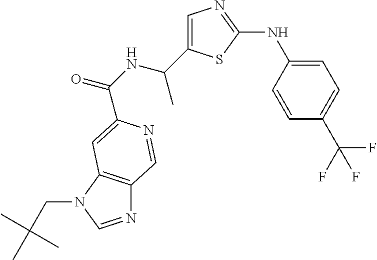 | ¹H-NMR (DMSO-D6, 500 MHz) δ 9.0 (s, 1H), 8.52 (s, 1H), 8.39 (s, 1H), 7.79 (d, J = 9 Hz, 2H), 7.65 (d, J = 9 Hz, 2H), 7.21 (s, 1H), 5.42-5.36 (m, 1H), 4.20 (s, 2H), 1.63 (d, J = 7.5 Hz, 3H), 0.98 (s, 9H); LCMS m/z = 503.7 [M + 1]. |
| 17 | 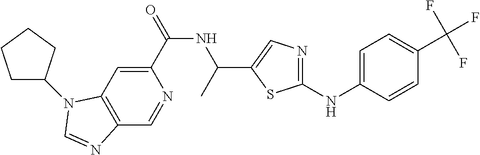 | ¹H-NMR (DMSO-D6, 500 MHz) δ 10.25 (s, 1H), 9.15 (d, J = 8.0 Hz, 1H), 9.00 (s, 1H), 8.65 (s, 1H), 8.28 (s, 1H), 7.71 (d, J = 8.5 Hz, 2H), 7.60 (d, J = 8.5 Hz, 2H), 7.20 (s, 1H), 5.42-5.35 (m, 1H), 5.14-4.98 (m, 1H), 2.26-2.20 (m, 2H), 2.0-1.92 (m, 2H), 1.85-1.82 (m, 2H), 1.69-1.65 (m, 2H), 1.63 (d, J = 7 Hz, 3H); LCMS m/z = 501.2 [M + 1]. |
| 18 | 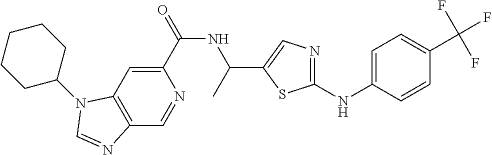 | ¹H-NMR (CD₃OD, 500 MHz) δ 9.0 (s, 1H), 8.60 (s, 1H), 7.71 (d, J = 8.5 Hz, 2H), 7.59 (d, J = 8.5 Hz, 2H), 7.23 (s, 1H), 5.42-5.35 (m, 1H), 4.48-4.42 (m, 1H), 2.23-2.19 (m, 2H), 2.14-1.98 (m, 4H), 1.98-1.95 (m, 1H), 1.83-1.80 (m, 2H), 1.67 (d, J = 6.5 Hz, 3H), 1.62-1.59 (m, 2H); LCMS m/z = 515.1 [M + 1]. |
| 19 | 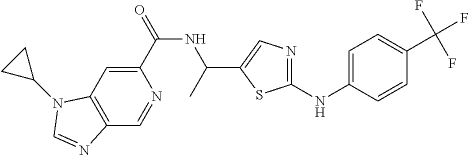 | ¹H-NMR (DMSO-D6, 500 MHz) δ 10.45 (s, 1H), 9.15 (d, J = 8.0 Hz, 1H), 8.98 (s, 1H), 8.48 (s, 1H), 8.25 (s, 1H), 7.79 (d, J = 9 Hz, 2H), 7.62 (d, J = 9 Hz, 2H), 7.21 (s, 1H), 5.42-5.36 (m, 1H), 3.65-3.61 (m, 1H), 1.63 (d, J = 7.5 Hz, 3H), 1.23-1.08 (m, 4H); LCMS m/z = 473.3 [M + 1]. |
| 20 | 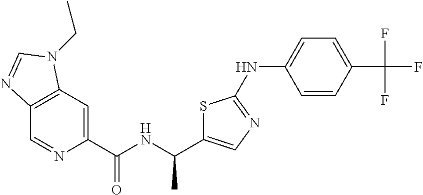 | ¹H-NMR (DMSO-D₆, 500 MHz) δ 10.42 (s, 1H), 9.04 (d, J = 8.0 Hz, 1H), 8.99 (s, 1H), 8.58 (s, 1H), 8.36 (s, 1H), 7.78 (d, J = 8.0 Hz, 2H), 7.62 (d, J = 8.0 Hz, 2H), 7.21 (s, 1H), 5.41 (q, J = 7.0 Hz, 2H), 4.45 (q, J = 6.5 Hz, 2H), 1.68 (d, J = 7.5 Hz, 3H), 1.42 (t, J = 7.0 Hz, 3H); LCMS m/z = 461.2 [M + 1]. |
| 21 | 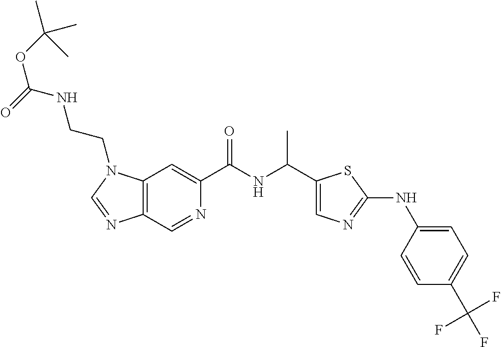 | ¹H-NMR (DMSO-D6, 500 MHz) δ 10.45 (s, 1H), 9.07 (d, J = 8.5 Hz, 1H), 8.97 (s, 1H), 8.47 (s, 1H), 8.39 (s, 1H), 7.77 (d, J = 8.5 Hz, 2H), 7.63 (d, J = 8.5 Hz, 2H), 7.21 (s, 1H), 6.99-6.97 (m, 1H), 5.39-5.36 (m, 1H), 4.38-4.35 (m, 2H), 3.25-3.22 (m, 2H), 1.65 (d, J = 7.0 Hz, 3H) 1.20 (s, 9H); LCMS m/z = 576[M + 1]. |
| 22 | 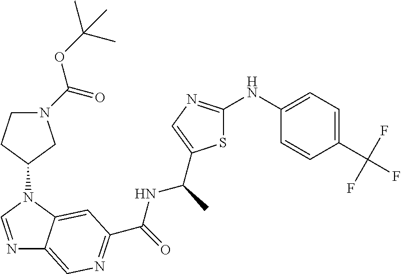 | ¹H-NMR (DMSO-D6 500 MHz) δ 8.99 (s, 1H), 8.54 (s, 1H), 8.37 (s, 1H), 7.75 (d, J = 8.5 Hz, 2H), 7.63 (d, J = 9 Hz, 2H), 7.22 (s, 1H), 5.37-5.34 (m, 2H), 3.87-3.83 (m, 2H), 3.60-3.55 (m, 3H), 2.41-2.39 (m, 1H), 1.66 (d, J = 7 Hz, 3H), 1.43 (s, 9H); LCMS m/z 602.1 [M + 1]. |
| 23 | 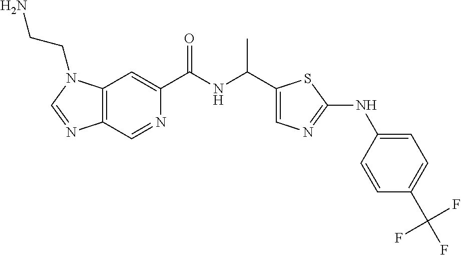 | ¹H-NMR (DMSO-D6, 500 MHz) δ 10.45 (s, 1H), 9.06 (d, J = 8.5 Hz, 1H), 8.96 (s, 1H), 8.50 (s, 1H), 8.40 (s, 1H), 7.77 (d, J = 8.5 Hz, 2H), 7.62 (d, J = 8.5 Hz, 2H), 7.21 (s, 1H), 5.39-5.36 (m, 1H), 4.33-4.31 (m, 2H), 2.94-2.92 (m, 2H), 1.65 (d, J = 7.0 Hz, 3H); LCMS m/z = 476 [M + 1]. |
| 24 | 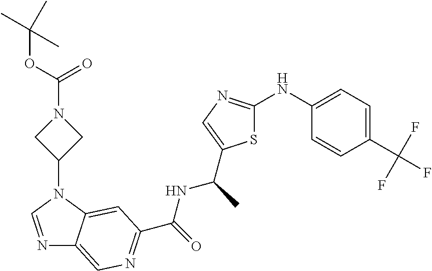 | ¹H-NMR (DMSO-D6, 500 MHz) δ 10.44 (s, 1H), 9.10 (d, J = 8.5 Hz, 1H), 9.0 (s, 1H), 8.77 (s, 1H), 8.36 (s, 1H), 7.76 (d, J = 8.5 Hz, 2H), 7.62 (d, J = 9 Hz, 2H), 7.20 (s, 1H), 5.57-5.54 (m, 1H), 5.38-5.35 (m, 1H), 4.47-4.44 (m, 2H), 4.29-4.27 (m, 2H), 1.65 (d, J = 7 Hz, 3H), 1.44 (s, 9H); LCMS m/z = 588.2 [M + 1]. |
| 25 | 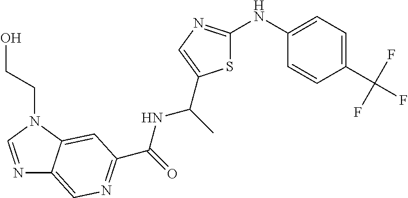 | ¹H-NMR (DMSO-D6, 500 MHz) δ 10.45 (s, 1H), 9.07 (d, J = 8.5 Hz, 1H), 8.97 (s, 1H), 8.47 (s, 1H), 8.39 (s, 1H), 7.77 (d, J = 8.5 Hz, 2H), 7.63 (d, J = 8.5 Hz, 2H), 7.21 (s, 1H), 5.39-5.36 (m, 1H), 4.99-4.98 (bs, 1H), 4.43-4.41 (m, 2H), 3.75-3.74 (m, 2H),1.65 (d, J = 7.0 Hz, 3H); LCMS m/z = 477.2 [M + 1]. |
| 26 | 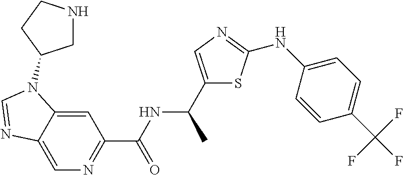 | ¹H-NMR (DMSO-D6, 500 MHz) δ 10.42 (s, 1H), 9.09 (d, J = 8.5 Hz, 2H), 8.99 (s, 1H), 8.67 (s, 1H), 8.42 (s, 1H), 7.74 (d, J = 8.5 Hz, 2H), 7.60 (d, J = 9 Hz, 2H), 7.18 (s, 1H), 5.48-5.34 (m, 2H), 3.77-3.36 (m, 5H), 2.61-2.58 (m, 1H), 1.63 (d, J = 7 Hz, 3H); LCMS m/z = 502.2 [M + 1]. |
| 27 | 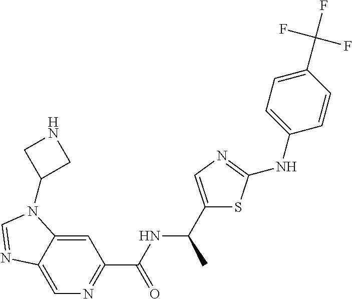 | ¹H-NMR (DMSO-D6, 500 MHz) δ 10.45 (s, 1H), 9.10 (d, J = 8.5 Hz, 1H), 9.0 (s, 1H), 8.74 (s, 1H), 8.57 (s, 1H), 7.77 (d, J = 8.5 Hz, 2H), 7.62 (d, J = 8.5 Hz, 2H), 7.21 (s, 1H), 5.56-5.54 (m, 1H), 5.39-5.36 (m, 1H), 4.13-4.09 (m, 2H), 4.01-3.97 (m, 2H), 1.65 (d, J = 7 Hz, 3H); LCMS m/z = 488 [M + 1]. |
| 28 | 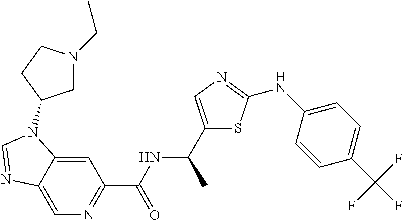 | ¹H-NMR (CD₃OD, 500 MHz) δ 8.96 (s, 1H), 8.62 (d, J = 9.5 Hz, 2H), 7.69 (d, J = 8.5 Hz, 2H), 7.54 (d, J = 8.5 Hz, 2H), 7.22 (s, 1H), 5.47-5.46 (m, 1H), 5.27-5.25 (m, 1H), 3.32-3.21 (m, 3H), 2.92-2.89 (m, 1H), 2.67-2.66 (m, 3H), 2.56-2.52 (m, 1H), 1.72 (d, J = 7 Hz, 3H), 1.21 (t, J = 7 Hz, 3H); LCMS m/z = 530 [M + 1]. |
| 29 | 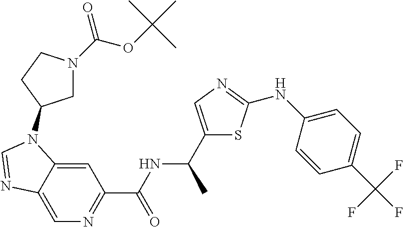 | ¹H-NMR (CD₃OD, 500 MHz) δ 8.99 (s, 1H), 8.48 (s, 1H), 8.43 (s, 1H), 7.69 (d, J = 8.5 Hz, 2H), 7.55 (d, J = 8.5 Hz, 2H), 7.22 (s, 1H), 5.48-5.46 (m, 1H), 5.35-5.33 (m, 1H), 3.99-3.60 (m, 4H), 2.59-2.48 (m, 2H), 1.73 (d, J = 7 Hz, 3H), 1.49 (s, 9H); LCMS m/z 602 [M + 1]. |
| 30 | 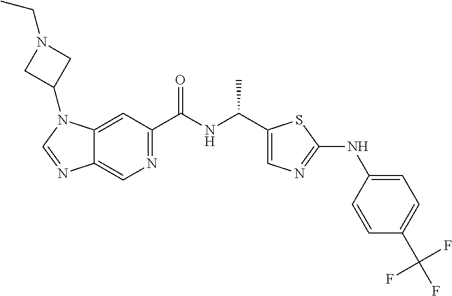 | ¹H-NMR (DMSO-D6, 500 MHz) δ 10.45 (s, 1H), 9.09 (d, J = 8.5 Hz, 1H), 9.07 (s, 1H), 8.74 (s, 1H), 8.55 (s, 1H), 7.77 (d, J = 8.5 Hz, 2H), 7.63 (d, J = 8.5 Hz, 2H), 7.21 (s, 1H), 5.38-5.29 (m, 2H), 3.76-3.73 (m, 1H), 3.49-3.48 (m, 2H), 2.57-2.54 (m, 2H), 1.65 (d, J = 7 Hz, 3H), 0.97-0.94 (m, 3H); LCMS m/z = 516 [M + 1]. |
| 31 | 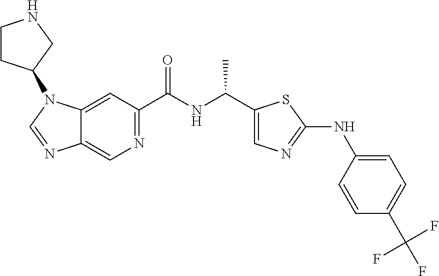 | ¹H-NMR (DMSO-D6, 500 MHz) δ 10.45 (s, 1H), 9.11 (d, J = 8.5 Hz, 1H), 9.01 (s, 1H), 8.69 (s, 1H), 8.44 (s, 1H), 7.76 (d, J = 8.5 Hz, 2H), 7.62 (d, J = 8.5 Hz, 2H), 7.20 (s, 1H), 5.48-5.46 (m, 1H), 5.37-5.36 (m, 1H), 3.88-3.57 (m, 5H), 2.61-2.60 (m, 1H), 1.64 (d, J = 6.5 Hz, 3H); LCMS m/z = 502 [M + 1]. |
| 32 | 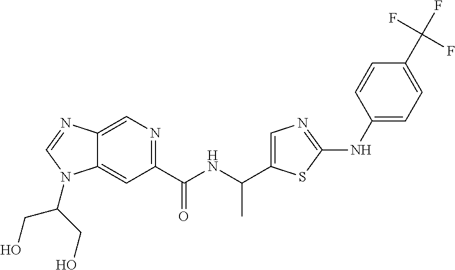 | ¹H-NMR (DMSO-D6, 500 MHz) δ 10.47 (s, 1H), 8.84 (d, J = 13 Hz, 1H), 8.27 (d, J = 12 Hz, 1H), 7.77 (d, J = 8.5 Hz, 2H), 7.63 (d, J = 9 Hz, 2H), 7.37 (d, J = 6.5 Hz, 1H), 7.18 (s, 1H),5.29-5.27 (m, 1H), 3.78 (m, 2H), 3.67 (m, 2H), 3.58 (m, 1H), 3.43- 3.42 (m, 2H), 1.63 (d, J = 7 Hz, 3H); LCMS m/z = 507 [M + 1]. |
| 33 | 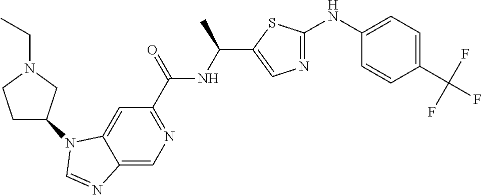 | LCMS m/z = 530 [M + 1] |
| 34 | 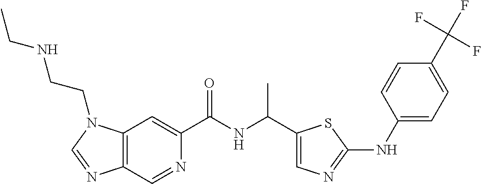 | 1H-NMR (DMSO-D6, 500 MHz) δ 8.98 (s, 1H), 8.45 (s, 1H), 8.38 (s, 1H), 7.76 (d, J = 8.5 Hz, 2H), 7.58 (d, J = 8.5 Hz, 2H), 7.20 (s, 1H), 5.38-5.37 (m, 1H), 4.41-4.39 (m, 2H), 2.98-2.96 (m, 2H), 1.63 (d, J = 7 Hz, 3H), 0.98-0.96 (m, 3H); LCMS m/z = 504 [M + 1]. |
| 35 | 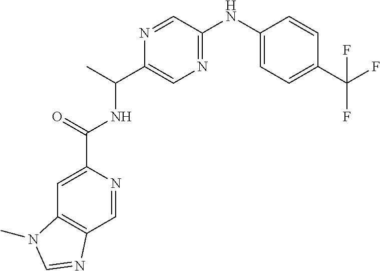 | ¹H-NMR (DMSO-D₆, 500 MHz) δ 9.91 (s, 1H), 9.03 (d, J = 8.5 Hz, 1H), 8.99 (s, 1H), 8.47 (s, 1H), 8.33 (s, 1H), 8.30 (s, 2H), 7.88 (d, J = 8.5 Hz, 2H), 7.62 (d, J = 8.5 Hz, 2H), 5.27 (q, J = 7.5 Hz, 1H), 3.93 (s, 3H), 1.54 (d, J = 7.0 Hz, 3H); LCMS m/z = 441.8 [M + 1]. |
| 38 | 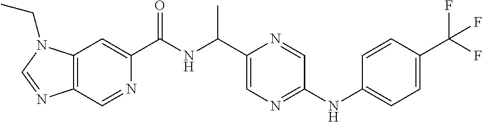 | 1H NMR (DMSO-D6, 500 MHz) δ 9.91 (s, 1H), 9.04 (d, J = 8.0 Hz, 1H), 8.99 (s, 1H), 8.56 (s, 1H), 8.35 (s, 1H), 8.30 (s, 2H), 7.88 (d, J = 8.5 Hz, 2H), 7.62 (d, J = 8.5 Hz, 2H), 5.27 (q, J = 7.5 Hz, 2H), 4.40 (q, J = 6.5 Hz, 2H), 1.54 (d, J = 7.0 Hz, 3H), 1.41 (t, J = 7.5 Hz, 3H); LCMS m/z = 456.1 [M + 1]. |
| 39 | 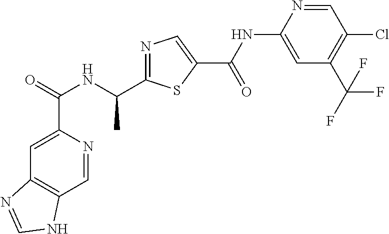 | 1H-NMR (DMSO-D6, 500 MHz) δ 11.71 (s, 1H), 9.51 (d, J = 8.5 Hz, 1H), 9.12 (s, 1H), 8.77 (d, J = 8.5 Hz, 2H), 8.57 (d, J = 8.5 Hz, 2H), 8.30 (s, 1H), 5.45-5.42 (m, 1H), 1.75 (d, J = 6.5 Hz, 3H); LCMS m/z = 495.7 [M + 1]. |
| 40 | 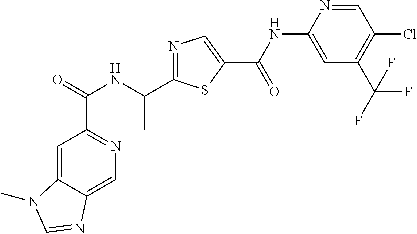 | 1H NMR (400 MHz, CDCl₃) d 9.13 (d, J = 0.8 Hz, 1H), 8.90 (d, J = 8.1 Hz, 1H), 8.67 (s, 1H), 8.49 (d, J = 0.8 Hz, 1H), 8.46 (s, 1H), 8.41 (s, 1H), 8.37 (s, 1H), 5.72-5.61 (m, 1H), 4.04 (s, 3H), 1.85 (d, J = 6.9 Hz, 3H); LCMS m/z = 510 (M + 1). |
| 41 | 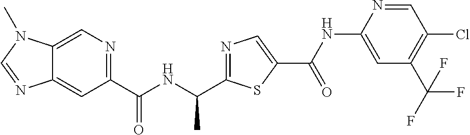 | 1H-NMR (DMSO-D6, 500 MHz) δ 11.71 (s, 1H), 9.50 (d, J = 8 Hz, 1H), 9.05 (s, 1H), 8.75 (d, J = 10.5 Hz, 2H), 8.53 (s, 2H), 8.30 (s, 1H), 5.48-5.45 (m, 1H), 4.01 (s, 3H), 1.70 (d, J = 7 Hz, 3H); LCMS m/z = 510 [M + 1]. |
| 42 | 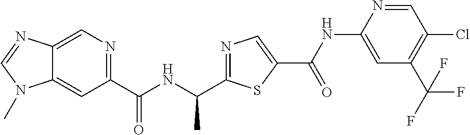 | 1H-NMR (DMSO-D6, 500 MHz) δ 11.71 (s, 1H), 9.52 (d, J = 8 Hz, 1H), 9.02 (s, 1H), 8.75 (d, J = 8.5 Hz, 2H), 8.52 (s, 2H), 8.49 (s, 1H), 8.37 (s, 1H), 5.47-5.45 (m, 1H), 3.94 (s, 3H), 1.70 (d, J = 7 Hz, 3H); LCMS m/z = 509.9 [M + 1]. |
| 43 | 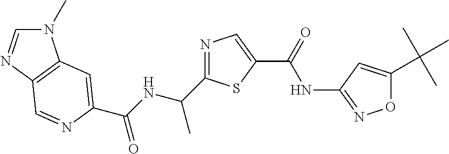 | 1H-NMR (CD3OD, 500 MHz) δ 9.03 (s, 1H), 8.47 (s, 1H), 8.42 (d, J = 8.5 Hz, 2H), 6.62 (s, 1H), 5.61-5.59 (m, 1H), 4.03 (s, 3H), 1.82 (d, J = 7 Hz, 3H), 1.37 (s, 9H); LCMS m/z = 454.1 [M + 1]. |
| 44 | 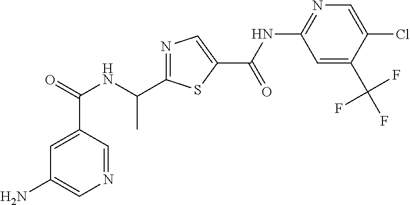 | LCMS m/z = 471 [M + 1] |
| 45 | 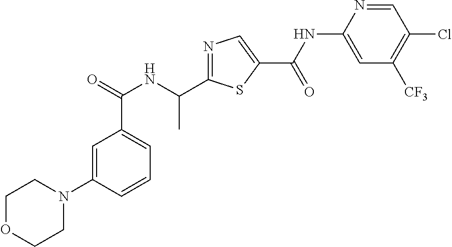 | 1H NMR (DMSO-d6, 400 MHz,) δ 11.83 (s, 1H), 9.26 (d, J = 8.0 Hz, 1H), 8.78 (s, 1H), 8.74 (s, 1H), 8.56 (s, 1H), 7.43 (s, 1H), 7.41-7.32 (m, 2H), 7.23-7.11 (m, 1H), 5.42 (m, 1H), 3.85-3.71 (m, 4H), 3.24-3.11 (m, 4H), 1.69-1.57 (d, J = 8.0 Hz, 3H); LCMS m/z = 540 [M + 1] |
| 46 | 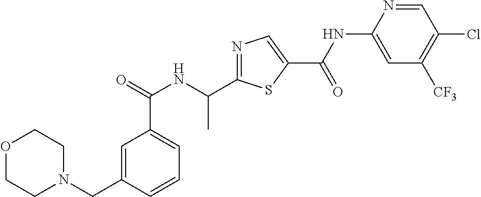 | 1H NMR (DMSO-d6, 400 MHz) δ 11.76 (s, 1H), 9.36 (d, J = 7.6 Hz, 1H), 8.79 (s, 1H), 8.76 (s, 1H), 8.55 (s, 1H), 8.00-8.08 (m, 2H), 7.80-7.53 (m, 2H), 5.45 (m, 1H), 4.43 (br. s., 2H), 3.96 (m, 2H), 3.66 (m, 2H), 3.32-3.00 (m, 4H), 1.66 (d, J = 8.0 Hz, 3H); LCMS m/z = 554 [M + 1] |
| 47 | 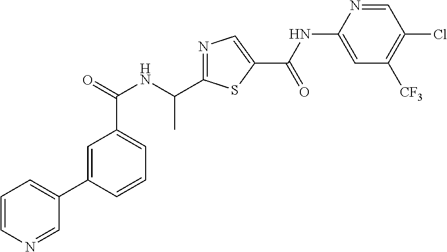 | 1H NMR (DMSO-d6, 400 MHz,) δ 11.75 (s, 1H), 9.39 (d, J = 7.6 Hz, 1H), 9.09 (br. s., 1H), 8.83-8.64 (m, 3H), 8.56 (s, 1H), 8.45-8.33 (m, 1H), 8.29 (s, 1H), 8.00 (d, J = 7.6 Hz, 2H), 7.78-7.62 (m, 2H), 5.48 (m, 1H), 1.68 (d, J = 8.0 Hz, 3H); LCMS m/z = 532 [M + 1] |
| 48 | 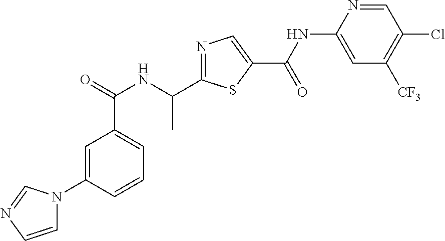 | 1H NMR (DMSO-d6, 400 MHz) δ 11.76 (s, 1H), 9.44 (brs, 1H), 9.43 (d, J = 7.6 Hz, 1H), 8.79 (s, 1H), 8.77 (s, 1H), 8.55 (s, 1H), 8.27 (br. s., 2H), 8.09 (d, J = 8.1 Hz, 1H), 8.00 (d, J = 8.1 Hz, 1H), 7.88-7.73 (m, 2H), 5.48 (m, 1H), 1.68 (d, J = 8.0 Hz, 3H); LCMS m/z = 521 [M + 1] |
| 49 | 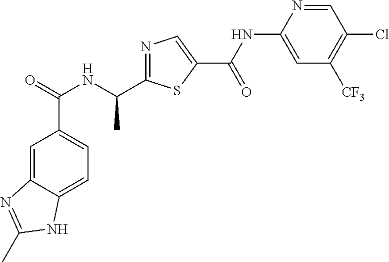 | 1H NMR (DMSO-d6, 400 MHz) δ 11.75 (s, 1H), 9.51 (brs, 1H), 8.78 (s, 1H), 8.77 (s, 1H), 8.76 (s, 1H), 8.55 (s, 1H), 8.23 (br. s., 1H), 7.97 (br. s., 1H), 7.78 (br. s., 1H), 5.46 (m, 1H), 2.73 (br. s., 3H), 1.67 (d, J = 8.0 Hz, 3H); LCMS m/z = 509 [M + 1] |
| 50 | 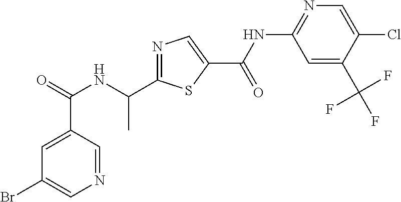 | LCMS m/z = 536 [M + 2] |
| 51 | 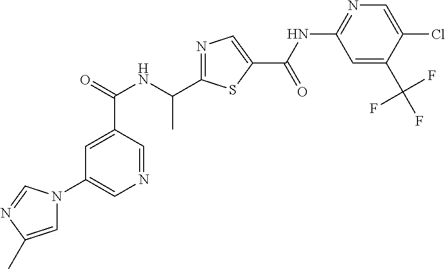 | LCMS m/z = 536 [M + 1] |
| 52 | 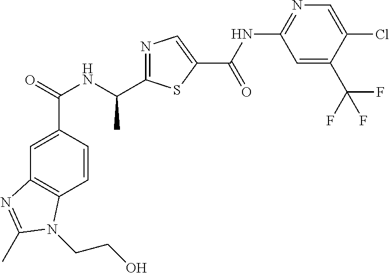 | 1H NMR (400 MHz, DMSO-d6) δ = 11.73 (s, 1H), 9.18 (d, J = 7.6 Hz, 1H), 8.77 (s, 1H), 8.74 (s, 1H), 8.55 (s, 1H), 8.18 (s, 1H), 7.80 (d, J = 8.6 Hz, 1H), 7.58 (d, J = 8.6 Hz, 1H), 5.52-5.37 (m, 1H), 4.96 (t, J = 5.3 Hz, 1H), 4.28 (t, J = 5.1 Hz, 2H), 3.72 (q, J = 5.1 Hz, 2H), 2.59 (s, 3H), 1.66 (d, J = 7.1 Hz, 3H); LCMS m/z = 553.2 [M + 1]. |
| 53 | 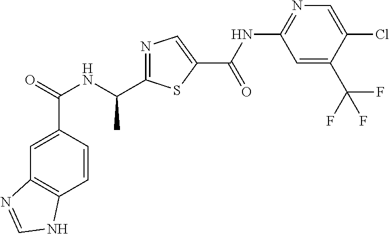 | 1H NMR (400 MHz, DMSO-d6) δ = 11.74 (s, 1H), 9.27 (d, J = 7.6 Hz, 1H), 8.77 (s, 1H), 8.75 (s, 1H), 8.55 (s, 1H), 8.51 (br. s., 1H), 8.27 (s, 1H), 7.85 (d, J = 8.1 Hz, 1H), 7.70 (d, J = 8.6 Hz, 1H), 5.53-5.38 (m, 1H), 1.66 (d, J = 7.1 Hz, 3H); LCMS m/z = 495.1 [M + 1]. |
| 54 | 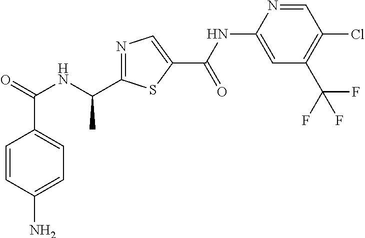 | LCMS m/z = 470 [M + 1] |
| 55 | 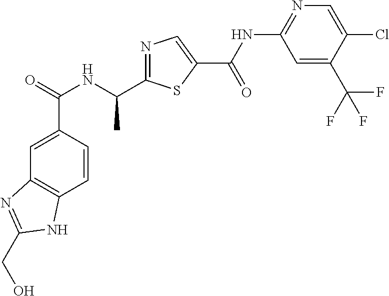 | 1H NMR (400 MHz, DMSO-d6) δ = 12.75- 12.51 (m, 1H), 11.74 (s, 1H), 9.21 (dd, J = 7.3, 20.0 Hz, 1H), 8.77 (s, 1H), 8.74 (s, 1H), 8.55 (s, 1H), 8.21 (s, 1H), 8.05 (s, 1H), 7.83-7.71 (m, 1H), 7.62 (d, J = 8.6 Hz, 1H), 7.52 (d, J = 8.6 Hz, 1H), 5.77 (dt, J = 5.7, 11.9 Hz, 1H), 5.45 (quin, J = 7.2 Hz, 1H), 4.73 (d, J = 5.1 Hz, 2H), 1.66 (d, J = 7.1 Hz, 3H); LCMS m/z = 525.1 [M + 1]. |
| 56 | 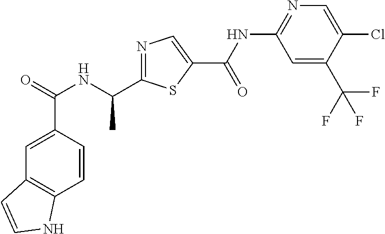 | 1H NMR (400 MHz, DMSO-d6) δ = 11.73 (s, 1H), 11.37 (br. s., 1H), 9.09 (d, J = 7.6 Hz, 1H), 8.77 (s, 1H), 8.74 (s, 1H), 8.55 (s, 1H), 8.23 (s, 1H), 7.70 (d, J = 8.6 Hz, 1H), 7.52-7.40 (m, 2H), 6.57 (br. s., 1H), 5.44 (t, J = 7.1 Hz, 1H), 1.65 (d, J = 7.1 Hz, 3H); LCMS m/z = 494.1 [M + 1]. |
| 57 | 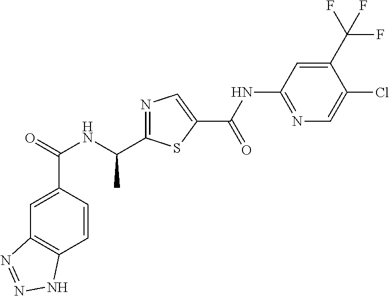 | 1H NMR (400 MHz, DMSO-d6) δ = 11.75 (s, 1H), 9.44 (d, J = 7.6 Hz, 1H), 8.78 (s, 1H), 8.75 (s, 1H), 8.66-8.47 (m, 2H), 8.00 (br. s., 2H), 5.47 (quin, J = 7.1 Hz, 1H), 1.68 (d, J = 7.1 Hz, 3H); LCMS m/z = 496.2 [M + 1]. |
| 58 | 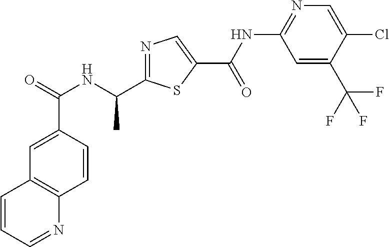 | 1H NMR (400 MHz, DMSO-d6) δ = 11.76 (s, 1H), 9.53 (d, J = 7.6 Hz, 1H), 9.04 (d, J = 2.0 Hz, 1H), 8.77 (d, J = 5.6 Hz, 2H), 8.62 (s, 1H), 8.61-8.51 (m, 2H), 8.25 (d, J = 8.6 Hz, 1H), 8.15 (d, J = 8.6 Hz, 1H), 7.68 (d, J = 4.0 Hz, 1H), 5.50 (quin, J = 7.1 Hz, 1H), 1.69 (d, J = 7.1 Hz, 3H); LCMS m/z = 506.2 [M + 1]. |
| 59 | 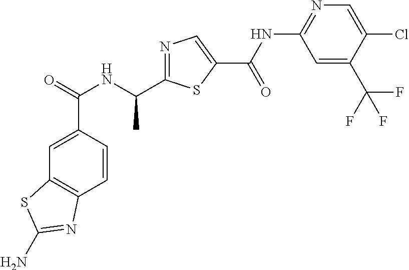 | 1H NMR (400 MHz, DMSO-d6) δ = 11.73 (s, 1H), 9.12 (d, J = 7.6 Hz, 1H), 8.77 (s, 1H), 8.76-8.70 (m, 1H), 8.56 (s, 1H), 8.26 (s, 1H), 7.95 (br. s., 2H), 7.83 (d, J = 8.6 Hz, 1H), 7.40 (d, J = 8.6 Hz, 1H), 5.42 (quin, J = 7.2 Hz, 1H), 1.64 (d, J = 7.1 Hz, 3H); LCMS m/z = 527.0 [M + 1]. |
| 60 | 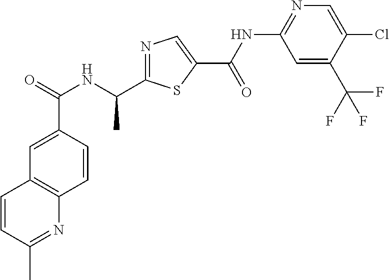 | 1H NMR (400 MHz, DMSO-d6) δ = 11.76 (s, 1H), 9.54 (d, J = 7.6 Hz, 1H), 8.78 (s, 1H), 8.77 (s, 1H), 8.64 (s, 2H), 8.56 (s, 1H), 8.29 (d, J = 8.6 Hz, 1H), 8.10 (d, J = 8.6 Hz, 1H), 7.69 (d, J = 8.6 Hz, 1H), 5.49 (quin, J = 7.2 Hz, 1H), 2.79 (s, 3H), 1.69 (d, J = 7.1 Hz, 3H); LCMS m/z = 520.2 [M + 1]. |
| 61 | 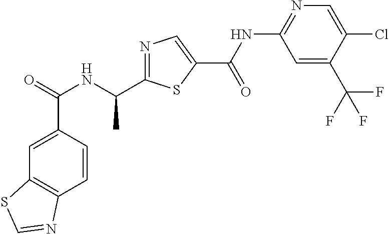 | 1H NMR (400 MHz, DMSO-d6) δ = 11.75 (s, 1H), 9.57 (s, 1H), 9.41 (d, J = 7.6 Hz, 1H), 8.78 (s, 1H), 8.75 (s, 2H), 8.56 (s, 1H), 8.21 (d, J = 8.6 Hz, 1H), 8.08 (d, J = 8.6 Hz, 1H), 5.47 (quin, J = 7.1 Hz, 1H), 1.67 (d, J = 7.1 Hz, 3H); LCMS m/z = 512.2 [M + 1]. |
| 62 | 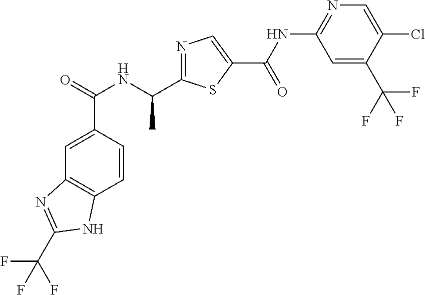 | 1H NMR (400 MHz, DMSO-d6) δ = 11.75 (s, 1H), 9.38 (d, J = 7.1 Hz, 1H), 8.78 (s, 1H), 8.75 (s, 1H), 8.56 (s, 1H), 8.35 (br. s., 1H), 7.98 (d, J = 8.6 Hz, 1H), 7.82 (d, J = 7.6 Hz, 1H), 5.47 (quin, J = 7.2 Hz, 1H), 1.67 (d, J = 7.1 Hz, 3H); LCMS m/z = 563.2 [M + 1]. |
| 63 | 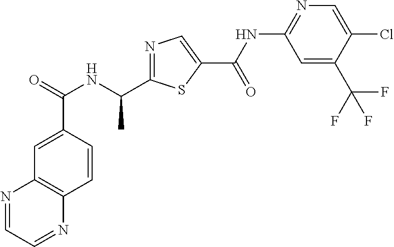 | 1H NMR (400 MHz, DMSO-d6) δ = 11.76 (s, 1H), 9.68 (d, J = 7.6 Hz, 1H), 9.07 (d, J = 4.0 Hz, 2H), 8.78 (s, 1H), 8.76 (s, 1H), 8.72 (s, 1H), 8.56 (s, 1H), 8.35-8.30 (m, 1H), 8.23 (d, J = 8.6 Hz, 1H), 5.51 (quin, J = 7.1 Hz, 1H), 1.70 (d, J = 6.6 Hz, 3H); LCMS m/z = 507.1 [M + 1]. |
| 64 | 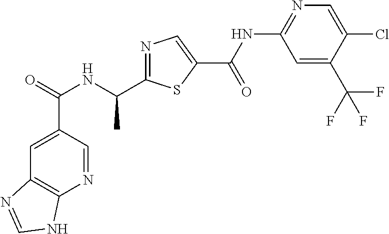 | 1H NMR (300 MHz, DMSO-d6) δ = 11.74 (s, 1H), 9.42 (d, J = 7.6 Hz, 1H), 8.95 (d, J = 1.9 Hz, 1H), 8.77 (s, 1H), 8.75 (s, 1H), 8.69 (s, 1H), 8.57 (d, J = 1.9 Hz, 1H), 8.55 (s, 1H), 5.48 (t, J = 7.2 Hz, 1H), 1.68 (d, J = 7.2 Hz, 3H); LCMS m/z = 594.1 [M + 1]. |
| 65 | 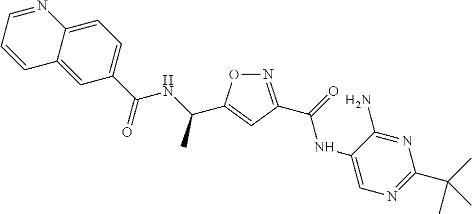 | 1H NMR (400 MHz, DMSO-d6) δ = 9.97 (s, 1H), 9.32 (d, J = 7.6 Hz, 1H), 9.00 (d, J = 2.5 Hz, 1H), 8.58 (s, 1H), 8.51 (d, J = 7.6 Hz, 1H), 8.28-8.17 (m, 1H), 8.11 (d, J = 9.1 Hz, 1H), 8.04 (s, 1H), 7.63 (dd, J = 4.3, 8.3 Hz, 1H), 6.84 (s, 1H), 6.69 (br. s., 2H), 5.50 (quin, J = 7.2 Hz, 1H), 1.64 (d, J = 7.1 Hz, 3H), 1.28 (s, 9H); LCMS m/z = 460.3 [M + 1]. |
| 66 | 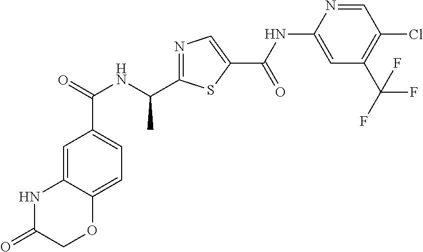 | 1H-NMR (DMSO-D6, 500 MHz) δ 11.79 (s, 1H), 10.90 (s, 1H), 9.20 (d, J = 8.5 Hz, 1H), 8.80 (s, 1H), 8.79 (s, 1H), 8.59 (s, 1H), 7.49 (d, J = 8.5 Hz, 1H), 7.43 (s, 1H), 7.02 (d, J = 8.5 Hz, 1H), 5.43-5.40 (m, 1H), 4.60 (s, 2H), 1.62 (d, J = 7 Hz, 3H); LCMS m/z = 525.7 [M + 1]. |
| 67 | 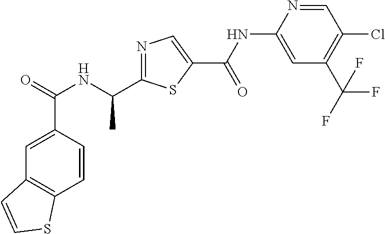 | 1H-NMR (CD3OD, 500 MHz) δ 8.62 (s, 1H), 8.59 (s, 1H), 8.50 (s, 1H), 8.42 (s, 1H), 8.05 (d, J = 8.5 Hz, 1H), 7.90 (d, J = 8.5 Hz, 1H), 7.71 (d, J = 8.5 Hz, 1H), 7.50 (d, J = 7.5 Hz, 1H), 5.59-5.56 (m, 1H), 1.78 (d, J = 7 Hz, 3H); LCMS m/z = 510.6 [M + 1]. |
| 68 | 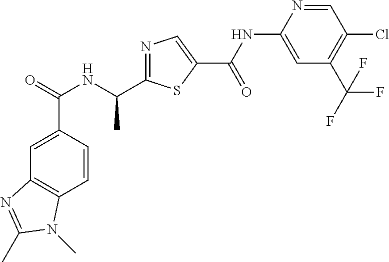 | 1H-NMR (CD3OD, 500 MHz) δ 8.63 (s, 1H), 8.59 (s, 1H), 8.50 (s, 1H), 8.21 (s, 1H), 7.91 (d, J = 8.5 Hz, 1H), 7.59 (d, J = 8.5 Hz, 1H), 5.59-5.56 (m, 1H), 3.90 (s, 3H), 2.62 (s, 3H), 1.78 (d, J = 7 Hz, 3H); LCMS m/z = 522.9 [M + 1]. |
| 69 | 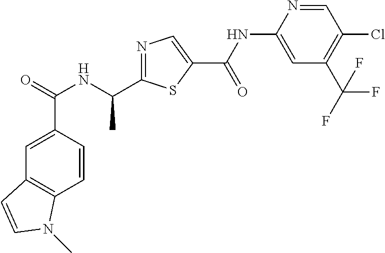 | 1H-NMR (CD3OD, 500 MHz) δ 8.63 (s, 1H), 8.59 (s, 1H), 8.50 (s, 1H), 8.21 (s, 1H), 7.79 (d, J = 8.5 Hz, 1H), 7.51 (d, J = 8.5 Hz, 1H), 7.31 (s, 1H), 6.61 (s, 1H), 5.59-5.56 (m, 1H), 3.84 (s, 3H), 1.78 (d, J = 7 Hz, 3H); LCMS m/z = 507.7 [M + 1]. |
| 70 | 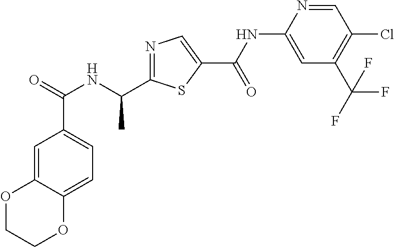 | 1H-NMR (DMSO-D6, 500 MHz) δ 11.79 (s, 1H), 9.10 (s, 1H), 8.79 (s, 1H), 8.77 (s, 1H), 8.51 (s, 1H), 7.52 (d, J = 8.5 Hz, 2H), 6.99 (d, J = 8.5 Hz, 1H), 5.39-5.37 (m, 1H), 4.25-4.22 (m, 4H), 1.62 (d, J = 7 Hz, 3H); LCMS m/z = 512.7 [M + 1]. |
| 71 | 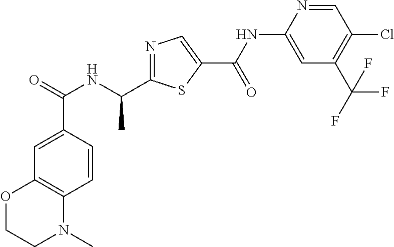 | ¹H-NMR (CD₃OD, 500 MHz) δ 8.61 (s, 1H), 8.59 (s, 1H), 8.56 (s, 1H), 7.50 (d, J = 9.0 Hz, 1H), 7.35 (s, 1H), 6.79 (d, J = 8.5 Hz, 1H), 5.50-5.48 (m, 1H), 4.28-4.27 (m, 2H), 3.40-3.38 (m, 2H), 3.17 (s, 3H), 1.78 (d, J = 7 Hz, 3H); LCMS m/z = 525.7 [M + 1]. |
| 72 | 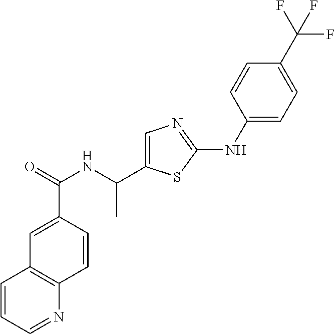 | 1H-NMR (DMSO-D6, 500 MHz): δ 10.44 (s, 1H), 9.16 (d, J = 9.0 Hz, 1H), 8.91 (s, 1H), 8.57 (s, 1H), 8.55 (d, J = 8 Hz, 1H), 8.18 (d, J = 8 Hz, 1H), 8.16-8.14 (m, 1H), 7.89-7.87 (m, 2H), 7.62-7.60 (m, 3H), 7.22 (s, 1H), 5.39-5.36 (m, 1H), 1.63 (d, J = 6 Hz, 3H); LCMS m/z = 442.7 [M + 1]. |
| 73 | 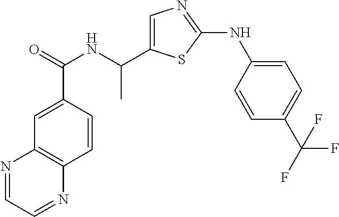 | 1H-NMR (DMSO-D6, 500 MHz): δ 10.48 (s, 1H), 9.31 (d, J = 6.5 Hz, 1H), 9.03 (s, 1H), 9.02 (s, 1H), 8.63 (s, 1H), 8.28 (d, J = 8 Hz, 1H), 8.18 (d, J = 8.5 Hz, 1H), 7.77 (d, J = 7.5 Hz, 2H), 7.62 (d, J = 7.5 Hz, 2H), 7.23 (s, 1H), 5.37-5.34 (m, 1H), 1.63 (d, J = 6 Hz, 3H); LCMS m/z = 444 [M + 1]. |
| 74 | 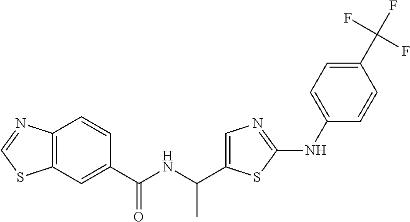 | ¹H NMR (400 MHz, DMSO-d₆) δ = 10.49 (s, 1H), 9.54 (s, 1H), 9.05 (d, J = 8.0 Hz, 1H), 8.68 (d, J = 1.3 Hz, 1H), 8.16 (d, J = 8.5 Hz, 1H), 8.03 (dd, J = 1.8, 8.5 Hz, 1H), 7.78 (d, J = 8.5 Hz, 2H), 7.63 (d, J = 8.8 Hz, 2H), 7.23 (d, J = 1.0 Hz, 1H), 5.47- 5.29 (m, 1H), 1.61 (d, J = 6.8 Hz, 3H); LCMS m/z = 449 [M + 1] |
| 75 | 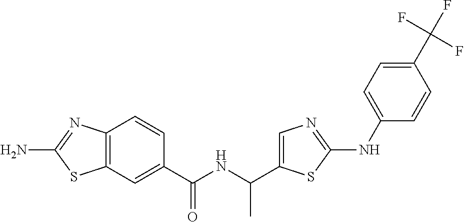 | ¹H NMR (400 MHz, DMSO-d₆) δ = 10.48 (br. s., 1H), 8.80 (d, J = 8.0 Hz, 1H), 8.35 (br. s., 2H), 8.24 (d, J = 1.5 Hz, 1H), 7.86- 7.71 (m, 3H), 7.63 (d, J = 8.8 Hz, 2H), 7.40 (d, J = 8.3 Hz, 1H), 7.20 (d, J = 1.0 Hz, 1H), 5.42-5.21 (m, 1H), 1.58 (d, 3H); LCMS m/z = 464 [M + 1] |
| 76 | 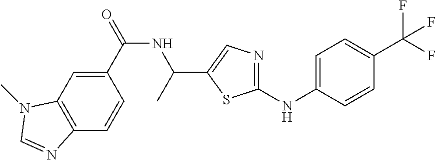 | 1H-NMR (DMSO-D6, 500 MHz): δ 10.45 (s, 1H), 8.86 (d, J = 8.5 Hz, 1H), 8.31 (s, 1H), 8.14 (s, 1H), 7.78 (d, J = 8.5 Hz, 2H), 7.70 (s, 1H), 7.69 (d, J = 8.0 Hz, 1H), 7.63 (d, J = 8.0 Hz, 2H), 7.21 (s, 1H), 5.42-5.35 (m, 1H), 3.88 (s, 3H), 1.62 (d, J = 6.5 Hz, 3H); LCMS m/z = 446 [M + 1]. |
| 77 | 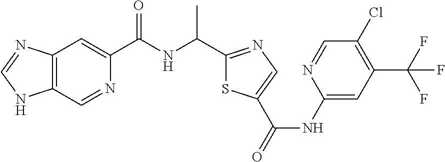 | 1H-NMR (DMSO-D6, 500 MHz) δ 13.23- 13.20 (bs, 1N—H), 11.72-11.70 (bs, 1N—H), 9.51 (s, 1H), 9.01 (s, 1H), 8.74 (d, J = 8.5 Hz, 2H), 8.50 (d, J = 8.5 Hz, 2H), 8.30 (s, 1H), 5.45-5.42 (m, 1H), 1.65 (d, J = 6.5 Hz, 3H); LCMS m/z = 495.8 [M + 1]. |
| 78 | 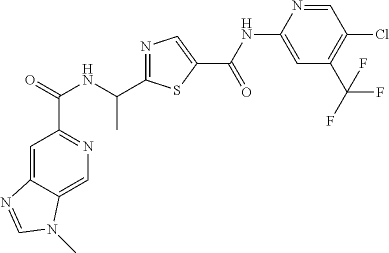 | 1H-NMR (DMSO-D6, 500 MHz) δ 11.71 (bs, 1N—H), 9.59-9.50 (m, 1H), 9.05 (s, 1H), 9.03 (s, 1H), 8.79 (d, J = 8.5 Hz, 2H), 8.59 (s, 1H), 8.58 (s, 1H), 8.40 (s, 1H), 5.45-5.42 (m, 1H), 4.10 (s, 1H), 3.99 (s, 1H), 1.75 (d, J = 6.5 Hz, 3H); LCMS m/z = 509.8 [M + 1]. |
| 79 | 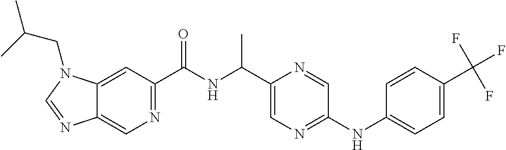 | ¹H-NMR (CDCl₃, 200 MHz) δ 9.09 (s, 1H), 9.08 (d, J = 7.5 Hz, 1H), 8.38 (s, 1H), 8.34 (s, 2H), 8.07 (s, 1H), 7.60 (dd, J = 7.5 Hz, 4H), 6.90 (bs, 1N—H), 5.44-5.42 (m, 1H), 4.08 (d, J = 7.5 Hz, 2H) 2.29-2.24 (m, 1H), 1.67 (d, J = 7 Hz, 3H), 0.98 (d, J = 7 Hz, 6H); LCMS m/z = 484.2 [M + 1]. |
| 80 | 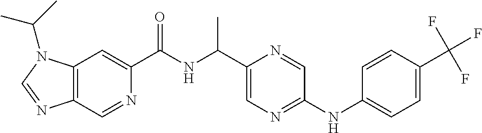 | ¹H-NMR (DMSO-D6, 500 MHz) δ 9.94 (s, 1H), 9.08 (d, J = 7.5 Hz, 1H), 9.01 (s, 1H), 8.69 (s, 1H), 8.38 (s, 1H), 8.38 (s, 2H), 7.89 (d, J = 8.5 Hz, 2H), 7.64 (d, J = 8.5 Hz, 2H), 5.37-5.34 (m, 1H), 4.97-4.96 (m, 1H), 1.58 (d, J = 7 Hz, 9H); LCMS m/z = 470 [M + 1]. |
| 81 | 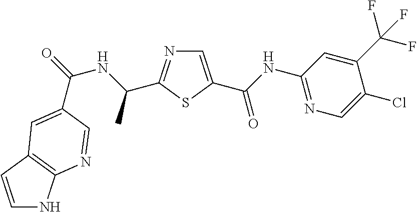 | 1H NMR (400 MHz, MeOD) δ 8.78 (d, J = 2.01 Hz, 1H), 8.61 (s, 1H), 8.54-8.60 (m, 2H), 8.52 (s, 1H), 7.51 (d, J = 3.50 Hz, 1H), 6.65 (d, J = 3.50 Hz, 1H), 5.50-5.68 (m, 1H), 1.77 (d, J = 7.03 Hz, 3H); LCMS m/z = 495.2 [M + 1]. |
| 82 | 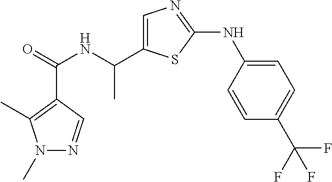 | 1H-NMR (DMSO-D6, 500 MHz): δ 10.48 (s, 1H), 8.25 (d, J = 9.0 Hz, 1H), 7.92 (s, 1H), 7.79 (d, J = 7.0 Hz, 2H), 7.61 (d, J = 7.0 Hz, 2H), 5.22-5.19 (m, 1H), 3.62 (s, 3H), 2.50 (s, 3H), 1.63 (d, J = 6 Hz, 3H); LCMS m/z = 410 [M + 1]. |
| 83 | 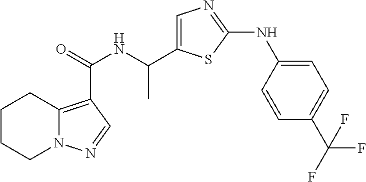 | 1H-NMR (DMSO-D6, 500 MHz): δ 10.44 (s, 1H), 8.26 (d, J = 8.0 Hz, 1H), 7.91 (s, 1H), 7.80 (d, J = 8 Hz, 2H), 7.61 (d, J = 8 Hz, 2H), 7.15 (s, 1H), 5.22-5.19 (m, 1H), 4.04-4.0 (m, 2H), 2.97-2.94 (m, 2H), 1.92- 1.91 (m, 2H), 1.76-1.75 (m, 2H), 1.63 (d, J = 6 Hz, 3H); LCMS m/z = 436 [M + 1]. |
| 84 | 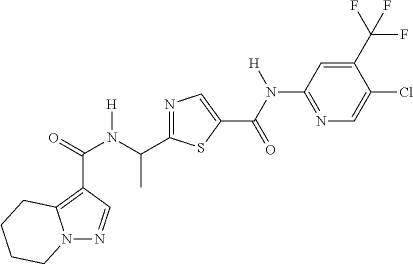 | 1H NMR (400 MHz, MeOD) δ 8.62 (s, 1H), 8.58 (s, 1H), 8.51 (s, 1H), 8.02 (s, 1H), 5.41-5.53 (m, 1H), 4.13-4.21 (m, 2H), 3.05-3.15 (m, 2H), 2.05-2.13 (m, 2H), 1.91 (m, 2H), 1.71 (d, J = 7.20 Hz, 3H); LCMS m/z = 499.2 [M + 1]. |
| 85 | 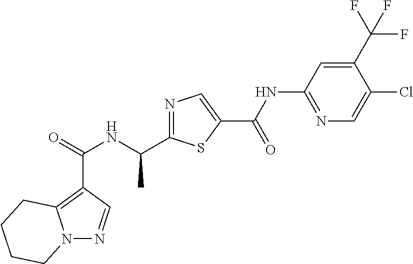 | LCMS m/z = 499 [M + 1] |
| 86 | 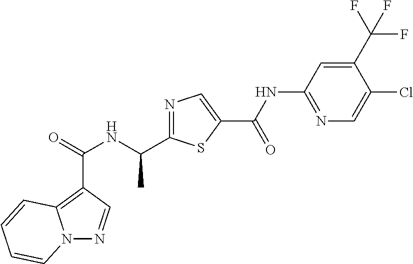 | LCMS m/z = 495 [M + 1] |
| 87 | 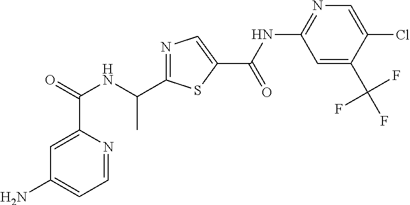 | LCMS m/z = 471 [M + 1] |
| 88 | 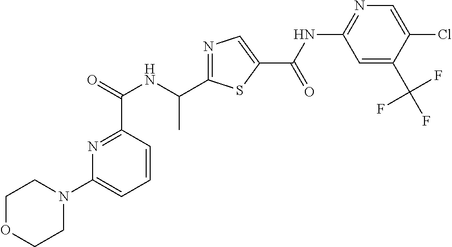 | LCMS m/z = 541 [M + 1] |
| 89 | 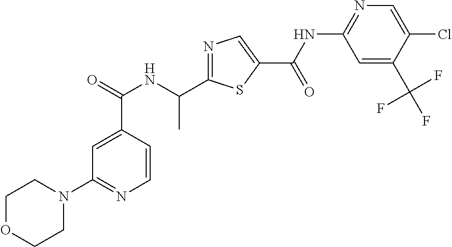 | LCMS m/z = 541 [M + 1] |
| 90 | 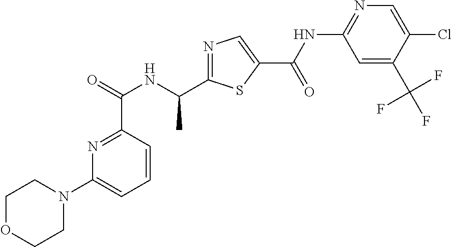 | LCMS m/z = 541 [M + 1] |
| 91 | 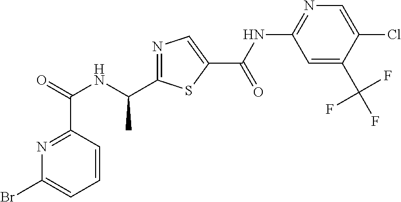 | LCMS m/z = 536 [M + 2] |
| 92 | 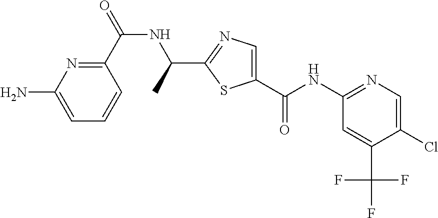 | LCMS m/z = 471 [M + 1] |
| 93 | 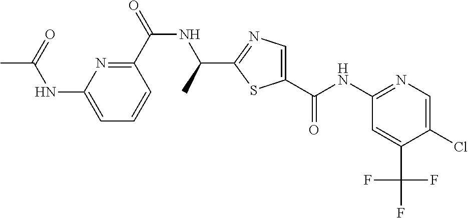 | LCMS m/z = 513 [M + 1] |
| 94 | 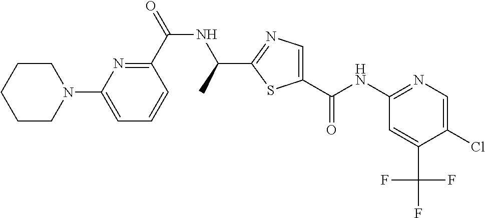 | LCMS m/z = 539 [M + 1] |
| 95 | 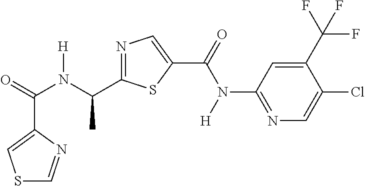 | 1H NMR (400 MHz, MeOD) δ 9.05 (d, J = 2.02 Hz, 1H), 8.60 (s, 1H), 8.56 (s, 1H), 8.50 (s, 1H), 8.33 (d, J = 2.02 Hz, 1H), 5.54 (q, J = 7.07 Hz, 1H), 1.76 (d, J = 7.07 Hz, 3H); LCMS m/z = 462.1 [M + 1]. |
| 96 | 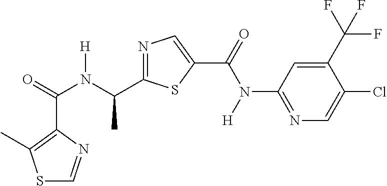 | 1H NMR (400 MHz, MeOD) δ 9.00 (s, 1H), 8.62 (s, 1H), 8.57 (s, 1H), 8.51 (s, 1H), 5.49 (q, J = 7.07 Hz, 1H), 2.68 (s, 3H), 1.72 (d, J = 7.07 Hz, 3H); LCMS m/z = 476.1 [M + 1]. |
| 97 | 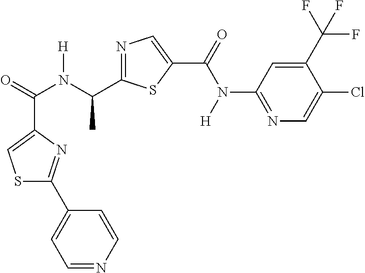 | 1H NMR (400 MHz, MeOD) δ 8.57-8.62 (m, 2H), 8.50 (s, 1H), 8.46 (s, 1H), 8.42 (s, 1H), 8.36 (s, 1H), 8.00-8.03 (m, 2H), 5.49 (q, J = 7.07 Hz, 1H), 1.71 (d, J = 7.07 Hz, 3H); LCMS m/z = 539.1 [M + 1]. |
| 98 | 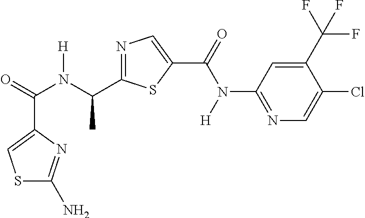 | 1H NMR (400 MHz, MeOD) δ 8.61 (s, 1H), 8.57 (s, 1H), 8.50 (s, 1H), 7.43 (s, 1H), 5.40-5.50 (m, 1H), 1.71 (d, J = 7.07 Hz, 3H); LCMS m/z = 477.1 [M + 1]. |
| 99 | 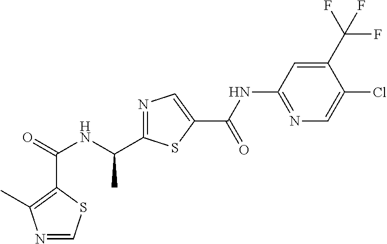 | LCMS m/z = 476 |
| 100 | 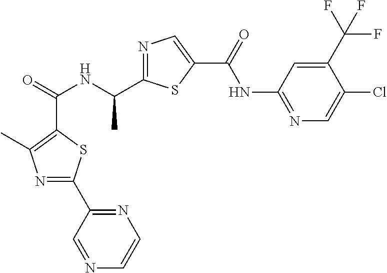 | LCMS m/z = 554 |
| 101 | 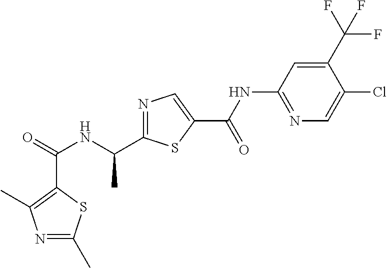 | LCMS m/z = 490 |
| 102 | 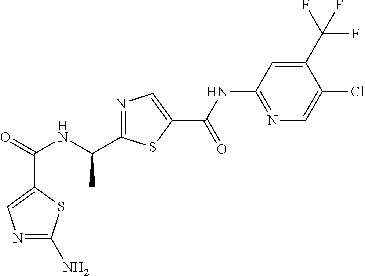 | LCMS m/z = 477 |
| 103 | 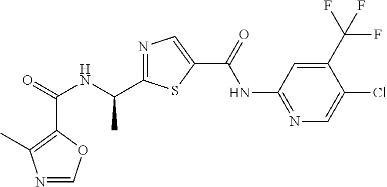 | LCMS m/z = 460 |
| 104 | 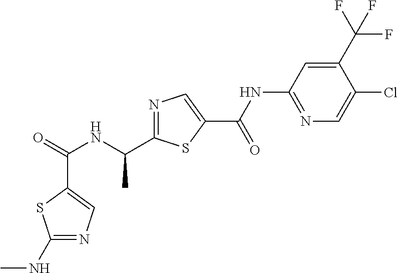 | LCMS m/z = 491 |
| 105 | 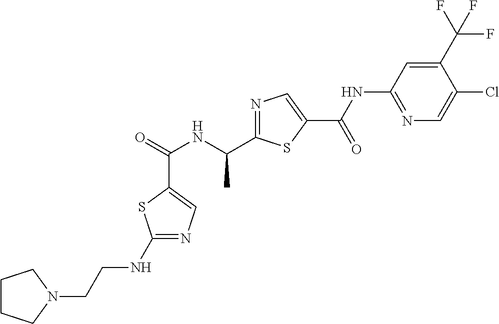 | LCMS m/z = 574 |
| 106 | 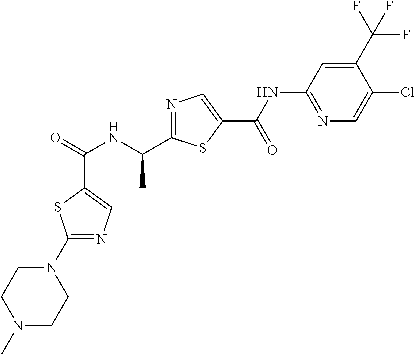 | LCMS m/z = 560 |
| 107 | 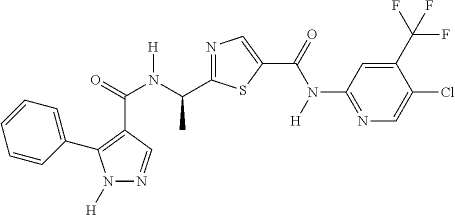 | 1H NMR (400 MHz, DMSO-d6) δ 11.73 (s, 1H), 8.84 (d, J = 7.58, 1H), 8.78 (s, 1H), 8.73 (s, 1H), 8.57 (s, 1H), 8.30 (d, J = 1.60 Hz, 1H), 8.06 (d, J = 1.64 Hz, 1H), 7.65- 7.80 (m, 2H), 7.27-7.49 (m, 3H), 5.25- 5.41 (m, 1H), 1.59 (d, J = 7.0 Hz, 3H); LCMS m/z = 521.2 [M + 1]. |
| 108 | 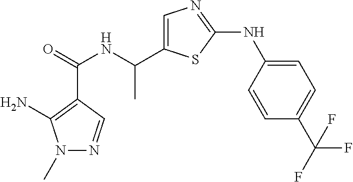 | 1H-NMR (DMSO-D6, 500 MHz): δ 10.50 (s, 1H), 8.10 (d, J = 9.0 Hz, 1H), 7.79 (d, J = 7.5 Hz, 2H), 7.62 (s, 1H), 7.60 (d, J = 7.5 Hz, 2H), 7.15 (s, 1H), 6.19 (s, 2H), 5.22- 5.19 (m, 1H), 3.62 (s, 3H), 1.63 (d, J = 6 Hz, 3H); m/z = 411 [M + 1]. |
| 109 | 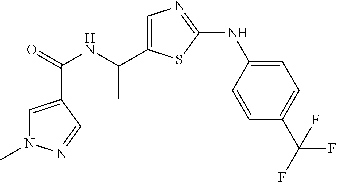 | 1H-NMR (DMSO-D6, 500 MHz): δ 10.42 (s, 1H), 9.42 (d, J = 8 Hz, 1H), 8.17 (s, 1H), 7.85 (s, 1H), 7.79 (d, J = 7.5 Hz, 2H), 7.63 (d, J = 7.5 Hz, 2H), 7.18 (s, 1H), 5.29- 5.20 (m, 1H), 3.82 (s, 3H), 1.58 (d, J = 6 Hz, 3H); m/z 395.9 [M + 1]. |
| 110 | 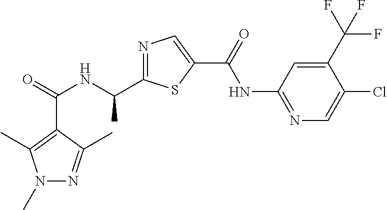 | LCMS m/z = 487 |
| 111 | 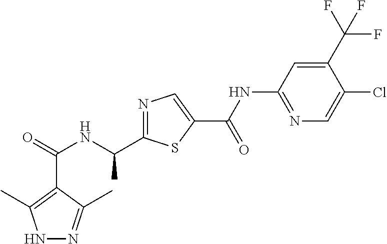 | LCMS m/z = 473 |
| 112 | 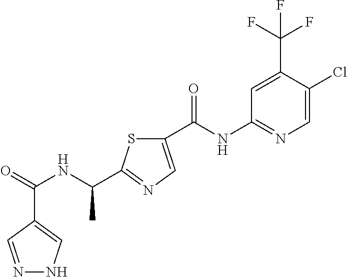 | LCMS m/z = 445 |
| 113 | 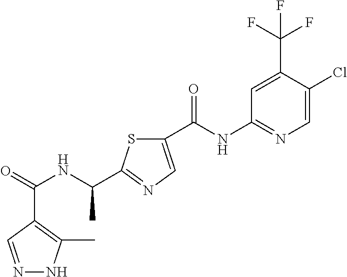 | LCMS m/z = 459 |
| 114 | 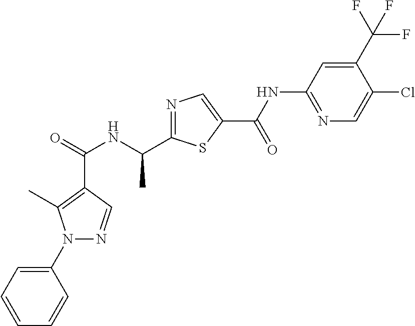 | LCMS m/z = 535 |
| 115 | 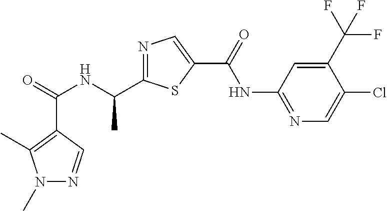 | LCMS m/z = 473 |
| 116 | 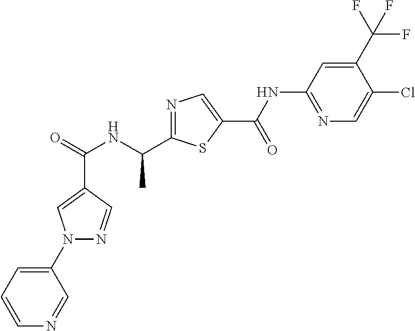 | LCMS m/z = 522 |
| 117 | 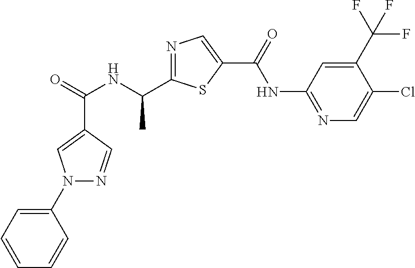 | LCMS m/z = 521 |
| 118 | 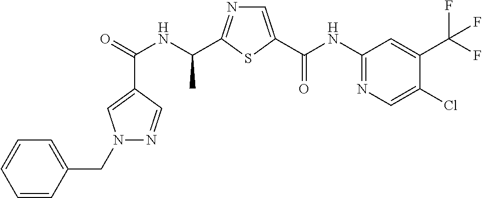 | LCMS m/z = 535 |
| 119 | 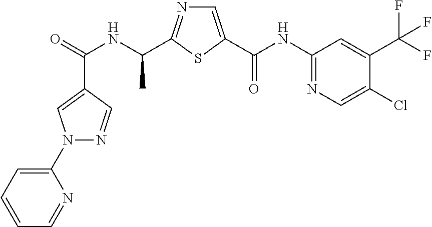 | LCMS m/z = 522 |
| 120 | 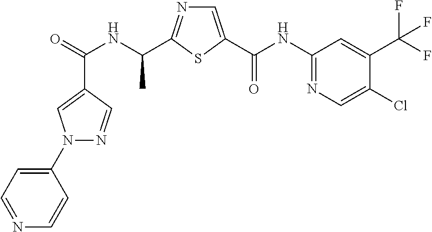 | LCMS m/z = 520 |
| 121 | 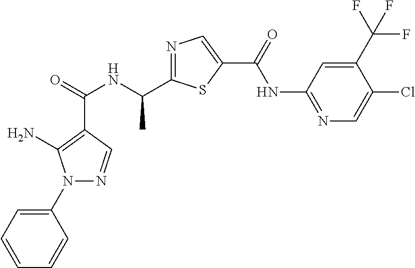 | LCMS m/z = 536 |
| 122 | 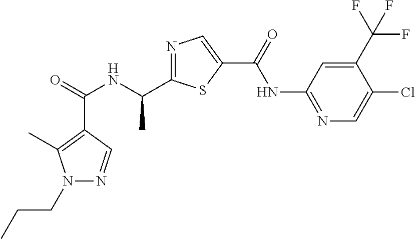 | LCMS m/z = 501 |
| 123 | 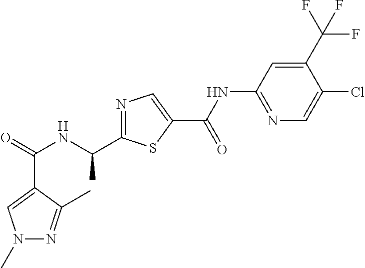 | LCMS m/z = 473 |
| 124 | 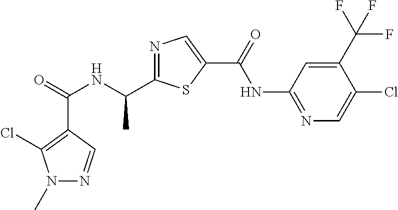 | LCMS m/z = 494 |
| 125 | 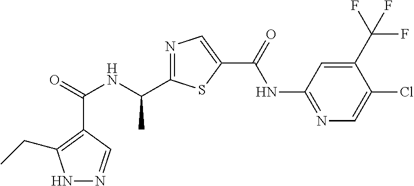 | LCMS m/z = 473 |
| 126 | 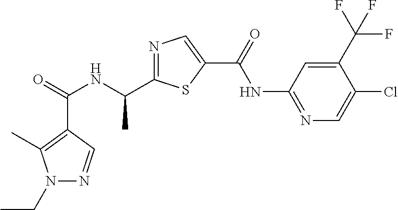 | LCMS m/z = 487 |
| 127 | 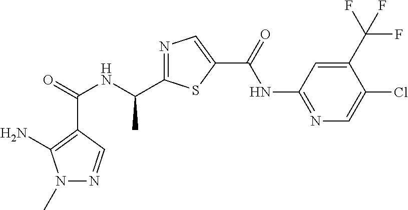 | LCMS m/z = 474 |
| 134 | 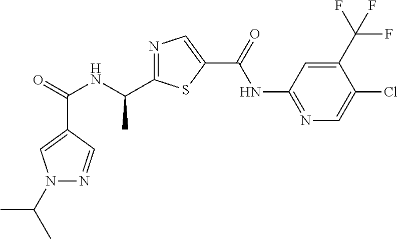 | LCMS m/z = 489 |
| 135 | 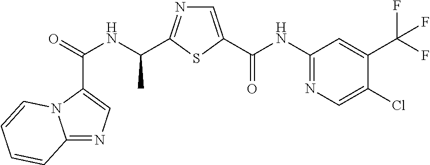 | LCMS m/z = 495 |
| 136 | 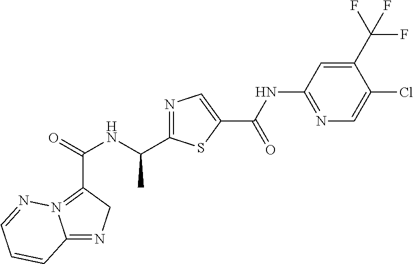 | LCMS m/z = 496 |
| 137 | 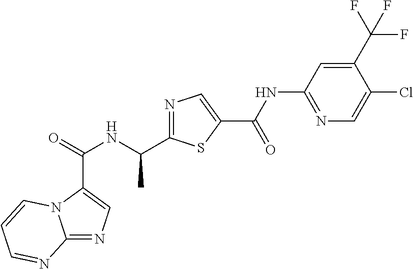 | LCMS m/z = 496 |
| 138 | 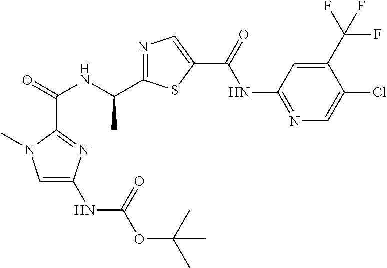 | ¹H NMR (400 MHz, MeOD) δ 8.61 (s, 1H), 8.56 (s, 1H), 8.49 (s, 1H), 7.32 (s, 1H), 5.43 (m, 1H), 3.66 (s, 3H), 1.71 (d, J = 7.03 Hz, 3H), 1.44-1.55 (m, 9H); LCMS m/z = 574.2 [M + 1]. |
| 139 | 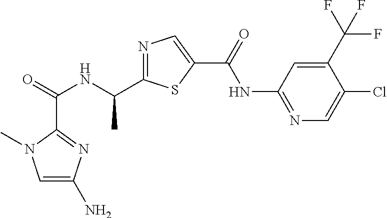 | LCMS m/z = 474.2 [M + 1]. |
| 140 | 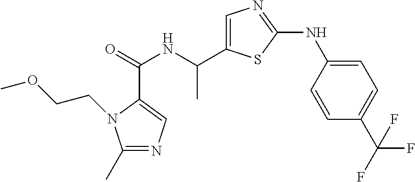 | ¹H-NMR (DMSO-D6, 500 MHz): δ 10.46 (s, 1H), 8.57 (d, J = 9.0 Hz, 1H), 7.77 (d, J = 8.5 Hz, 2H), 7.62 (d, J = 8.5 Hz, 2H), 7.48 (s, 1H), 7.15 (s, 1H), 5.22-5.20 (m, 1H), 4.39-4.37 (m, 2H), 3.55-3.53 (m, 2H), 3.17 (s, 3H), 2.31 (s, 3H), 1.53 (d, J = 6 Hz, 3H); LCMS m/z = 453.9 [M + 1]. |
| 141 | 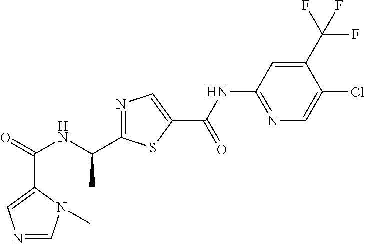 | LCMS m/z = 459 |
| 142 | 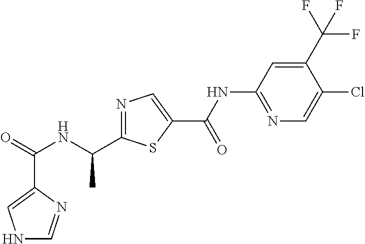 | LCMS m/z = 445 |
| 143 | 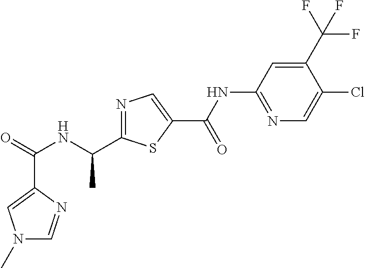 | LCMS m/z = 459 |
| 144 | 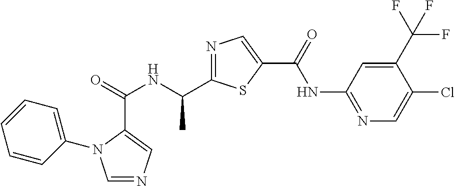 | LCMS m/z = 521 |
| 145 | 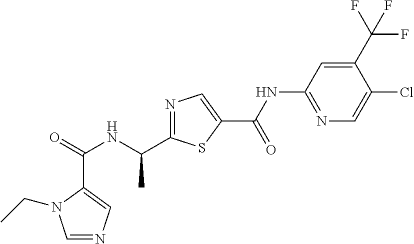 | LCMS m/z = 473 |
| 146 | 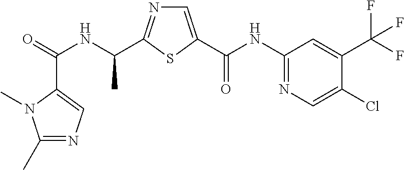 | LCMS m/z = 473 |
| 147 | 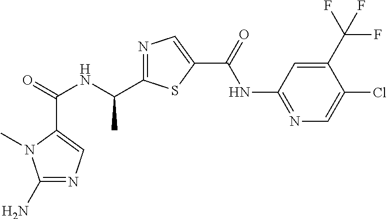 | LCMS m/z = 474 |
| 148 | 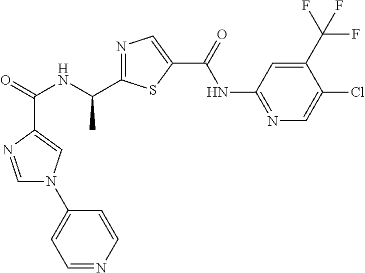 | LCMS m/z = 522 |
| 149 | 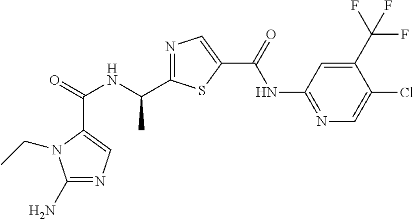 | LCMS m/z = 488 |
| 150 | 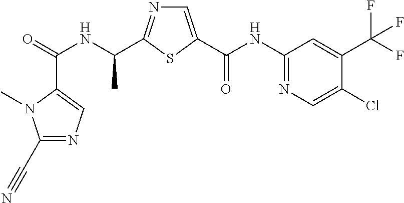 | LCMS m/z = 484 |
| 151 | 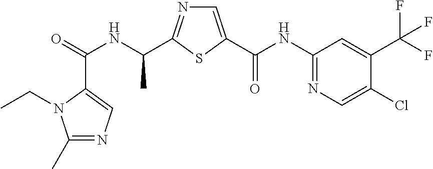 | LCMS m/z = 487 |
| 152 | 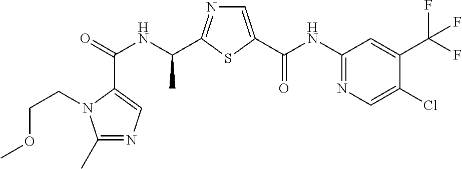 | LCMS m/z = 517 |
| 153 | 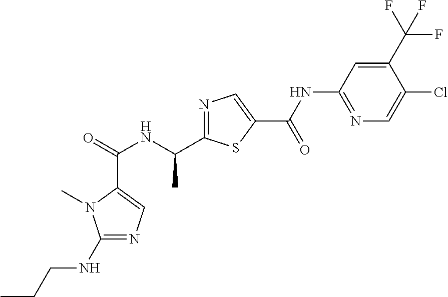 | LCMS m/z = 516 |
| 154 | 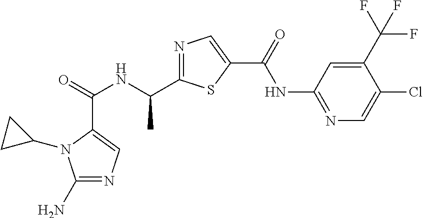 | LCMS m/z = 500 |
| 155 | 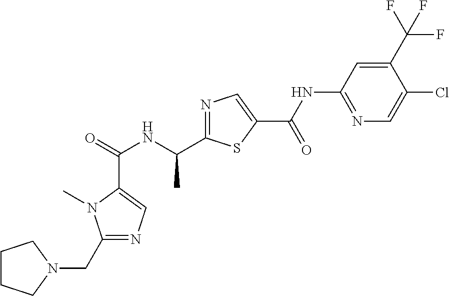 | LCMS m/z = 542 |
| 156 | 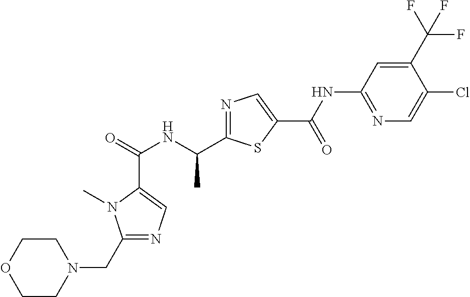 | LCMS m/z = 558 |
| 157 | 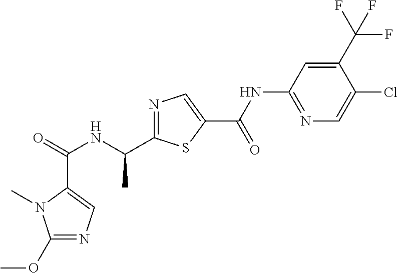 | LCMS m/z = 489 |
| 158 | 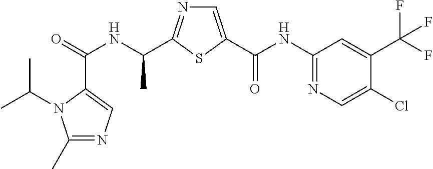 | LCMS m/z = 501 |
| 159 | 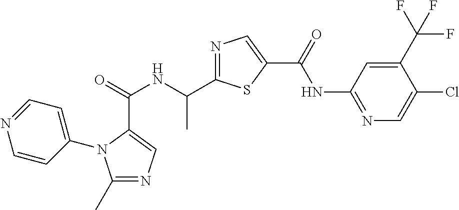 | LCMS m/z = 536 |
| 160 | 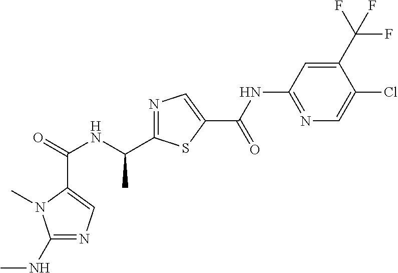 | LCMS m/z = 488 |
| 161 | 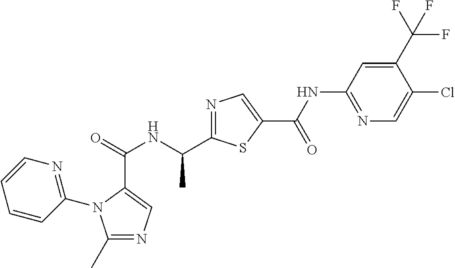 | LCMS m/z = 536 |
| 162 | 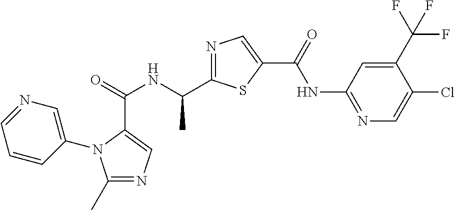 | LCMS m/z = 536 |
| 163 | 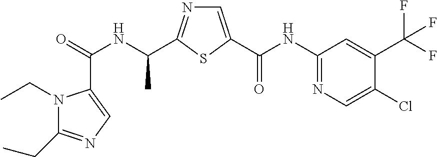 | LCMS m/z = 501 |
| 164 | 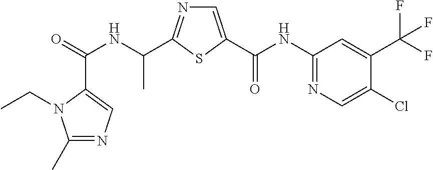 | LCMS m/z = 487 |
| 165 | 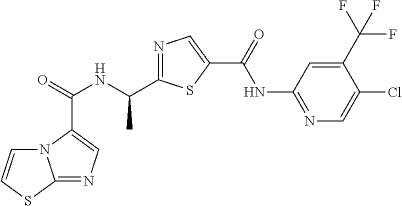 | LCMS m/z = 501 |
| 166 | 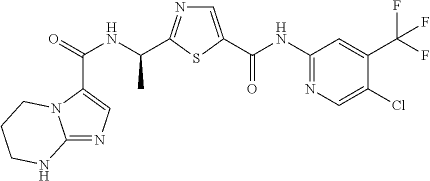 | LCMS m/z = 500 |
| 167 | 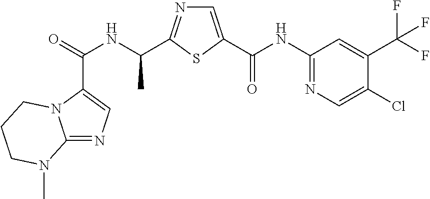 | LCMS m/z = 514 |
| 168 | 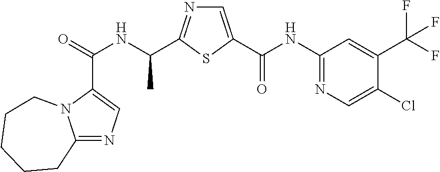 | LCMS m/z = 513 |
| 169 | 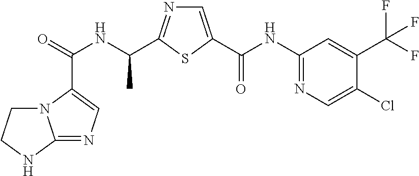 | LCMS m/z = 486 |
| 170 | 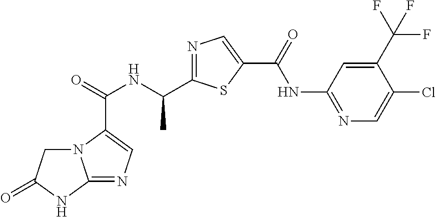 | LCMS m/z = 500 |
| 171 | 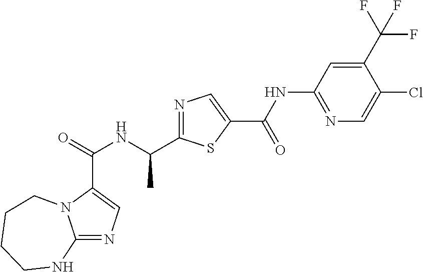 | LCMS m/z = 514 |
| 172 | 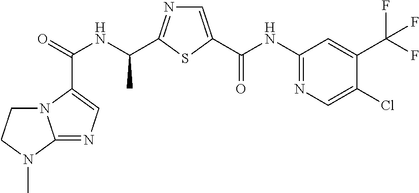 | LCMS m/z = 500 |
| 173 | 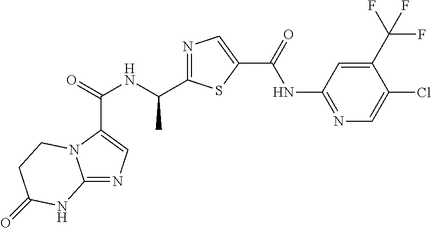 | LCMS m/z = 514 |
| 174 | 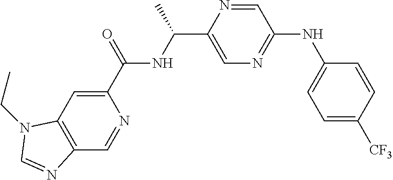 | ¹H NMR (CD₃OD, 500 MHz) δ 9.0 (s, 1H), 8.51 (s, 1H), 8.39 (s, 1H), 8.25 (s, 1H), 7.85 (d, J = 8.5 Hz, 2H), 7.59 (d, J = 8.5 Hz, 2H), 5.38-5.37 (m, 1H), 4.42 (q, J = 8.5 Hz, 2H), 1.73 (d, J = 7 Hz, 3H), 1.58 (t, J = 8 Hz, 3H); LCMS m/z = 456 [M + 1]. |
| 175 | 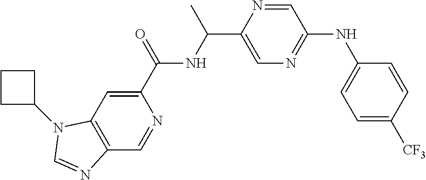 | ¹H NMR (DMSO-D₆, 500 MHz) δ 9.98 (s, 1H), 9.12 (d, J = 8.0 Hz, 1H), 9.06 (s, 1H), 8.32 (s, 3H), 7.88 (d, J = 8.5 Hz, 2H), 7.64 (d, J = 8.5 Hz, 2H), 5.28-5.25 (m, 1H), 5.18-5.15 (m, 1H), 2.55 (bs, 4H), 1.90 (m, 2H), 1.57 (t, J = 7.5 Hz, 3H); LCMS m/z = 481.9 [M + 1]. |
| 176 | 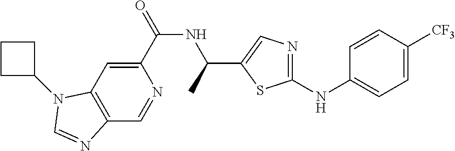 | ¹H-NMR (DMSO-D₆, 500 MHz) δ 10.45 (s, 1H), 9.09 (d, J = 7.0 Hz, 1H), 8.98 (s, 1H), 8.73 (s, 1H), 8.31 (s, 1H), 7.78 (d, J = 8.5 Hz, 2H), 7.63 (d, J = 8.5 Hz, 2H), 7.21 (s, 1H), 5.38-5.36 (m, 1H), 5.19-5.17 (m, 1H), 2.54 (bs, 4H), 1.92-1.89 (m, 2H), 1.63 (d, J = 7.0 Hz, 3H); LCMS m/z = 487.1 [M + 1]. |
| 177 | 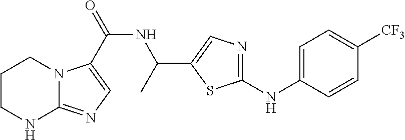 | ¹H-NMR (CD₃OD, 200 MHz) δ 7.71 (d, J = 7 Hz, 2H), 7.55 (d, J = 7 Hz, 2H), 7.32 (bs, 1H), 7.13 (s, 1H), 5.32-5.29 (m, 2H),4.28-4.25 (m, 2H), 2.09-2.07 (m, 2H), 1.63 (d, J = 7 Hz, 3H); LCMS m/z = 437 [M + 1]. |
| 178 | 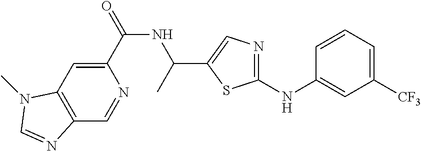 | ¹H-NMR (DMSO-D₆, 500 MHz) δ 10.38 (s, 1H), 9.15 (d, J = 7.0 Hz, 1H), 8.98 (s, 1H), 8.52 (s, 1H), 8. 39 (s, 1H), 8.19 (s, 1H), 7.72 (d, J = 7.0 Hz, 1H), 7.50-7.48 (m, 1H), 7.22 (s, 2H), 5.41-5.38 (m, 1H), 3.98 (s, 3H), 1.63 (d, J = 7.0 Hz, 3H); LCMS m/z = 447.1 [M + 1]. |
| 179 | 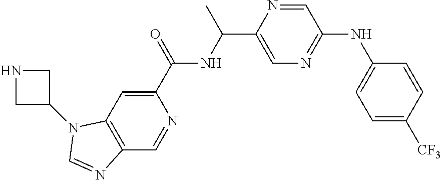 | ¹H-NMR (DMSO-D₆, 500 MHz) δ 9.94 (s, 1H), 9.08-9.06 (m, 3H), 8.87 (s, 1H), 8.48 (s, 1H), 8.31 (s, 2H), 7.89 (d, J = 8.5 Hz, 2H), 7.64 (d, J = 8.5 Hz, 2H), 5.77-5.74 (m, 1H), 5.30-5.27 (m, 1H), 4.58 (bs, 2H), 4.49 (bs, 2H), 1.55 (d, J = 7.0 Hz, 3H); LCMS m/z = 483.1 [M + 1]. |
| 180 | 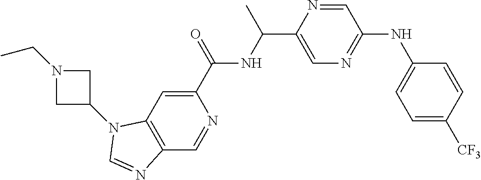 | ¹H-NMR (DMSO-D₆, 500 MHz) δ 9.97 (s, 1H), 9.03 (d, J = 8.5 Hz, 1H), 9.0 (s, 1H), 8.78 (s, 1H), 8.56 (s, 1H), 8.30 (s, 2H), 7.89 (d, J = 8.0 Hz, 2H), 7.61 (d, J = 8.0 Hz, 2H), 5.27-5.21 (m, 2H), 3.78-3.75 (m, 2H), 3.57-3.54 (m, 2H), 2.58-2.55 (m, 2H), 1.65 (d, J = 7.0 Hz, 3H), 1.1-0.9 (m, 3H); LCMS m/z = 511 [M + 1]. |
| 181 | 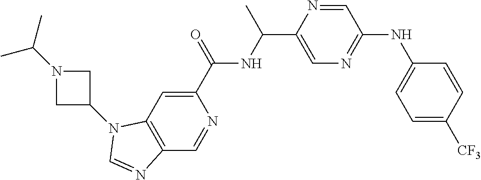 | ¹H-NMR (DMSO-D₆, 500 MHz) δ 9.92 (s, 1H), 9.05-9.02 (m, 2H), 8.73 (s, 1H), 8.53 (s, 1H), 8.31 (s, 2H), 7.89 (d, J = 8.0 Hz, 2H), 7.63 (d, J = 8 Hz, 2H), 5.28-5.20 (m, 2H), 3.75-3.72 (m, 2H), 3.46-3.44 (m, 2H), 1.55 (d, J = 6.5 Hz, 3H), 0.93-0.92 (m, 6H); LCMS m/z = 525 [M + 1]. |
| 182 | 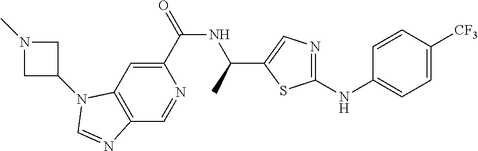 | ¹H-NMR (DMSO-D₆, 500 MHz) δ 10.45 (s, 1H), 9.08 (d, J = 7.5 Hz, 1H), 8.99 (s, 1H), 8.74 (s, 1H), 8.55 (s, 1H), 7.77 (d, J = 8.5 Hz, 2H), 7.62 (d, J = 8.5 Hz, 2H), 7.21 (s, 1H), 5.38-5.35 (m, 1H), 5.24-5.21 (m, 1H), 3.77-3.74 (m, 2H), 3.54-3.53 (m, 2H), 2.39 (s, 3H), 1.65 (d, J = 7 Hz, 3H); LCMS m/z = 502 [M + 1]. |
| 183 | 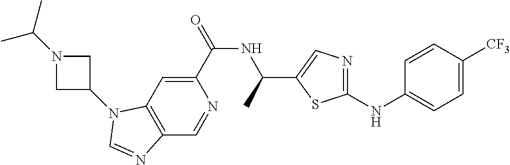 | ¹H-NMR (DMSO-D₆, 500 MHz) δ 10.44 (s, 1H), 9.07 (d, J = 7.5 Hz, 1H), 8.98 (s, 1H), 8.72 (s, 1H), 8.52 (s, 1H), 7.76 (d, J = 8.5 Hz, 2H), 7.62 (d, J = 8.5 Hz, 2H), 7.20 (s, 1H), 5.37-5.34 (m, 1H), 5.22-5.20 (m, 1H), 3.75-3.72 (m, 2H), 3.50-3.45 (m, 2H), 1.64 (d, J = 7 Hz, 3H), 0.93 (d, J = 6.5 Hz, 6H); LCMS m/z = 530 [M + 1]. |
| 184 | 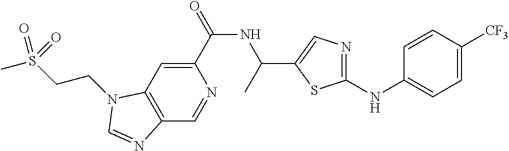 | ¹H-NMR (DMSO-D₆, 500 MHz) δ 10.48 (s, 1H), 9.12 (d, J = 7.5 Hz, 1H), 8.97 (s, 1H), 8.58 (s, 1H), 8. 46 (s, 1H), 7.79 (d, J = 8.0 Hz, 2H), 7.62 (d, J = 8.0 Hz, 2H), 7.22 (s, 1H), 5.39-5.37 (m, 1H), 4.83 (t, J = 7.0 Hz, 2H), 3.79 (t, J = 7.0 Hz, 2H), 3.12 (s, 3H), 1.65 (d, J = 7.5 Hz, 3H); LCMS m/z = 539 [M + 1]. |
| 185 | 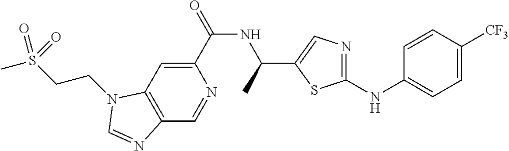 | ¹H-NMR (DMSO-D₆, 500 MHz) δ 10.46 (s, 1H), 9.15 (d, J = 7.0 Hz, 1H), 8.98 (s, 1H), 8.57 (s, 1H), 8. 48 (s, 1H), 7.79 (d, J = 8.0 Hz, 2H), 7.63 (d, J = 8.0 Hz, 2H), 7.21 (s, 1H), 5.39-5.37 (m, 1H), 4.83 (t, J = 7.0 Hz, 2H), 3.79 (t, J = 7.0 Hz, 2H), 3.12 (s, 3H), 1.65 (d, J = 7.5 Hz, 3H); LCMS m/z = 538.9 [M + 1]. |
| 186 | 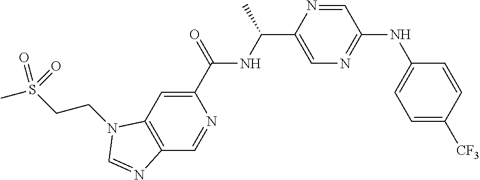 | ¹H-NMR (DMSO-D₆, 500 MHz) δ 10.46 (s, 1H), 9.11 (d, J = 7.5 Hz, 1H), 9.03 (s, 1H), 8.53 (s, 1H), 8. 40 (s, 1H), 8.33 (d, J = 7.5 Hz, 2H), 783 (d, J = 7.5 Hz, 2H), 7.63 (d, J = 7.5 Hz, 2H), 5.25-5.23 (m, 1H), 4.83 (t, J = 7.0 Hz, 2H), 3.79 (t, J = 7.0 Hz, 2H), 3.02 (s, 3H), 1.59 (d, J = 7.0 Hz, 3H); LCMS m/z = 534 [M + 1]. |
| 193 | 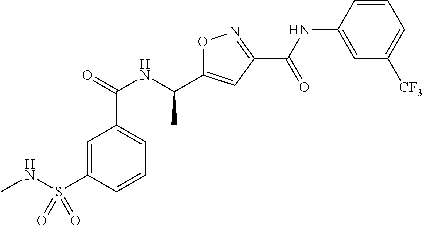 | |
| 194 | 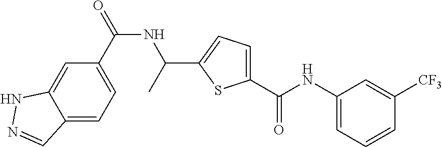 | |
| 195 | 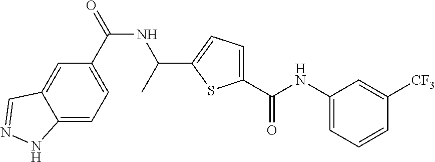 | |
| 196 | 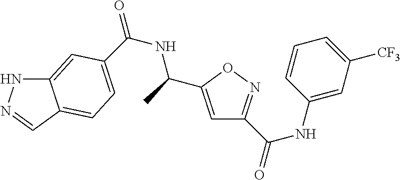 | |
| 197 | 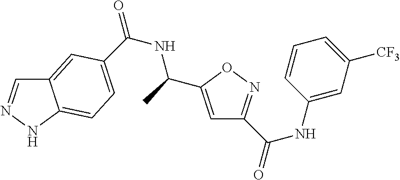 | |

### Examples 36 and 37

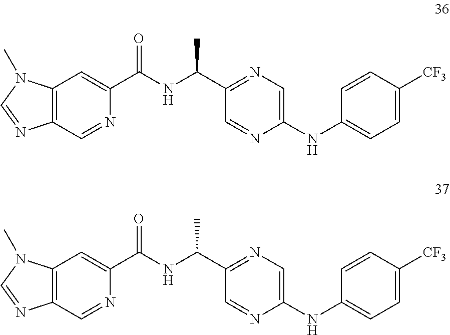

Synthesis of Examples 36 and 37. Examples 36 and 37 were prepared from 262 mg of Example 35 by preparatory chiral super-critical fluid chromatography on a Chiralpak IA (2×15 cm) with an isocratic eluant of 40% EtOH(0.1% Et₂NH)/CO₂ at 100 bar, a flow rate of 75 mL/min, an injection vol of 2 mL of a 10 mg/80 mL EtOH solution, and monitoring by UV detection at 220 nM to yield 158 mg (>99% ee) of Example 36 as the first eluting peak and 143 mg (>99% ee) of Example 37 as the second eluting peak. Enantiomeric purity was determined by analytical SCF chromatography Chiralpak IA (15×0.46 cm) with an isocratic eluant of 40% EtOH(0.1% Et₂NH)/CO₂ at 100 bar, a flow rate of 3 mL/min, and monitoring by UV detection at 220 nM.

Example 36: ¹H NMR (400 MHz, DMSO-d₆) δ 9.93 (s, 1H), 9.05 (d, J=8.3 Hz, 1H), 9.00 (d, J=1.0 Hz, 1H), 8.49 (s, 1H), 8.35 (d, J=1.3 Hz, 1H), 8.32 (s, 2H), 7.88 (d, J=8.5 Hz, 2H), 7.63 (d, J=8.5 Hz, 2H), 5.29 (dq, J=6.8, 8.0 Hz, 1H), 3.95 (s, 3H), 1.54 (d, J=7.0 Hz, 3H); LCMS m/z=442.2 [M+1]. Analytical Chiral SCFC Rt=3.30 min.

Example 37: ¹H NMR (400 MHz, DMSO-d₆) δ 9.93 (s, 1H), 9.05 (d, J=8.3 Hz, 1H), 9.00 (d, J=1.0 Hz, 1H), 8.49 (s, 1H), 8.35 (d, J=1.0 Hz, 1H), 8.32 (s, 2H), 7.88 (d, J=8.5 Hz, 2H), 7.63 (d, J=8.5 Hz, 2H), 5.29 (dq, J=6.8, 8.3 Hz, 1H), 3.95 (s, 3H), 1.54 (d, J=6.8 Hz, 3H); LCMS m/z=442.2 [M+1]. Analytical Chiral SCFC Rt=4.83 min

### Example 128

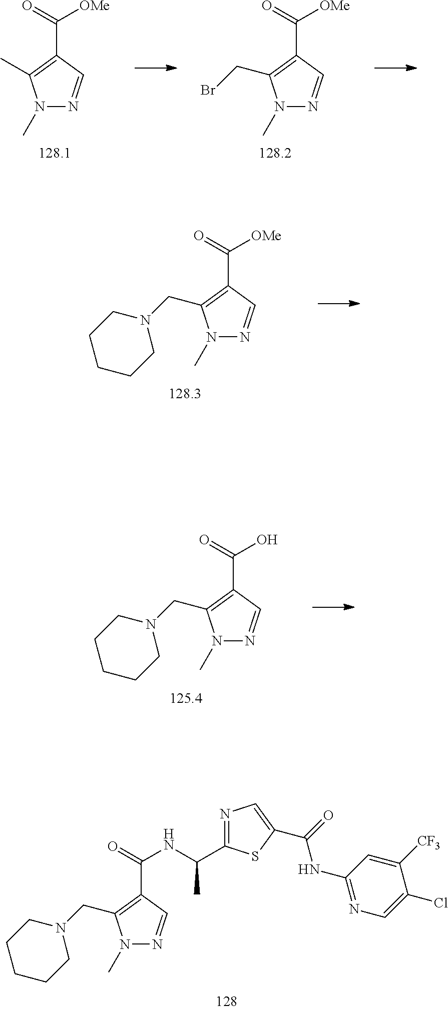

Synthesis of Compound 128.2. A solution of 265 mg (1.72 mmole) of compound 128.1 in 6 mL of CCl₄ was treated with 338 mg (1.9 mmole) of N-bromosuccinimide and 14 mg (0.09 mmole) of AIBN. The reaction mixture was heated at 80° C. for 3 hr, cooled to room temperature, and filtered through a medium frit, rinsing with CH₂Cl₂. The filtrate was concentrated and purified by flash column chromatography (SiO₂, 100% hexanes then gradient to 20% EtOAc/hexanes) to afford 353 mg (88%) of compound 128.2.

Synthesis of Compound 128.3. A solution of 59 mg (0.26 mmole) of compound 128.2 in 1 mL of CH₃CN was treated with 30 μL (0.3 mmole) of piperidine and 54 μL of triethylamine. The reaction mixture was heated at 50° C. for 16 hr and then loaded directly only a silica gel column for purification. Elution with 2:1 EtOAc/hexanes followed by 4:1 EtOAc/hexanes afforded 56 mg (92%) of compound 128.3.

Synthesis of Compound 128.4. The compound 128.4 was prepared as described previously in Scheme E.

Synthesis of Example 128. The compound of Example 128 was synthesized as described previously in the Table 1 general amide bond formation procedure. LCMS m/z=556 [M+1].

TABLE 5
The following compounds of the present invention, set forth in Table 5,
below, were prepared as described in Example 128 using the appropriate amine.

| Example | Structure | Characterization Data |
|---|---|---|
| 129 | 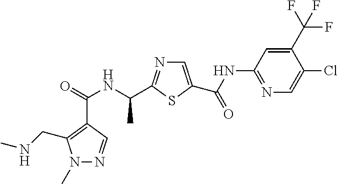 | LCMS m/z = 502 [M + 1] |
| 130 | 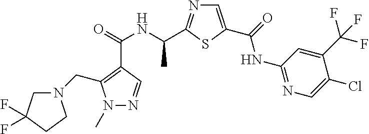 | LCMS m/z = 578 [M + 1] |
| 131 | 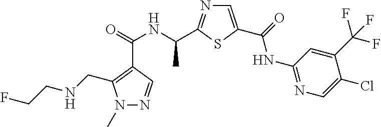 | LCMS m/z = 534 [M + 1] |
| 132 | 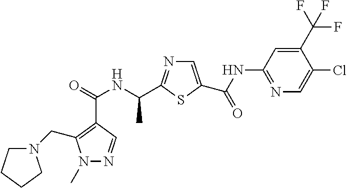 | LCMS m/z = 542 [M + 1] |
| 133 | 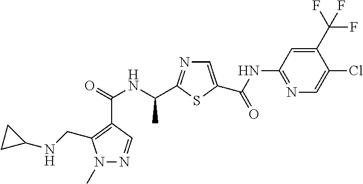 | LCMS m/z = 528 [M + 1] |

### Examples 187 and 188

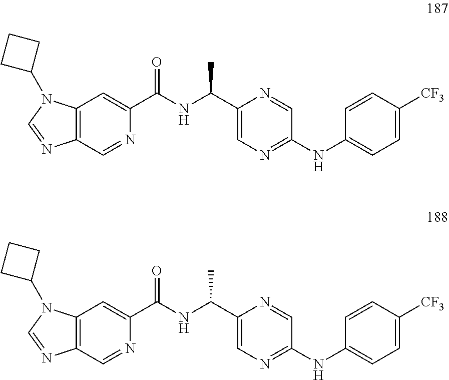

Synthesis of Examples 187 and 188. Examples 187 and 188 were prepared from the compound of Example 175 by preparatory chiral super-critical fluid chromatography on a Chiralpak IA column (2×15 cm, #808041) with an isocratic eluant of 40% EtOH(0.1% Et₂NH)/CO₂ at 100 bar, a flow rate of 50 mL/min, an injection vol of 2 mL of a 3 mg/mL MeOH solution, and monitoring by UV detection at 220 nM to yield 42 mg (100% ee) of Example 187 as the first eluting peak and 56 mg (100% ee) of Example 188 as the second eluting peak. Enantiomeric purity was determined by analytical SCF chromatography (Chiralpak IA (25×0.46 cm) with an isocratic eluant of 40% EtOH/CO₂ at 100 bar, a flow rate of 3 mL/min, and monitoring by UV detection at 220 nM.

Example 187: LCMS m/z=482.30. Analytical Chiral SCFC Rt=2.04 min, 100% ee

Example 188: LCMS m/z=482.30. Analytical Chiral SCFC Rt=2.83 min, 100% ee.

### Example 189

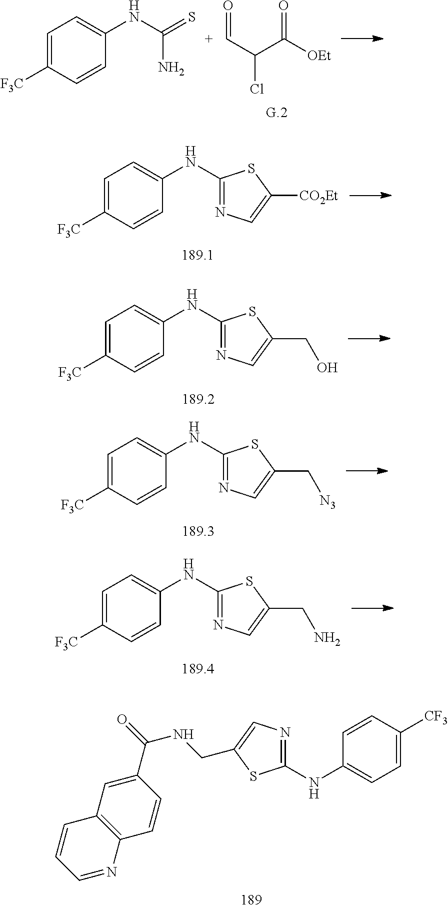

Synthesis of Compound 189.1. A room temperature solution of [4-(trifluoromethyl)-phenyl]thiourea (10 g, 45.45 mmol) in ethanol (100 mL) was treated with G.2 (10.26 g, 68.18 mmol, Plouvier, B.; Bailly, C.; Houssin, R.; Henichart, J. P. Heterocycles 1991, 32, 693-701), and the reaction mixture was heated at reflux for 16 hr. The ethanol solvent was distilled off and the residue was dissolved in EtOAc. The organic layer was washed with sodium bicarbonate solution, water, and brine, dried over anhydrous Na₂SO₄, filtered, and concentrated under vacuum. Purification by flash column chromatography (SiO₂, 100% hexane to 12% EtOAc/Hexane) afforded compound 189.1 as a yellow solid (10 g, 69.63%). ¹H NMR (CDCl₃, 200 MHz) δ 9.3-9.4 (br s, 1H, D₂O exchangeable), 8.0 (s, 1H), 7.6-7.7 (d, 2H), 7.3-7.4 (d, 2H), 4.2-4.4 (q, 2H), 1.3-1.4 (m, 3H); LCMS m/z=317 [M+1].

Synthesis of Compound 189.2. A solution of compound 189.1 (4 g, 12.65 mmol) in dry CH₂Cl₂ (60 mL) was cooled to −78° C. under a N₂ atmosphere, and treated with DIBAL-H (38 mL, 1M solution in toluene, 38 mmol). The reaction was stirred at −78° C. for 2 hr, then quenched by addition of saturated NH₄Cl solution, and slowly warmed to room temperature. The reaction mixture was filtered through celite, and the filter cake was washed with CH₂Cl₂. The organic layer was separated and dried over anhydrous Na₂SO₄, filtered, and concentrated under vacuum. Purification by flash column chromatography (SiO₂, 100% hexanes to 25% Ethyl acetate/hexanes) afforded compound 189.2 as white solid (1.8 g, 52%). ¹H NMR (DMSO-D₆, 200 MHz) δ: 10.5 (s, 1H, D₂O exchangeable), 7.7-7.8 (d, 2H), 7.5-7.6 (d, 2H), 7.1 (s, 1H), 5.3 (t, 1H, D₂O exchangeable), 4.5 (s, 2H); LCMS m/z=274.9 [M+1].

Synthesis of Compound 189.3. A solution of compound 189.2 (1.8 g, 6.57 mmol) in toluene (30 mL) and THF (10 mL) was cooled in an ice bath at 0° C., and treated with diphenylphosphonic azide (2.835 g, 13.139 mmol) and DBU (2 g, 13.139 mmol). The reaction mixture was stirred overnight at room temperature. The mixture was concentrated under vacuum, and the residue was purified by flash column chromatography to obtain compound 189.3 (1 g, 51%) as yellow solid. ¹H NMR (CDCl₃, 200 MHz) δ: 7.6-7.7 (d, 2H), 7.5-7.6 (d, 2H), 7.3 (s, 1H), 4.4 (s, 2H); LCMS m/z=300 [M+1].

Synthesis of Compound 189.4. A solution of compound 189.3 (500 mg, 1.672 mmol) in THF (20 mL) and water (1 mL) was treated with triphenylphosphine (657 mg, 2.508 mmol). The mixture was stirred overnight at room temperature. Solvents were evaporated and the residue was purified by column chromatography (SiO₂, 100% CH₂Cl₂ to 2.5% MeOH/CH₂Cl₂) to obtain compound 189.4 as a brown colour solid. (300 mg, 65.78%). ¹HNMR: (DMSO-D6, 200 MHz) δ: 10.4-10.6 (br s, 1H), 7.7-7.9 (d, 2H), 7.6-7.7 (d, 2H), 7.1 (s, 1H), 3.9 (s, 2H); LCMS m/z=274 [M+1].

Synthesis of Example 189. The compound of Example 189 was prepared as described in the Table 1 general amide bond coupling procedure using quinoline-6-carboxylic acid. ¹H-NMR (DMSO-D6, 500 MHz) δ 10.45 (s, 1H), 9.38 (s, 1H), 8.99 (s, 1H), 8.50 (s, 1H), 8.45 (d, J=8.5 Hz, 1H), 8.18 (d, J=8.5 Hz, 1H), 8.11 (d, J=9 Hz, 1H), 7.80 (d, J=8.5 Hz, 2H), 7.61 (d, J=8.5 Hz, 2H), 7.22 (s, 1H), 4.59 (s, 2H); LCMS m/z=428.9 [M+1].

### Example 190

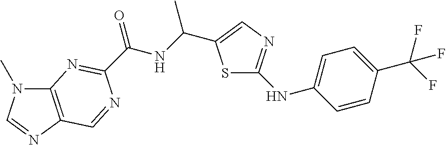

Synthesis of Example 190. The compound of Example 190 was prepared as previously described in Scheme F, using 2-chloro-9-methyl-9H-purine in place of 6-bromo-1-ethyl-1H-imidazo[4,5-c]pyridine D.4, and the Table 1 general amide bond formation procedure. ¹H-NMR (DMSO-D6, 500 MHz): δ 10.49 (s, 1H), 9.25-9.24 (m, 2H), 8.70 (s, 1H), 7.78 (d, J=8.5 Hz, 2H), 7.64 (d, J=8.5 Hz, 2H), 7.24 (s, 1H), 5.38-5.36 (m, 1H), 3.97 (s, 3H), 1.63 (d, J=7 Hz, 3H); LCMS m/z=448 [M+1].

### Example 191

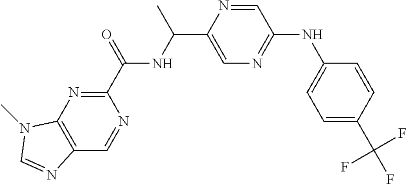

Synthesis of Example 191. The compound of Example 191 was prepared as previously described in Scheme F, using 2-chloro-9-methyl-9H-purine in place of 6-bromo-1-ethyl-1H-imidazo[4,5-c]pyridine D.4, and the Table 1 general amide bond formation procedure. ¹H-NMR (CD₃OD, 500 MHz) δ 9.19 (s, 1H), 8.68 (s, 1H), 8.59 (s, 1H), 8.25 (s, 1H), 8.21 (s, 1H), 7.95 (d, J=8.5 Hz, 2H), 7.58 (d, J=8.5 Hz, 2H), 5.29-5.26 (m, 1H), 4.01 (s, 3H), 1.63 (d, J=7 Hz, 3H); LCMS m/z=443.2 [M+1].

### Example 192

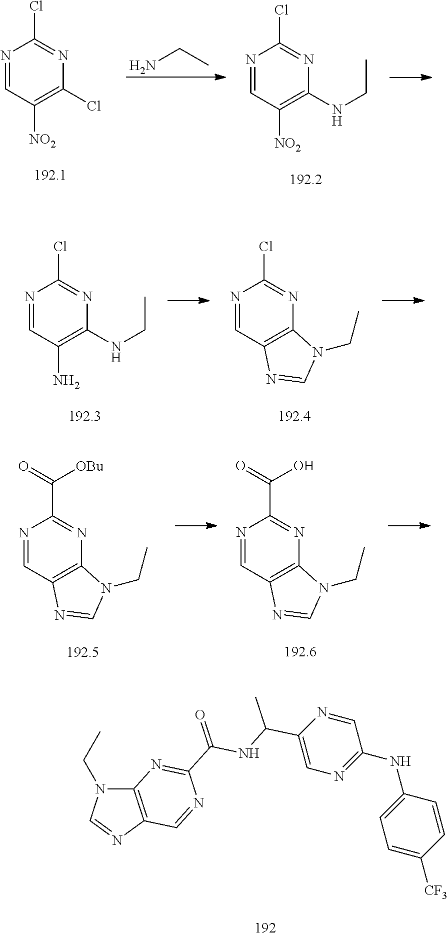

Synthesis of Compound 192.2. To a stirred solution of 2,4-dichloro-5-nitropyrimidine 192.1 (0.5 g, 2.5 mmol) in THF (5 ml), was added ethyl amine (2.5 ml, 5.1 mmol) with a syringe slowly. The reaction mixture was stirred at room temperature for 4 hr. After the consumption of starting material (by TLC), the crude material was diluted with water (20 ml) and extracted with EtOAc (3×20 ml). The combined organic layer was dried over anhydrous sodium sulphate, and evaporated under reduced pressure. The resulting crude material was purified by column chromatography [silica gel (60-120 mesh, 100 g), gradient 7-10% EtOAc/Hexane] to afford 192.2 (210 mg, 40% yield) as a yellow solid. ¹H NMR (CDCl₃, 200 MHz) δ 9.04 (s, 1H), 8.39 (bs, 1H), 3.77-3.67 (m, 2H), 1.34 (t, J=7.2 Hz, 3H); LCMS m/z=203 [M+1].

Synthesis of Compound 192.3. Compound 192.3 was prepared as previously described in Scheme D. ¹H NMR (CDCl₃, 200 MHz) δ 7.61 (s, 1H), 4.81 (bs, 1H), 3.54 (q, J=7.2 Hz, 2H), 2.09 (bs, 1H), 1.27 (t, J=6.6 Hz, 3H); LCMS m/z=173.1 [M+1].

Synthesis of Compound 192.4. Compound 192.4 was prepared as previously described in Scheme D. ¹H NMR (CD₃OD, 200 MHz) δ 8.94 (s, 1H), 8.54 (s, 1H), 4.38 (q, J=7.7 Hz, 2H), 1.55 (t, J=7.7 Hz, 3H); LCMS m/z=183.1 [M+1].

Synthesis of Compound 192.5. Compound 192.5 was prepared as previously described in Scheme F. LCMS m/z=249.2 [M+1].

Synthesis of Compound 192.6. Compound 192.6 was prepared as previously described in Scheme F. LCMS m/z=193 [M+1].

Synthesis of Example 192. The compound of Example 192 was prepared as previously described. ¹H NMR (DMSO-D₆, 500 MHz) δ 9.92 (s, 1H), 9.25 (s, 1H), 9.15 (d, J=8.5 Hz, 1H), 8.80 (s, 1H), 8.32 (d, J=7.0 Hz, 2H), 7.89 (d, J=8.5 Hz, 2H), 7.64 (d, J=9.0 Hz, 2H), 5.26 (q, J=7.5 Hz, 1H), 4.38 (q, J=7.0 Hz, 1H), 1.56 (d, J=6.5 Hz, 3H), 1.49 (d, J=7.5 Hz, 3H); LCMS m/z=457.3 [M+1].

### Example 198

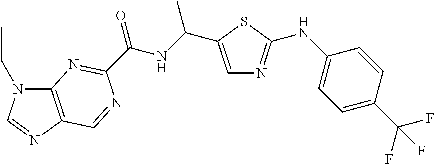

Synthesis of Example 198. The compound of Example 198 was prepared as previously described in Example 192 using compound A.6. in place of compound B.5. ¹H NMR (CD₃OD, 500 MHz) δ 9.20 (s, 1H), 8.75 (s, 1H), 7.78 (d, J=9.5 Hz, 1H), 7.68 (d, J=9.5 Hz, 2H), 7.24 (s, 1H), 5.43 (q, J=7.0 Hz, 1H), 4.52 (q, J=7.5 Hz, 1H), 1.78 (d, J=7.0 Hz, 3H), 1.59 (d, J=8.0 Hz, 3H); LCMS m/z=462.0 [M+1].

### Example 199

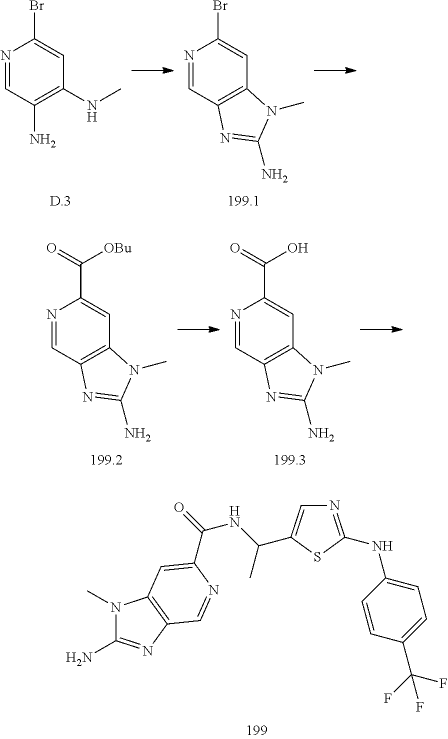

Synthesis of Compound 199.1. A solution of compound D.3 (600 mg, 2.9 mmol) and EtOH (20 ml) was treated with cyanogen bromide (944 mg, 8.9 mmol) in a sealed tube at room temperature and stirred for 12 hr at 100° C. After the consumption of the starting material (by TLC), the reaction mixture was filtered through a celite bed and concentrated under reduced pressure. The crude material was purified by column chromatography [silica gel (60-120 mesh, 200 g), gradient (5-10% MeOH/CH₂Cl₂)] to afford compound 199.1 (400 mg, 59%) as a brown solid. ¹H-NMR (DMSO-d₆, 200 MHz) δ 8.10 (s, 1H), 7.42 (s, 1H), 6.99 (bs, 2H), 3.50 (s, 3H).

Synthesis of Compound 199.2. The mixture of compound 199.1 (150 mg, 0.66 mmol), BINAP (82 mg, 0.132 mmol), DIPEA (0.14 ml, 0.85 mmol), Pd(CH₃CN)₂Cl₂ (34 mg, 0.132 mol) in 1,4-dioxane/n-butanol (5 ml of 1:1) in a steel bomb was stirred at 100° C. for 16 hr under CO gas (150 psi). After consumption of the starting material (by TLC), the reaction mixture was cooled to room temperature. The volatiles were removed under reduced pressure. The resulting crude material was purified by column chromatography [silica gel (60-120 mesh, 100 g), gradient (1-5% MeOH/CH₂Cl₂)] to afford compound 199.2 (100 mg, 61%) as a brown solid. ¹H-NMR (DMSO-d₆, 200 MHz) δ 8.40 (s, 1H), 7.91 (s, 1H), 7.10 (bs, 2H), 4.26 (t, J=6.6 Hz, 2H), 3.58 (s, 3H), 1.72-1.65 (m, 2H), 1.44-1.40 (m, 2H), 0.94 (t, J=6.6 Hz, 3H). LCMS m/z=249 [M+1].

Synthesis of Compound 199.3. To a stirred solution of compound 199.2 (100 mg, 0.40 mmol) in THF/water (2 ml of 1:1) was added LiOH (25 mg, 0.60 mmol) at 0° C. and the reaction mixture was stirred at room temperature for 12 hr. After consumption of starting material (by TLC), the reaction mixture was concentrated under reduced pressure, the residue was evaporated with toluene (3×5 ml) and washed with ether (5 ml) to afford compound 199.3 (60 mg, crude) as a brown solid. ¹H-NMR (DMSO-d₆, 200 MHz) δ 8.11 (s, 1H), 7.77 (s, 1H), 3.53 (s, 3H).

Synthesis of Example 199. The compound of Example 199 was prepared as previously described. ¹H-NMR (DMSO-D₆, 500 MHz) δ 10.43 (s, 1H), 8.79 (d, J=9.0 Hz, 1H), 8.32 (s, 1H), 7.88 (s, 1H), 7.76 (d, J=9.0 Hz, 2H), 7.61 (d, J=8.5 Hz, 2H), 7.17 (s, 1H), 7.00 (s, 2H), 5.31 (q, J=7.0 Hz, 1H), 3.57 (s, 3H), 1.61 (d, J=7.0 Hz, 3H). LCMS m/z=462 [M+1].

### Example 200

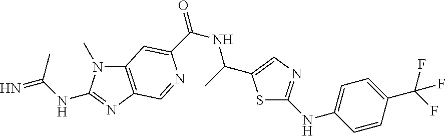

Synthesis of Example 200. The compound of Example 200 was prepared as described in Example 199 except using acetonitrile as solvent in place of 1,4-dioxane during the Pd-catalyzed carbonylation step. ¹H-NMR (DMSO-D6, 500 MHz) δ 10.43 (s, 1H), 9.90 (s, 1H), 8.91 (d, J=8.5 Hz, 1H), 8.68 (s, 1H), 8.63 (s, 1H), 8.07 (s, 1H), 7.76 (d, J=8.5 Hz, 2H), 7.61 (d, J=8.5 Hz, 2H), 7.19 (s, 1H), 5.35-5.32 (m, 1H), 3.69 (s, 3H), 2.15 (s, 3H), 1.62 (d, J=7.0 Hz, 3H); LCMS m/z=503 [M+1].

### Example 201

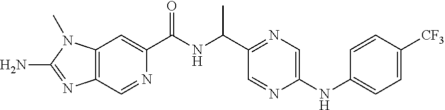

Synthesis of Example 201. The compound of Example 201 was prepared as described in Example 199 except using compound B.5. ¹H-NMR (DMSO-D₆, 500 MHz) δ 9.92 (s, 1H), 8.85 (d, J=8.5 Hz, 1H), 8.38 (s, 1H), 8.31 (d, J=8.0 Hz, 2H), 7.91 (d, J=7.0 Hz, 3H), 7.61 (d, J=9.0 Hz, 2H), 7.00 (s, 1H), 5.27-5.21 (m, 1H), 3.59 (s, 3H), 1.54 (d, J=6.5 Hz, 3H); LCMS m/z=457 [M+1].

### Example 202

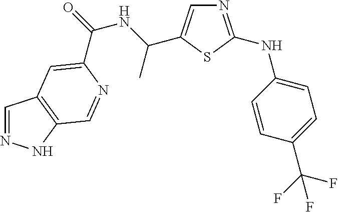

Synthesis of Example 202. The compound of Example 202 was prepared as described previously in Scheme F using 1-(5-chloro-1H-pyrazolo[3,4-c]pyridin-1-yl)ethanone in place of 6-bromo-1-ethyl-1H-imidazo[4,5-c]pyridine F.1. ¹H-NMR (DMSO-D6, 500 MHz) δ 13.98 (s, 1H), 10.44 (s, 1H), 9.1 (s, 1H), 9.0 (d, J=8.5 Hz, 1H), 8.55 (s, 1H), 8.50 (s, 1H), 7.76 (d, J=8.5 Hz, 2H), 7.61 (d, J=8.5 Hz, 2H), 7.20 (s, 1H), 5.39-5.35 (m, 1H), 1.63 (d, J=7.0 Hz, 3H); LCMS m/z=433 [M+1].

### Example 203

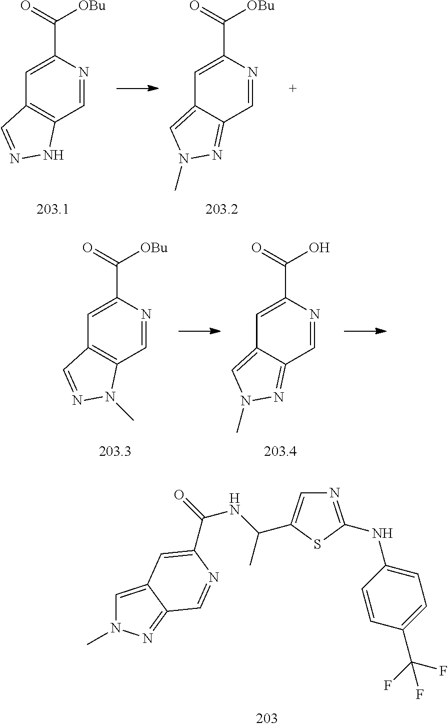

Synthesis of Compound 203.1. Compound 203.1 was prepared as described previously in Scheme F using 1-(5-chloro-1H-pyrazolo[3,4-c]pyridin-1-yl)ethanone in place of 6-bromo-1-ethyl-1H-imidazo[4,5-c]pyridine F.1. ¹H-NMR (DMSO-D₆, 200 MHz) δ 13-0.97 (bs, 1H), 9.11 (s, 1H), 8.56 (s, 1H), 8.40 (s, 1H), 4.30 (t, J=6.6 Hz, 2H), 1.75-1.49 (m, 2H), 1.45-1.38 (m, 2H), 0.98 (t, J=7.5 Hz, 3H); LCMS m/z=220 [M+1].

Synthesis of Compound 203.2. To a stirred solution of compound 203.1 (50 mg, 0.23 mmol), in DMF (5 ml) was added K₂CO₃ (94 mg, 0.68 mmol) and MeI (0.02 ml, 0.3 mmol) were added at 0° C. The resultant reaction mixture was stirred at room temperature for 5 hr. After completion of the starting material (by TLC), the reaction mixture was partitioned between EtOAc and water. The combined organic extracts were dried over sodium sulphate and concentrated under reduced pressure, the crude material was purified by column chromatography [silica gel (60-120 mesh, 20 g) gradient 1-2% MeOH/CH₂Cl₂] to afford 30 mg of compound 203.2 as a brown solid, along with 30 mg of compound 203.3. ¹H-NMR (CDCl₃, 200 MHz) δ 9.03 (s, 1H), 8.56 (s, 1H), 8.17 (s, 1H), 4.45 (t, J=7.0 Hz, 2H), 4.25 (s, 3H), 1.87-1.80 (m, 2H), 1.55-1.47 (m, 2H), 0.99 (t, J=7.2 Hz, 3H); LCMS m/z=234 [M+1].

Synthesis of Compound 203.4. Compound 203.4 was prepared as described previously in Scheme F using compound 203.2. ¹H-NMR (DMSO-D₆, 200 MHz) δ 8.89 (s, 1H), 8.28 (s, 1H), 8.19 (s, 1H).

Synthesis of Example 203. The compound of Example 203 was prepared as described previously in the Table general amide bond coupling procedure. ¹H-NMR (DMSO-D₆, 500 MHz) δ 10.46 (s, 1H), 9.18 (s, 1H), 9.05 (s, 1H), 8.46 (s, 1H), 8.35 (s, 1H), 7.78 (d, J=8.5 Hz, 2H), 7.63 (d, J=8.5 Hz, 2H), 7.21 (s, 1H), 5.39-5.36 (m, 1H), 4.23 (s, 3H), 1.65 (d, J=6.5 Hz, 3H); LCMS m/z=447 [M+1].

TABLE 6
The following compounds of the present invention, set forth in Table 6, below, were prepared by the
general amide bond coupling method described above using the appropriate amine from Scheme A, B, or
C and the appropriate carboxylic acids that were prepared as described in Example 203.

| Example | Structure | Characterization Data |
|---|---|---|
| 204 | 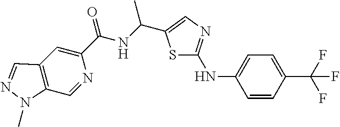 | ¹H-NMR (DMSO-D6, 500 MHz) δ 10.46 (s, 1H), 9.18 (s, 1H), 9.05 (d, J = 8.5 Hz, 1H), 8.46 (s, 1H), 8.35 (s, 1H), 7.78 (d, J = 8.5 Hz, 2H), 7.63 (d, J = 8.5 Hz, 2H), 7.21 (s, 1H), 5.38-5.35 (m, 1H), 4.23 (s, 3H), 1.65 (d, J = 7.0 Hz, 3H); LCMS m/z = 447.1 [M + 1]. |
| 205 | 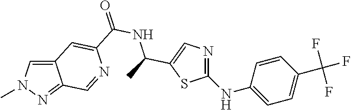 | ¹H-NMR (DMSO-D6, 500 MHz) δ 10.44 (s, 1H), 9.13 (s, 1H), 8.94 (d, J = 8.5 Hz, 1H), 8.66 (s, 1H), 8.42 (s, 1H), 7.76 (d, J = 8.5 Hz, 2H), 7.61 (d, J = 8.5 Hz, 2H), 7.20 (s, 1H), 5.37-5.34 (m, 1H), 4.28 (s, 3H), 1.63 (d, J = 7 Hz, 3H); LCMS m/z = 447.1 [M + 1]. |
| 206 | 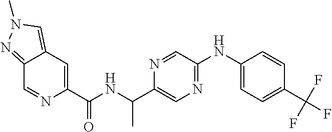 | ¹H-NMR (DMSO-D6, 500 MHz) δ 9.93 (s, 1H), 9.18 (s, 1H), 8.96 (d, J = 8.5 Hz, 1H), 8.67 (s, 1H), 8.42 (s, 1H), 8.31 (s, 2H), 7.89 (d, J = 8.5 Hz, 2H), 7.63 (d, J = 9 Hz, 2H), 5.29-5.26 (m, 1H), 4.29 (s, 3H), 1.54 (d, J = 6.5 Hz, 3H); LCMS m/z = 442.1 [M + 1]. |
| 207 | 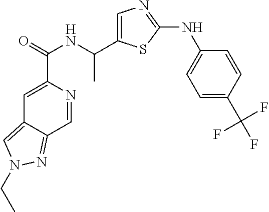 | ¹H-NMR (DMSO-D6, 500 MHz): δ 10.45 (s, 1H), 9.14 (s, 1H), 8.97 (d, J = 9 Hz, 1H), 8.77 (s, 1H), 8.45 (s, 1H), 7.76 (d, J = 8.5 Hz, 2H), 7.61 (d, J = 8.5 Hz, 2H), 7.20 (s, 1H), 5.38-5.35 (m, 1H), 4.59 (q, J = 7.5 Hz, 2H), 1.63 (d, J = 7.0 Hz, 3H), 1.56 (t, J = 7.0 Hz, 3H); LCMS m/z = 461 [M + 1]. |

### Example 208

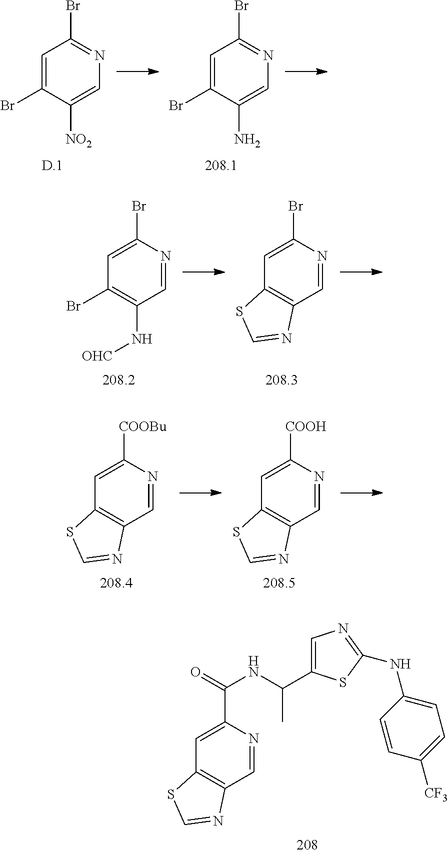

Synthesis of Compound 208.1. To a stirred solution of compound D.1 (500 mg, 1.77 mmol) in AcOH (20 ml), was added iron powder (400 mg, 7.27 mmol). The reaction mixture was heated at 60° C. for 2 hr. After completion of the starting material (by TLC), the reaction mixture was filtered on celite bed and washed with ethyl acetate. The filtrate was concentrated under reduced pressure, and the crude material was diluted with NaHCO₃ solution (100 ml) and extracted with ethyl acetate (3×20 ml). The combined organic extracts was washed with water and dried over anhydrous sodium sulphate, concentrated under reduced pressure to afford compound 208.1 (350 mg, 78.47%, crude) as brown solid, which was used for the next step any further purification. ¹H-NMR (CDCl₃, 500 MHz) δ 7.94 (s, 1H), 7.54 (s, 1H), 7.26 (s, 1H), 3.50 (bs, 2H); LCMS m/z=259 [M+1].

Synthesis of Compound 208.2. To a stirred solution of compound 208.1 (350 mg) in formic acid (2.2 ml) was added acetic anhydride (1.2 ml) at 0° C. and stirred at room temperature for 5 hr. After completion of the starting material (by TLC), the reaction mixture was concentrated under reduced pressure to afford compound 208.2 (250 mg, 64%) of a white solid which was used immediately in the next step without further purification. ¹H-NMR (CDCl₃, 500 MHz) δ 9.37 (s, 1H), 8.52 (s, 1H), 7.73 (s, 1H), 7.25 (bs, 1H).

Synthesis of Compound 208.3. The compound 208.2 was dissolved in toluene (10 ml) and treated with Lawesson's reagent (260 mg, 0.6428 mmol). The reaction was heated at 55° C. for 16 hr. After completion of the starting material (by TLC), solvent was distilled off, the residue was diluted with water and extracted with ethyl acetate. Ethyl acetate layer was washed with aqueous NaHCO₃, dried over anhydrous sodium sulfate and solvent was evaporated. The crude was purified by column chromatography to obtain compound 140.3 (150 mg, 65%). ¹H-NMR (CDCl₃, 500 MHz) δ 9.20 (s, 1H), 9.03 (s, 1H), 8.12 (s, 1H). LCMS m/z=217 [M+2]⁺.

Synthesis of Compound 208.4. Compound 208.4 was prepared as described previously in Scheme F using compound 208.3. ¹H-NMR (CDCl₃, 500 MHz) δ 9.50 (s, 1H), 9.21 (s, 1H), 8.78 (s, 1H), 4.48-4.39 (m, 2H), 1.89-1.74 (m, 2H), 1.52-1.40 (m, 2H), 0.895 (t, J=7.4 Hz, 3H); LCMS m/z=237 [M+1].

Synthesis of Compound 208.5. Compound 208.5 was prepared as described previously in Scheme F using compound 208.4. ¹H-NMR (D₂O, 500 MHz) δ 9.45 (s, 1H), 9.22 (s, 1H), 8.67 (s, 1H).

Synthesis of Example 208. The compound of Example 208 was prepared as described previously in the Table 1 general amide bond coupling procedure. ¹H-NMR (DMSO-D₆, 500 MHz) δ 10.45 (s, 1H), 9.64 (s, 1H), 9.37 (s, 1H), 9.22 (s, 1H), 9.21 (s, 1H), 8.92 (s, 1H), 7.76 (d, J=8.5 Hz, 2H), 7.61 (d, J=8.5 Hz, 2H), 7.21 (s, 1H), 5.39-5.37 (m, 1H), 1.64 (d, J=7 Hz, 3H); LCMS m/z=450.1 [M+1].

### Example 209

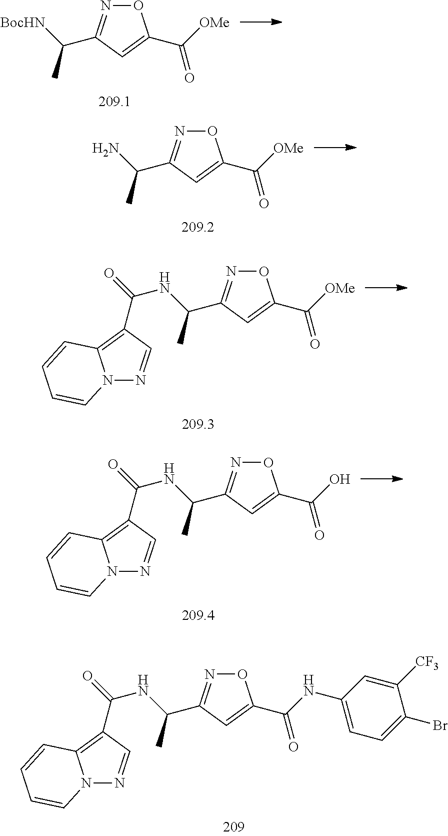

Synthesis of Compound 209.2. A mixture of 3-(1-tert-Butoxycarbonylamino-ethyl)-isoxazole-5-carboxylic acid methyl ester 209.1 (10.19 g, 37.7 mmol) and 4.0 M of Hydrogen chloride in 1,4-dioxane (90 mL) was stirred at 50° C. for 15 minutes. The reaction mixture was concentrated under vacuum to give 7.91 g of compound 209.2 as a solid that was used without further purification. ¹H-NMR (300 MHz, DMSO) δ 9.06 (bs, 3H), 7.61 (s, 1H), 4.65 (q, J=7.1 Hz, 1H), 3.92 (s, 3H), 1.59 (d, J=6.9 Hz, 3H).

Synthesis of Compound 209.3. Compound 209.3 was prepared as previously described in the Table 1 general amide bond formation conditions using H-pyrazolo[1,5-a]pyridine-3-carboxylic acid. ¹H-NMR (300 MHz, CDCl₃) δ 8.53 (d, J=6.8 Hz, 1H), 8.32 (d, J=8.9 Hz, 1H), 8.26 (s, 1H), 7.40 (dd, J=8.0, 7.1 Hz, 1H), 7.00 (s, 1H), 6.96 (t, J=6.7 Hz, 1H), 6.53 (d, J=7.5 Hz, 1H), 5.55 (m, 1H), 3.96 (s, 3H), 1.71 (d, J=7.1 Hz, 1H); LCMS m/z=314.6 [M+H]⁺.

Synthesis of Compound 209.4. A round bottom flask was charged with compound 209.3 (4.69 g, 14.9 mmol), 80 mL of anhydrous tetrahydrofuran, and 80 mL of water. The solution was cooled to 0° C. in an ice bath and lithium hydroxide, monohydrate (0.751 g, 17.9 mmol) was added. The reaction mixture was stirred for 3 hr at 0° C. The volatiles ere removed in vacuo, and the aqueous layer was acidified with 1.0 N HCl to pH between 3 and 4. The white precipitate was filtered and was dried in vacuo to give 4.49 g of compound 209.4 that was used without further purification. ¹H-NMR (300 MHz, CDCl₃) δ 8.77 (d, J=6.9 Hz, 1H), 8.64 (d, J=8.0 Hz, 1H), 8.63 (s, 1H), 8.19 (d, J=8.7 Hz, 1H), 7.47 (t, J=7.7 Hz, 1H), 7.12 (s, 1H), 7.07 (dt, J=6.9, 1.4 Hz, 1H), 5.37 (quint, J=7.6 Hz, 1H), 3.40 (bs, 1H), 1.56 (d, J=6.9 Hz, 3H); LCMS m/z=300.53 [M+H]⁺.

Synthesis of Example 209. A vial was carged with (R)-3-(1-(H-pyrazolo[1,5-a]pyridine-3-carboxamido)ethyl)isoxazole-5-carboxylic acid 209.4 (30.03 mg, 0.1 mmol), 2-chloro-1-methylpyridinium iodide (33.2 mg, 0.13 mmol), and anhydrous CH₂Cl₂ (1.5 mL). The reaction mixture was stirred for 10 minutes, then 4-bromo-3-(trifluoromethyl)-aniline (31.2 mg, 0.130 mmol) and N,N-diisopropylethylamine (69.7 uL, 0.40 mmol) was added. The reaction mixture was stirred overnight at room temperature. The crude reaction mixture was washed with saturated aqueous NaHCO₃, and the aqueous layer was extracted with CH₂Cl₂ (3×2 mL). The organic layers were collected, combined, and concentrated in vacuo. The crude residue was purified by mass directed preparatory HPLC. Final analysis by LCMS was consistent with desired product. LCMS m/z=522 [M+1].

TABLE 7
The following compounds of the present invention, set forth in Table 7,
below, were prepared as previously described in Example 209.

| Example | Structure | Characterization Data |
|---|---|---|
| 210 | 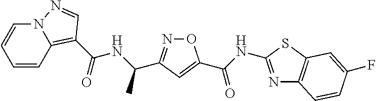 | LCMS m/z = 451 [M + 1] |
| 211 | 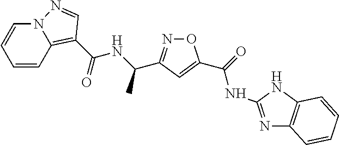 | LCMS m/z = 416 [M + 1] |
| 212 | 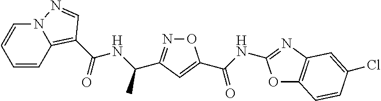 | LCMS m/z = 451 [M + 1] |
| 213 | 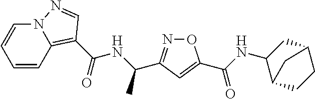 | LCMS m/z = 394 [M + 1] |
| 214 | 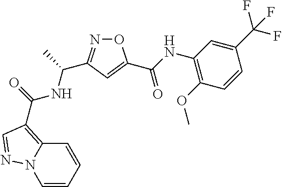 | LCMS m/z = 474 [M + 1] |
| 215 | 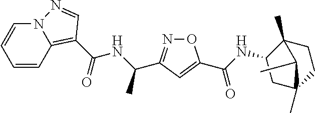 | LCMS m/z = 436 [M + 1] |
| 216 | 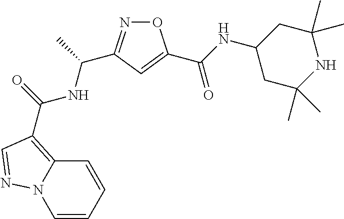 | LCMS m/z = 439 [M + 1] |
| 217 | 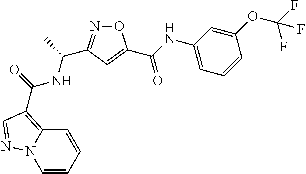 | LCMS m/z = 460 [M + 1] |
| 218 | 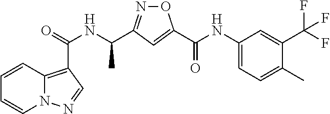 | LCMS m/z = 458 [M + 1]. |
| 219 | 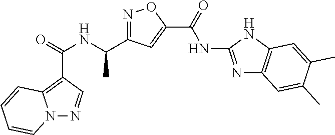 | ¹H NMR (300 MHz, DMSO-d₆) δ 12.46 (s, 2H), 8.79 (d, J = 6.97 Hz, 1H), 8.58-8.70 (m, 2H), 8.23 (d, J = 8.76 Hz, 1H), 7.38-7.62 (m, 1H), 7.19 (s, 2H), 7.01-7.14 (m, 1H), 6.89 (s, 1H), 5.38 (t, J = 7.54 Hz, 1H), 2.26 (s, 6H), 1.58 (d, J = 7.06 Hz, 3H); LCMS m/z = 444 [M + 1]. |
| 220 | 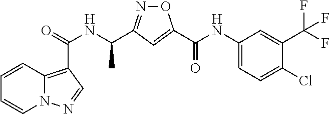 | ¹H NMR (300 MHz, DMSO-d₆) δ 11.18 (s, 1H), 8.88 (dt, J = 1.04, 6.97 Hz, 1H), 8.78 (s, 1H), 8.74 (s, 1H), 8.38 (d, J = 2.45 Hz, 1H), 8.27-8.36 (m, 1H), 8.14 (s, 1H), 7.84 (s, 1H), 7.51- 7.66 (m, 1H), 7.37 (s, 1H), 7.12-7.23 (m, 1H), 5.50 (s, 1H), 1.69 (d, J = 7.16 Hz, 3H); LCMS m/z = 478 [M + 1]. |

### Example 221

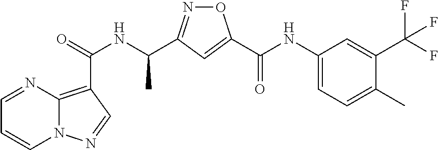

Synthesis of Example 221. The compound of Example 221 was prepared as described previously in Example 209 utilizing pyrazolo[1,5-a]pyrimidine-3-carboxylic acid. ¹H NMR (300 MHz, CDCl₃) δ 8.74-8.80 (m, 1H), 8.66 (s, 1H), 8.60-8.65 (m, 1H), 8.27-8.36 (m, 1H), 8.17 (br. s., 1H), 7.82 (br. s., 1H), 7.66-7.76 (m, 1H), 6.96-7.02 (m, 1H), 5.54-5.63 (m, 1H), 2.43 (d, J=1.79 Hz, 3H), 1.72 (d, J=6.97 Hz, 3H); LCMS m/z=459 [M+1].

### Example 222

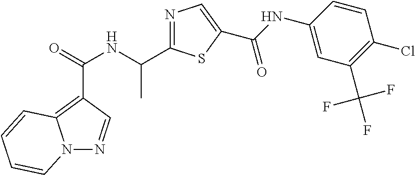

Synthesis of Example 222. The compound of Example 222 was prepared as described previously in Table 1 general amide coupling procedure utilizing H-pyrazolo[1,5-a]pyridine-3-carboxylic acid and compound J.6. LCMS m/z=494 [M+1].

### Example 223

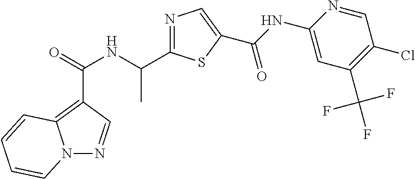

Synthesis of Example 223. The compound of Example 223 was prepared as described previously in Table 1 general amide coupling procedure utilizing H-pyrazolo[1,5-a]pyridine-3-carboxylic acid and compound C.5. ¹H NMR (400 MHz, MeOD) δ 8.64 (dd, J=0.90, 6.95 Hz, 1H), 8.59 (s, 1H), 8.57 (s, 1H), 8.55 (s, 1H), 8.51 (s, 1H), 8.21-8.27 (m, 1H), 7.48 (ddd, J=0.90, 6.95, 8.91 Hz, 1H), 7.08 (td, J=1.33, 6.92 Hz, 1H), 5.51-5.59 (m, 1H), 1.75 (d, J=7.07 Hz, 3H); LCMS m/z=495 [M+1].

### Example 224

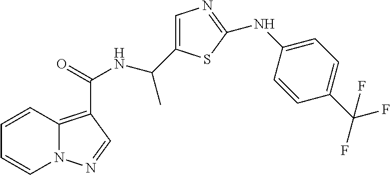

Synthesis of Example 224. The compound of Example 224 was prepared as described previously in Table 1 general amide coupling procedure utilizing H-pyrazolo[1,5-a]pyridine-3-carboxylic acid and Compound A.6. ¹H-NMR (DMSO-D6, 500 MHz) δ 10.45 (s, 1H), 8.88 (d, J=9 Hz, 1H), 8.81 (d, J=8.5 Hz, 2H), 8.20 (d, J=8.5 Hz, 1H), 7.77 (d, J=8.0 Hz, 2H), 7.64 (d, J=8.5 Hz, 2H), 7.45 (d, J=8.5 Hz, 1H)), 7.10 (s, 1H), 7.08 (d, J=8.5 Hz, 1H)), 5.20-5.18 (m, 1H), 1.63 (d, J=7 Hz, 3H); LCMS m/z=431 [M+1].

### Example 225

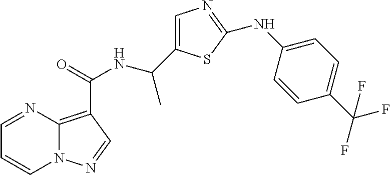

Synthesis of Example 225. The compound of Example 225 was prepared as described previously in Table 1 general amide coupling procedure utilizing pyrazolo[1,5-a]pyrimidine-3-carboxylic acid and compound A.6. ¹H-NMR (DMSO-D6, 500 MHz) δ 10.45 (s, 1H), 9.26 (d, J=9 Hz, 1H), 8.81 (s, 1H), 8.59 (s, 1H), 8.20 (d, J=8.0 Hz, 1H), 7.78-7.75 (m, 2H), 7.62-7.59 (m, 2H), 7.22-7.20 (m, 2H), 5.39-5.35 (m, 1H), 1.63 (d, J=7 Hz, 3H); LCMS m/z=433 [M+1].

### Example 226

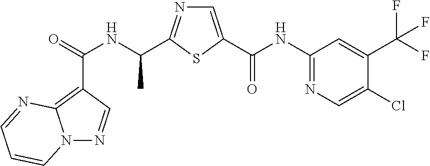

Synthesis of Example 226. The compound of Example 226 was prepared as described previously in Table 1 general amide coupling procedure utilizing pyrazolo[1,5-a]pyrimidine-3-carboxylic acid and compound C.5. LCMS m/z=496 [M+1].

### Example 227

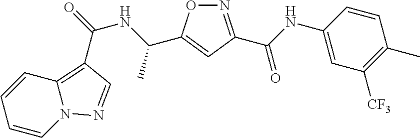

Synthesis of Example 227. The compound of Example 227 was prepared as previously described in Scheme H and Table 1 using 4-methyl-3-trifluoromethyl-aniline. ¹H NMR (300 MHz, DMSO-d₆) δ 10.90 (s, 1H), 8.72-8.83 (m, 2H), 8.65 (s, 1H), 8.17 (m, 2H), 7.87-7.99 (m, 1H), 7.40-7.54 (m, 2H), 7.03-7.14 (m, 1H), 6.84 (s, 1H), 5.47 (m, 1H), 2.40 (s, 3H), 1.60 (m, 3H); LCMS m/z=458 [M+1].

### Example 228

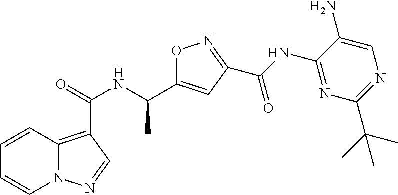

Synthesis of Example 228. The compound of Example 228 was prepared as previously described in Scheme H and Table 1 using 2-tert-butyl-pyrimidine-4,5-diamine. ¹H NMR (400 MHz, MeOD) δ 8.64 (d, J=6.90 Hz, 1H), 8.52 (s, 1H), 8.35 (s, 1H), 8.24 (d, J=8.91 Hz, 1H), 7.49 (ddd, J=1.07, 6.90, 8.91 Hz, 1H), 7.08 (td, J=1.07, 6.90 Hz, 1H), 6.76 (s, 1H), 5.42-5.59 (m, 1H), 1.71 (d, J=7.15 Hz, 3H), 1.44 (s, 9H); LCMS m/z=449 [M+1].

### Example 229

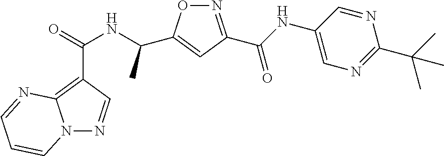

Synthesis of Example 229. The compound of Example 229 was prepared as previously described in Scheme H and Table 1 using 2-tert-butyl-pyrimidin-5-amine. ¹H NMR (400 MHz, MeOD) δ 9.10 (s, 2H), 8.64 (d, J=7.07 Hz, 1H), 8.52 (s, 1H), 8.25 (d, J=8.97 Hz, 1H), 7.48 (dd, J=6.88, 8.97 Hz, 1H), 7.07 (td, J=1.33, 6.92 Hz, 1H), 6.78 (s, 1H), 5.55 (d, J=7.10 Hz, 1H), 1.70 (d, J=7.07 Hz, 3H), 1.33-1.47 (m, 9H); LCMS m/z=434 [M+1].

### Example 230

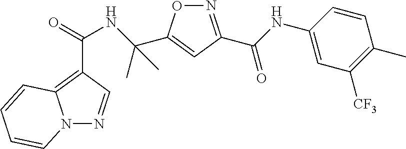

Synthesis of Example 230. The compound of Example 230 was prepared as previously described in Scheme H and Table 1 using tert-butyl 1-hydroxy-2-methylpropan-2-ylcarbamate and 4-methyl-3-trifluoromethyl-aniline. ¹H NMR (300 MHz, DMSO-d₆) δ 10.88 (s, 1H), 8.78 (d, J=6.97 Hz, 1H), 8.74 (s, 1H), 8.41 (s, 1H), 8.18 (s, 1H), 8.09 (d, J=1.32 Hz, 1H), 7.91-7.99 (m, 1H), 7.40-7.50 (m, 2H), 7.00-7.13 (m, 1H), 6.76 (s, 1H), 2.40-2.45 (m, 3H), 1.77 (s, 6H); LCMS m/z=472 [M+1].

### Example 231

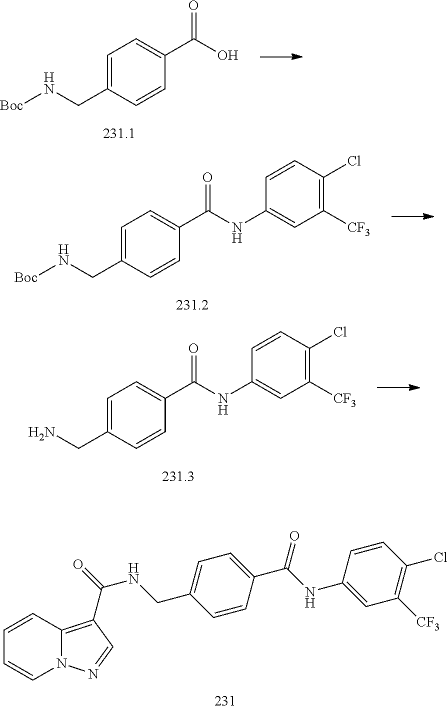

Synthesis of Compound 231.2. The compound 231.2 was prepared as previously described in Example 209 using 4-((tert-butoxycarbonylamino)methyl)benzoic acid and 4-chloro-3-trifluoromethyl-aniline. LCMS m/z=429 [M+1].

Synthesis of Compound 231.3. The compound 231.3 was prepared as previously described in Table 1 general tert-butyl carbamate deprotection method. LCMS m/z=329 [M+1].

Synthesis of Example 231. The compound of Example 231 was prepared as previously described in Table 1 general amide bond formation procedure using H-pyrazolo[1,5-a]pyridine-3-carboxylic acid. ¹H NMR (400 MHz, DMSO-d₆) δ 10.59 (s, 1H), 8.85 (t, J=5.96 Hz, 1H), 8.79 (d, J=7.03 Hz, 1H), 8.62 (s, 1H), 8.37 (s, 1H), 8.23 (d, J=8.91 Hz, 1H), 8.08-8.13 (m, 1H), 7.95 (d, J=8.28 Hz, 2H), 7.71 (d, J=8.91 Hz, 1H), 7.43-7.54 (m, 3H), 7.01-7.11 (m, 1H), 4.58 (d, J=5.90 Hz, 2H); LCMS m/z=473 [M+1].

### Example 232

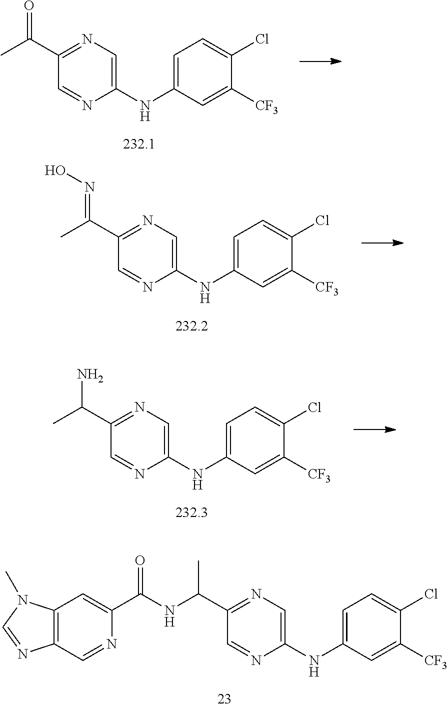

Synthesis of Compound 232.1. Compound 232.1 was prepared as described previously in Scheme B utilizing 4-chloro-3-trifluoromethyl-aniline. ¹H-NMR (DMSO-D₆, 200 MHz) δ 10.62 (bs, 1H), 8.72 (s, 1H), 8.27 (s, 2H), 8.09 (d, J=16.0 Hz, 1H), 7.70 (d, J=6.6 Hz, 1H), 2.50 (s, 3H).

Synthesis of Compound 232.2. Compound 232.2 was prepared as described previously in Scheme A. ¹H-NMR (CD₃OD, 200 MHz) δ 8.64 (s, 1H), 8.23 (s, 1H), 8.15 (s, 1H), 7.97 (d, J=12.0 Hz, 1H), 7.51 (d, J=8.8 Hz, 1H), 2.26 (s, 3H).

Synthesis of Compound 232.3. Compound 232.3 was prepared as described previously in Scheme A. LCMS m/z=300 [M+1].

Synthesis of Example 232. The compound of Example 232 was prepared as described previously in Table 1 general amide coupling procedure. ¹H-NMR (CD₃OD, 500 MHz) δ 8.99 (s, 1H), 8.40 (d, J=14.8 Hz, 2H) 8.28 (d, J=13.0 Hz, 2H), 8.21 (s, 1H), 7.92 (d, J=8.0 Hz, 1H), 7.49 (d, J=8.0 Hz, 1H), 5.34 (q, J=7.0 Hz, 1H), 4.00 (s, 3H), 1.66 (d, J=6.5 Hz, 3H); LCMS m/z=476 [M+1].

TABLE 8
The following compounds of the present invention, set forth in Table 8,
below, were prepared as previously described in Example 232, using compound 232.3 and the
appropriate carboxylic acid prepared as previously described in Table 4.

| Example | Structure | Characterization Data |
|---|---|---|
| 233 | 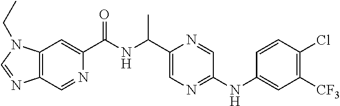 | ¹H-NMR (DMSO-D₆, 500 MHz) δ 9.98 (s, 1H), 9.03 (d, J = 8.0 Hz, 1H), 9.00 (s, 1H), 8.78 (s, 1H), 8.57 (s, 1H), 8.52 (s, 2H), 8.46 (s, 1H), 7.92 (d, J = 8.5 Hz, 2H), 7.61 (d, J = 8.5 Hz, 2H), 5.27-5.21 (m, 1H), 4.42 (q, J = 7.0 Hz, 2H), 1.53 (d, J = 7.0 Hz, 3H), 1.41 (t, J = 7.0 Hz, 3H); LCMS m/z = 490.3 [M + 1]. |
| 234 | 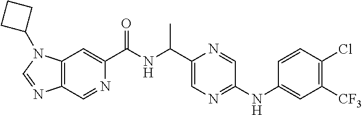 | ¹H-NMR (DMSO-D₆, 500 MHz) δ 10.01 (s, 1H), 9.05 (d, J = 8.0 Hz, 1H), 9.01 (s, 1H), 8.76 (s, 1H), 8.31 (d, J = 8.5 Hz, 4H), 7.93 (d, J = 7.5 Hz, 1H), 7.62 (d, J = 7.5 Hz, 1H), 5.29-5.27 (m, 1H), 5.18-5.15 (m, 1H), 2.77-2.55 (m, 4H), 1.93-1.91 (m, 2H), 1.53 (d, J = 7.0 Hz, 3H); LCMS m/z = 516.2 [M + 1]. |
| 235 | 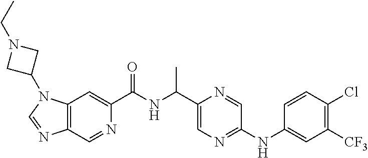 | ¹H-NMR (DMSO-D₆, 500 MHz) δ 9.01 (s, 1H), 8.83 (s, 1H), 8.78 (s, 1H), 8.38 (d, J = 7.5 Hz, 1H), 8.28 (s, 1H), 8.21 (s, 1H), 8.19 (s, 1H), 7.88 (d, J = 7.5 Hz, 1H), 7.45 (d, J = 7.5 Hz, 1H), 5.75-5.73 (m, 1H), 5.32 (d, J = 7.5 Hz, 1H), 4.53 (d, J = 7.5 Hz, 2H), 4.51 (d, J = 7.5 Hz, 2H), 3.38 (m, 3H), 1.63 (d, J = 7.5 Hz, 3H), 1.39 (t, J = 7.5 Hz, 1H), 1.30 (t, J = 7.5 Hz, 3H); LCMS m/z = 545 [M + 1] |

### Examples 236 and 237

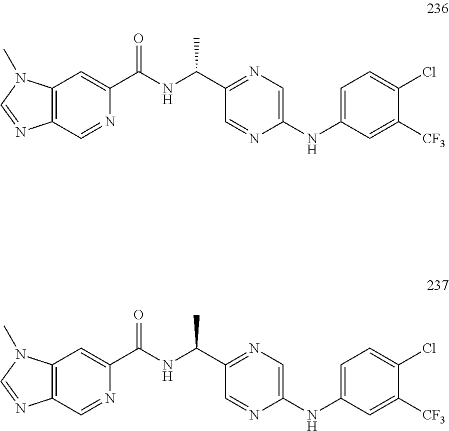

Synthesis of Examples 236 and 237. Examples 236 and 237 were prepared from the compound of Example 232 by preparatory chiral super-critical fluid chromatography on a Chiralcel OJ-H (3×15 cm, #17174) with an isocratic eluant of 25% EtOH(0.1% Et₂NH)/CO₂ at 100 bar, a flow rate of 65 mL/min, an injection vol of 4 mL of a 100 mg/80 mL MeOH/CH₂Cl₂ solution, and monitoring by UV detection at 220 nM to yield 32 mg (>99% ee) of Example 236 as the first eluting peak and 36 mg (>99% ee) of Example 237 as the second eluting peak. Enantiomeric purity was determined by analytical SCF chromatography Chiralcel OJ-H (25×0.46 cm) with an isocratic eluant of 30% EtOH(0.1% Et₂NH)/CO₂ at 100 bar, a flow rate of 3 mL/min, and monitoring by UV detection at 220 nM.

Example 236: LCMS m/z=476 [M+1]. Analytical Chiral SCFC Rt=1.74 min

Example 237: LCMS m/z=476 [M+1]. Analytical Chiral SCFC Rt=2.42 min.

### Examples 238 and 239

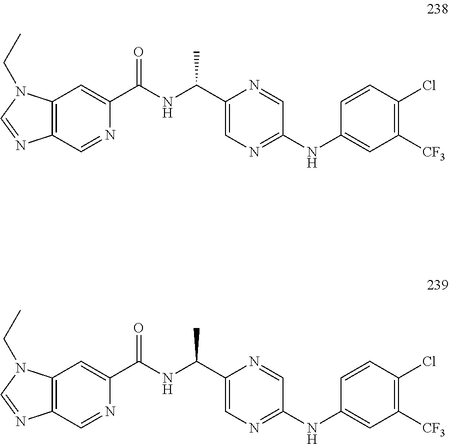

Synthesis of Examples 238 and 239. Examples 238 and 239 were prepared from the compound of Example 233 by preparatory chiral super-critical fluid chromatography on a Chiralcel OJ-H (3×15 cm, #17174) with an isocratic eluant of 25% EtOH(0.1% Et₂NH)/CO₂ at 100 bar, a flow rate of 50 mL/min, an injection vol of 0.5 mL of a 5 mg/mL EtOH solution, and monitoring by UV detection at 220 nM to yield 29 mg (>99% ee) of Example 238 as the first eluting peak and 31 mg (>98% ee) of Example 239 as the second eluting peak. Enantiomeric purity was determined by analytical SCF chromatography Chiralcel OJ-H (25×0.46 cm) with an isocratic eluant of 30% EtOH(0.1% Et₂NH)/CO₂ at 100 bar, a flow rate of 3 mL/min, and monitoring by UV detection at 220 nM.

Example 238: LCMS m/z=437 [M+1]. Analytical Chiral SCFC Rt=1.44 min, 100% ee.

Example 239: LCMS m/z=437 [M+1]. Analytical Chiral SCFC Rt=1.81 min, 99.4% ee.

### Example 240

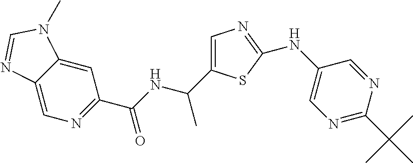

Synthesis of Example 240. To a reaction vial was charged with compound K.4 (10 mg, 0.03 mmol), 2-tert-butyl-pyrimidin-5-ylamine (20.1 mg, 0.133 mmol), Pd₂ dba₃ (8.1 mg, 0.0089 mmol), Xantphos (12 mg, 0.021 mmol), Cesium Carbonate (30 mg, 0.093 mmol) and anhydrous 1,4-Dioxane (2.0 mL, 26 mmol). The mixture was degassed with nitrogen for 15 min, followed by heating in a microwave at 145° C. for 60 min. This resulting mixture was purified via Gilson HPLC (XBridge RP18 5 uM 19 mm×150 mm Column, flow rate 24 mL/min, from 20% B (MeCN with 0.1% TFA) to 70% B in 20 min), affording the 5.5 mg of the TFA salt of Example 240. ¹H NMR (400 MHz, DMSO-d₆) δ 9.13 (d., J=8.5 Hz, 1H), 9.00 (d, J=0.75 Hz, 1H), 8.97 (s, 2H), 8.54 (s, 1H), 8.40 (d, J=1.0 Hz 1H), 7.19 (d, J=1.0 Hz 1H), 5.36 (m, 1H), 2.54 (s, 3H), 1.64 (d, J=6.8 Hz, 3H), 1.32 (s, 9H); LCMS m/z=437 [M+1].

### Examples 241 and 242

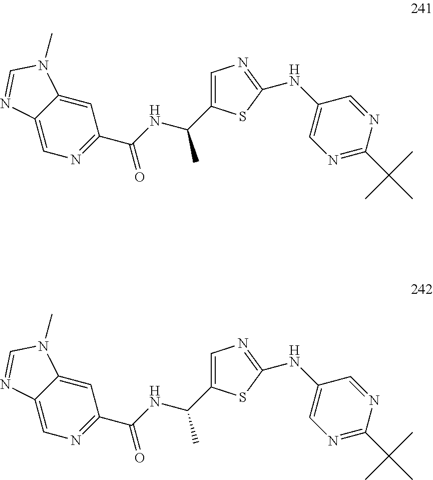

Synthesis of Examples 241 and 242. The compounds of Examples 241 and 242 were prepared by preparatory chiral super-critical fluid chromatography as described in Example 135.

Example 241: LCMS m/z=437 [M+1]. Analytical Chiral SCFC Rt=5.24 min.

Example 242: LCMS m/z=437 [M+1]. Analytical Chiral SCFC Rt=6.08 min.

TABLE 9
The following compounds of the present invention, set forth in Table 9,
below, were prepared as previously described in Example 240, using compound K.4 or L.4 and
the appropriate arylamine or heteroarylamine.

| Example | Structure | Characterization Data |
|---|---|---|
| 243 | 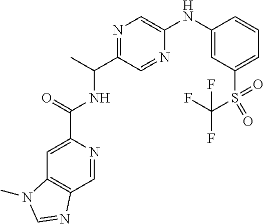 | ¹H-NMR (CD₃OD, 500 MHz) δ 8.98 (s, 1H), 8.68 (s, 1H), 8.36 (d, J = 17 Hz, 1H), 8.32 (s, 1H), 8.24 (s, 1H), 8.12 (d, J = 7.5 Hz, 1H), 7.67-7.60 (m, 2H), 5.34 (d, J = 7.0 Hz, 1H), 4.01 (d, J = 8.0 Hz, 3H), 1.65 (d, J = 7.0 Hz, 3H); LCMS m/z = 506 [M + 1]. |
| 244 | 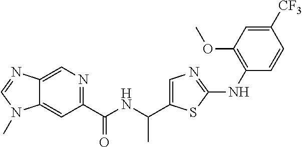 | ¹H-NMR (DMSO-D₆, 500 MHz) δ 9.72 (s, 1H), 9.03 (d, J = 8.5 Hz, 1H), 8.95 (s, 1H), 8.59 (d, J = 8.5 Hz, 1H), 8.46 (s, 1H), 8.33 (s, 1H), 7.26 (d, J = 8.5 Hz, 1H), 7.20 (d, J = 11 Hz, 2H), 5.37-5.34 (m, 1H), 3.93 (s, 3H), 3.90 (s, 3H), 1.62 (d, J = 7.0 Hz, 3H); LCMS m/z = 477 [M + 1]. |
| 245 | 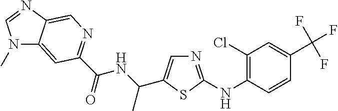 | ¹H-NMR (DMSO-D₆, 500 MHz) δ 8.94 (s, 1H), 8.53 (d, J = 8.5 Hz, 1H), 8.42 (s, 1H), 8.30 (s, 1H), 7.76 (s, 1H), 7.62 (d, J = 9 Hz, 1H), 7.22 (s, 1H), 5.35-5.34 (m, 1H), 3.90 (s, 3H), 1.62 (d, J = 7 Hz, 3H); LCMS m/z = 481 [M + 1]. |
| 246 | 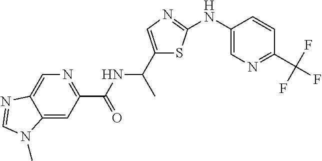 | ¹H-NMR (CD₃OD, 500 MHz) δ 8.96 (s, 1H), 8.77 (s, 1H), 8.42-8.38 (m, 1H), 7.71 (d, J = 9 Hz, 1H), 7.28 (s, 1H), 5.49-5.48 (m, 1H), 4.0 (s, 3H), 1.74 (d, J = 7.0 Hz, 3H); LCMS m/z = 448 [M + 1]. |
| 247 | 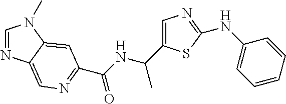 | LCMS m/z 379 [M + 1] |
| 248 | 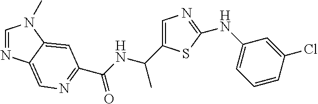 | LCMS m/z = 413 [M + 1] |
| 249 | 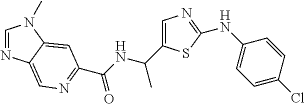 | LCMS m/z = 413 [M + 1] |
| 250 | 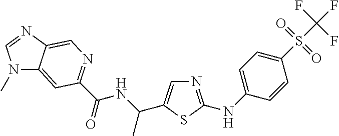 | ¹H-NMR (DMSO-D₆, 500 MHz) δ 10.99 (s, 1H), 9.13 (d, J = 8.5 Hz, 1H), 8.96 (s, 1H), 8.47 (s, 1H), 8.34 (s, 1H), 7.98 (d, J = 9 Hz, 2H), 7.94 (d, J = 9 Hz, 2H), 7.30 (s, 1H), 5.41-5.38 (m, 1H), 3.94 (s, 3H), 1.65 (d, J = 7 Hz, 3H); LCMS m/z = 511 [M + 1]. |
| 251 | 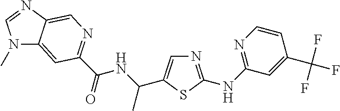 | ¹H-NMR (CD₃OD, 500 MHz) δ 8.97 (s, 1H), 8.48 (s, 1H), 8.39 (s, 2H), 7.33 (s, 1H), 7.27 (s, 1H), 7.12 (d, J = 10 Hz, 1H), 5.54-5.52 (m, 1H), 4.0 (s, 3H), 1.76 (d, J = 7 Hz, 3H); LCMS m/z = 448 [M + 1]. |
| 252 | 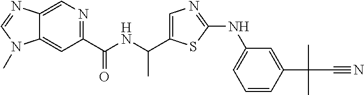 | ¹H-NMR (CD₃OD, 500 MHz) δ 8.96 (s, 1H), 8.39 (d, J = 8.5 Hz, 2H), 7.71 (s, 1H), 7.46 (d, J = 6 Hz, 1H), 7.34-7.30 (m, 1H), 7.18 (s, 1H), 7.12 (d, J = 7.5 Hz, 1H), 5.46- 5.45 (m, 1H), 4.0 (s, 3H), 1.72 (d, J = 6.5 Hz, 3H), 1.71 (s, 6H); LCMS m/z = 446 [M + 1]. |
| 253 | 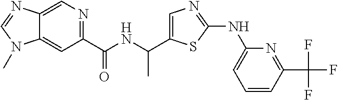 | ¹H-NMR (DMSO-D₆, 500 MHz) δ 11.75- 11.72 (bs, 1H), 9.05 (d, J = 9 Hz, 1H), 8.95 (s, 1H), 8.60 (s, 1H), 8.46 (s, 1H), 8.35 (s, 1H), 7.99 (d, J = 8.5 Hz, 1H), 7.33 (s, 1H), 7.17 (d, J = 10 Hz, 1H), 5.44-5.42 (m, 1H), 3.94 (s, 3H), 1.66 (d, J = 7.0 Hz, 3H); LCMS m/z = 448 [M + 1]. |
| 254 | 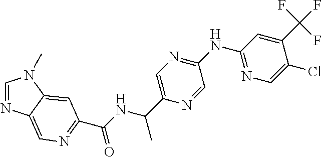 | ¹H-NMR (CD₃OD, 500 MHz) δ 9.0 (s, 1H), 8.83 (s, 1H), 8.42-8.41 (m, 2H), 8.39 (s, 2H), 8.25 (s, 1H), 5.36-5.34 (m, 1H), 4.0 (s, 3H), 1.64 (d, J = 7 Hz, 3H); LCMS m/z = 477 [M + 1]. |
| 255 | 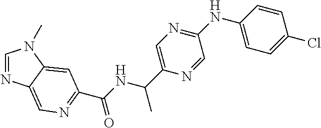 | ¹H-NMR (CD₃OD, 500 MHz) δ 9.02 (s, 1H), 8.39 (s, 1H), 8.38 (s, 1H), 8.21 (s, 1H), 8.20 (s, 1H), 7.63 (d, J = 8.5 Hz, 2H), 7.23 (d, J = 8.5 Hz, 2H), 5.25-5.23 (m, 1H), 4.01 (s, 3H), 1.63 (d, J = 7 Hz, 3H); LCMS m/z = 408.1 [M + 1]. |
| 256 | 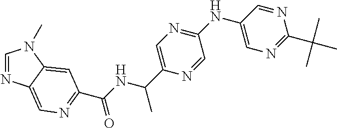 | ¹H-NMR (CD₃OD, 500 MHz) δ 9.08 (s, 2H), 8.98 (s, 1H), 8.39 (s, 1H), 8.28 (s, 1H), 8.23 (s, 1H), 8.22 (s, 1H), 5.34-5.32 (m, 1H), 3.99 (s, 3H), 1.65 (d, J = 7 Hz, 3H), 1.38 (s, 9H); LCMS m/z = 432.2 [M + 1]. |
| 257 | 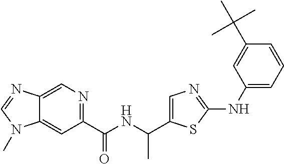 | ¹H-NMR (DMSO-D₆, 500 MHz) δ 9.93 (s, 1H), 9.0 (d, J = 6.5 Hz, 1H), 8.96 (s, 1H), 8.48 (s, 1H), 8.35 (s, 1H), 7.52 (s, 1H), 7.45 (d, J = 7.5 Hz, 1H), 7.20-7.17 (m, 1H), 7.13 (s, 1H), 6.95 (d, J = 7.5 Hz, 1H), 5.36-5.33 (m, 1H), 3.95 (s, 3H), 1.63 (d, J = 6.5 Hz, 3H), 1.25 (s, 9H); LCMS m/z = 435.3 [M + 1]. |
| 258 | 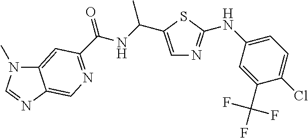 | ¹H-NMR (CD₃OD, 500 MHz) δ 8.96 (s, 1H), 8.41 (d, J = 8.5 Hz, 2H), 8.18 (s, 1H), 7.71 (d, J = 8.5 Hz, 1H), 7.35 (d, J = 8.5 Hz, 1H) 7.21 (s, 1H) 5.50-5.49 (m, 1H), 4.02 (s, 3H), 1.75 (d, J = 7 Hz, 3H); LCMS m/z = 481 [M + 1]. |
| 259 | 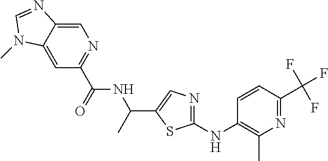 | ¹H-NMR (CD₃OD, 500 MHz) δ 8.98 (s, 1H), 8.75 (d, J = 8.5 Hz, 2H), 8.41 (d, J = 9 Hz, 1H), 7.60 (d, J = 8.5 Hz, 1H), 7.25 (s, 1H), 5.50-5.49 (m, 1H), 4.02 (s, 3H), 2.58 (s, 3H) 1.75 (d, J = 7 Hz, 3H); LCMS m/z = 462 [M + 1]. |
| 260 | 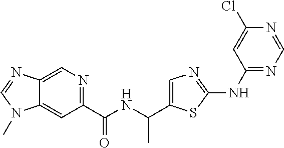 | ¹H-NMR (CD₃OD, 500 MHz) δ 8.96 (s, 1H), 8.41 (s, 1H), 8.28 (s, 2H), 7.29 (s, 1H), 7.10 (s, 1H), 5.50-5.49 (m, 1H), 4.02 (s, 3H), 1.75 (d, J = 7 Hz, 3H); LCMS m/z = 415 [M + 1]. |
| 261 | 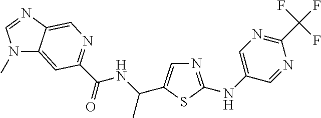 | ¹H-NMR (CD₃OD, 500 MHz) δ 9.23 (s, 2H), 8.97 (s, 1H), 8.42 (d, J = 8.5 Hz, 2H), 7.36 (s, 1H), 5.54-5.52 (m, 1H), 4.02 (s, 3H), 1.78 (d, J = 7 Hz, 3H); LCMS m/z = 449 [M + 1]. |
| 262 | 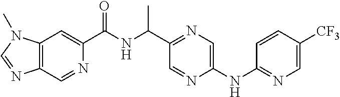 | ¹H-NMR (CD₃OD, 500 MHz) δ 9.10 (s, 1H), 8.96 (s, 1H), 8.55 (s, 1H), 8.39 (s, 1H), 8.36 (d, J = 8.5 Hz, 2H), 7.91 (d, J = 9 Hz, 1H), 7.79 (d, J = 8.5 Hz, 1H), 5.48- 5.46 (m, 1H), 4.01 (s, 3H), 1.63 (d, J = 7 Hz, 3H); LCMS m/z = 443 [M + 1]. |
| 263 | 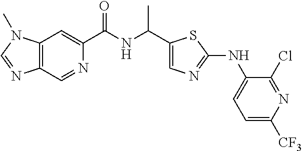 | ¹H-NMR (CD₃OD, 500 MHz) δ 9.04 (bs, 1H), 8.93 (s, 1H), 8.40 (s, 1H), 8.37 (s, 1H), 7.68 (d, J = 8.0 Hz, 1H), 7.38 (s, 1H), 5.36-5.32 (m, 1H), 4.00 (s, 3H), 1.71 (d, J = 7.5 Hz, 3H); LCMS m/z = 482 [M + 1]. |
| 264 | 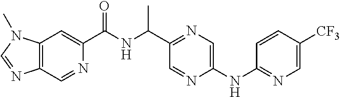 | ¹H-NMR (DMSO-D₆, 500 MHz) δ 9.01 (s, 1H), 8.91 (s, 1H), 8.46 (d, J = 10 Hz, 2H), 8.35- 8.32 (m, 2H), 7.83 (d, J = 9.0 Hz, 2H), 5.29- 5.28 (m, 1H), 3.94 (s, 3H), 1.57 (d, J = 7.0, 3H); LCMS m/z = 443 [M + 1]. |
| 265a | 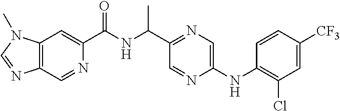 | ¹H-NMR (DMSO-D₆, 500 MHz) δ 9.14 (s, 1H), 9.08 (d, J = 8.5 Hz, 1H), 9.00 (s, 1H), 8.60 (s, 1H), 8.48 (s, 1H), 8.36- 8.34 (m, 2H), 8.31 (s, 1H), 7.86 (s, 1H), 7.65 (d, J = 9.0 Hz, 1H), 5.31-5.28 (m, 1H), 3.94 (s, 3H), 1.55 (d, J = 7.0, 3H); LCMS m/z = 476 [M + 1]. |
| 265b | 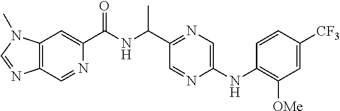 | ¹H-NMR (DMSO-D₆, 500 MHz) δ 9.04 (d, J = 8.5 Hz, 1H), 8.99 (d, J = 10 Hz, 1H), 8.59 (s, 1H), 8.53 (d, J = 8.5 Hz, 1H), 8.47 (s, 1H), 8.33 (s, 1H), 8.28 (s, 1H), 7.26 (s, 1H), 5.28-5.25 (m, 1H), 3.96 (s, 3H), 3.93 (s, 3H), 1.53 (d, J = 7.0, 3H); LCMS m/z = 472 [M + 1]. |

### Examples 266 and 267

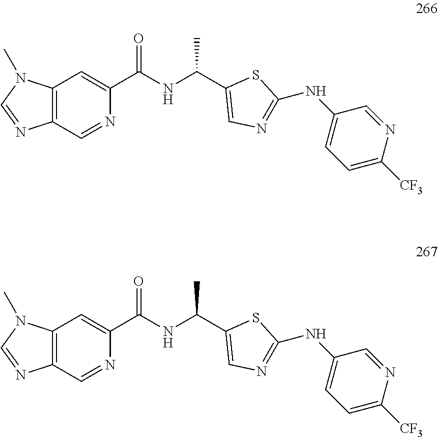

Synthesis of Examples 266 and 267. Examples 266 and 267 were prepared from the compound of Example 246 by preparatory chiral super-critical fluid chromatography on a Chiralpak IC (3×15 cm) with an isocratic eluant of 40% EtOH(0.1% Et₂NH)/CO₂ at 100 bar, a flow rate of 85 mL/min, an injection vol of 0.8 mL of a 10 mg/mL MeOH solution, and monitoring by UV detection at 220 nM to yield 36 mg (>99% ee) of Example 266 as the first eluting peak and 34 mg (>98% ee) of Example 267 as the second eluting peak. Enantiomeric purity was determined by analytical SCF chromatography Chiralpak IC (15×0.46 cm) with an isocratic eluant of 40% EtOH(0.1% Et₂NH)/CO₂ at 100 bar, a flow rate of 3 mL/min, and monitoring by UV detection at 220 nM.

Example 266: LCMS m/z=448 [M+1]. Analytical Chiral SCFC Rt=3.72 min, 99.2% ee.

Example 267: LCMS m/z=448 [M+1]. Analytical Chiral SCFC Rt=4.17 min, 99.0% ee.

### Example 268

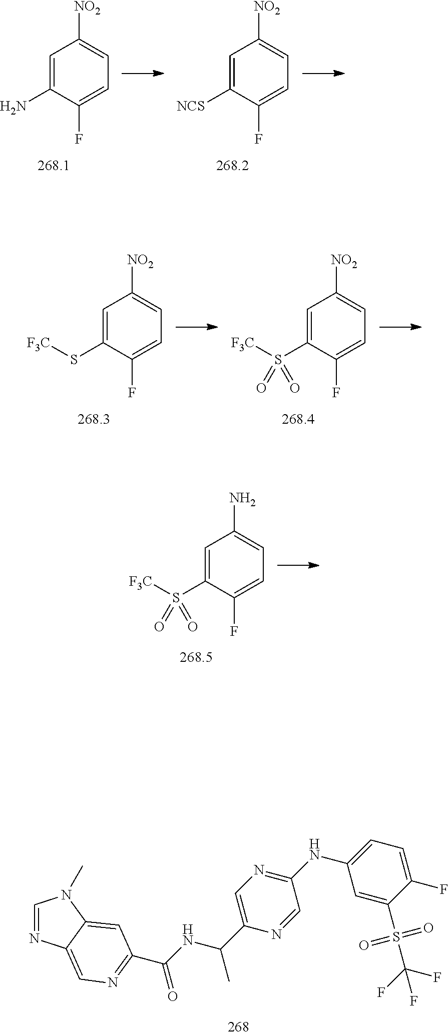

Synthesis of Compound 268.2. To a stirred solution of compound 268.1 (1.0 g, 6.41 mmol) in 4.2 g of concentrated H₂SO₄ was added NaNO₂ (1.5 g (0.023 mol) in 5 mL of H₂O) at 0° C. for a period of 20 min, followed by the addition of CuSO₄ (2.9 g (0.018 mo)l in 16 mL of H₂O) and FeSO₄ (5.2 g (0.035 mol) in 10 mL of H₂O) at 0° C. KSCN (1.2 g (0.013 mol) in 5 mL of H₂O) was added to the reaction mixture at 0° C. for a period of 2 hr. The resulting reaction mixture was stirred at room temperature for 2 hr. After completion of the starting material (by TLC), the resultant reaction mixture was filtered through celite bed and the filtrate was extracted with CH₂Cl₂. The organic layer was washed with water (20 ml) and dried over anhydrous sodium sulphate and evaporated under reduced pressure. The crude material was purified by column chromatography [silica gel (60-120 mesh, 40 g), 20 mm diameter, 350 mm length gradient (5-10% EtOAc/Hexane)] to afford compound 268.2 (100 mg, 7%) as pale yellow liquid. ¹H-NMR (CDCl₃, 500 MHz) δ 8.58-8.56 (m, 1H), 8.37-8.34 (m, 1H), 7.40 (d, J=9.0 Hz, 8.5 Hz, 1H); ¹³C-NMR (CDCl₃, 500 MHz) δ 164.32, 162.24, 144.88, 127.54, 117.50, 115.01, 107.1; ¹⁹F-NMR (CDCl₃, 500 MHz) δ −98.22.

Synthesis of Compound 268.3. To a stirred solution of compound 268.2 (1.0 g, 0.0041 mol) in THF (10 ml) was added TMS-CF₃ (2.3 g, 0.0166 mol) and (n-Bu)₄NF (433 mg, 0.00166 mol) at 0° C. The resulting reaction mixture was stirred at room temperature for 4 hr. After completion of the starting material (by TLC), the reaction mixture was quenched with water (15 ml) and extracted with EtOAc (2×10 ml). The combined organic layer was dried over anhydrous sodium sulphate and evaporated under reduced pressure. The crude material was purified by column chromatography [silica gel (60-120 mesh, 20 g) 20 mm diameter, 300 mm length and eluted with (5% EtOAc/Hexane)] to afford compound 268.3 (500 mg, 50%) as pale yellow liquid. ¹H-NMR (CD₃OD, 200 MHz) δ 8.64-8.61 (m, 1H), 8.55-8.50 (m, 1H), 7.40 (dd, J=7.6 Hz, 7.4 Hz, 1H); LCMS m/z 243.3 [M+1].

Synthesis of Compound 268.4. To a stirred solution of compound 268.3 (500 mg, 0.0021 mol) in H₂SO₄ (2.5 ml, 0.010 mol) was added CrO₃ (1 g, 0.010 mol) at room temperature. The resulting reaction mixture was stirred for 2 hr at room temperature. After completion of the starting material (by TLC), the resulting reaction mixture was quenched with cold water (5 ml) and extracted with EtOAc (2×10 ml). The organic layer was washed with water (20 ml) and dried over anhydrous sodium sulphate and evaporated under reduced pressure to afford compound 268.4 (300 mg, 53%) as pale yellow liquid. ¹H-NMR (CDCl₃, 200 MHz) δ 8.96-8.91 (m, 1H), 8.78-8.70 (m, 1H), 7.66 (dd, J=8.8 Hz, 8.6 Hz, 1H); ¹⁹F-NMR (CDCl₃, 500 MHz): δ −77.82, −93.68.

Synthesis of Compound 268.5. To the solution of compound 268.4 (500 mg, 0.0017 mol) in acetic acid (5 ml) was added Fe powder (484 mg, 0.0087 mol). The resulting reaction mixture was stirred at 70° C. for 16 hr. After completion of the starting material (by TLC), the reaction mixture was distilled off, the crude material quenched with water (20 ml) and extracted with CH₂Cl₂ (2×20 ml). The combined organic layer was washed with water (20 ml) and dried over anhydrous sodium sulphate. The solvent was evaporated under reduced pressure to afford compound 268.5 (180 mg, 40%) as pale yellow liquid. ¹H-NMR (CDCl₃, 200 MHz) δ 7.29 (bs, 1H), 7.04 (bs, 1H), 6.74-6.68 (m, 1H), 4.01-3.82 (bs, 1H).

Synthesis of Example 268. The compound of Example 268 was prepared as previously described in Example 240 using compound L.4 ¹H-NMR (CD₃OD, 500 MHz) δ 8.97 (s, 1H), 8.58-8.57 (m, 1H), 8.38 (s, 1H), 8.35 (s, 1H), 8.28 (s, 1H), 8.21 (s, 1H), 8.19-8.15 (m, 1H), 7.44-7.40 (m, 1H), 7.68 (d, J=8.5 Hz, 1H), 5.33-5.32 (m, 1H), 3.98 (s, 3H), 1.64 (d, J=7 Hz, 3H); LCMS m/z=524 [M+1].

### Example 269

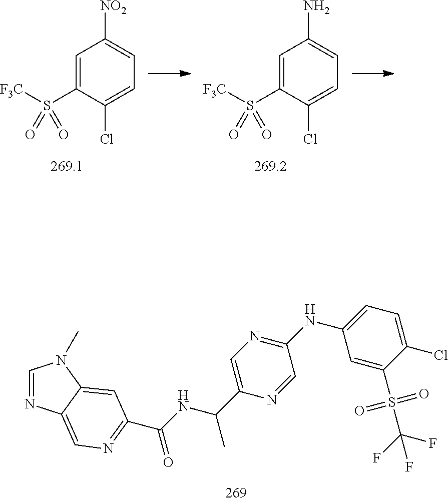

Synthesis of Compound 269.2. The compound 269.2 was prepared as previously described in Example 268 using compound 269.1 ¹H-NMR (CDCl₃, 200 MHz) δ 7.29 (bs, 1H), 7.04 (bs, 1H), 6.74-6.68 (m, 1H), 4.01-3.82 (bs, 1H).

Synthesis of Example 269. The compound of Example 269 was prepared as previously described in Example 240 using compound L.4. ¹H-NMR (CD₃OD, 500 MHz) δ 9.0 (s, 1H), 8.79 (s, 1H), 8.41 (s, 1H), 8.34 (s, 1H), 8.25 (s, 1H), 8.19 (s, 1H), 8.18 (d, J=7.5 Hz, 1H), 7.68 (d, J=8.5 Hz, 1H), 5.37-5.35 (m, 1H), 4.01 (s, 3H), 1.67 (d, J=7 Hz, 3H); LCMS m/z=540 [M+1].

### Example 270

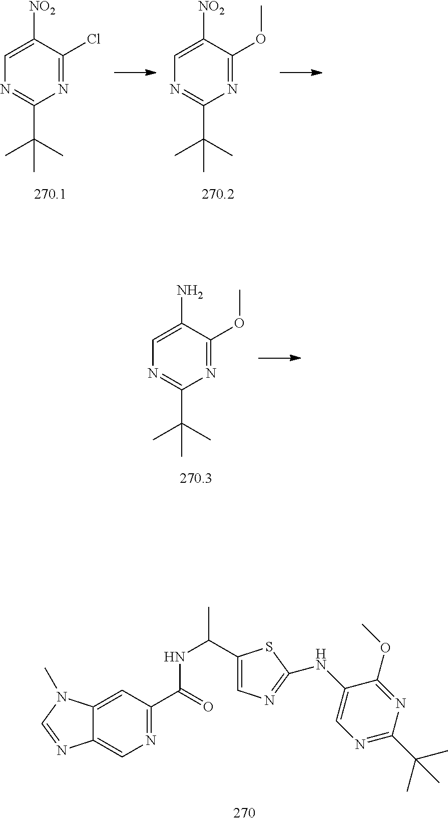

Synthesis of Compound 270.2. To a solution of compound 270.1 (1 g, 4.6 mmol, WO2006065703) in MeOH (3 ml) was added triethylamine (1 ml, 2 eq) in a sealed tube and stirred at 80° C. for 2 hr. After completion of the starting material (by TLC), the reaction mixture was cooled to room temperature and evaporated under reduced pressure. The crude material was diluted with water (15 ml) and extracted with EtOAc (2×15 ml). The combined organic layers was washed with brine solution and dried over Na₂SO₄. The solvent was evaporated under reduced pressure to afford compound 270.2 (700 mg, 71%) as yellowish oil. ¹H-NMR (CDCl₃, 200 MHz): δ 9.12 (s, 1H), 4.18 (s, 1H), 1.41 (s, 9H). LCMS m/z=212 [M+1].

Synthesis of Compound 270.3. To a solution of compound 270.2 (500 mg, 0.0023 mol) in 1,4-dioxane:water (6 ml of 1:1) was added sodium dithionate (1 g, 0.0057 mol) and Na₂CO₃ (645 mg, 0.0053 mol) at 0° C. and stirred at 0° C. for 3 hr. After the completion of starting material (by TLC), the reaction mixture was diluted with water (10 ml), and extracted with ethyl acetate (2×20 ml). The combined organic layers were washed with brine solution, dried over anhydrous sodium sulphate, and concentrated under reduced pressure. The crude material was purified by column chromatography [silica gel (60-120 mesh; 30 g) gradient 5-15% EtOAc/Hexane] to afford compound 270.3 (80 mg, 18% yield) as white solid. ¹H-NMR (CDCl₃, 200 MHz) δ 7.91 (s, 1H), 4.02 (s, 1H), 3.65 (bs, 2H), 1.35 (s, 9H); LCMS m/z=182 [M+1].

Synthesis of Example 270. To a suspension of NaH (31 mg, 0.0012 mol) in anhydrous 1,4-dioxane (4 ml) was added compound 270.3 (112 mg, 0.00062 mol) at 0° C. and stirred for 20 min. Then compound K.4 (100 mg, 0.000031 mol) was added and heated at 110° C. for 5 hr. After completion of the starting material (by TLC), the reaction mixture was cooled to room temperature, diluted with water (5 ml), and extracted with EtOAc (2×10 ml). The combined organic layers was washed with brine solution and dried over Na₂SO₄. The solvent was evaporated under reduced pressure. The resulting crude material was purified by column chromatography [silica gel (60-120 mesh; 20 g): gradient 5-15% isopropanol/CH₂Cl₂] to afford Example 270 (42 mg, 37%) as an off-white solid. ¹H-NMR (CD₃OD, 500 MHz) δ 9.31 (s, 1H), 8.96 (s, 1H), 8.39 (s, 1H), 8.37 (s, 1H), 7.20 (s, 1H) 5.46-5.45 (m, 1H), 4.07 (s, 3H), 4.01 (s, 3H), 1.72 (d, J=7 Hz, 3H), 1.37 (s, 9H); LCMS m/z=467 [M+1].

### Example 271

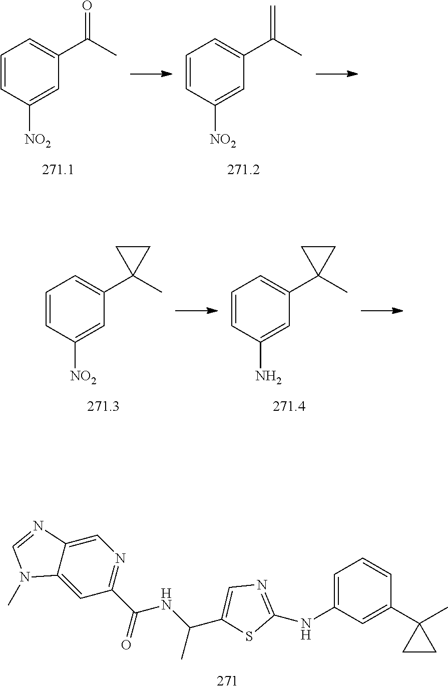

Synthesis of Compound 271.2. To a stirred solution of (methyl triphenylphosphonium bromide (16.2 g, 45.41 mmol) in dry THF (100 ml) at −10° C., potassium tert-butoxide (5.1 g, 45.41 mmol) was slowly added and reaction was stirred 30 minutes at −10° C. A solution of 3-nitro-acetophenone 271.1 (5.0 g, 30.3 mmol) in dry THF (10 mL) was added at −10° C. and the reaction mixture was stirred at room temperature for 1 hr. After completion, the reaction mixture was quenched with saturated aqueous sodium bicarbonate solution and extracted twice with EtOAc. The combined organic layer was washed with water, dried over anhydrous Na₂SO₄ and concentrated. The crude compound obtained was purified by column chromatography using 100% hexanes with gradient to 2% EtOAc/hexane as eluent. Compound 271.2 (3 g, 60%) was obtained as yellow colour liquid. ¹H-NMR (CDCl₃) δ 8.3 (s, 1H), 8.1-8.2 (d, 1H), 7.75-7.8 (d, 1H), 7.5 (t, 1H) 5.5 (s, 1H) 5.25 (s, 1H) 2.2. (s, 3H).

Synthesis of Compound 271.3. To a stirred solution of compound 271.2 (3.0 g, 18.4 mmol) in dry 1,2-ethanedichloride (60 mL) under nitrogen atmosphere at 0° C., diethyl zinc (46 mL, 1M solution in hexane) and diiodomethane (7.42 mL, 92 mmol) were added. The reaction was stirred at 0° C. for 0.5 hr and at room temperature for 2 hr. Reaction was quenched with saturated ammonium chloride solution and extrated twice with CH₂Cl₂. The combined organic layers were dried over anhydrous Na₂SO₄ and concentrated. The residue was purified by filter column to obtain 1.5 g as a 2:1 mixture of compound 271.3 and starting material. This mixture was taken in 1:1 THF:H₂O (10 mL), and treated with OsO₄ (catalytic) and NMO (1.1 g, 9.2 mmol.). The reaction mass was stirred at room temperature for 12 hr. Reaction was diluted with water, extracted with EtOAc, dried and concentrated. Residue was purified by column chromatography to using hexane to obtain 0.9 g of compound 271.3 (27%). ¹H-NMR (CDCl₃) δ 8.1 (s, 1H), 8.0-8.1 (d, 1H), 7.5-7.6 (d, 1H), 7.4-7.5 (t, 1H), 1.45 (s, 3H), 0.95-1.0 (m, 2H), 0.9-0.95 (m, 2H).

Synthesis of Compound 271.4 To a stirred solution of compound 271.3 (1.8 g, 10.1 mmol) in 1:1 MeOH:water (20 mL) was added sodium dithionate (4.42 g, 25.4 mmol) and sodium carbonate (2.69 g, 25.4 mmol), and stirred for 2 hr at room temperature. After completion of the reaction the volatiles were removed under vacuum and the aqueous layer was acidified and extracted with ethyl acetate. Organic layer was dried over anhydrous Na₂SO₄ and concentrated. The crude compound obtained was purified by column chromatography using EtOAC 3-4% in Hexane as eluent. Compound 271.4 (700 mg, 46%) was obtained as brown liquid. ¹H-NMR (CDCl₃) δ 7.1-7.2 (t, 1H), 6.65-6.8 d, 1H) 6.65 (s, 1H), 6.5-6.6 (d, 1H), 3.4-3.8 (bs, 2H, D₂O exchangeable), 1.4 (s, 3H), 0.95-1.0 (m, 2H), 0.9-0.95 (m 2H); LCMS m/z 148 [M+1].

Synthesis of Example 271. The compound of Example 271 was prepared as previously described in Example 240. ¹H-NMR (DMSO-D6, 500 MHz) δ 9.92 (s, 1H), 9.0 (d, J=8.5 Hz, 1H), 8.96 (s, 1H), 8.48 (s, 1H), 8.35 (s, 1H), 7.41 (s, 1H), 7.39 (d, J=8 Hz, 1H), 7.17-7.13 (m, 2H), 6.77 (d, J=7.5 Hz, 1H), 5.34-5.32 (m, 1H), 3.95 (s, 3H), 1.63 (d, J=7 Hz, 3H), 1.34 (d, J=6.5 Hz, 3H), 0.78 (d, J=6.5 Hz, 2H), 0.73-0.71 (m, 2H); LCMS m/z=433.1 [M+1].

### Example 272

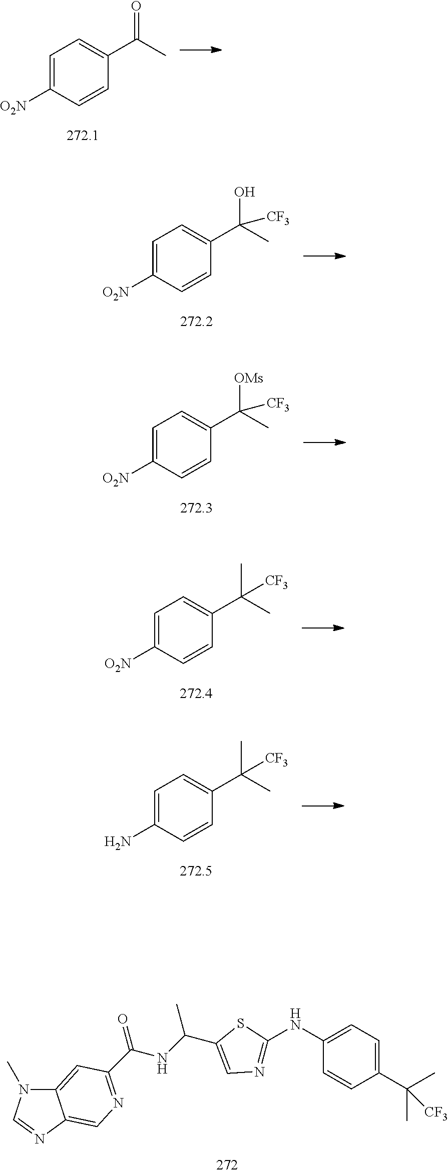

Synthesis of Compound 272.2. To a stirred solution of compound 272.1 (20 g, 0.12 mol) in THF (200 ml) were added TMS-CF₃ (53 ml, 0.18 mol), TBAF (60 ml, 3 vol) at 0° C., and the resulting reaction mixture was stirred at room temperature for 1 hr. After completion of the starting material (by TLC), volatiles were removed under reduced pressure. The crude material was quenched with water (100 ml) and extracted with EtOAc (2×100 ml). The combined organic layers were washed, dried over anhydrous sodium sulphate. The solvent was evaporated under reduced pressure to afford compound 272.2 (20 g, 70%) as a red syrup which was used for next step without any further purification. ¹H-NMR (CDCl₃, 200 MHz) δ 8.31 (d, J=12 Hz, 2H), 7.78 (d, J=12 Hz, 2H), 3.25 (bs, 1H), 1.83 (s, 3H).

Synthesis of Compound 272.3. To a stirred solution of compound 272.2 (20 mg, 0.085 mol) in CH₂Cl₂ (200 ml), were added triethylamine (15.9 ml, 0.011 mol) and methanesulfonyl chloride (10.7 mg, 0.093 mol) at 0° C. The reaction mixture was stirred at room temperature for 2 hr. After completion of the starting material (by TLC), the reaction mixture was quenched with water (100 ml) and extracted with CH₂Cl₂ (2×100 ml). The combined organic layers were washed with brine and dried over anhydrous sodium sulphate. The solvent was evaporated under reduced pressure. The crude residue was purified by column chromatography [silica gel (60-120 mesh, 300 g), gradient (6-17% EtOAc/Hexane)] to afford compound 272.3 (22 mg, 83%) as a red solid. ¹H-NMR (CDCl₃, 200 MHz) δ 8.25 (d, J=13 Hz, 2H), 7.76 (d, J=13 Hz, 2H), 3.21 (s, 3H), 2.35 (s, 3H).

Synthesis of Compound 272.4. A solution of compound 272.3 (5 g, 0.022 mol) in cyclohexane: CH₂Cl₂ (65 ml of 3:1) was treated with Al(CH₃)₃ (9.6 ml, 0.134 mol) at 0° C. The resulting reaction mixture was stirred at 60° C. for 5 hr. After completion of the starting material (by TLC), the reaction mixture was cooled to room temperature and quenched with ice cold water (50 ml), and extracted with CH₂Cl₂ (2×50 ml). The combined organic layers were dried over anhydrous sodium sulphate. The solvent was evaporated under reduced pressure. The crude material was purified by column chromatography [silica gel (60-120 mesh, 50 g), (Hexane)] to afford compound 272.4 (800 mg, 21% with 3.01% HPLC purity) as a red oil. This material further purified by preparative reverse-phase HPLC to afford compound 272.4 (30 mg). ¹H-NMR (CDCl₃, 500 MHz) δ 8.29 (d, J=12 Hz, 2H), 7.56 (d, J=12 Hz, 2H), 2.05 (s, 6H).

Synthesis of Compound 272.5. A solution of compound 272.4 (600 mg, 0.0025 mol) in methanol (6 ml) was treated with 10% Pd/C (60 mg, 10 mol %), and stirred under hydrogen balloon pressure at room temperature for 5 hr. After the completion of the starting material (by TLC), the mixture was filtered through a celite bed, which was washed with EtOAc (20 ml). The filtrate was evaporated under reduced pressure and crude material was purified by column chromatography [silica gel (60-120 mesh, 20 g), gradient (6-18% EtOAc/Hexane)] to afford compound 272.5 (250 mg, 50% yield with 56% HPLC purity) as the red oil. ¹H-NMR (DMSO-d₆, 500 MHz) δ 7.19 (d, J=11 Hz, 2H), 6.58 (d, J=11 Hz, 2H), 5.10 (bs, 2H), 1.43 (s, 6H). LCMS m/z 204.1 [M+1].

Synthesis of Example 272. The compound of Example 272 was prepared as previously described in Example 240. ¹H-NMR (CD₃OD, 500 MHz): δ 8.98 (s, 1H), 8.45 (s, 1H), 8.41 (s, 1H), 7.55 (d, J=8.5 Hz, 2H), 7.45 (d, J=8.5 Hz, 2H), 7.22 (s, 1H), 5.42-5.41 (m, 1H), 4.00 (s, 3H), 1.73 (d, J=7 Hz, 3H), 1.57 (s, 6H). LCMS m/z=489 [M+1].

### Example 273

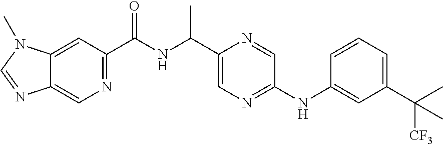

Synthesis of Example 273. The compound of Example 273 was prepared as described in Example 272 using 1-(3-nitrophenyl)ethanone. ¹H-NMR (CD₃OD, 500 MHz) δ 8.97 (s, 1H), 8.38 (s, 1H), 8.36 (s, 1H), 8.19 (s, 1H), 8.16 (s, 1H), 7.82 (s, 1H), 7.68 (d, J=8 Hz, 1H), 7.30-7.27 (m, 1H), 7.15 (d, J=7.5 Hz, 1H), 5.30-5.29 (m, 1H), 3.98 (s, 3H), 1.63 (d, J=7 Hz, 3H), 1.57 (s, 6H); LCMS m/z=484 [M+1].

### Example 274

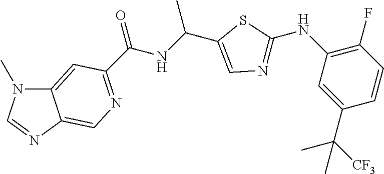

Synthesis of Example 274. The compound of Example 274 was prepared as previously described in Example 272 using 1-(4-fluoro-3-nitrophenyl)ethanone. ¹H-NMR (CD₃OD, 400 MHz) δ 8.91 (s, 1H), 8.45-8.42 (m, 2H), 7.22 to 7.13 (m, 3H), 5.43-5.41 (m, 1H), 3.91 (s, 3H), 2.76-2.74 (d, 3H), 1.58 (s, 6H); LCMS m/z=507 [M+1].

### Example 275

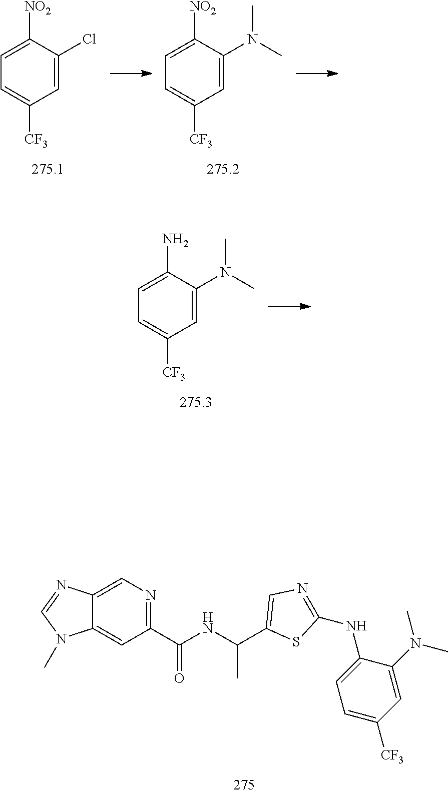

Synthesis of Compound 275.2. To a stirred solution of 2-chloro-1-nitro-4-(trifluoromethyl)benzene 275.1 (200 mg, 0.00088 mol) in THF (0.4 ml) was added dimethyl amine (0.2 ml, 0.0041 mol) in a sealed tube and the reaction mixture was stirred at 100° C. for 16 hr. After completion of the starting material (by TLC), the reaction mixture was cooled to room temperature and volatiles were evaporated under reduced pressure. The crude material was diluted with water (15 ml) and extracted with EtOAc (2×15 ml). The combined organic layers were washed with brine solution, dried over anhydrous Na₂SO₄ and concentrated under reduced pressure. The residue was purified by preparative TLC to afford compound 275.2 (160 mg, 77%) as yellow syrup. ¹H-NMR (CDCl₃, 200 MHz) δ 7.83 (d, J=8.8 Hz, 1H), 7.22 (s, 1H), 6.99 (d, J=8.8 Hz, 1H), 2.94 (s, 6H), LCMS m/z 216 [M+1-F].

Synthesis of Compound 275.3. To a solution of compound 275.2 (800 mg, 0.0034 mol) in methanol (1.6 ml) was added 10% Pd/C (50 mg, 0.0057 mol) at room temperature and stirred under hydrogen balloon pressure at room temperature for 16 hr. After completion of the starting material (by TLC), the reaction mixture was filtered through a celite, rinsing with MeOH. The filtrate was concentrated under reduced pressure. The crude material was purified by column chromatography [silica gel (60-120 mesh; 40 g) gradient 2-4% EtOAc/Hexane] to afford compound 275.3 (650 mg, 93% yield) as a brown color syrup. LCMS m/z=205 [M+1].

Synthesis of Example 275. The compound of Example 275 was prepared as previously described in Example 240. ¹H-NMR (DMSO-D6, 500 MHz) δ 9.53 (s, 1H), 9.02 (d, J=8.5 Hz, 1H), 8.95 (s, 1H), 8.46 (d, J=7.5 Hz, 1H), 8.33 (s, 1H), 7.34 (d, J=9.5 Hz, 1H), 7.18 (s, 1H), 5.37-5.35 (m, 1H), 3.93 (s, 3H), 2.61 (s, 6H), 1.63 (d, J=7.0 Hz, 3H); LCMS m/z=490.2 [M+1].

### Example 276

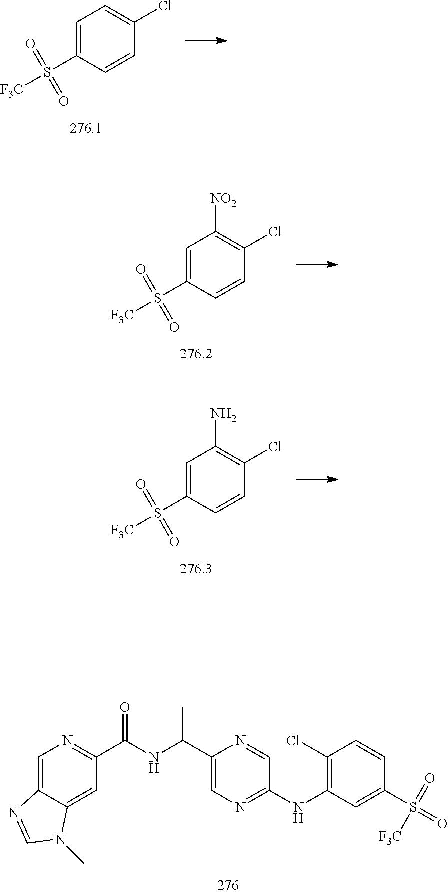

Synthesis of Compound 276.2. To a stirred solution of compound 276.1 (500 mg, 0.002049 mol), in Oleum (2.5 g, 0.014 mol) was added fuming HNO₃ (5 ml). The resulting reaction mixture was stirred at 100° C. for 24 hr. After completion of the starting material (by TLC), the reaction mixture was quenched with water (10 ml) and the extracted ∘ was extracted with CH₂Cl₂ (2×10 ml). The organic layer was washed with water (20 ml) and dried over anhydrous sodium sulphate and evaporated under reduced pressure. The crude material was purified by column chromatography [silica gel (60-120 mesh, 40 g), 30 mm diameter, 500 mm length gradient (5-15% EtOAc/Hexane)] to afford compound 276.2 (2 g, 24%) as colorless liquid. ¹H-NMR (CDCl₃, 500 MHz) δ 8.53 (bs, 1H), 8.19-8.14 (m, 1H), 7.94 (d, J=8.8 Hz, 1H).

Synthesis of Compound 276.3. To the solution of compound 276.2 (1 g, 0.001730 mol) in acetic acid (40 ml), was added iron powder (1.2 g, 0.001730 mol), and the resulting reaction mixture was stirred at 70° C. for 16 hr. After completion of the starting material (by TLC), the reaction mixture was distilled off, the crude reaction material was quenched with water (20 ml) and extracted with CH₂Cl₂. The organic layer was washed with water (20 ml) and dried over anhydrous sodium sulphate. The solvent was evaporated under reduced pressure to afford compound 276.3 (0.8 g, 89%) as as pale yellow liquid. ¹H-NMR (DMSO-D₆, 200 MHz): δ 7.66 (d, J=8.4 Hz, 1H), 7.46 (bs, 1H), 7.17-7.12 (m, 1H).

Synthesis of Example 276. The compound of Example 276 was prepared as previously described in Example 240 using compound L.4. ¹H-NMR (CD₃OD, 500 MHz) δ 9.21 (s, 1H), 8.99 (s, 1H), 8.52 (s, 1H), 8.39 (s, 1H), 8.36 (s, 1H), 8.34 (s, 1H), 7.84 (d, J=8.5 Hz, 1H), 7.65 (d, J=6.5 Hz, 1H), 5.37-5.36 (m, 1H), 3.99 (s, 3H), 1.67 (d, J=7 Hz, 3H); LCMS m/z=540 [M+1].

### Example 277

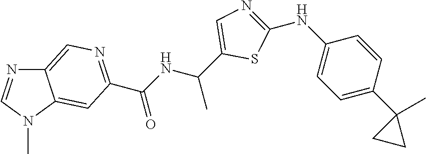

Synthesis of Example 277. The compound of Example 277 was prepared as previously described in Example 271 using 1-(4-nitrophenyl)ethanone. ¹H-NMR (CD₃OD, 500 MHz) δ 8.97 (s, 1H), 8.40 (d, J=8.5 Hz, 2H), 7.35 (d, J=8.5 Hz, 2H), 7.20 (d, J=8.5 Hz, 2H), 7.12 (s, 1H), 5.44-5.43 (m, 1H), 4.01 (s, 3H), 1.71 (d, J=7 Hz, 3H), 1.39 (s, 3H), 0.81 (s, 1H), 0.70 (s, 1H); LCMS m/z=433 [M+1].

### Example 278

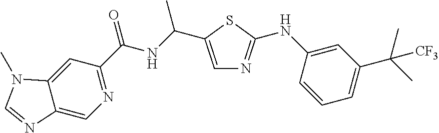

Synthesis of Example 278. The compound of Example 278 was prepared as described previously in Example 272 using 1-(3-nitrophenyl)ethanone. ¹H-NMR (CD₃OD, 500 MHz) δ 8.96 (s, 1H), 8.40 (s, 1H), 8.38 (s, 1H), 7.64 (s, 1H), 7.49 (d, J=9.5 Hz, 1H), 7.30-7.27 (m, 1H), 7.16-7.14 (m, 2H), 5.45-5.44 (m, 1H), 4.0 (s, 3H), 1.72 (d, J=7 Hz, 3H), 1.56 (s, 6H); LCMS m/z=489 [M+1].

### Example 279

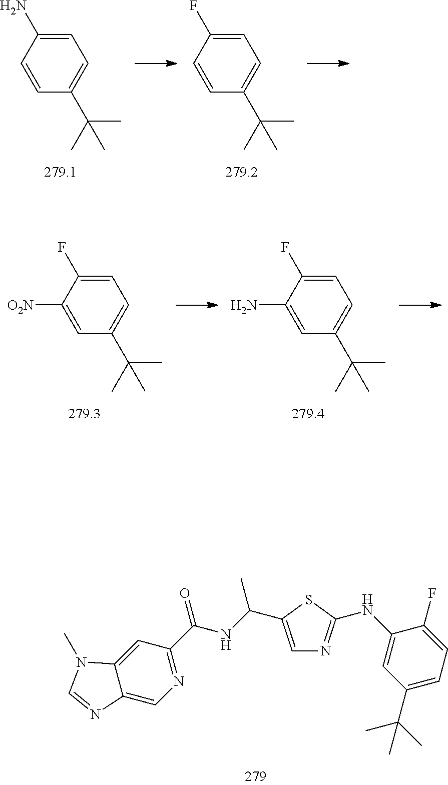

Synthesis of Compound 279.2. To an ice cold mixture of 4-tert-butyl-aniline 279.1 (1 g, 0.006 mol) in 1N HCl (15 ml) was added sodium nitrite (912 mg 0.013 mol in 5 ml of water) at 0° C. and stirred 0° C. for 15 min. NaBF₄ (1.4 g, 0.0134 mol in 5 ml water) was added slowly to the above reaction mixture at 0° C. with stirring until a solid was obtained. The solid precipitate was collected by filtration and the solid residue was dried well. The solid was heated up to 140° C. (solid decomposition). The reaction mixture was diluted with water (30 ml) and extracted with EtOAc (2×20 ml). The combined organic layer was washed with brine solution, dried over anhydrous Na₂SO₄ and concentrated under reduced pressure. The crude material was purified by column chromatography [silica gel (60-120 mesh; 20 g) gradient 2-4% EtOAc/Hexane] to afford compound 279.2 (500 mg, 50%) as yellow color oil. ¹H-NMR (CDCl₃, 200 MHz) δ 7.37-7.32 (m, 2H), 7.01-6.92 (m, 2H), 1.30 (s, 9H).

Synthesis of Compound 279.3. To an ice cold mixture of compound 279.2 (500 mg) in H₂SO₄ (1 ml, 2 vol) was added HNO₃ (2.5 ml, 5 vol) at 0° C. and stirred at room temperature for 2 hr. After the completion of starting material (by TLC), the reaction mixture was diluted with water (15 ml) and extracted with EtOAc (2×10 ml). The combined organic layer was washed with brine solution, dried over anhydrous Na₂SO₄ and concentrated under reduced pressure. The crude material was purified by column chromatography [silica gel (60-120 mesh; 10 g) gradient 5-10% EtOAc/Hexane] to afford compound 279.3 (100 mg, 15%). ¹H-NMR (CDCl₃, 200 MHz) δ 8.07-8.02 (m, 1H), 7.68-7.60 (m, 1H), 7.26-7.16 (m, 1H), 1.33 (s, 9H).

Synthesis of Compound 279.4. To a solution of compound 279.3 (300 mg, 0.0015 mol) in AcOH (1.5 ml) was added iron powder (425 mg, 0.0077 mol) at room temperature, and the reaction mixture was stirred at room temperature for 2 hr. After the completion of starting material (by TLC), the reaction mixture was quenched with saturated NaHCO₃ solution and extracted with EtOAc (2×10 ml). The organic layer was washed with brine solution and dried over anhydrous sodium sulphate, and concentrated under reduced pressure to afford compound 279.4 (150 mg, 60% yield) as yellow solid. ¹H-NMR (CDCl₃, 200 MHz) 6.94-6.65 (m, 3H), 3.65 (bs, 2H), 1.26 (s, 9H).

Synthesis of Example 279. The compound of Example 279 was prepared as previously described in Example 240. ¹H-NMR (DMSO-D6, 500 MHz) δ 9.67 (s, 1H), 8.99 (d, J=8.5 Hz, 1H), 8.97 (s, 1H), 8.46 (s, 1H), 8.33 (d, J=9.5 Hz, 1H), 7.13 (s, 1H), 7.10-7.06 (m, 1H), 6.97 (s, 1H), 5.35-5.32 (m, 1H), 3.94 (s, 3H), 1.63 (d, J=6.5 Hz, 3H), 1.25 (s, 9H); LCMS m/z=453 [M+1].

### Example 280

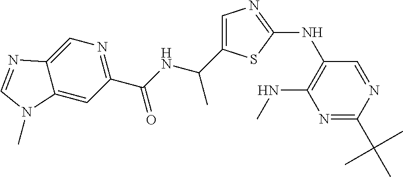

Synthesis of Example 280. The compound of Example 280 was prepared as described previously in Example 275 using compound 270.1 and methylamine. ¹H-NMR (CD₃OD, 500 MHz) δ 8.97 (s, 1H), 8.41 (s, 1H), 8.38 (s, 1H), 8.19 (s, 1H), 7.08 (s, 1H), 5.54-5.52 (m, 1H), 4.01 (s, 3H), 3.03 (s, 3H), 1.70 (d, J=7 Hz, 3H), 1.38 (s, 9H); LCMS m/z=466 [M+1].

### Example 281

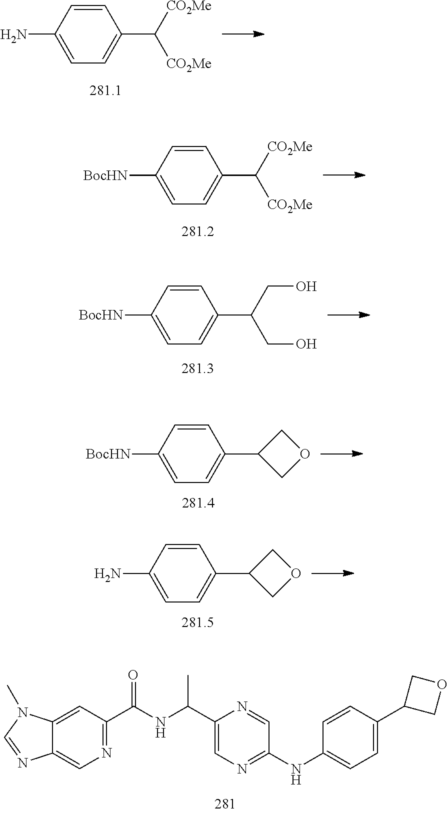

Synthesis of Compound 280.2. To a stirred solution of compound 280.1 (650 mg, 0.0029 mol) in MeOH (10 ml) was added di-tert-butyl dicarbonate (698 mg, 0.0032 mol) and triethylamine (324 mg, 0.0032 mol). The reaction mixture was stirred at room temperature for 6 hr. After completion of the starting material (by TLC), the reaction mixture was concentrated under reduced pressure and obtained crude material was diluted with water (20 ml) and extracted with ethyl acetate (3×20 ml). The combined organic layers was dried over anhydrous Na₂SO₄ and concentrated under reduced pressure to afford a residue, which was purified by column chromatography [SiO₂, 60-120 mesh (100 g), gradient (10%-20% EtOAc/Hexane)] to yield compound 280.2 (320 mg, 34%) as a white solid. ¹H-NMR (CDCl₃, 200 MHz) δ 7.38 (d, J=8.5 Hz, 4H) 6.50 (bs, 1N—H), 4.60 (s, 1H), 3.79 (s, 3H), 1.46 (s, 9H).

Synthesis of Compound 280.3. To a solution of compound 280.2 (100 mg, 0.3 mmol) in THF/EtOH (2 ml of 1:1) was added NaBH₄ (23 mg, 0.61 mmol) and LiCl (26 mg, 0.61 mmol) at 0° C. The resulting reaction mixture was stirred at 0° C. for 2 hr. After completion of the starting material (by TLC), the reaction mixture was concentrated under reduced pressure. The resulting crude material was diluted with water (100 ml) and extracted with ethyl acetate (3×50 ml). The combined organic layers was dried over anhydrous Na₂SO₄ and concentrated under reduced pressure to afford compound 280.3 (65 mg, 79% yield) as a white solid. This crude compound was used for the next step without further purification. ¹H-NMR (CDCl₃, 200 MHz) δ 7.39 (d, J=8.5 Hz, 2H), 7.19 (d, J=8.5 Hz, 2H), 6.49-6.48 (bs, 1N—H), 3.98-3.95 (m, 4H), 3.18-3.15 (m, 1H), 1.79-1.75 (bs, 2O—H), 1.46 (s, 9H); LCMS m/z=268 [M+1].

Synthesis of Compound 280.4. To a stirred solution of compound 280.3 (100 mg, 0.00037 mol) in THF (5 ml) was added n-butyl lithium (71 mg, 0.00112 mol) and stirred at 0° C. for 30 min. Tosyl chloride (71 mg, 0.00037 mol) was added to the above reaction mixture and stirred for 1 hr at 0° C., n-butyl lithium (24 mg, 0.00037 mol) was added to the above reaction mixture and stirred at 60° C. for 5 hr. After completion of the starting material (by TLC), the reaction mixture was quenched with water (50 ml) and extracted with ethyl acetate (3×50 ml). The combined organic layers was dried over anhydrous Na₂SO₄ and concentrated under reduced pressure. This crude material was purified by preparative TLC to afford compound 280.4 (15 mg, 16.6%) as a brown thick gum. ¹H-NMR (CDCl₃, 500 MHz) δ 7.26 (dd, J=8.5 Hz, 4H), 6.40 (bs, 1N—H), 4.97-4.95 (m, 2H), 4.63-4.60 (m, 2H), 4.15-4.10 (m, 1H), 1.43 (s, 9H).

Synthesis of Compound 280.5. The compound 280.5 was prepared as previously described in the Table 1 general tert-butyl carbamate deprotection procedure. ¹H-NMR (CD₃OD, 500 MHz) δ 7.19 (d, J=8.5 Hz, 2H), 6.78 (d, J=8.5 Hz, 2H), 5.10-5.08 (m, 2H), 4.66-4.65 (m, 2H), 4.17-4.15 (m, 1H); LCMS m/z=149 [M+1].

Synthesis of Example 280. The compound of Example 280 was prepared as previously described in Scheme L and Example 240. ¹H-NMR (CD₃OD, 500 MHz) δ 9.0 (s, 1H), 8.41 (s, 1H), 8.38 (s, 1H), 8.20 (d, J=9 Hz, 2H), 7.68 (d, J=9 Hz, 2H), 7.38 (d, J=8.5 Hz, 1H), 5.32-5.31 (m, 1H), 5.10-5.08 (m, 2H), 4.78-4.75 (m, 2H), 4.25-4.24 (m, 1H), 4.01 (s, 3H), 1.65 (d, J=7 Hz, 3H); LCMS m/z=430 [M+1].

### Example 282

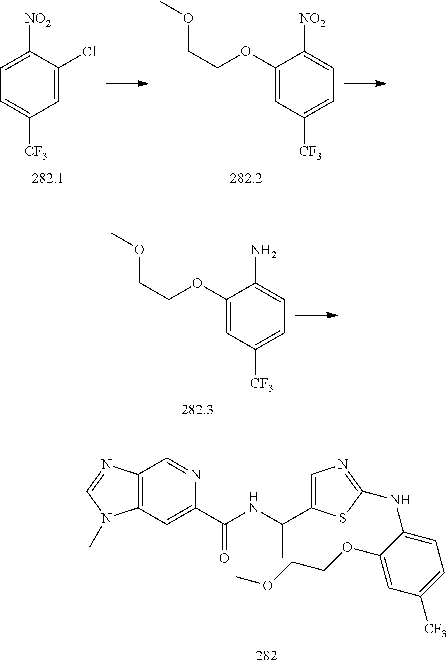

Synthesis of Compound 282.2. A mixture of 2-chloro-4-(trifluoromethyl)-1-nitrobenzene 282.1 (200 mg, 0.00088 mol), NaOEt (90 mg, 0.00133 mol) and 2-methoxy ethanol (4 ml) in a sealed tube was heated at 90° C. for 3 hr. After completion of the starting material (by TLC), the reaction mixture was diluted with water (20 ml) and extracted with EtOAc (3×20 ml). The combined organic layers was dried over anhydrous Na₂SO₄ and concentrated under reduced pressure to afford compound 282.2 (165 mg, 70% yield) as brown liquid that was used for the next step without any further purification. ¹H-NMR (CDCl₃, 200 MHz) δ 7.90 (d, J=9 Hz, 1H), 7.40 (s, 1H), 7.35 (d, J=9 Hz, 1H), 4.38-4.36 (m, 2H), 3.83-3.82 (m, 2H), 3.45 (s, 3H).

Synthesis of Compound 282.3. To a stirred solution of compound 282.2 (160 mg, 0.00063 mol) in AcOH (3.2 ml) was added Iron powder (202 mg, 0.0036 mol). The reaction mixture was stirred at room temperature for 3 hr. After completion of the starting material (by TLC), the reaction mixture was filtered through celite bed and washed with EtOAc. The filtrate was concentrated under reduced pressure and the obtained crude material was diluted with NaHCO₃ solution (100 ml) and extracted with EtOAc (3×50 ml). The combined organic extracts was dried over anhydrous Na₂SO₄ and concentrated under reduced pressure to afford compound 282.3 (110 mg, 78.5%) as a brown thick mass that was used for the next step without any further purification. ¹H-NMR (CDCl₃, 200 MHz) δ 7.10 (d, J=9 Hz, 1H), 6.97 (s, 1H), 6.72 (d, J=9 Hz, 1H), 4.21-4.19 (m, 2H), 3.79-3.78 (m, 2H), 3.42 (s, 3H); LCMS m/z=236 [M+1].

Synthesis of Example 282. The compound of Example 282 was prepared as previously described in Example 240. ¹H-NMR (DMSO-D6, 500 MHz) δ 9.50 (s, 1H), 9.10 (d, J=8.5 Hz, 1H), 8.97 (s, 1H), 8.58 (d, J=8.5 Hz, 1H), 8.45 (s, 1H), 8.38 (s, 1H), 7.30 (s, 1H), 7.29 (d, J=8.5 Hz, 1H), 7.20 (s, 1H), 5.40-5.39 (m, 1H), 4.29-4.28 (m, 2H), 3.97 (s, 3H), 3.79-3.78 (m, 2H), 1.73 (d, J=7 Hz, 3H); LCMS m/z=521 [M+1].

### Example 283

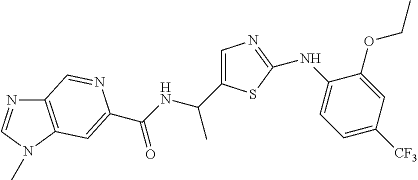

Synthesis of Example 283. The compound of Example 283 was prepared as previously described in Example 282 using ethanol. ¹H-NMR (CD₃OD, 500 MHz) δ 8.96 (s, 1H), 8.39 (d, J=8 Hz, 2H), 8.28 (d, J=8.5 Hz, 1H), 7.23 (s, 1H), 7.20 (d, J=9 Hz, 1H), 7.16 (s, 1H), 5.48-5.47 (m, 1H), 4.21 (q, J=7.5 Hz, 2H), 3.99 (s, 3H), 1.73 (d, J=7 Hz, 3H), 1.48 (t, J=7.5 Hz, 3H); LCMS m/z=491 [M+1].

### Example 284

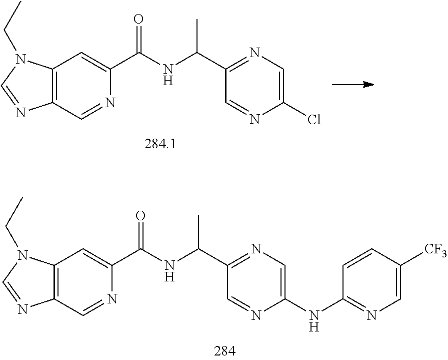

Synthesis of Compound 284.1. Compound 284.1 was prepared as previously described in Scheme L using compound F.3. ¹H-NMR (CD₃OD, 500 MHz) δ 9.0 (s, 1H), 8.65 (s, 1H), 8.58 (s, 1H), 8.45 (s, 1H), 8.39 (s, 1H), 5.43-5.41 (m, 1H), 4.43-4.41 (m, 2H), 1.73 (d, J=7 Hz, 3H), 1.59-1.57 (m, 3H); LCMS m/z=331 [M+1].

Synthesis of Example 284. The compound of Example 284 was prepared as previously described in Example 240 utilizing 2-amino-5-trifluoromethylpyridine. ¹H-NMR (CD₃OD, 500 MHz) δ 9.10 (s, 1H), 9.01 (s, 1H), 8.55 (s, 1H), 8.51 (s, 1H), 8.39 (s, 2H), 7.89 (d, J=8.5 Hz, 1H), 7.78 (d, J=9 Hz, 1H), 5.39-5.38 (m, 1H), 4.42 (q, J=8.5 Hz, 2H), 1.65 (d, J=7 Hz, 3H), 1.58 (t, J=8 Hz, 3H); LCMS m/z=457 [M+1].

### Example 285

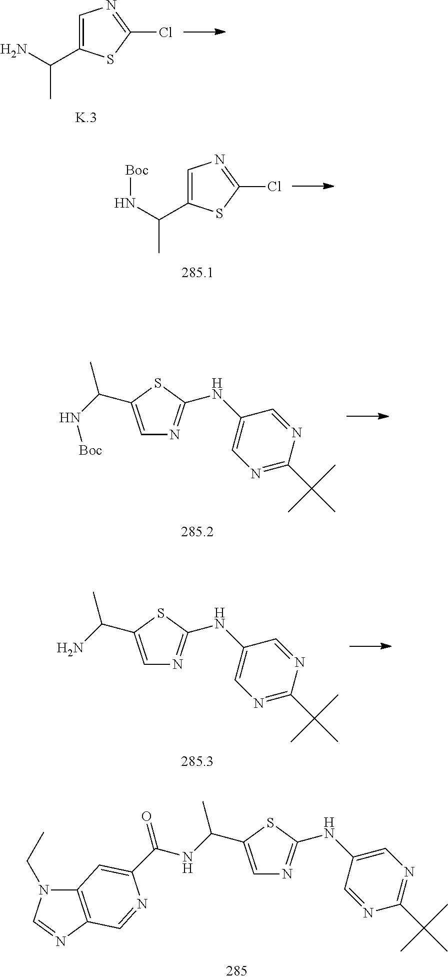

Synthesis of Compound 285.1. The solution of compound K.3 (600 mg, 3.74 mmol) in CH₂Cl₂ (10 ml) was added TEA (1 ml, 7.4 mmol), (Boc)₂O (968 ml, 4.44 mmol) at 5° C. The resulting reaction mixture was stirred at room temperature for 6 hr. After completion of the starting material (by TLC), the reaction mixture was diluted with water. The organic layer was dried over Na₂SO₄ and concentrated under reduced pressure, the resulting crude was purified by column chromatography [silica gel (60-120 mesh, 60 g), gradient (15-20% EtOAc/Hexane)] to afford compound 285.1 (800 mg, 82%) as a light green solid. ¹H-NMR (CDCl₃, 200 MHz) δ 7.36 (s, 1H), 4.99-4.94 (m, 1H), 4.81-4.80 (bs, 1H), 1.60 (d, J=8 Hz, 3H), 1.45 (s, 9H). LCMS m/z=263 [M+1].

Synthesis of Compound 285.2. The Compound 285.2 was prepared as described previously in Example 240. LCMS m/z=378.2 [M+1].

Synthesis of Compound 285.3. The compound 285.3 was prepared as described previously in the Table 1 general tert-butyl carbamate deprotection procedure. ¹H-NMR (CDCl₃, 200 MHz) δ 8.85 (s, 2H), 7.10 (s, 1H), 4.34-4.4.32 (m, 1H), 1.54-1.40 (m, 12H); LCMS m/z=278 [M+1].

Synthesis of Example 285. The compound of Example 285 was prepared as described previously in Table 1 general amide bond formation procedure. ¹H-NMR (DMSO-D6, 500 MHz) δ 10.29 (s, 1H), 9.05 (d, J=8.5 Hz, 1H), 8.96 (s, 3H), 8.56 (s, 1H), 8.36 (s, 1H), 7.17 (s, 1H), 5.35-5.32 (m, 1H), 4.42 (q, J=6.5 Hz, 2H), 1.63 (d, J=6 Hz, 3H), 1.42 (t, J=6.5 Hz, 3H), 1.32 (s, 9H); LCMS m/z=451 [M+1].

### Example 286

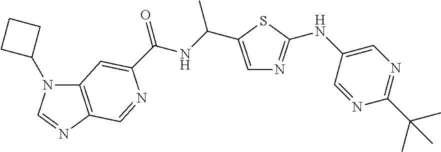

Synthesis of Example 286. The compound of Example 286 was prepared as described previously in Example 285 using the appropriate carboxylic acid prepared as described in Scheme D using cyclobutylamine. ¹H-NMR (DMSO-D₆, 500 MHz) δ 10.28 (s, 1H), 9.03 (d, J=7.5 Hz, 1H), 8.97 (s, 3H), 8.77 (s, 1H), 8.30 (s, 1H), 7.19 (s, 1H), 5.39-5.35 (m, 1H), 5.29-5.25 (m, 1H), 2.58 (bs, 4H), 1.92-1.87 (m, 2H), 1.64 (d, J=7.0 Hz, 3H), 1.38 (s, 9H)); LCMS m/z=477 [M+1].

### Example 287

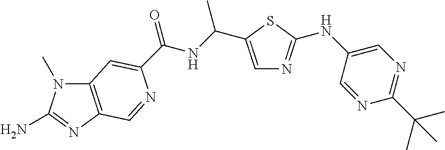

Synthesis of Example 287. The compound of Example 287 was prepared as described previously in Example 285 using the carboxylic acid 199.3. ¹H-NMR (DMSO-D₆, 500 MHz) δ 10.29 (s, 1H), 8.97 (s, 3H), 8.80 (d, J=8.5 Hz, 1H), 8.33 (s, 1H), 7.89 (s, 1H), 7.15 (s, 1H), 7.01 (s, 1H), 5.33-5.30 (m, 1H), 3.58 (s, 3H), 1.61 (d, J=6.5 Hz, 3H), 1.32 (s, 9H)); LCMS m/z=452 [M+1].

### Example 288

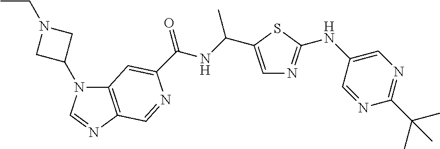

Synthesis of Example 288. The compound of Example 288 was prepared as described previously in Example 285 using the appropriate carboxylic acid prepared as described previously in Table 1. ¹H-NMR (DMSO-D₆, 500 MHz) δ 10.26 (s, 1H), 9.05 (d, J=7.0 Hz, 1H), 9.01 (s, 1H), 8.98 (s, 2H), 7.77 (s, 1H), 7.56 (s, 1H), 7.19 (s, 1H), 5.38-5.34 (m, 1H), 5.30-5.27 (m, 1H), 3.76-3.74 (m, 2H), 3.52-3.47 (m, 2H), 2.57-2.55 (m, 2H), 1.63 (d, J=7.0 Hz, 3H), 1.38 (s, 9H), 0.98 (t, J=7.0 Hz, 3H)); LCMS m/z=506 [M+1].

### Example 289

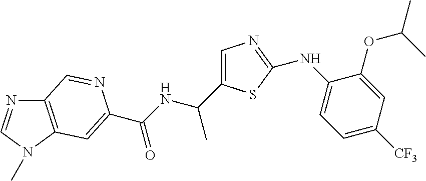

Synthesis of Example 289. The compound of Example 289 was prepared as previously described in Example 282 using isopropanol. ¹H-NMR (CD₃OD, 500 MHz) δ 8.97 (s, 1H), 8.39 (d, J=8.0 Hz, 2H), 7.22 (s, 1H), 7.08 (d, J=8.0 Hz, 2H), 5.48-5.44 (m, 1H), 4.75 (q, J=6.5 Hz, 1H), 4.00 (s, 3H), 1.76 (d, J=7 Hz, 3H), 1.41 (d, J=7.0 Hz, 6H); LCMS m/z=505 [M+1].

### Example 290

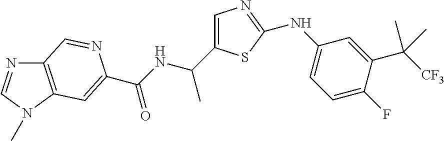

Synthesis of Example 290. The compound of Example 290 was prepared as previously described in Example 272 using 2-fluoro-5-nitro-acetophenone. ¹H-NMR (CD₃OD, 500 MHz) δ 8.95 (s, 1H), 8.38 (d, J=9.5 Hz, 2H), 7.65-7.63 (m, 1H), 7.52-7.49 (m, 1H), 7.13 (s, 1H), 7.04-7.00 (m, 1H), 5.44-5.42 (m, 1H), 3.99 (s, 3H), 1.71 (d, J=8 Hz, 3H), 1.63 (s, 6H); LCMS m/z=507 [M+1].

### Example 291

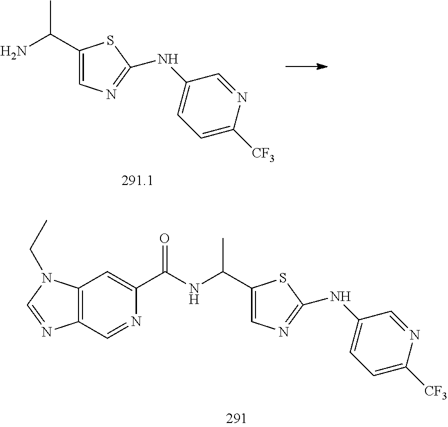

Synthesis of Compound 291.1. Compound 291.1 was prepared as previously described in Example 285 using 4-amino-1-trifluoromethylpyridine. LCMS m/z=289 [M+1].

Synthesis of Example 291. The compound of Example 291 was prepared as previously described in the Table 1 general amide bond formation procedure using compound F.3. ¹H-NMR (DMSO-D₆, 500 MHz) δ 10.74 (s, 1H), 9.11 (d, J=8.5 Hz, 1H), 8.98 (s, 1H), 8.77 (s, 1H), 8.57 (s, 1H), 8.42 (d, J=10.5 Hz, 1H), 8.37 (s, 1H), 7.82 (d, J=9.0 Hz, 1H), 7.26 (s, 1H), 5.40-5.37 (m, 1H), 4.42 (q, J=7.5 Hz, 2H), 1.65 (d, J=7.0 Hz, 3H), 1.44 (t, J=7.0 Hz, 3H); LCMS m/z=462 [M+1].

TABLE 10
The following compounds of the present invention, set forth in Table 10,
below, were prepared as previously described in the Table 4 general amide bond formation
procedure, using compound 291.1 and the appropriate carboxylic acid.

| Example | Structure | Characterization Data |
|---|---|---|
| 292 | 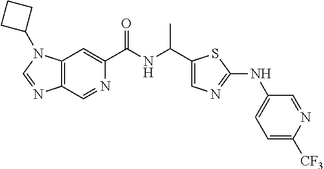 | ¹H-NMR (DMSO-D₆, 500 MHz) δ 10.79 (s, 1H), 9.17 (d, J = 7.5 Hz, 1H), 8.99 (s, 1H), 8.78 (s, 1H), 8.75 (s, 1H), 8.43 (d, J = 7.5 Hz, 1H), 8.32 (s, 1H), 7.83 (d, J = 7.5 Hz, 1H), 7.26 (s, 1H), 5.39- 5.36 (m, 1H), 5.18-5.16 (m, 1H), 2.56 (s, 4H), 1.90 (t, J = 7.0 Hz, 2H), 1.65 (d, J = 7.0 Hz, 3H); LCMS m/z = 488 [M + 1]. |
| 293 | 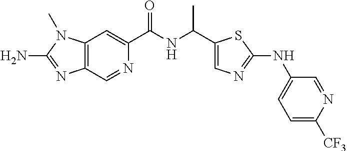 | ¹H-NMR (CD₃OD, 500 MHz) δ 8.79 (s, 1H), 8.41 (s, 2H), 7.97 (s, 1H), 7.72 (d, J = 7.5 Hz, 1H), 7.25 (s, 1H), 5.45-5.43 (m, 1H), 3.63 (s, 3H), 1.73 (d, J = 7.0 Hz, 3H); LCMS m/z = 463 [M + 1]. |
| 294 | 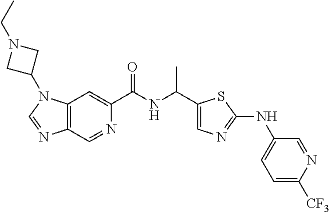 | ¹H-NMR (CD₃OD, 500 MHz) δ 9.03 (s, 1H), 8.81 (s, 1H), 8.73 (s, 1H), 8.53 (s, 1H), 8.45 (d, J = 7.5 Hz, 1H), 7.73 (d, J = 7.5 Hz, 1H), 7.31 (s, 1H), 5.51-5.49 (m, 1H), 5.32-5.29 (m, 1H), 3.98 (t, J = 7.5 Hz, 2H), 3.69 (s, 2H), 2.75 (q, J = 7.5 Hz, 3H), 1.78 (d, J = 7.5 Hz, 3H), 1.15 (t, J = 7.5 Hz, 3H); LCMS m/z = 517 [M + 1]. |

### Example 295

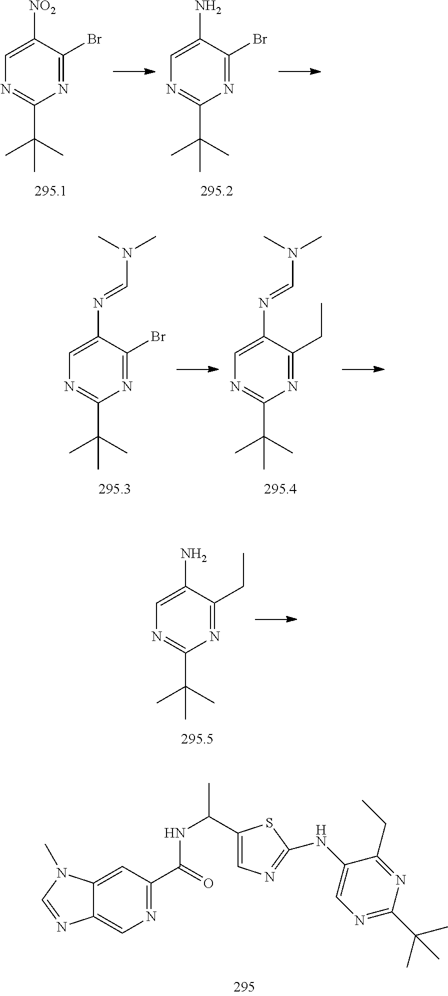

Synthesis of Compound 295.1. The compound 295.1 was prepared as described previously for compound 270.1 using POBr₃.

Synthesis of Compound 295.2. The compound 295.2 was prepared as described previously for compound 275.3 using Fe/AcOH.

Synthesis of Compound 295.3. A solution of 75 mg (0.33 mmole) of compound 295.2 in 5 mL of MeOH was treated with 230 μL (1.76 mmole) of N,N-dimethylformamide dimethyl acetal, and the reaction mixture was heated at 90° C. for 2 hr. After cooling to room temperature, the mixture was diluted with H₂O and extracted with EtOAc (2×). The combined organics were dried over Na₂SO₄, filtered, and concentrated to provide compound 295.3 as a red syrup that was used directly without further purification.

Synthesis of Compound 295.4. A solution of 75 mg (0.26 mmole) of compound 295.3 in 1 mL of anhydrous DMF was treated with 11 mg (0.05 mmole) of Pd(OAc)₂, 48 mg (0.16 mmole) of tri-o-tolylphosphine, and 81 mg (0.66 mmole) of Et₂Zn. The reaction mixture was heated at 90° C. for 10 min and then excess reactives were quenched by the dropwise addition on H₂O. The mixture was extracted with EtOAc (2×), and the combined organics were dried over Na₂SO₄, filtered, and concentrated. Purification by flash column chromatography (SiO₂, 50% EtOAc/hexanes) provided 50 mg (80%) of compound 295.4.

Synthesis of Compound 295.5. A solution of 50 mg (0.21 mmole) of compound 295.4 in 1.5 mL of EtOH and 0.5 mL of 6 N HCl was heated at 90° C. for 2 hr. The reaction mixture was cooled to room temperature and made basic by addition of saturated aqueous NHCO₃. The aqueous mixture was extracted with EtOAc (2×), and the combined organics were dried over Na₂SO₄, filtered, and concentrated. Purification by flash column chromatography (SiO₂, 20% EtOAc/hexanes) provided 30 mg (78%) of compound 295.5.

Synthesis of Example 295. The compound of Example 295 was prepared from compound 295.5 as previously described in Example 272. ¹H-NMR (CD₃OD, 500 MHz) δ 8.98 (s, 1H), 8.91 (s, 1H), 8.41 (s, 1H), 8.39 (s, 1H), 7.09 (s, 1H), 5.43-5.40 (m, 1H), 4.01 (s, 3H), 2.79 (q, 2H), 1.71 (d, J=7.0 Hz, 3H), 1.53 (s 9H), 1.31 (t, J=7 Hz, 3H); LCMS m/z=465 [M+1].

### Example 296

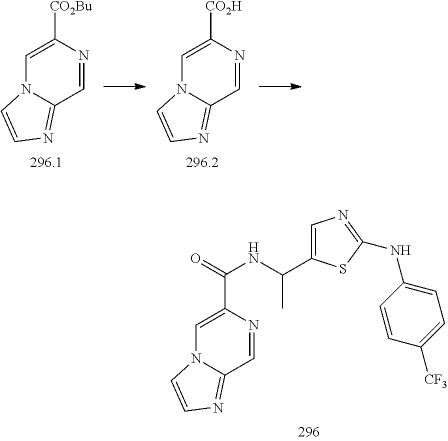

Synthesis of Compound 296.2. Compound 296.2 was prepared as previously described in Scheme F using 6-bromo-imidazo[1,2-a]pyrazine. ¹H-NMR (CDCl₃, 200 MHz) δ 9.22 (s, 1H), 9.01 (s, 1H), 7.93 (s, 1H), 7.82 (s, 1H), 4.47 (t, J=7.5 Hz, 2H), 1.87-1.78 (m, 2H), 1.76-1.63 (m, 2H), 0.98 (t, J=7.0 Hz, 3H); LCMS m/z 220 [M+1].

Synthesis of Example 296. The compound of example 296 was prepared as previously described in Scheme F and the Table 1 general amide bond formation procedure. ¹H-NMR (DMSO-D6, 500 MHz) δ 10.46 (s, 1H), 9.28 (s, 1H), 9.08 (d, J=8.0 Hz, 2H), 8.29 (s, 1H), 7.91 (s, 1H), 7.77 (d, J=9 Hz, 2H), 7.62 (d, J=9 Hz, 2H), 7.20 (s, 1H), 5.42-5.36 (m, 1H), 1.63 (d, J=7.5 Hz, 3H), 0.85 (d, J=7 Hz, 6H); LCMS m/z=433 [M+1].

### Example 297

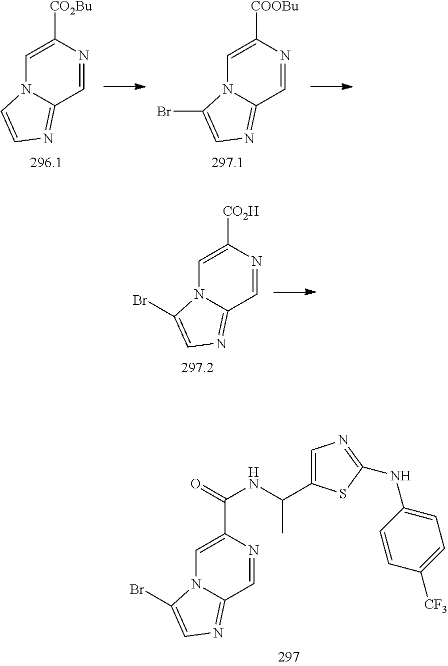

Synthesis of Compound 297.1. To a solution of compound 296.1 (300 mg, 1.369 mmol) in chloroform (10 ml) was added NBS (365 mg, 2.054 mmol) portion wise, catalytic amount of AIBN at 0° C. under inert atmosphere. The resulting mixture was stirred at 80° C. for 12 hr. After completion starting material (by TLC), the reaction mass was distilled off, diluted with EtOAc, and washed with saturated NaHCO₃ solution (3×10 ml). The combined organic layers was washed with brine, dried over anhydrous Na₂SO₄ and concentrated under reduced pressure. The crude residue was purified by column chromatography [silica gel (60-120 mesh, 35 g), gradient (1-2% MeOH/CH₂Cl₂)] to afford compound 297.1 (300 mg, 73.5%) as an off white solid. LCMS m/z=300 [M+2].

Synthesis of Example 297. The compound of example 297 was prepared as previously described in Scheme F and the Table 1 general amide bond formation procedure. ¹H-NMR (DMSO-D₆, 500 MHz) δ 10.44 (s, 1H), 9.21 (d, J=8.0 Hz, 1H), 9.08 (s, 1H), 8.79 (s, 1H), 8.10 (s, 1H), 7.78 (d, J=8.0 Hz, 2H), 7.58 (d, J=8.0 Hz, 2H), 7.20 (s, 1H), 5.42-5.38 (m, 1H), 1.64 (d, J=7.5 Hz, 3H), 1.47 (s, 9H), 1.44-1.41 (m, 1H); LCMS m/z=513 [M+2].

### Example 298

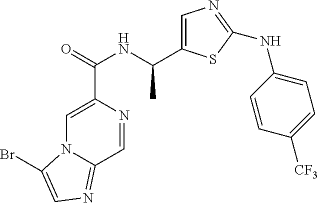

Synthesis of Example 298. The compound of example 298 was prepared as previously described in Example 297 using compound R-A-6. ¹H-NMR (DMSO-D₆, 500 MHz) δ 10.46 (s, 1H), 9.23 (d, J=8.0 Hz, 1H), 9.13 (s, 1H), 8.79 (s, 1H), 8.10 (s, 1H), 7.77 (d, J=8.5 Hz, 2H), 7.64 (d, J=8.5 Hz, 2H), 7.20 (s, 1H), 5.43-5.40 (m, 1H), 1.69 (d, J=7.0 Hz, 3H); LCMS m/z=513 [M+2].

### Example 299

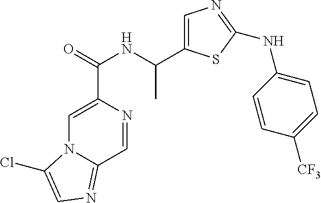

Synthesis of Example 299. The compound of example 299 was prepared as previously described in Example 297 using N-chlorosuccinimide. ¹H-NMR (DMSO-D₆, 500 MHz) δ 10.45 (s, 1H), 9.20 (d, J=8.5 Hz, 1H), 9.14 (s, 1H), 8.77 (s, 1H), 8.07 (s, 1H), 7.76 (d, J=8.5 Hz, 2H), 7.61 (d, J=8.5 Hz, 2H), 7.20 (s, 1H), 5.38-5.35 (m, 1H), 1.63 (d, J=7.0 Hz, 3H); LCMS m/z=467 [M+1].

### Example 300

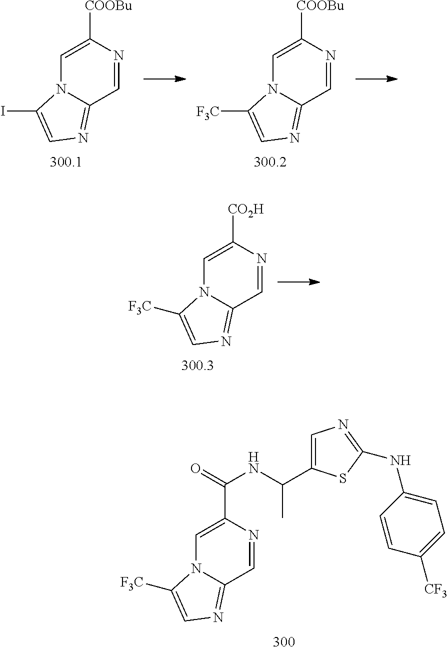

Synthesis of Compound 300.1. Compound 300.1 was prepared as previously described in Example 297 using N-iodosuccinimide. ¹H NMR (200 MHz, CHLOROFORM-d) δ 9.07 (d, J=1.5 Hz, 1H), 8.80 (d, J=1.5 Hz, 1H), 8.11 (s, 1H), 4.36 (t, J=6.4 Hz, 2H), 1.74 (d, J=7.7 Hz, 2H), 1.42 (d, J=8.1 Hz, 2H), 0.95 (t, J=7.3 Hz, 3H).

Synthesis of Compound 300.2. A solution of 50 mg (0.14 mmole) of compound 300.1 in 1.5 mL of anhydrous DMF was treated with 3 mg (0.02 mmole) of CuI and 55 mg (0.28 mmole) and heated at 80° C. under microwave irradiation for 30 min. The reaction mixture was diluted with 15 mL of water and extracted with diethyl ether (3×30 mL). The combined organics were washed with cold water (3×50 mL). The organic layer was dried over Na₂SO₄, filtered, concentrated, and purified by preparatory thin-layer chromatography (SiO2, 100% EtOAc) to afford 40 mg (48%) of compound 300.2.

Synthesis of Compound 300.3. Compound 300.3 was prepared as previously described in Scheme F.

Synthesis of Example 300. The compound of Example 300 was prepared as previously described in the Table 1 general amide bond formation procedure. ¹H-NMR (DMSO-D₆, 500 MHz) δ 10.47 (s, 1H), 9.36 (s, 1H), 9.32 (d, J=8 Hz, 1H), 8.81 (s, 1H), 8.54 (s, 1H), 7.78 (d, J=8.0 Hz, 2H), 7.63 (d, J=8.0 Hz, 2H), 7.21 (s, 1H), 5.41-5.38 (m, 1H), 1.65 (d, J=7.0 Hz, 3H); LCMS m/z=501 [M+1].

### Example 301

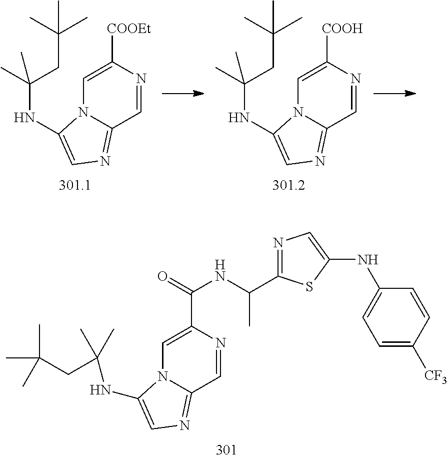

Synthesis of Compound 301.1. To a stirred solution of ethyl 5-aminopyrazine-2-carboxylate (200 mg, 0.985 mmol) in ethanol/CH₂Cl₂ (10 ml) were added formaldehyde (0.35 ml, 4.926 mmol) and scandinium triflate (48 mg, 0.0985 mmol) under N₂ atmosphere. The resulting reaction mixture was stirred for at room temperature for 50 minutes. 2-Isocyano-2,4,4-trimethylpentane (0.17 ml, 0.985 mmol) was added to the above reaction mixture and stirred at room temperature for 48 hr. After the completion of the starting material (by TLC), the reaction mixture was concentrated under reduced pressure. The resulting crude compound was diluted with water (50 ml) and extracted with ethyl acetate (3×20 ml). The combined organic extracts was dried over Na₂SO₄ and concentrated under reduced pressure to give compound 301.1 (200 mg, crude). This crude material was used for the next step without any further purification. LCMS m/z=319 [M+1].

Synthesis of Compound 301.2. The compound 301.2 was prepared as described previously in Scheme F. LCMS m/z=291 [M+1].

Synthesis of Example 301. The compound of Example 301 was prepared as previously described in the Table 1 general amide bond formation procedure. ¹H-NMR (DMSO-D6, 500 MHz) δ 10.46 (s, 1H), 9.05 (s, 1H), 8.97 (d, J=8.5 Hz, 1H), 8.80 (s, 1H), 7.78 (d, J=8.5 Hz, 2H), 7.63 (d, J=9 Hz, 2H), 7.40 (s, 1H), 7.20 (s, 1H), 5.62 (s, 1H), 5.39-5.36 (m, 1H), 1.71 (s, 2H), 1.63 (d, J=7.0 Hz, 3H), 1.35-1.33 (m, 6H), 0.96 (s, 9H); LCMS m/z=560 [M+1].

### Example 302

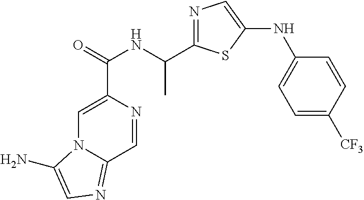

Synthesis of Example 302. To a stirred solution of the compound of Example 301 (100 mg, 0.02 mmol) in dry CH₂Cl₂ (5 ml) was added TFA (2 ml) at 0° C. The resulting reaction mixture was stirred at room temperature for 1 hr. After completion of the starting material (by TLC), the reaction mixture was concentrated under reduced pressure and diluted with NaHCO₃ solution (100 ml) and extracted with CH₂Cl₂ (3×30 ml). The combined organic extracts was dried over Na₂SO₄ and concentrated under reduced pressure and the resulting crude material was purified by preparative TLC to afford Example 302 (36 mg, 45%) as an yellow solid. ¹H-NMR (DMSO-D₆, 500 MHz) δ 10.24 (s, 1H), 8.95 (d, J=9.0 Hz, 2H), 8.68 (s, 1H), 7.69 (d, J=9.0 Hz, 2H), 7.60 (d, J=9.0 Hz, 2H), 7.20 (s, 1H), 7.15 (s, 1H), 6.10-5.95 (bs, 2H), 5.40-5.25 (m, 1H), 1.65 (d, J=7 Hz, 3H); LCMS m/z=448 [M+1].

### Examples 303 and 304

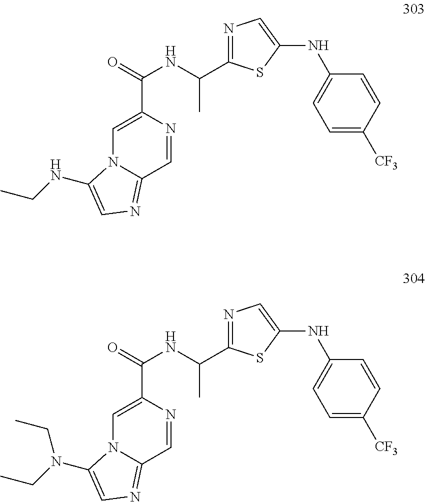

Synthesis of Example 303 and Example 304. The compounds of Examples 303 and 304 were prepared as previously described in the Table 1 general reductive amination procedure using acetaldehyde.

Example 303: ¹H-NMR (DMSO-D₆, 500 MHz) δ 10.04 (s, 1H), 9.09 (d, J=8.5 Hz, 1H), 8.97 (s, 1H), 8.62 (s, 1H), 7.78 (d, J=8.5 Hz, 2H), 7.63 (d, J=9 Hz, 2H), 7.21 (s, 1H), 5.39-5.36 (m, 1H), 3.15-3.10 (m, 2H), 1.64 (d, J=7.0 Hz, 3H), 1.00-0.097 (m, 3H); LCMS m/z=476.2 [M+1].

Example 304: ¹H-NMR (DMSO-D₆, 500 MHz) δ 10.45 (s, 1H), 9.10 (d, J=8.5 Hz, 1H), 8.99 (s, 1H), 7.78 (d, J=8.5 Hz, 2H), 7.71 (s, 1H), 7.63 (d, J=8.5 Hz, 2H), 7.21 (s, 1H), 5.39-5.36 (m, 1H), 3.15-3.10 (m, 4H), 1.64 (d, J=7.0 Hz, 3H), 1.00-0.097 (m, 6H); LCMS m/z=504 [M+1].

### Example 305

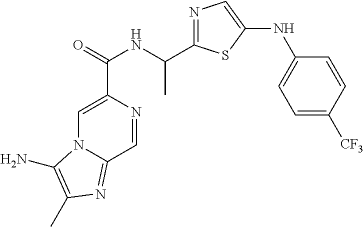

Synthesis of Example 305. The compound of Example 305 was prepared as previously described in Example 301 using acetaldehyde. ¹H-NMR (DMSO-D₆, 500 MHz) δ 10.45 (s, 1H), 8.89 (d, J=9.0 Hz, 1H), 8.78 (s, 1H), 8.60 (s, 1H), 7.76 (d, J=9.0 Hz, 2H), 7.62 (d, J=9.0 Hz, 2H), 7.19 (s, 1H), 5.73 (s, 2H), 5.37-5.34 (m, 1H), 2.31 (s, 3H), 1.64 (d, J=7 Hz, 3H); LCMS m/z=462 [M+1].

### Example 306

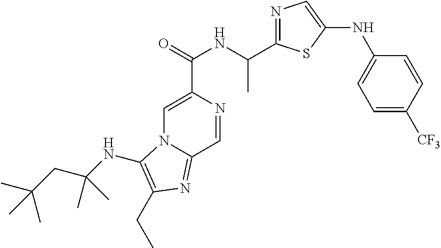

Synthesis of Example 306. The compound of Example 306 was prepared as previously described in Example 301 using propionaldehyde. ¹H-NMR (DMSO-D₆, 500 MHz) δ 10.46 (s, 1H), 9.05 (d, J=8.5 Hz, 1H), 8.97 (s, 1H), 8.80 (s, 1H), 7.78 (d, J=8.5 Hz, 2H), 7.63 (d, J=9 Hz, 2H), 7.21 (s, 1H), 5.39-5.36 (m, 1H), 4.62 (s, 1H), 2.29 (m, 2H), 1.71 (s, 2H), 1.63 (d, J=7.0 Hz, 3H), 1.35-1.33 (m, 6H), 1.10-0.96 (m, 12H); LCMS m/z=588 [M+1].

### Example 307

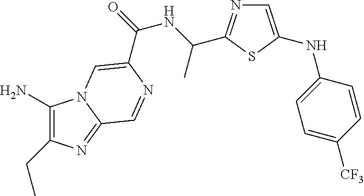

Synthesis of Example 307. The compound of Example 307 was prepared from Example 306 as previously described from Example 302. ¹H-NMR (DMSO-D₆, 500 MHz) δ 10.44 (s, 1H), 8.84 (d, J=8.5 Hz, 1H), 8.79 (s, 1H), 8.62 (s, 1H), 7.76 (d, J=8.5 Hz, 2H), 7.61 (d, J=8.5 Hz, 2H), 7.18 (s, 1H), 5.72 (s, 2H), 5.36-5.33 (m, 1H), 2.72 (q, J=7.5 Hz, 2H), 1.61 (d, J=6.5 Hz, 3H), 1.20 (t, J=7.5 Hz, 3H); LCMS m/z=476 [M+1].

### Example 308

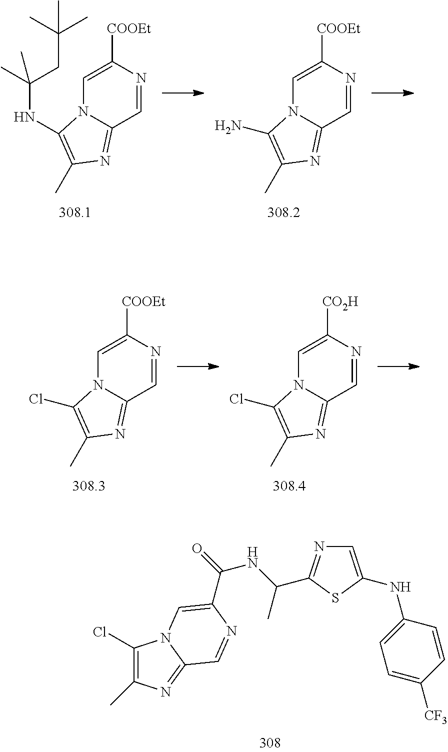

Synthesis of Compound 308.1. Compound 308.1 was prepared as previously described in Example 301 using acetaldehyde.

Synthesis of Compound 308.2. Compound 308.2 was prepared from compound 308.1 as previously described from Example 302. ¹H-NMR (DMSO-D₆, 200 MHz) δ 8.94 (s, 1H), 8.78 (s, 1H), 5.8 (bs, 2H), 4.4 (q, J=7.6 Hz, 2H), 2.43 (s, 3H), 1.36 (t, J=7.6 Hz, 3H).

Synthesis of Compound 308.3. To a stirred solution of compound 308.2 (150 mg, 0.681 mmol) in AcOH (0.4 ml, 0.024 mmol) were added concentrated HCl (0.16 ml, 0.0545 mmol), NaCl (187 mg, 3.238 mmol) followed by the addition of NaNO₂ (94 mg, 1.363 mmol in water) at 0° C. and stirred at 0° C. for 10 min. The resulting mixture was stirred at room temperature for 1 hr. After completion of starting material (by TLC), the reaction mixture was diluted with saturated solution of Urea (81 mg, 1.363 mmol) at 0° C. and stirred for additional 20 min. The resulting mixture was neutralized with solid NaHCO₃ and extracted with EtOAc (2×10 ml). The combined organic extract was washed with brine solution, dried over Na₂SO₄. The solvent was evaporated under reduced pressure to get crude. The resulting crude material was washed with pentane (2×10 ml) to afford compound 308.3 (120 mg, 74%) as white solid. ¹H-NMR (DMSO-D₆, 500 MHz) δ 9.05 (s, 1H), 8.78 (s, 1H), 4.4 (q, J=7.8 Hz, 2H), 2.44 (s, 3H), 1.36 (t, J=7.8 Hz, 3H); LCMS m/z=240 [M+1].

Synthesis of Compound 308.4. Compound 308.4 was prepared as previously described in Example 301. ¹H-NMR (DMSO-d₆, 500 MHz) δ 13.40 (bs, 1H), 9.01 (s, 1H), 8.78 (s, 1H), 2.41 (s, 3H).

Synthesis of Example 308. The compound of Example 308 was prepared as previously described in Table 1 general amide bond formation procedure. ¹H-NMR (DMSO-D₆, 500 MHz) δ 10.44 (s, 1H), 9.09 (d, J=8.5 Hz, 1H), 9.0 (s, 1H), 8.77 (s, 1H), 7.77 (d, J=8.5 Hz, 2H), 7.63 (d, J=8.5 Hz, 2H), 7.10 (s, 1H), 5.29-5.25 (m, 1H), 1.65 (d, J=7.0 Hz, 3H); LCMS m/z=481 [M+1].

### Example 309

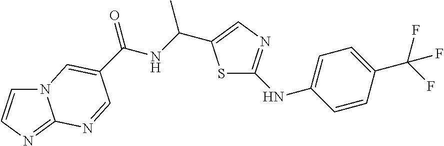

Synthesis of Example 309. The compound of Example 309 was prepared as described previously in Scheme F and Table 1 using 6-bromoimidazo[1,2-a]pyrimidine. ¹H-NMR (DMSO-D₆, 500 MHz) δ 10.45 (s, 1H), 9.45 (s, 1H), 9.08 (d, J=8.0 Hz, 1H), 8.98 (s, 1H), 8.01 (s, 1H), 7.77 (d, J=8.5 Hz, 2H), 7.64 (d, J=8.5 Hz, 2H), 7.13 (s, 1H), 5.29-5.25 (m, 1H), 1.65 (d, J=7.0 Hz, 3H); LCMS m/z=433 [M+1].

### Example 310

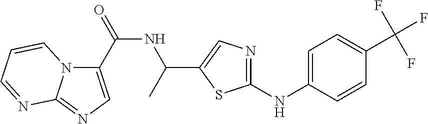

Synthesis of Example 310. The compound of Example 310 was prepared as described previously in Scheme F and Table 1 using 3-bromoimidazo[1,2-a]pyrimidine. ¹H-NMR (CD₃OD, 500 MHz) δ 9.83 (d, J=7 Hz, 1H), 8.72 (d, J=7 Hz, 1H), 8.43 (s, 1H), 7.69 (d, J=8.5 Hz, 2H), 7.55 (d, J=8.5 Hz, 2H), 7.27-7.25 (m, 1H), 5.49-5.48 (m, 1H), 1.71 (d, J=7 Hz, 3H); LCMS m/z=433 [M+1].

### Example 311

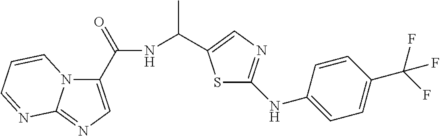

Synthesis of Example 311. The compound of Example 311 was prepared as described previously in Scheme F and Table 1 general amide bond formation procedure using 3-bromoimidazo[1,2-a]pyrazine. ¹H-NMR (CD₃OD, 500 MHz) δ 9.43 (d, J=7.0 Hz, 1H), 9.14 (s, 1H), 8.45 (s, 1H), 8.11 (d, J=7.5 Hz, 1H), 7.70 (d, J=7.5 Hz, 2H), 7.55 (d, J=7.5 Hz, 2H), 7.22 (s, 1H), 5.51-5.49 (m, 1H), 1.71 (d, J=7 Hz, 3H); LCMS m/z=433 [M+1].

### Example 312

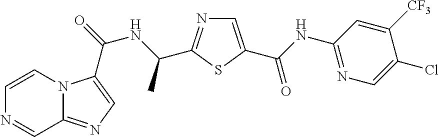

Synthesis of Example 312. The compound of Example 312 was prepared as described previously in the Table 1 general amide bond formation procedure using compound R-C.5. LCMS m/z=496 [M+1].

### Example 313

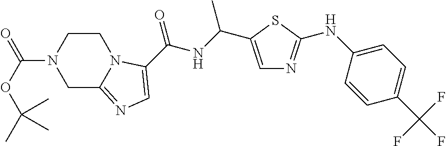

Synthesis of Example 313. The compound of Example 313 was prepared as described previously in Scheme F and Table 1 general amide bond formation procedure using tert-butyl 3-bromo-5,6-dihydroimidazo[1,2-a]pyrazine-7(8H)-carboxylate and compound A.6. ¹H-NMR (DMSO-D₆, 500 MHz) δ 10.46 (s, 1H), 8.63 (d, J=8 Hz, 1H), 7.78 (d, J=8 Hz, 2H), 7.65 (d, J=8.5 Hz, 2H), 7.61 (s, 1H), 7.16 (s, 1H), 5.27-5.24 (m, 1H), 4.55 (s, 2H), 4.27-4.25 (m, 2H), 3.74-3.73 (m, 2H), 1.55 (d, J=7 Hz, 3H), 1.43 (s, 9H); LCMS m/z=537.2 [M+1].

### Example 314

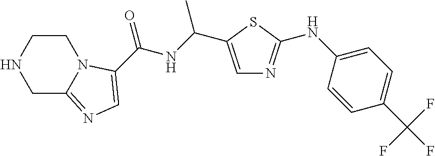

Synthesis of Example 314. The compound of Example 314 was prepared from Example 313 as described previously in Table 1 general tert-butylcarbamate deprotection procedure. ¹H-NMR (CD₃OD, 500 MHz) δ 7.74 (s, 1H), 7.68 (d, J=8.5 Hz, 2H), 7.57 (d, J=9 Hz, 2H), 7.17 (s, 1H), 5.37-5.36 (m, 1H), 4.65-4.62 (m, 2H), 4.52 (s, 2H), 3.73-3.71 (m, 2H), 1.64 (d, J=7 Hz, 3H); LCMS m/z=437.2 [M+1].

### Example 315

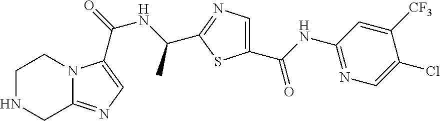

Synthesis of Example 315. The compound of Example 315 was prepared as described previously in Example 190 using compound R-C.5. LCMS m/z=500 [M+1].

### Example 316

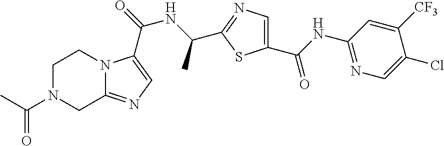

Synthesis of Example 316. The compound of Example 316 was prepared as described previously in Scheme F and the Table 1 general amide bond formation procedure using 1-(3-bromo-5,6-dihydroimidazo[1,2-a]pyrazin-7(8H)-yl)ethanone and compound R-C.5. LCMS m/z=542 [M+1].

### Example 317

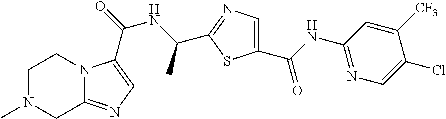

Synthesis of Example 317. The compound of Example 317 was prepared from Example 315 as described previously in Table 1 general reductive amination procedure using formaldehyde. LCMS m/z=514 [M+1].

### Example 318

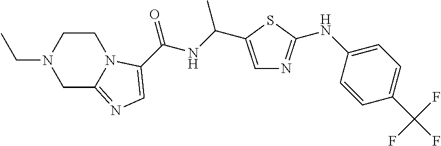

Synthesis of Example 318. The compound of Example 318 was prepared from Example 315 as described previously in Table 1 general reductive amination procedure using acetaldehyde. ¹H-NMR (CD₃OD, 500 MHz) δ 7.69 (d, J=8.5 Hz, 2H), 7.59 (s, 1H) 7.55 (d, J=8.5 Hz, 2H), 7.14 (s, 1H), 5.35-5.34 (m, 1H), 4.37-4.34 (m, 2H), 3.71 (s, 2H), 2.95-2.92 (m, 2H), 2.68-2.64 (m, 2H), 1.63 (d, J=7 Hz, 3H), 1.20 (t, J=7.5 Hz, 3H); LCMS m/z=465 [M+1].

### Example 319

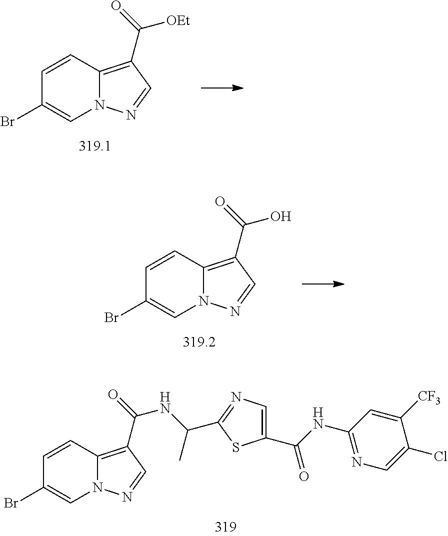

Synthesis of Compound 319.1. Pyrazolo[1,5-a]pyridine-3-carboxylic acid ethyl ester (1.00 g, 0.00526 mol) was dissolved in acetic acid (50 mL, 0.9 mol) and treated with bromine (0.8 mL, 0.02 mol). The reaction was heated at 80° C. for 6 hrs and then at room temperature overnight. An additional 3 equivalents of bromine were added and the reaction heated at 80° C. for an additional 7 hrs. Solvent was removed in vacuo to give an orange oil which was purified by column chromatography with EtOAc as eluant. Further purification by reverse phase HPLC gave the compound 319.1 in 25% yield. ¹H NMR (300 MHz, DMSO-d₆) δ 9.24 (s, 1H), 8.40 (s, 1H), 7.95-7.97 (m, 1H), 7.92-7.94 (m, 1H), 4.20-4.29 (m, 2H), 1.24-1.30 (m, 3H); LCMS m/z=2689 and 271 [M+1].

Synthesis of Compound 319.2 Compound 319.1 (70 mg, 0.0003 mol) was dissolved in Tetrahydrofuran (2 mL, 0.02 mol) and 1.0 M of Sodium hydroxide in Water (3 mL, 0.003 mol) was added at room temperature. Ethanol (1 mL, 0.02 mol) was added dropwise until a monophasic solution was obtained. The reaction was stirred for 8 hrs at room temperature. The organics were removed in vacuo and concentrated aqueous HCl was added to acidify solution. Compound 319.2 precipitated out of the acidic media, was collected by filtration on a medium frit, and was used without further purification. ¹H NMR (400 MHz, CD₃OD) δ 8.93 (s, 1H), 8.36 (s, 1H), 8.08 (d, J=9.47 Hz, 1H), 7.63 (d, J=9.47 Hz, 1H).

Synthesis of Example 319. The compound of Example 319 was prepared as described previously in Table 1 general amide bond formation procedure using compound C.5. ¹H NMR (400 MHz, DMSO-d₆) δ 11.73 (s, 1H), 9.21-9.27 (m, 1H), 9.04 (d, J=7.83 Hz, 1H), 8.77 (s, 1H), 8.74 (s, 1H), 8.71 (s, 1H), 8.55 (s, 1H), 8.15 (d, J=9.40 Hz, 1H), 7.64 (d, J=9.40 Hz, 1H), 5.38-5.53 (m, 1H), 1.65 (d, J=7.07 Hz, 3H); LCMS m/z=573 and 575 [M+1].

### Example 320

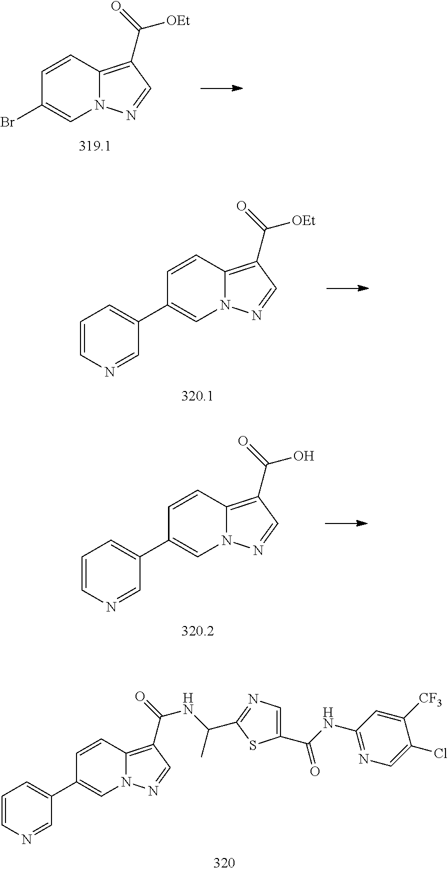

Synthesis of Compound 320.1. Compound 319.1 (50 mg, 0.0002 mol), 3-(4,4-Dimethyl-1,3,2-dioxaboretan-2-yl)-pyridine (30.0 mg, 0.00018 mol), 1,2-Dimethoxyethane (1.0 mL, 0.0096 mol), saturated aqueous sodium bicarbonate solution (0.2 mL, 0.002 mol) and tetrakis(triphenylphosphine)palladium(0) (8.0 mg, 0.0069 mmol) were added to a microwave vial and flushed with nitrogen gas. The vial was capped and the reaction was heated under microwave irradiation on 300 watts at 120° C. for 20 minutes. Solvent was removed in vacuo and the crude reaction filtered through a plug of celite flushing with 50% methanol/50% methylene chloride. Purification by reverse phase HPLC afforded compound 320.1 in 69% yield. ¹H NMR (400 MHz, CD₃OD) δ 9.09 (s, 1H), 8.93-8.97 (m, 1H), 8.63 (m, 1H), 8.47 (s, 1H), 8.28 (s, 1H), 8.24 (d, J=1.64, Hz, 1H), 7.92 (d, J=1.64 Hz, 1H), 7.54-7.70 (m, 1H), 4.42 (q, J=7.12 Hz, 2H), 1.45 (t, J=7.12 Hz, 3H); LCMS m/z=268 [M+1].

Synthesis of Compound 320.2. Compound 320.1 (100 mg, 0.0004 mol) was added to tetrahydrofuran (2 mL, 0.02 mol). 1.0 M of Sodium hydroxide in water (4 mL, 0.004 mol) was added followed by ethanol (4 mL, 0.07 mol) and the reaction stirred for 8 hrs. Organic solvents were removed in vacuo and concentrated hydrogen chloride (0.1 mL, 0.004 mol) added. The resulting solution was filtered to afford compound 320.2 in 57% yield. ¹H NMR (400 MHz, MeOD) δ 8.97 (s, 1H), 8.93-8.96 (m, 1H), 8.60 (m, 1H), 8.38 (s, 1H), 8.36 (s, 1H), 8.22 (d, J=1.70 Hz, 1H), 7.77 (dd, J=1.70, 9.22 Hz, 1H), 7.59 (d, J=7.96 Hz, 1H); LCMS m/z=240 [M+1].

Synthesis of Example 320. The compound of Example 320 was prepared as described previously in Table 1 general amide bond formation procedure using compound C.5. ¹H NMR (400 MHz, DMSO-d₆) δ 11.72-11.76 (m, 1H), 11.74 (s, 1H), 9.31-9.32 (m, 1H), 9.01-9.06 (m, 2H), 8.75-8.78 (m, 3H), 8.63 (dd, J=1.38, 4.89 Hz, 1H), 8.55 (s, 1H), 8.28-8.31 (m, 1H), 8.22-8.27 (m, 1H), 7.94 (dd, J=1.63, 9.29 Hz, 1H), 5.43-5.53 (m, 1H), 1.67 (d, J=7.15 Hz, 3H); LCMS m/z=572 [M+1].

TABLE 11
The following compounds of the present invention, set forth in Table 11,
below, were prepared as previously described in Example 320 using the corresponding boronic acid.

| Example | Structure | Characterization Data |
|---|---|---|
| 321 | 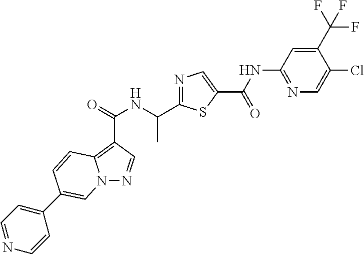 | ¹H NMR (400 MHz, DMSO-d₆) δ 11.73 (s, 1H), 9.61 (s, 1H), 9.08 (d, J = 7.65 Hz, 1H), 8.80-8.91 (m, 3H), 8.75 (d, J = 5.52 Hz, 2H), 8.54 (s, 1H), 8.33 (d, J = 9.41 Hz, 1H), 8.27 (d, J = 5.53 Hz, 2H), 8.07 (d, J = 9.41 Hz, 1H), 5.47 (t, J = 7.04 Hz, 1H), 1.66 (d, J = 7.03 Hz, 3H); LCMS m/z = 572.2 [M + 1]. |
| 322 | 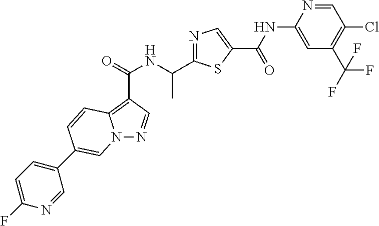 | ¹H-NMR (CDCl₃, 500 MHz) δ 8.72 (s, 1H), 8.65 (s, 1H), 8.50 (s, 1H), 8.42 (d, J = 8.5 Hz, 2H), 8.25 (d, J = 9 Hz, 2H), 8.15-8.12 (m, 1H), 7.60 (d, J = 8.5 Hz, 1H), 7.15 (d, J = 8.5 Hz, 1H), 6.59 (d, J = 7.5 Hz, 1H), 5.70-5.68 (m, 1H), 1.83 (d, J = 7 Hz, 3H); LCMS m/z = 590.2 [M + 1]. |
| 323 | 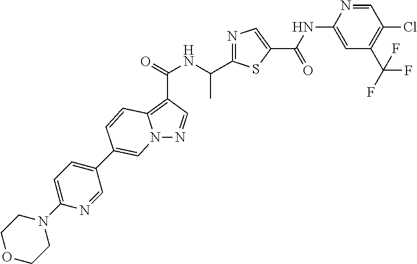 | ¹H-NMR (CDCl₃, 500 MHz) δ 8.68 (s, 2H), 8.50-8.46 (m, 3H), 8.38 (d, J = 8.5 Hz, 1H), 8.30 (s, 1H), 8.28 (s, 1H), 7.78 (d, J = 8.5 Hz, 1H), 7.60 (d, J = 7.5 Hz, 1H), 6.79 (d, J = 7.5 Hz, 1H), 6.59 (d, J = 8.5 Hz, 1H), 5.70-5.68 (m, 1H), 3.89- 3.85 (m, 4H), 3.60-3.58 (m, 4H), 1.83 (d, J = 7 Hz, 3H); LCMS m/z 657.07 [M + 1]. |
| 324 | 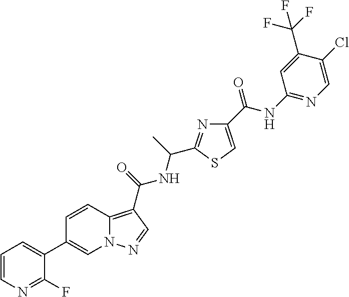 | ¹H-NMR (CDCl₃, 500 MHz) δ 8.80 (s, 1H), 8.69 (s, 1H), 8.50 (s, 1H), 8.42 (d, J = 8.5 Hz, 2H), 8.30 (s, 3H), 7.99-7.97 (m, 1H), 7.60 (d, J = 7.5 Hz, 1H), 7.39 (d, J = 7.5 Hz, 1H), 6.61 (d, J = 8.5 Hz, 1H), 5.70-5.68 (m, 1H), 1.83 (d, J = 7 Hz, 3H); LCMS m/z = 589.8 [M + 1]. |
| 325 | 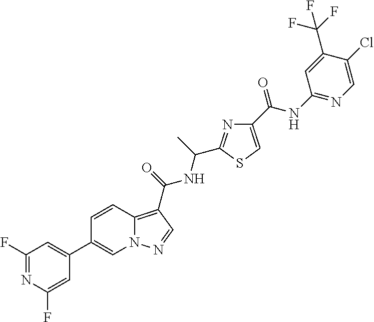 | ¹H-NMR (DMSO-D6, 500 MHz) δ 11.75 (s, 1H), 9.60 (s, 1H), 9.19 (d, J = 8 Hz, 1H), 8.80 (s, 1H), 8.79 (d, J = 8.5 Hz, 2H), 8.56 (s, 1H), 8.32 (d, J = 8.5 Hz, 1H), 8.08 (d, J = 8 Hz, 1H), 7.80 (s, 2H), 5.49-5.47 (m, 1H), 1.71 (d, J = 7 Hz, 3H); LCMS m/z 608 [M + 1]. |
| 326 | 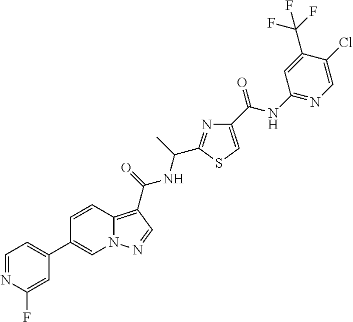 | ¹H-NMR (DMSO-D6, 500 MHz) δ 11.75 (s, 1H), 9.50 (s, 1H), 9.05 (s, 1H), 8.80 (s, 1H), 8.79-8.77 (m, 3H), 8.50 (s, 1H), 8.40 (d, J = 8.5 Hz, 2H), 8.05 (s, 1H), 8.0 (s, 1H), 7.79 (s, 1H), 5.49-5.47 (m, 1H), 1.62 (d, J = 7 Hz, 3H); LCMS m/z = 589.7 [M + 1]. |
| 327 | 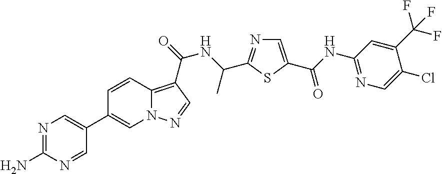 | ¹H-NMR (DMSO-D6, 500 MHz) δ 11.77 (s, 1H), 9.18 (s, 1H), 8.89 (d, J = 8.5 Hz, 1H), 8.79 (d, J = 8.5 Hz, 2H), 8.65 (s, 3H), 8.23 (d, J = 8 Hz, 1H), 7.82 (d, J = 8 Hz, 1H), 6.83 (s, 3H), 5.49-5.47 (m, 1H), 1.64 (d, J = 7 Hz, 3H); LCMS m/z = 587.9 [M + 1]. |
| 328 | 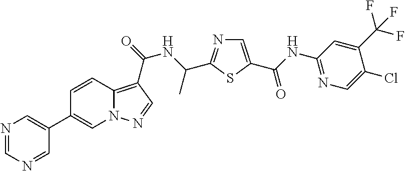 | ¹H-NMR (CDCl₃, 500 MHz) δ 9.30 (s, 1H), 9.01 (s, 2H), 8.79 (s, 1H), 8.63 (s, 1H), 8.50 (d, J = 8.5 Hz, 2H), 8.43 (s, 1H), 8.35 (s, 1H), 8.29 (s, 1H), 7.62 (d, J = 8 Hz, 1H), 6.63 (d, J = 7.5 Hz, 1H), 5.66-5.62 (m, 1H), 1.79 (d, J = 7 Hz, 3H); LCMS m/z = 572.6 [M + 1]. |
| 329 | 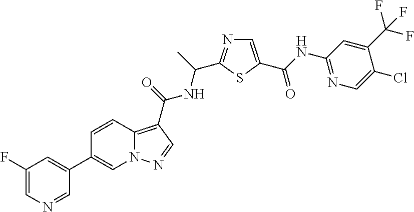 | ¹H-NMR (CDCl₃, 500 MHz) δ 8.79 (s, 1H), 8.77 (s, 1H), 8.69 (s, 1H), 8.59 (s, 1H), 8.50-8.47 (m, 3H), 8.30 (d, J = 8.5 Hz, 2H), 7.73-7.70 (m, 2H), 6.60 (d, J = 7.5 Hz, 1H), 5.70-5.68 (m, 1H), 1.80 (d, J = 7 Hz, 3H); LCMS m/z = 589.9 [M + 1]. |
| 330 | 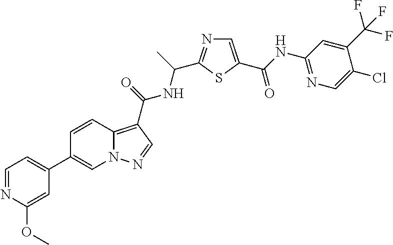 | ¹H-NMR (CDCl₃, 500 MHz) δ 8.80 (s, 1H), 8.69 (s, 1H), 8.50 (s, 1H), 8.43-8.41 (m, 3H), 8.30 (d, J = 8.5 Hz, 2H), 7.73- 7.70 (m, 2H), 7.69 (d, J = 7.5 Hz, 1H), 7.10 (d, J = 8.5 Hz, 1H), 6.99 (s, 1H), 6.60 (d, J = 8.5 Hz, 1H), 5.70-5.68 (m, 1H), 4.00 (s, 3H), 1.80 (d, J = 7 Hz, 3H); LCMS m/z 601.7 [M + 1]. |
| 331 | 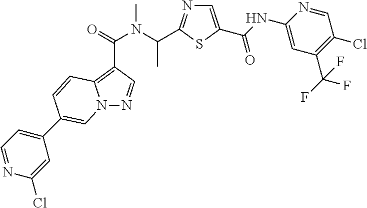 | ¹H-NMR (CDCl₃, 500 MHz) δ 8.80 (s, 1H), 8.65 (s, 1H), 8.50-8.47 (m, 4H), 8.30 (d, J = 8.5 Hz, 2H), 7.63 (d, J = 7.5 Hz, 1H), 7.60 (s, 1H), 7.50 (s, 1H), 6.61 (d, J = 8.5 Hz, 1H), 5.70-5.68 (m, 1H), 1.80 (d, J = 7 Hz, 3H); LCMS m/z 605.5 [M + 1]. |

### Example 332

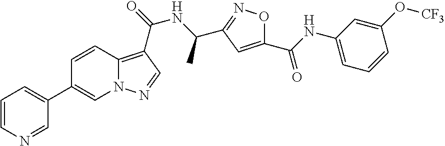

Synthesis of Example 332. The compound of Example 332 was prepared as described previously in Example 320 using (R)-3-(1-aminoethyl)-N-(3-(trifluoromethoxy)-phenyl)-isoxazole-5-carboxamide, which was prepared as described in Scheme H utilizing 3-trifluoromethoxy-aniline. ¹H NMR (300 MHz, DMSO-d₆) δ 11.33 (s, 1H), 8.83-9.05 (m, 5H), 8.42 (s, 1H), 8.27 (s, 1H), 8.13 (d, J=8.29 Hz, 1H), 7.88 (s, 2H), 7.70 (s, 1H), 7.52 (s, 1H), 7.29 (s, 1H), 5.63 (s, 1H), 1.82 (d, J=7.06 Hz, 3H); LCMS m/z=537 [M+1].

### Example 333

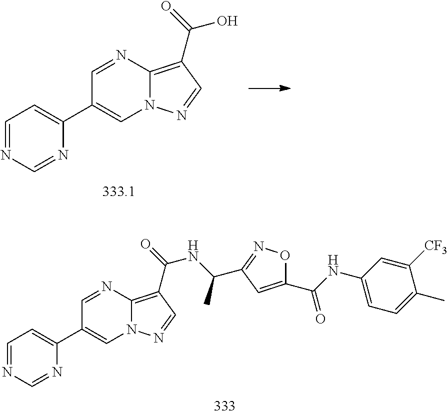

Synthesis of Compound 331.1. 3-Hydroxy-2-pyrimidin-4-yl-propenal (0.350 g, 0.00233 mol) and 3-amino-4-pyrazolecarboxylic acid (0.30 g, 0.0024 mol) were dissolved in ethanol (20 mL, 0.3 mol)/acetic acid (1 mL, 0.02 mol) and heated to 80° C. The reaction was heated for 8 hrs, then cooled to room temperature and stirred overnight. The material was filtered and washed with ethanol to afford compound 331.1 in 59% yield. ¹H NMR (300 MHz, DMSO-d₆) δ 12.52 (s, 1H), 9.97 (d, J=2.0 Hz, 1H), 9.48 (d, J=2.0 Hz, 1H), 9.26 (s, 1H), 8.91 (d, J=5.37 Hz, 1H), 8.64 (s, 1H), 8.28 (d, J=5.37 Hz, 1H).

Synthesis of Example 331. The compound of Example 331 was prepared as described previously in Example 320 using (R)-3-(1-aminoethyl)-N-(3-(trifluoromethyl)-4-methyl-phenyl)-isoxazole-5-carboxamide, which was prepared as described in Scheme H utilizing 3-trifluoromethyl-4-methyl-aniline. ¹H NMR (300 MHz, CHLOROFORM-d) δ 9.65-9.78 (m, 1H), 9.40-9.49 (m, 2H), 9.02 (d, J=5.37 Hz, 1H), 8.88 (s, 1H), 8.38-8.48 (m, 1H), 8.33 (s, 1H), 7.95 (s, 1H), 7.77-7.92 (m, 2H), 5.67-5.75 (m, 1H), 2.57 (s, 3H), 1.87 (d, J=7.06 Hz, 3H); LCMS m/z=537 [M+1].

### Example 334

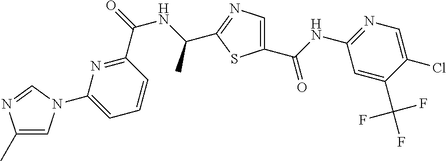

Synthesis of Example 334. To a flame dried sealed reaction vial was added Cs₂CO₃ (64 mg, 0.20 mmol), CuI (1.8 mg, 0.0094 mmol), 2-oxo-cyclohexanecarboxylic acid ethyl ester (0.003 mL, 0.019 mmol), and DMSO (0.50 mL). After flushing with N₂ for 3 minutes, the mixture stirred for 30 min at 25° C. Then a solution of 4-methylimidazole (9.2 mg, 0.11 mmol) and Example 91 (50 mg, 0.094 mmol) in DMSO (1.5 mL) was added and the mixture was heated at 60° C. for 19 hr. The mixture was purified via preparative reverse-phase HPLC (flow rate 20, from 10% B (MeCN with 0.1% formic acid) to 95% B in 10 min), affording Example 334 as a gray solid (14 mg, yield 28%). ¹H NMR (400 MHz, DMSO-d₆) δ=11.76 (s, 1H), 9.58 (d., J=8.6 Hz, 1H), 8.76 (m, 2H), 8.55 (s, 1H), 8.22 (m, 1H), 8.13 (m, 1H), 8.02 (d, J=7.6 Hz, 2H), 5.52 (m, 1H), 2.54 (s, 3H), 1.75 (d, J=7.1 Hz, 3H); LCMS m/z=536 [M+1].

### Example 335

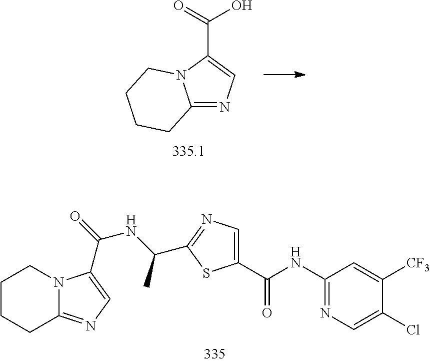

Synthesis of Compound 335.1. To a mixture of imidazo[1,2-a]pyridine-3-carboxylic acid (81 mg, 0.5 mmol) in 5 mL EtOH was added PtO₂ (20 mg, 0.09 mmol, 0.18 equiv) and conc HCl (0.45 mL). The mixture was stirred under a hydrogen atmosphere (balloon) for 4 hours, filtered through celite and concentrated to provide 67 mg (80%) of compound 335.1, which was used without further purification.

Synthesis of Example 335. The compound of Example 335 was prepared as previously described in Table 1 General Amide Bond Formation procedure using compound R-C.5. LCMS m/z=499 [M+1].

### Example 336

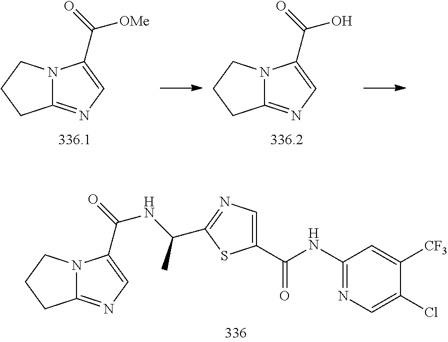

Synthesis of Compound 336.1. Compound 336.1 was prepared as previously described in Scheme F, using 3-bromo-6,7-dihydro-5H-pyrrolo[1,2-a]imidazole.

Synthesis of Compound 336.2. Hydrolysis of Compound 336.1 was performed as previously described in Scheme F to afford Compound 336.2, which was used without purification.

Synthesis of Example 336. The compound of Example 336 was prepared as previously described in Table 1 General Amide Bond Formation procedure using compound R-C.5. LCMS m/z=485 [M+1].

### Example 337

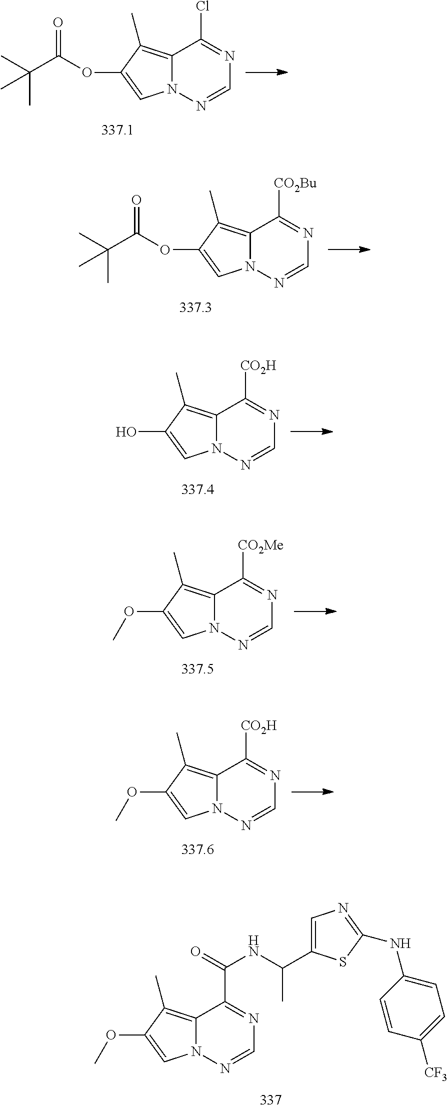

Synthesis of Compound 337.3. The compound 337.3 was prepared as previously described in Scheme F. ¹H-NMR (CDCl₃, 200 MHz) δ 8.43 (s, 1H), 8.16 (s, 1H), 4.51-4.47 (m, 2H), 2.31 (s, 3H), 1.83-1.77 (m, 2H), 1.51-1.49 (m, 2H), 1.39 (s, 9H), 1.02-0.98 (m, 3H); LCMS m/z 334 [M+1].

Synthesis of Compound 337.4. The compound 337.4 was prepared as previously described in Scheme E. LCMS m/z 194 [M+1].

Synthesis of Compound 337.5. To a stirred solution of compound 337.4 (50 mg, 0.22 mmol), in DMF (3 ml) was added Cs₂CO₃ (91 mg, 0.28 mmol) and MeI (17 mg, 0.28 mmol) were added at 0° C. The resulting reaction mixture was stirred at room temperature for 1 hr. After completion of the starting material (by TLC), the reaction mixture was diluted with water (10 ml) and extracted with EtOAc (3×20 ml). The combined organic extracts were dried over sodium sulphate and concentrated under reduced pressure to afford compound 337.5 (50 mg, crude) as a light brown solid which was used for the next step without any further purification. LCMS m/z 222 [M+1].

Synthesis of Compound 337.6. The compound 337.6 was prepared as previously described in Scheme E. ¹H-NMR (CD₃OD, 200 MHz) δ 8.19 (s, 1H), 7.79 (s, 1H), 3.90 (s, 3H), 2.28 (s, 3H); LCMS m/z 208 [M+1].

Synthesis of Example 337. The compound of Example 337 was prepared as previously described in the Table 1 general amide bond formation procedure. ¹H-NMR (CDCl₃, 500 MHz) δ 8.25 (s, 1H), 7.99 (d, J=8.5 Hz, 1H), 7.70 (s, 1H), 7.59 (d, J=8.5 Hz, 2H), 7.45 (d, J=8.5 Hz, 2H), 7.24 (s, 1H), 5.55-5.54 (m, 1H), 3.98 (s, 3H), 2.58 (s, 3H), 1.77 (d, J=7 Hz, 3H); LCMS m/z=477 [M+1].

### Example 338

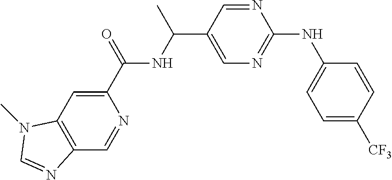

Synthesis of Example 338. The compound of Example 338 was prepared as previously described in Scheme B and Table 1 general amide bond formation procedure utilizing 1-(2-chloropyrimidin-5-yl)ethanone. ¹H NMR (400 MHz, DMSO-d₆) δ 10.09 (s, 1H), 9.20 (d., J=8.3 Hz, 1H), 9.00 (s, 1H), 8.66 (s, 2H), 8.49 (s, 1H), 8.33 (s, 1H), 7.97 (d, J=8.5 Hz, 2H), 7.62 (d, J=8.5 Hz, 2H), 5.21 (m, 1H), 3.94 (s, 3H), 1.62 (d, J=7.0 Hz, 3H); LCMS m/z=442 [M+1].

### Example 339

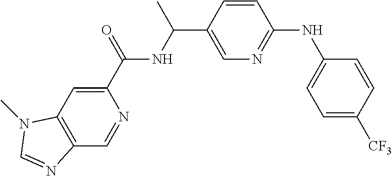

Synthesis of Example 339. The compound of Example 339 was prepared as previously described in Scheme B and Table 1 general amide bond formation procedure utilizing 1-(2-chloropyridin-5-yl)ethanone. ¹H NMR (400 MHz, DMSO-d₆) δ=9.58 (s, 1H), 9.14 (d, J=8.5 Hz, 1H), 9.04 (s, 1H), 8.56 (s, 1H), 8.39 (s, 1H), 8.27 (d, J=2.3 Hz, 1H), 7.90-7.75 (m, 3H), 7.58 (d, J=8.5 Hz, 2H), 6.93 (d, J=8.5 Hz, 1H), 5.20 (m, 1H), 1.57 (d, J=7.0 Hz, 3H); LCMS m/z=441 [M+1].

### Example 340

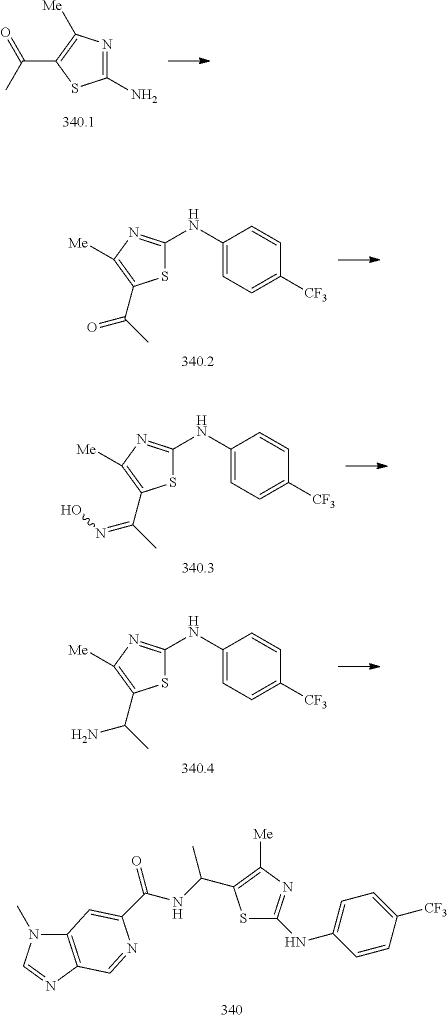

Synthesis of Compound 340.2. A reaction vial was charged with 200. mg (1.28 mmol) of 1-(2-amino-4-methylthiazol-5-yl)ethanone, 0.28 mL (1.92 mmol) of 1-bromo-4-trifluoromethyl-benzene, 330 mg (0.36 mmol) of Pd₂(dba)₃, 510 mg (0.88 mmol) of Xantphos, 1.0 g (3.1 mmol) of cesium carbonate, and 4 mL of anhydrous 1,4-dioxane. The mixture was degassed with N₂ for 15 min, followed by heating at 145° C. in microwave for 60 min. The reaction mixture was filtered through a medium frit and the solid was washed with CH₂Cl₂. The filtrate was concentrated under vacuum, and the residue was purified by flash column chromatography (SiO2, 0% EtOAc/hexanes gradient to 10% EtOAc/hexanes) to afford 300 mg of compound 340.2 (60% yield). ¹H NMR (400 MHz, CDCl₃) δ 7.65 (d, J=8.3 Hz, 2H), 7.50 (d, J=8.3 Hz, 2H), 2.65 (s, 3H), 2.50 (s, 3H); LCMS m/z=301 [M+1].

Synthesis of Compound 340.3 A reaction vial was charged with 98 mg (1.41 mmol) of hydroxylamine hydrochloride, 200 mg (0.66 mmol) of compound 340.2, and 4.3 mL of methanol and 0.22 mL (2.6 mmol) of pyridine. The solution was stirred at room temperature for 24 hours followed by removal of all the volatiles under vacuum. The residue was triturated with water for 16 hr. The solid was collected by filtration and dried under vacuum to provide 160 mg of compound 340.3 as a light yellow solid (76%). ¹H NMR (400 MHz, CDCl₃-d) δ=7.58 (d, J=8.5 Hz, 2H), 7.45-7.40 (m, J=8.5 Hz, 2H), 2.48 (s, 3H), 2.32 (s, 3H); LCMS m/z=316 [M+1]

Synthesis of Compound 340.4 A solution of 80 mg (0.25 mmol) of compound 340.3 in 20 mL of ethanol was treated with 200 mg of Raney Nickel slurry in water. The mixture was stirred under a 30 PSI H₂ atmosphere for 48 hours. The solid catalyst was removed via filtration over celite, and the filtrate was concentrated under vacuum to give 57 mg of Compound 340.4 as a brown gum. LCMS m/z=302 [M+1]

Synthesis of Example 340. The compound of Example 340 was prepared as previously described in the Table 1 general amide bond formation procedure. ¹H NMR (CD₃OD, 400 MHz) δ=8.96 (s, 1H), 8.39 (s, 1H), 8.34 (s, 1H), 7.69 (d, J=8.5 Hz, 2H), 7.54 (d, 2H), 5.70-5.36 (m, 1H), 3.99 (s, 3H), 2.35 (s, 3H), 1.65 (d, 4H); LCMS m/z=461 [M+1].

### Example 341

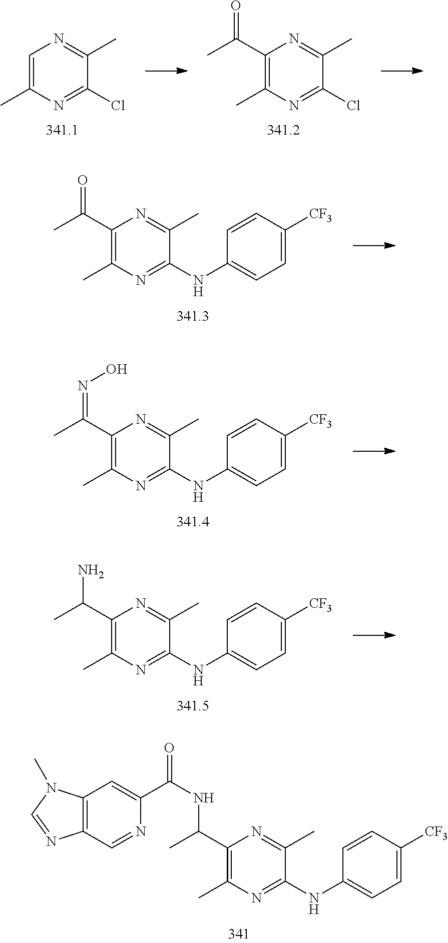

Synthesis of Compound 341.2. A mixture of 2.0 mL (16.6 mmol) of 3-chloro-2,5-dimethylpyrazine and 5.6 mL (100 mL) of acetaldehyde in 1.5 mL (28.2 mmol) of concentrated H₂SO₄ and 8 mL of water was chilled in an ice bath, and then treated concurrently with 9.5 mL (69.4 mmol) of tert-butyl hydroperoxide and a solution of 27.8 g (100 mmol) of iron(II) sulfate in 66 mL of. The mixture was stirred for 24 hours, and then treated with 7.5 g (59.4 mmol) of sodium sulfite. The mixture was washed with 4×40 mL CH₂Cl₂. The combined organics were concentrated under vacuum and the residue was purified via flash column chromatography (SiO₂, 100% CH₂Cl₂). Product containing frctions were concentrated under vacuum with no additional heating to afford 1.17 g of compound 341.2 (38%) as a volatile light yellow solid. ¹H NMR (400 MHz, CDCl₃) δ=2.77 (s, 3H), 2.68 (s, 3H), 2.67 (s, 3H); LCMS m/z=185 [M+1].

Synthesis of Compound 341.3. Compound 341.3 was prepared as previously described in Scheme B. LCMS m/z=310 [M+1].

Synthesis of Compound 341.4. Compound 341.4 was prepared as previously described in Example 340. LCMS m/z=325 [M+1].

Synthesis of Compound 341.5 Compound 341.5 was prepared as previously described in Example 340. LCMS m/z=311 [M+1].

Synthesis of Example 341. The compound of Example 341 was prepared as previously described in the Table 1 general amide bond formation procedure. ¹H NMR (CD₃OD, 400 MHz) δ=9.07 (s, 1H), 8.73 (s, 1H), 8.57 (s, 1H), 7.87 (d, J=8.5 Hz, 2H), 7.54 (d, J=8.5 Hz, 2H), 5.65-5.43 (m, 1H), 4.06 (s, 3H), 2.58 (s, 6H), 1.59 (d, 3H); LCMS m/z=470 [M+1].

### Example 342

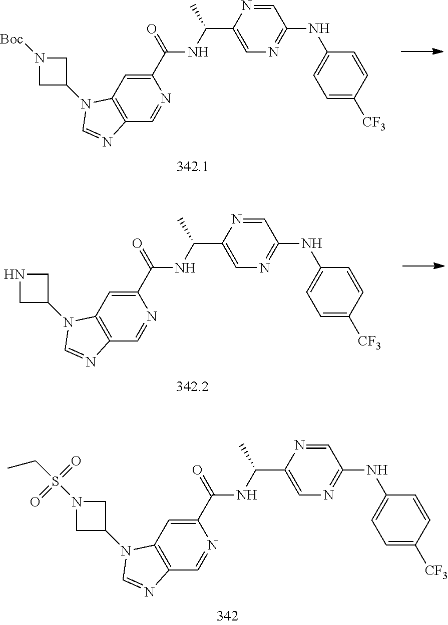

Synthesis of Compound 342.2 The compound 342.2 was prepared from compound 342.1 as previously described in the Table 1 general t-butyl carbamate deprotection procedure. ¹H NMR (500 MHz, DMSO-d₆) δ 8.21 (d, J=1.0 Hz, 1H), 7.91 (s, 1H), 7.65 (d, J=1.0 Hz, 1H), 7.47 (d, J=1.4 Hz, 1H), 7.42 (d, J=1.4 Hz, 1H), 7.07 (d, J=8.5 Hz, 2H), 6.74 (d, J=8.7 Hz, 2H), 4.76 (d, J=7.5 Hz, 1H), 4.52 (q, J=7.0 Hz, 1H), 3.34 (d, J=7.3 Hz, 4H), 0.83 (d, J=6.9 Hz, 3H)

Synthesis of Example 342. A solution of 50 mg (0.1 mmole) of compound 342.2 in 5 mL of CH₂Cl₂ was cooled in a dry ice/acetone bath and treated with 13 mg (0.1 mmole) of ethanesulfonyl chloride. After starting material had been completely consumed, the reaction mixture was diluted with H₂O and extracted with CH₂Cl₂. The organic layer was dried over Na₂SO₄, filtered, and concentrated. Purification by preparatory TLC (SiO₂, 5% MeOH/CH₂Cl₂) afforded 10 mg (15%) of the compound of Example 342 as a pale yellow solid. ¹H-NMR (CD₃OD, 500 MHz) δ 9.12 (s, 1H), 8.72 (s, 2H), 8.29 (s, 1H), 8.25 (s, 1H), 7.85 (d, J=8.0 Hz, 2H), 7.58 (d, J=8.0 Hz, 2H), 5.59-5.57 (m, 1H), 5.33-5.30 (m, 1H), 4.59 (t, J=7 Hz, 2H), 4.39-4.37 (m, 2H), 3.22 (q, 2H), 1.63 (d, J=7.5 Hz, 3H), 1.41 (t, J=7 Hz, 3H); LCMS m/z=575 [M+1].

TABLE 12
The following compounds of the present invention, set forth in Table 12, below, were prepared as previously described
in Example 342 and the appropriate sulfonyl chloride, acid chloride, or alkyl halide.

| Example | Structure | Characterization Data |
|---|---|---|
| 343 | 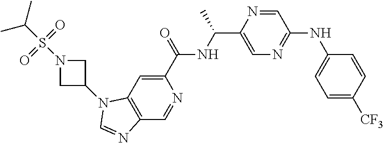 | ¹H-NMR (CD₃OD, 500 MHz) δ 9.08 (s, 1H), 8.72 (s, 1H), 8.59 (s, 1H), 8.29 (s, 1H), 8.21 (s, 1H), 7.83 (d, J = 8.0 Hz, 2H), 7.53 (d, J = 8.0 Hz, 2H), 5.59-5.57 (m, 1H), 5.32-5.29 (m, 1H), 4.62 (t, J = 7 Hz, 2H), 4.39-4.37 (m, 2H), 1.63 (d, J = 7 Hz, 3H), 1.42 (d, J = 7.0 Hz, 6H); LCMS m/z = 589 [M + 1]. |
| 344 | 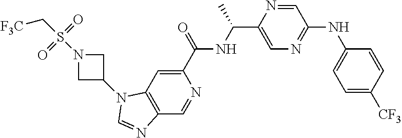 | ¹H-NMR (CD₃OD, 500 MHz) δ 9.08 (s, 1H), 8.65 (s, 1H), 8.58 (s, 1H), 8.29 (s, 1H), 8.23 (s, 1H), 7.85 (d, J = 8.5 Hz, 2H), 7.52 (d, J = 8.5 Hz, 2H), 5.59-5.56 (m, 1H), 5.32-5.30 (m, 1H), 4.73 (t, J = 7 Hz, 2H), 4.45-4.43 (m, 2H), 1.63 (d, J = 7.5 Hz, 3H); LCMS m/z = 629 [M + 1]. |
| 345 | 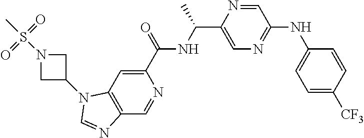 | ¹H-NMR (CD₃OD, 500 MHz) δ 9.05 (s, 1H), 8.68 (s, 1H), 8.59 (s, 1H), 8.25 (s, 1H), 8.23 (s, 1H), 7.83 (d, J = 8.0 Hz, 2H), 7.54 (d, J = 8.0 Hz, 2H), 5.52-5.50 (m, 1H), 5.33-5.30 (m, 1H), 4.59-4.56 (m, 2H), 4.42-4.40 (m, 2H), 3.15 (s, 3H), 1.63 (d, J = 7.0 Hz, 3H); LCMS m/z = 561 [M + 1]. |
| 346 | 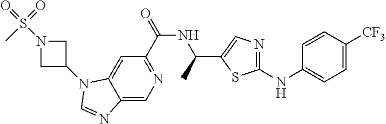 | ¹H-NMR (CD₃OD, 500 MHz) δ 9.01 (s, 1H), 8.65 (s, 1H), 8.61 (s, 1H), 7.73 (d, J = 8 Hz, 2H), 7.58 (d, J = 8 Hz, 2H), 7.21 (s, 1H), 5.61-5.58 (m, 1H), 5.50-5.43 (m, 1H), 4.63-4.59 (m, 2H), 4.41-4.38 (m, 2H), 3.10 (s, 3H), 1.73 (d, J = 6.5 Hz, 3H); LCMS m/z = 566 [M + 1]. |
| 347 | 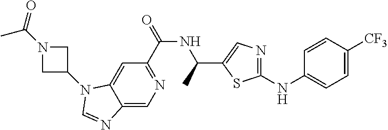 | ¹H-NMR (CD₃OD, 500 MHz) δ 9.01 (s, 1H), 8.69 (s, 1H), 8.42 (s, 1H), 7.69 (d, J = 8.5 Hz, 2H), 7.55 (d, J = 8.5 Hz, 2H), 7.22 (s, 1H), 5.61-5.58 (m, 1H), 5.47-5.46 (m, 1H), 4.83-4.63 (m, 2H), 4.45-4.42 (m, 2H), 2.01 (s, 3H), 1.73 (d, J = 6.5 Hz, 3H); LCMS m/z = 530 [M + 1]. |
| 348 | 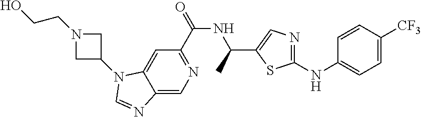 | ¹H-NMR (CD₃OD, 500 MHz) δ 8.98 (s, 1H), 8.71 (s, 1H), 8.52 (s, 1H), 7.69 (d, J = 8.5 Hz, 2H), 7.54 (d, J = 8.5 Hz, 2H), 7.21(s, 1H), 5.47-5.46 (m, 1H), 5.29-5.27 (m, 1H), 4.03-3.97 (m, 2H), 3.75-3.72 (m, 2H), 3.65-3.63 (m, 2H), 2.81-2.79 (m, 2H), 1.72 (d, J = 7 Hz, 3H;. LCMS m/z = 532 [M + 1]. |
| 349 | 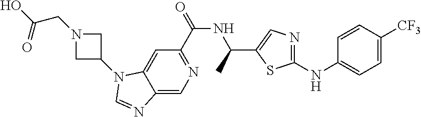 | ¹H-NMR (CD₃OD, 500 MHz) δ 8.98 (s, 1H), 9.72 (s, 1H), 8.49 (s, 1H), 7.69 (d, J = 8.5 Hz, 2H), 7.53 (d, J = 8.5 Hz, 2H), 7.23 (s, 1H), 5.46-5.43 (m, 1H), 5.19 (bs, 1H), 4.15-4.12 (m, 2H), 3.50 (s, 4H), 1.72 (d, J = 7.5 Hz, 3H); LCMS m/z = 485 [M − CH₂CO₂H]. |

### Example 350

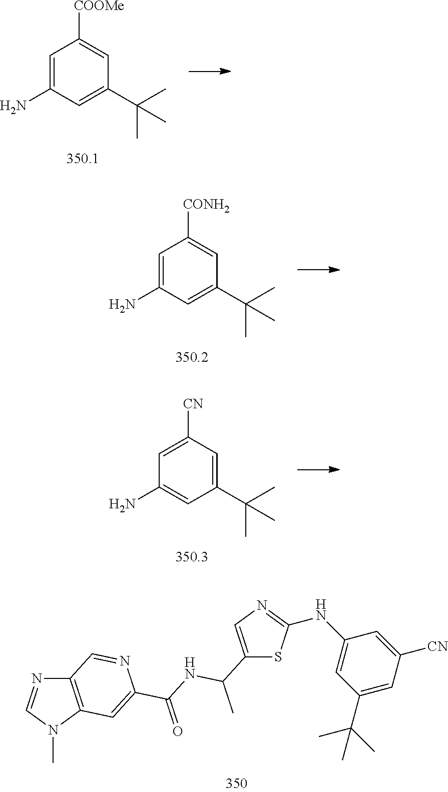

Synthesis of Compound 350.1. Compound 350.1 was prepared by esterification of 3-amino-5-t-butyl-benzoic acid with methanol.

Synthesis of Compound 350.2. Compound 350.2 was prepared from Compound 350.1 as described in Example 355.

Synthesis of Compound 350.3. Compound 350.2 (100 mg, 0.5 mmole) was treated with 1 mL of POCl₃ and heated at 90° C. for 2 hr. The reaction mixture was diluted with ice cold water and made basic by addition of saturated aqueous NAHCO₃. The aqueous layer was extracted twice with EtOAc. The combined organic layers were dried over Na₂SO₄, filtered, and concentrated to afford 30 mg (33%) of Compound 350.3.

Synthesis of Example 350. The compound of Example 350 was prepared as previously described in Example 240. ¹HNMR (CD₃OD, 500 MHz) δ 8.97 (s, 1H), 8.40 (d, J=8.0 Hz, 2H), 7.92 (bs, 1H), 7.77 (bs, 1H), 7.31 (s, 1H), 7.21 (s, 1H), 5.49-5.45 (m, 1H), 4.00 (s, 3H), 1.78 (d, J=7 Hz, 3H), 1.38 (s, 9H); LCMS m/z=460.2 [M+1].

### Example 351

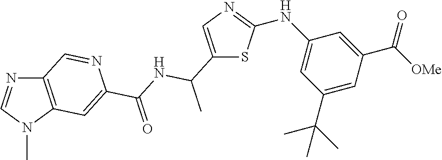

Synthesis of Example 351. The compound of Example 351 was prepared as previously described in Example 240 using compound 350.1. ¹H-NMR (DMSO-D₆, 500 MHz) δ 10.16 (s, 1H), 9.02 (d, J=9 Hz, 1H), 8.95 (s, 1H), 8.46 (s, 1H), 8.34 (s, 1H), 8.13 (s, 1H), 7.81 (s, 1H), 7.51 (s, 1H), 7.17 (s, 1H), 5.35-5.33 (m, 1H), 3.93 (s, 3H), 3.82 (s, 3H), 1.63 (d, J=7 Hz, 3H), 1.27 (s, 9H)); LCMS m/z=493 [M+1].

### Example 352

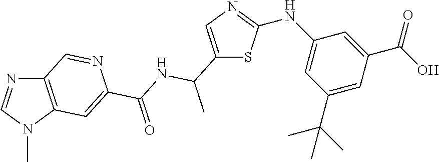

Synthesis of Example 352. The compound of Example 352 was prepared from the compound of Example 351 as described in Example 354. ¹H-NMR (DMSO-D₆, 500 MHz) δ 12.04 (bs, 1H), 10.14 (s, 1H), 9.03 (d, J=9 Hz, 1H), 8.97 (s, 1H), 8.48 (s, 1H), 8.35 (s, 1H), 8.11 (s, 1H), 7.80 (s, 1H), 7.52 (s, 1H), 7.18 (s, 1H), 5.37-5.35 (m, 1H), 3.95 (s, 3H), 1.64 (d, J=7 Hz, 3H), 1.28 (s, 9H)); LCMS m/z=480 [M+1].

### Example 353

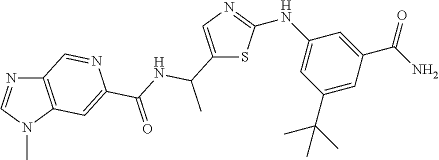

Synthesis of Example 353. The compound of Example 353 was prepared from the compound of Example 351 as described in Example 355. ¹H-NMR (DMSO-D₆, 500 MHz) δ 10.16 (s, 1H), 9.05 (d, J=9 Hz, 1H), 8.98 (s, 1H), 8.51 (s, 1H), 8.38 (s, 1H), 7.89 (s, 1H), 7.87 (s, 1H), 7.73 (s, 1H), 7.44 (s, 1H), 7.24 (s, 1H), 7.17 (s, 1H), 5.37-5.34 (m, 1H), 3.95 (s, 3H), 1.64 (d, J=7 Hz, 3H), 1.28 (s, 9H)); LCMS m/z=478 [M+1].

### Example 354

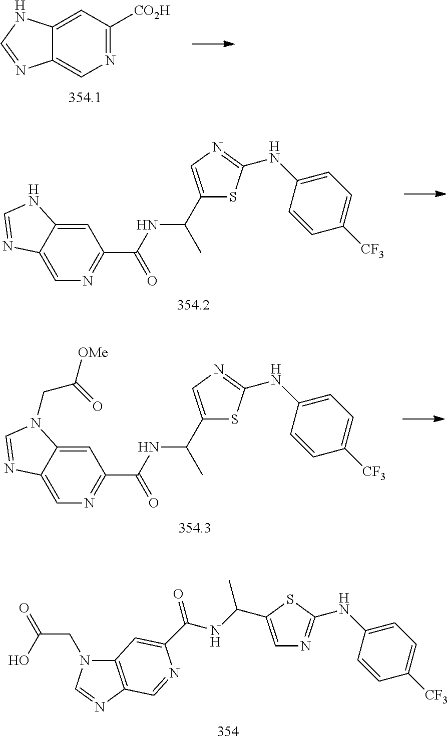

Synthesis of Compound 354.2. The compound 354.2 was prepared as previously described in the Table 1 general amide bond formation procedure using compound 354.1 which was prepared as described in Scheme E using L-histadine. ¹H NMR (500 MHz, DMSO-d₆) δ 10.45 (s, 1H), 9.08 (d, J=8.6 Hz, 1H), 8.99 (s, 1H), 8.49 (s, 1H), 8.37 (s, 1H), 7.76 (d, J=8.6 Hz, 2H), 7.62 (d, J=8.8 Hz, 2H), 7.21 (s, 1H), 5.37 (d, J=7.8 Hz, 1H), 5.32 (s, 1H), 1.64 (d, J=7.0 Hz, 3H).

Synthesis of Compound 354.3. A solution of 100 mg (0.23 mmole) of compound 354.2 in 5 mL of DMF was treated with 85 μL (0.35 mmole) of methyl bromoacetate and 32 mg (0.23 mmole) of K₂CO₃, and stirred at room temperature for 15 min. The mixture was diluted with H₂O and extracted with EtOAc. The organic layer was concentrated and the residue purified by flash column chromatography (SiO₂, 10% MeOH/CH₂Cl₂) to afford 20 mg (65%) of compound 354.3 as a white solid. ¹H NMR (500 MHz, DMSO-d₆) δ 8.98 (s, 1H), 8.45 (s, 1H), 8.27 (s, 1H), 7.70 (d, J=8.8 Hz, 2H), 7.59 (d, J=8.6 Hz, 2H), 7.19 (s, 1H), 5.32 (q, J=6.6 Hz, 1H), 5.26 (s, 1H), 3.87 (s, 3H), 1.61 (d, J=7.0 Hz, 3H).

Synthesis of Example 354. A solution of 20 mg (0.04 mmole) of compound 354.3 in 4 mL of CH₂Cl₂ was treated with two drops of TFA and stirred at room temperature for 4 hr. The reaction mixture was concentrated. The solid residue was washed with diethyl ether and then purified by flash column chromatography (SiO₂, 10% MeOH/CH₂Cl₂) to afford the compound of Example 354 as a yellow-white solid. ¹H-NMR (DMSO-D₆, 500 MHz) δ 13.45 (bs, 1H), 10.42 (s, 1H), 9.15 (d, J=7.5 Hz, 1H), 8.95 (s, 1H), 8.46 (s, 1H), 8.38 (s, 1H), 7.76 (d, J=8.0 Hz, 2H), 7.63 (d, J=8.0 Hz, 2H), 7.21 (s, 1H), 5.24-5.28 (m, 1H), 5.25 (bs. 2H), 1.67 (d, J=7.0 Hz, 3H); LCMS m/z=491 [M+1].

### Example 355

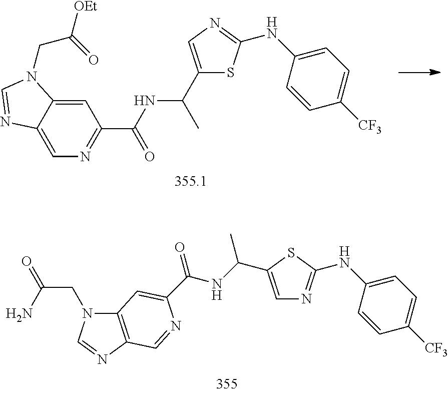

Synthesis of Compound 355.1. The Compound 355.1 was prepared as previously described in Example 354 using ethyl bromoacetate.

Synthesis of Example 355. Compound 355.1 (10 mg, 0.02 mmole) was treated with 3 mL of aqueous ammonia in a sealed tube and stirred at room temperature for 2 hr and then at 80° C. for an additional 2 hr. The reaction mixture was concentrated to dryness under vacuum, and the residue was washed with CH₂Cl₂ and Et₂O to afford 15 mg of the compound of Example 355 as a white solid. ¹H-NMR (DMSO-D₆, 500 MHz) δ 10.41 (s, 1H), 9.11 (d, J=7.5 Hz, 1H), 8.97 (s, 1H), 8.42 (s, 1H), 8.28 (s, 1H), 7.77-7.74 (m, 4H), 7.23 (s, 1H), 5.38-5.36 (m, 1H), 4.54 (bs, 2H), 4.78 (bs, 2H), 1.67 (d, J=7.0 Hz, 3H).

### Example 356

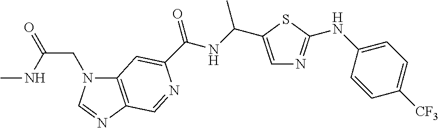

Synthesis of Example 356. The compound of Example 356 was prepared as previously described in Example 355 using methylamine. ¹H-NMR (DMSO-D₆, 500 MHz) δ 10.46 (s, 1H), 9.12 (d, J=7.0 Hz, 1H), 8.98 (s, 1H), 8.49 (s, 1H), 8.33 (s, 2H), 7.76 (d, J=8.0 Hz, 2H), 7.62 (d, J=8.0 Hz, 2H), 7.22 (s, 1H), 5.38-5.36 (m, 1H), 5.09 (s. 2H), 2.62 (s, 3H), 1.65 (d, J=7.0 Hz, 3H); LCMS m/z=503 [M+1].

### Example 357

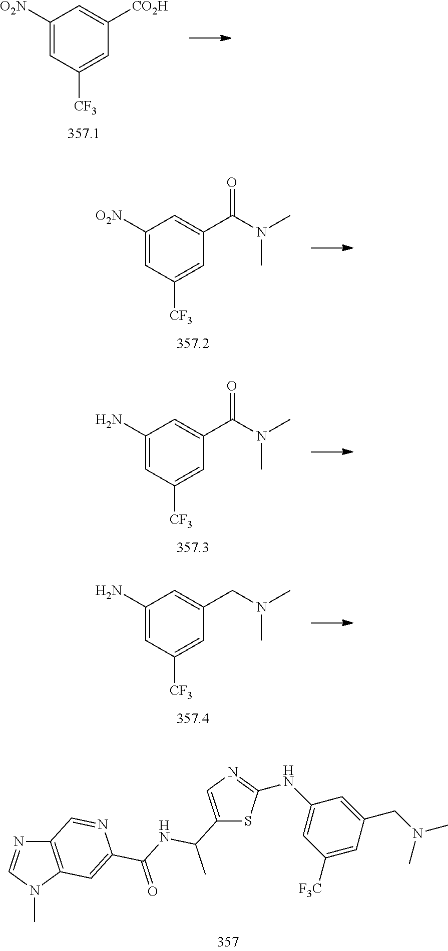

Synthesis of 357.2. A solution of 3-nitro-5-trifluoromethylbenzoic acid 357.1 (2 g, 8.5 mmol), dimethylamine hydrochloride (1.0 g, 12.7 mmol), EDCI (4.0 g, 21.2 mmol), HOBT (574 mg, 4.2 mmol) and DIPEA (1.4 g, 11.0 mol) in DMF (20 ml) was stirred at 80° C. for 16 hr. The reaction mixture was diluted with water (50 ml) and extracted with ethyl acetate (3×100 ml). The combined organic layers was washed with water (3×50 ml), dried over Na₂SO₄ and concentrated under reduced pressure. The resulting crude material was purified by column chromatography to give 357.2 as a brown liquid (1.4 g, 63%): ¹H-NMR (CDCl₃, 200 MHz) δ 8.61 (s, 1H), 8.58 (s, 1H), 8.11 (s, 1H), 3.23 (s, 3H), 3.13 (s, 3H); LCMS m/z=263 [M+1].

Synthesis of 357.3. A solution of 357.2 (1.3 g, 4.9 mmol), sodium dithionite (3.4 g, 19.8 mol), sodium carbonate (1.0 g, 9.9 mol) in MeOH (13 ml) and water (13 ml) was stirred at room temperature for 2 hr. The volatiles were removed under reduced pressure and extracted with ethyl acetate (3×100 ml). The combined organic layers was dried over Na₂SO₄ and concentrated under reduced pressure to obtain 357.3 as a light yellow solid (600 mg, 54.5%). ¹H-NMR (CDCl₃, 200 MHz) δ 7.0 (s, 1H), 6.90 (s, 1H), 6.80 (s, 1H), 3.23 (s, 3H), 3.13 (s, 3H); LCMS m/z=233 [M+1].

Synthesis of 357.4. A solution of 500 mg (1.91 mmole) of compound 357.3 in 10 mL on anhydrous THF was cooled in an ice bath and treated with 144 mg (3.8 mmole) of LiAlH₄. After addition was complete, the ice bath was removed and the reaction mixture was heated at reflux for 2 hr. After cooling to room temperature, excess hydride was quenched by the addition of aqueous NH₄Cl. The aqueous mixture was extracted with EtOAc. The organic layer was dried over Na₂SO₄, concentrated, and the residue was purified by preparatory TLC (SiO₂, 10% MeOH/CH₂Cl₂) to afford compound 357.4 as a thick brown gum.

Synthesis of Example 357. The compound of Example 357 was prepared as previously described in Example 240. ¹H-NMR (CD₃OD, 500 MHz) δ 8.98 (s, 1H), 8.41 (d, J=8 Hz, 2H), 7.95 (s, 1H), 7.69 (s, 1H), 7.23 (d, J=8 Hz, 2H), 5.49-5.47 (m, 1H), 4.01 (s, 3H), 3.59 (s, 2H), 2.31 (s, 6H), 1.74 (d, J=7.0 Hz, 3H); LCMS m/z=504 [M+1].

### Biological Assays

### (1) Biochemical FRET Assay

Method utilized for measuring the phosphorylation of MEK by wild-type (WT) B-Raf as a method for quantifying the ability of molecules to inhibit the enzymatic activity of WT-B-Raf.

In the assay methods described below, the following definitions apply:

“HEPES” refers to 4-(2-hydroxyethyl)-1-piperazineethanesulfonic acid;

“MEK” refers to mitogen activated extracellular signal-related kinase kinase;

“DTT” refers to dithiothreitol;

“APC” refers to allophycocyanin;

“TR-FRET” refers to time resolved fluorescence energy transfer;

“PBS” refers to phosphate buffered saline;

“PMSF” refers to phenyl methyl sulfonamide; and

“BSA” refers to bovine serum albumin.

TABLE 13
Reagents

| | | | Catalog | |
|---|---|---|---|---|
| Name | Units/Amount | Source | Number | Storage |
| Biotin-MEK1 | DB021505 | Biogen Idec. | In house | −80° C. |
| (15:1) | 767 μg/mL | | | |
| | (10.8 μM) | | | |
| ATP | 10 mM, 500 μl | Gibco BRL | 8330-019 | −20° C. |
| B-Raf (WT) | 12 μg/480 μl | Upstate | 14-530M | −80° C. |
| | 54% Pure | | | |
| | (2.1 μM) | | | |
| DMSO | 100% | Fisher | D128-500 | RT |
| Streptavidin | 14.8 μM SA | Prozyme | PJ25S | 4° C., in |
| Allophycocyanin | (2.20 mg/ml) | | | the dark |
| (SA-APC) | | | | |
| Polyclonal | 265 μg/ml | Cell | 9121 | −20° C. |
| Antiphospho | (1.8 μM) | Signaling | | |
| MEK1/2 (Ser | | Technologies | | |
| 217/221) | | Inc. | | |
| Antibody | | | | |
| Lance Eu- | 880 μg/ml | Perkin Elmer | AD083 | 4° C. |
| W1024 Anti | (5.5 μM) | | | |
| Rabbit IgG | | | | |
| LANCE 10X | N/A | Perkin Elmer | CR97-100 | 4° C. |
| Detection Buffer | | | | |
| SuperBlock in | N/A | Pierce | 37535 | 4° C. |
| TBS | | | | |

TABLE 14
Buffers

| Master Buffer | Storage |
|---|---|
| 50 mM Hepes, 60 mM NaCl, | 4° C. |
| 3 mM MgCl₂ | |
| 1M Dithiothreitol (DTT) | −20° C. in aliquots of 150 μl |
| 1M MnCl₂ | 4° C. |
| 20% BSA, 0.002% Sodium | 4° C. |
| Azide. | |
| 20% Tween-20 | room temperature (~25° C.) |
| 1M EDTA in dH₂O | room temperature (~25° C.) |

Equipment and Materials: Analyst AD, LJL BioSystems, ID1615; 96 well ½ Area Black Polystyrene plates. Costar 3694.

### Assay Protocol:

1. Add 10 μL 4.5× B-Raf WT
2. Add 10 μL 4.5× Test compound/DMSO
3. Add 25 μL mixture of 1.8×ATP/Biotin MEK
4. Incubate at room temperature for 90 minutes.
5. Add 5 μL of 150 mM EDTA to stop the reaction (final concentration of 15 mM; final volume of stopped reaction is 50 μA).
6. Add 50 μL of 2× detection reagents (SA-APC, Anti p-MEK1/2, Eu-AntiRabbit IgG).
7. Incubate at room temperature for 90 minutes.
8. Read on Analyst.

TABLE 15

Reagents used for Kinase reaction:
50 μM ATP
0.125 nM B-Raf (WT)
12.5 nM Biotin-MEK (15:1)
1% DMSO
50 mM Hepes, 60 mM NaCl, 3 mM MgCl₂, 2 mM DTT,
0.25 mM MnCl₂, 0.01% BSA, 0.01% Tween-20
Reagents used for Detection Reaction
20 nM SA-APC
2.5 nM Polyclonal Anti p-MEK1/2 (Ser217/221)
2.5 nM Eu-AntiRabbit IgG
1X Lance Detection Buffer
10% Superblock in TBS

### WT Raf

Inhibitors were diluted 4-fold in 100% DMSO and added to a final concentration of 10 μM to 40 μM to a solution containing 12.5 nM biotin-MEK, 0.125 nM WT Raf in 50 mM HEPES, pH 7.4, 60 mM NaCl, 3 mM MgCl₂, 2 mM DTT, 0.25 mM MnCl₂, 0.01% BSA, and 0.01% Tween-20 and incubated for 2 hours at room temperature. The kinase reaction was started by the addition of 50 μM ATP to a final volume of 45 μl and allowed to progress for 60 minutes. The reaction was stopped with 15 mM EDTA and 20 nM Streptavidin-APC, 2.5 nM Polyclonal anti p-MEK1/2 (Ser217/221), 2.5 nM Eu-labeled anti-rabbit IgG were added in Lance detection buffer and 5% Superblock in PBS for a final volume of 100 μl. The detection reaction was incubated for 90 minutes at room temperature and then read on an Analyst plate reader using standard TR-FRET (time resolved fluorescence resonance energy transfer) settings for Eu and APC.

### Mutant Raf

Inhibitors were diluted 4-fold in 100% DMSO and added to a final concentration of 10 μM to 40 μM to a solution containing 100 nM biotin-MEK, 0.125 nM V599E Raf in 50 mM HEPES, pH 7.4, 60 mM NaCl, 3 mM MgCl₂, 2 mM DTT, 0.25 mM MnCl₂, 0.01% BSA, and 0.01% Tween-20 and incubated for 20 minutes at room temperature. The kinase reaction was started by the addition of 25 μM ATP to a final volume of 45 μl and allowed to progress for 60 minutes. The reaction was stopped with 15 mM EDTA and 20 nM Streptavidin-APC, 2.5 nM Polyclonal anti p-MEK1/2 (Ser217/221), 2.5 nM Eu-labeled anti-rabbit IgG were added in Lance detection buffer and 5% Superblock in PBS for a final volume of 100 μl. The detection reaction was incubated for 90 minutes at room temperature and then read on an Analyst plate reader using standard TR-FRET (time resolved fluorescence resonance energy transfer) settings for Eu and APC.

### C-Raf

Inhibitors were diluted 4-fold in 100% DMSO and added to a final concentration of 10 μM to 40 μM to a solution containing 50 nM biotin-MEK, 0.075 nM C-Raf in 50 mM HEPES, pH 7.4, 60 mM NaCl, 3 mM MgCl₂, 2 mM DTT, 0.25 mM MnCl₂, 0.01% BSA, and 0.01% Tween-20 and incubated for 20 minutes at room temperature. The kinase reaction was started by the addition of 10 μM ATP to a final volume of 45 μl and allowed to progress for 60 minutes. The reaction was stopped with 15 mM EDTA and 20 nM Streptavidin-APC, 2.5 nM Polyclonal anti p-MEK1/2 (Ser217/221), 2.5 nM Eu-labeled anti-rabbit IgG were added in Lance detection buffer and 5% Superblock in PBS for a final volume of 100 μl. The detection reaction was incubated for 90 minutes at room temperature and then read on an Analyst plate reader using standard TR-FRET (time resolved fluorescence resonance energy transfer) settings for Eu and APC.

Certain compounds of the present invention were assayed using the above Biochemical FRET assay and were found to be inhibitors of Raf kinase. Table 16 shows the activity of selected compounds of this invention in the FRET assay. Compounds having an activity designated as “A” provided an IC₅₀≦100 nM; compounds having an activity designated as “B” provided an IC₅₀ of 100-1000 nM; and compounds having an activity designated as “C” provided an IC₅₀ of 1000-10,000 nM.

TABLE 16

| Example | Raf (mut) inhibition |
|---|---|
| 1 | A |
| 2 | A |
| 3 | A |
| 4 | A |
| 5 | A |
| 6 | A |
| 7 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 37 | A |
| 41 | A |
| 42 | A |
| 43 | A |
| 44 | A |
| 49 | A |
| 51 | B |
| 52 | A |
| 54 | B |
| 55 | A |
| 56 | A |
| 57 | A |
| 58 | A |
| 62 | B |
| 65 | B |
| 67 | B |
| 68 | A |
| 71 | A |
| 72 | A |
| 73 | A |
| 74 | A |
| 75 | A |
| 76 | A |
| 77 | A |
| 82 | A |
| 86 | A |
| 87 | B |
| 89 | B |
| 90 | A |
| 91 | B |
| 92 | A |
| 93 | B |
| 94 | B |
| 95 | A |
| 96 | A |
| 97 | A |
| 98 | A |
| 99 | A |
| 101 | A |
| 103 | A |
| 106 | A |
| 107 | A |
| 108 | A |
| 109 | A |
| 110 | A |
| 111 | A |
| 118 | A |
| 119 | A |
| 121 | B |
| 123 | B |
| 125 | B |
| 126 | A |
| 127 | A |
| 128 | B |
| 129 | C |
| 130 | B |
| 131 | B |
| 132 | B |
| 133 | B |
| 134 | B |
| 138 | B |
| 140 | A |
| 148 | A |
| 150 | A |
| 153 | B |
| 155 | A |
| 156 | A |
| 167 | A |
| 174 | A |
| 175 | A |
| 176 | A |
| 177 | A |
| 179 | A |
| 180 | A |
| 181 | A |
| 182 | A |
| 183 | A |
| 185 | A |
| 187 | B |
| 188 | A |
| 189 | A |
| 190 | A |
| 198 | A |
| 199 | A |
| 201 | A |
| 203 | A |
| 207 | A |
| 209 | B |
| 210 | B |
| 211 | A |
| 212 | A |
| 213 | C |
| 214 | B |
| 215 | C |
| 216 | C |
| 217 | A |
| 218 | A |
| 219 | B |
| 220 | A |
| 221 | B |
| 222 | B |
| 223 | B |
| 224 | A |
| 225 | A |
| 227 | A |
| 228 | B |
| 229 | B |
| 230 | B |
| 231 | B |
| 233 | A |
| 234 | A |
| 238 | A |
| 241 | A |
| 243 | A |
| 244 | A |
| 245 | A |
| 261 | A |
| 262 | A |
| 263 | A |
| 264 | A |
| 265a | A |
| 265b | A |
| 266 | B |
| 267 | A |
| 268 | A |
| 270 | A |
| 273 | A |
| 276 | A |
| 279 | A |
| 280 | A |
| 282 | A |
| 283 | A |
| 285 | A |
| 286 | A |
| 287 | A |
| 289 | A |
| 290 | A |
| 291 | A |
| 292 | A |
| 295 | B |
| 296 | A |
| 298 | A |
| 299 | A |
| 300 | A |
| 309 | B |
| 310 | A |
| 311 | A |
| 316 | B |
| 317 | B |
| 318 | A |
| 320 | A |
| 332 | B |
| 333 | B |
| 334 | B |
| 339 | A |
| 340 | A |
| 341 | B |
| 346 | A |
| 347 | A |
| 348 | A |
| 350 | A |
| 351 | A |
| 352 | B |
| 353 | A |
| 354 | A |
| 356 | A |

### (2) Mechanistic Cellular Assay for Raf Kinase Activity

The following method was utilized for quantifying the amount of phospho-ERK in melanoma derived WM-266-4 cells (one allele each of wild type BRaf and mutant BRaf (V600D) as an indicator of Raf kinase activity in cells treated with various kinase inhibitors.

TABLE 17

| Materials Needed | Catalog Number |
|---|---|
| WM-266-4 cells | (ATCC number: CRL-1676) |
| RPMI 1640 cell culture medium | |
| Fetal Bovine Serum (FBS) | |
| Phosphate Buffered Saline (PBS) | |
| 96-well tissue culture plates | |
| Tissue culture 37° C. incubator | |
| 96-well V-bottom plates | |
| Rotary plate shaker (e.g., BELLCO | |
| GLASS Mini Orbital Shaker) | |
| Bio-Plex suspension array system | |
| Bio-Plex Cell Lysis Kit | (Bio Rad Catalog #171-304011) |
| Phenyl methyl sulphonyl fluoride | |
| (PMSF) | |
| Bio-Plex Phospho-ERK1/2 Assay Kit | (Bio Rad Catalog #171-V22238) |

### Day 1: Cell Seeding

(1) Detached adhered WM-266-4 cells from flask using 0.25% Trypsin. Resuspended cells in growth media (90% RPMI 1640, 10% FBS) and determine cell density.

(2) Seeded cells @ 10,000 cells/well in 96-well (flat bottom) tissue culture plates (36,000 cells/cm²). Added growth media to a final volume of 200 uL/well and incubated overnight at 37° C.

### Day 2: Cell Treatment

(1) Prepared compound dilutions (1000× in DMSO) as follows. Starting with a stock of 5 mM compound in DMSO, diluted serially 3-fold in DMSO for a total of eight concentrations (5 mM, 1.67 mM, 0.556 mM, 0.185 mM, 0.062 mM, 0.021 mM, 0.007 mM, 0.002 mM).

(2) Prepared compound-containing media by adding 1 mL treatment media (100% RPMI 1640 without FBS) to 1 μL of compound dilution (from step 3).

(3) Removed plates (from step 2) from incubator. Aspirated media and replace with 150 μL compound-containing media. Incubate for 1-2 hr at 37° C.

(4) Removed plates (from step 5) from incubator and treated each as follows: aspirated compound-containing media and replaced with 300 μL ice-cold 1×PBS, aspirated PBS and replaced with 45 μL lysis buffer (Biorad Bio-Plex lysis buffer containing 0.4% v/v lysis buff. Factor 1, 0.2% v/v lysis buff. Factor 2, and PMSF to 2 mM final concentration), and then placed plate on ice until all plates were treated.

(5) After all plates were processed (step 6), placed plates on an orbital shaker and shook at room temperature for at least 15 min.

(6) Finally, removed plates from shaker, and transfered 40 μL/well of lysate from each to new corresponding 96-well V-bottom plates. At this point, samples may be frozen and stored @−80 C.°.

### Day 2: Bioplex Assay

(1) Thaw (if necessary) plates (from step 8) and added 40 μL of Phospho-Protein Assay Buffer to each 40 μL lysate for a 1:1 dilution.

(2) Prepared phospho-ERK1,2 Bioplex beads by diluting 1:50 with Bioplex Wash Buffer (mixing 49 μL Wash Buffer with 1 μL of phospho-ERK1,2 Bioplex beads for each sample to be analyzed). Protected from light by wrapping tube in aluminum foil and kept at room temperature.

(3) Prepared Filter Plate by adding 100 μL/well Bioplex Wash Buffer and removed by vacuum filtration.

(4) Add 50 μL of bead solution (from step 10) to each well of a prepared Filter Plate (from step 11) and vacuum filter. Wash/filter 2× with 100 μL/well Wash Buffer.

(5) Added 50 μL of each lysate to appropriate well of the Filter Plate (from step 12). For this and all subsequent plate incubation steps, placed plate on an inverted plate cover (reduces background), and wrapped in aluminum foil (to protect from light). Shook overnight at room temperature. Included positive (control lysate) and negative (lysis buffer) controls.

### Day 3: Bioplex Assay Continued

(1) Prepared detection antibody (phospho-ERK1,2 Ab) by diluting 1:25 with Detection Antibody Dilution Buffer Buffer (mixing 24 μL Detection Antibody Dilution Buffer with 1 μL of phospho-ERK1,2 Ab for each sample to be analyzed).

(2) Removed plate (from step 13) from shaker and vacuum filter. Washed/filter plate 3× with 100 μL/well Wash Buffer. Added 25 μL of diluted antibody to each well. Incubated on shaker at RT for 30-45 min.

(3) Prepared streptavidin-PE by diluting 1:100 with Wash Buffer (mixing 49.5 μL Wash Buffer with 0.5 μL of 100× streptavidin-PE for each sample to be analyzed). Protected from light.

(4) Removed plate (from step 15) from shaker and vacuum filter. Washed/filter plate 3× with 100 μL/well Wash Buffer. Add 50 μL of diluted streptavidin-PE solution (from step 16) to each sample well. Incubated on shaker for 10-20 min.

(5) Removed plate from shaker and vacuum filter. Wash/filter plate 3× with 100 μL/well Bead Resuspension Buffer. After last wash resuspended beads in 125 μL Bead Resuspension Buffer. Place plate on shaker for 2-3 minutes to ensure beads are well resuspended.

(6) Quantified phospho-ERK by reading plate in the Bio-Plex plate reader (run start-up and calibration programs before this step) using bead region 38 (pERK1,2) and counting 50 beads per region.

WM-266-4 cells were seeded at a density of 10,000 cells/well in RPMI 1640 cell culture media containing 10% FBS in a 96-well flat bottom and incubated overnight at 37° C. Inhibitors were diluted 3-fold in DMSO, added to serum free RPMI 1640 cell culture media to a final concentration range of 5 μM to 2 nM, and used to treat the previously seeded WM-266-4 cells for 1-2 hr at 37° C. Cells were washed with ice-cold PBS, treated with 45 μl of lysis buffer (Bio-Rad Bio-Plex Lysis Buffer, Cat #171-304011, containing 0.4% v/v lysis buffer factor 1, 0.2% v/v lysis buffer Factor 2, and 2 mM PMSF) for 15 minutes on an orbital shaker at room temperature. Phosphorylated ERK was detected using a phospho-ERK Bioplex kit (Bio-Rad, Cat #171-304011) per the manufacturer's instructions and detected on a Bio-Plex plate reader counting 50 beads per region.

Certain compounds of the present invention were assayed using the above Cellular Assay for Raf Kinase Activity and were found to be inhibitors of Raf kinase. Table 18 shows the activity of selected compounds of this invention in the cellular assay. Compounds having an activity designated as “A” provided an IC₅₀≦100 nM; compounds having an activity designated as “B” provided an IC₅₀ of 100-1000 nM; and compounds having an activity designated as “C” provided an IC₅₀ of 1000-10,000 nM.

TABLE 18

| Example | pERK EC₅₀ |
|---|---|
| 1 | C |
| 2 | A |
| 3 | C |
| 4 | A |
| 5 | B |
| 6 | A |
| 7 | A |
| 8 | A |
| 9 | A |
| 10 | B |
| 11 | B |
| 12 | A |
| 13 | A |
| 14 | A |
| 15 | A |
| 16 | B |
| 17 | A |
| 18 | B |
| 19 | A |
| 20 | A |
| 21 | B |
| 22 | B |
| 23 | B |
| 24 | A |
| 25 | B |
| 26 | B |
| 27 | B |
| 28 | A |
| 29 | A |
| 30 | A |
| 31 | B |
| 32 | C |
| 33 | B |
| 34 | B |
| 35 | A |
| 36 | C |
| 37 | A |
| 38 | A |
| 39 | A |
| 40 | A |
| 41 | B |
| 42 | A |
| 43 | A |
| 44 | C |
| 45 | C |
| 46 | C |
| 47 | B |
| 48 | B |
| 49 | B |
| 50 | C |
| 51 | C |
| 52 | C |
| 53 | B |
| 54 | C |
| 55 | C |
| 56 | B |
| 57 | C |
| 58 | A |
| 59 | A |
| 60 | B |
| 61 | A |
| 62 | C |
| 63 | A |
| 64 | B |
| 65 | C |
| 66 | A |
| 67 | C |
| 68 | C |
| 69 | C |
| 70 | B |
| 71 | C |
| 72 | C |
| 73 | B |
| 74 | C |
| 75 | B |
| 76 | C |
| 77 | A |
| 79 | B |
| 80 | B |
| 81 | A |
| 82 | B |
| 83 | A |
| 84 | A |
| 85 | A |
| 86 | B |
| 87 | C |
| 88 | B |
| 89 | B |
| 90 | B |
| 91 | C |
| 92 | C |
| 93 | C |
| 94 | C |
| 95 | C |
| 96 | C |
| 97 | C |
| 98 | C |
| 99 | C |
| 100 | C |
| 101 | C |
| 102 | A |
| 103 | C |
| 104 | B |
| 105 | B |
| 106 | C |
| 107 | B |
| 108 | B |
| 109 | C |
| 110 | C |
| 111 | C |
| 112 | B |
| 113 | B |
| 114 | C |
| 115 | A |
| 116 | B |
| 117 | C |
| 118 | C |
| 119 | C |
| 120 | B |
| 121 | B |
| 122 | B |
| 123 | C |
| 124 | A |
| 125 | B |
| 126 | A |
| 127 | A |
| 128 | B |
| 129 | C |
| 130 | B |
| 131 | B |
| 132 | B |
| 133 | B |
| 134 | B |
| 135 | A |
| 136 | B |
| 137 | B |
| 138 | C |
| 140 | C |
| 141 | B |
| 142 | C |
| 143 | C |
| 144 | B |
| 145 | B |
| 146 | A |
| 147 | A |
| 148 | C |
| 149 | A |
| 150 | C |
| 151 | A |
| 152 | A |
| 153 | C |
| 154 | B |
| 155 | C |
| 156 | C |
| 157 | C |
| 158 | A |
| 159 | A |
| 160 | C |
| 161 | B |
| 162 | A |
| 163 | B |
| 164 | B |
| 165 | B |
| 166 | A |
| 167 | B |
| 168 | A |
| 169 | A |
| 170 | B |
| 171 | A |
| 172 | B |
| 173 | A |
| 174 | A |
| 175 | A |
| 176 | A |
| 177 | B |
| 178 | B |
| 179 | B |
| 180 | B |
| 181 | B |
| 182 | B |
| 183 | A |
| 184 | B |
| 185 | B |
| 186 | B |
| 187 | C |
| 188 | A |
| 189 | C |
| 190 | B |
| 191 | C |
| 192 | B |
| 194 | C |
| 195 | C |
| 198 | B |
| 199 | A |
| 200 | C |
| 201 | A |
| 202 | C |
| 203 | A |
| 204 | C |
| 205 | A |
| 206 | B |
| 207 | B |
| 208 | A |
| 209 | A |
| 211 | C |
| 212 | C |
| 214 | B |
| 217 | B |
| 218 | A |
| 219 | B |
| 220 | B |
| 221 | B |
| 222 | A |
| 223 | A |
| 224 | B |
| 225 | C |
| 226 | B |
| 227 | B |
| 229 | B |
| 231 | C |
| 232 | A |
| 233 | B |
| 234 | B |
| 235 | B |
| 236 | A |
| 237 | C |
| 238 | A |
| 239 | C |
| 240 | A |
| 241 | A |
| 242 | C |
| 243 | A |
| 244 | A |
| 245 | A |
| 246 | B |
| 247 | C |
| 248 | B |
| 249 | B |
| 250 | B |
| 251 | C |
| 252 | B |
| 253 | B |
| 254 | C |
| 255 | B |
| 256 | B |
| 257 | A |
| 258 | B |
| 259 | B |
| 260 | C |
| 261 | B |
| 262 | B |
| 263 | B |
| 264 | B |
| 265a | B |
| 265b | B |
| 266 | C |
| 267 | A |
| 268 | A |
| 269 | A |
| 270 | A |
| 271 | B |
| 272 | B |
| 273 | A |
| 274 | A |
| 275 | B |
| 276 | A |
| 277 | B |
| 278 | A |
| 279 | A |
| 280 | B |
| 281 | C |
| 282 | B |
| 283 | B |
| 284 | B |
| 285 | A |
| 286 | A |
| 287 | A |
| 288 | B |
| 289 | B |
| 290 | A |
| 291 | B |
| 292 | A |
| 293 | B |
| 294 | B |
| 295 | A |
| 296 | B |
| 297 | A |
| 298 | A |
| 299 | B |
| 300 | C |
| 301 | C |
| 302 | B |
| 303 | B |
| 304 | B |
| 305 | B |
| 306 | C |
| 307 | C |
| 308 | C |
| 309 | C |
| 310 | C |
| 311 | B |
| 312 | A |
| 313 | C |
| 314 | B |
| 315 | B |
| 316 | C |
| 317 | C |
| 318 | C |
| 319 | B |
| 320 | A |
| 321 | A |
| 322 | B |
| 323 | A |
| 324 | B |
| 325 | B |
| 326 | B |
| 327 | A |
| 328 | A |
| 329 | A |
| 330 | B |
| 331 | A |
| 332 | A |
| 333 | B |
| 334 | C |
| 335 | A |
| 336 | A |
| 337 | B |
| 339 | B |
| 340 | C |
| 341 | C |
| 342 | A |
| 343 | A |
| 344 | B |
| 345 | A |
| 346 | A |
| 347 | B |
| 348 | A |
| 349 | C |
| 350 | C |
| 351 | B |
| 352 | C |
| 353 | C |
| 354 | C |
| 355 | C |
| 356 | B |
| 357 | C |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I: 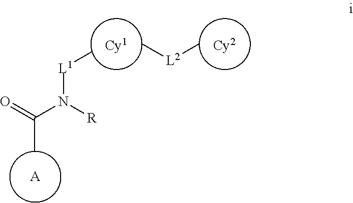
or a pharmaceutically acceptable salt thereof, wherein:
Cy¹ is phenylene, 5-6 membered saturated or partially unsaturated carbocyclylene, 7-10 membered saturated or partially unsaturated bicyclic carbocyclylene, a 5-6 membered saturated or partially unsaturated heterocyclylene ring having 1-2 heteroatoms independently selected from nitrogen, oxygen, and sulfur, a 7-10 membered saturated or partially unsaturated bicyclic heterocyclylene ring having 1-3 heteroatoms independently selected from nitrogen, oxygen, and sulfur, 8-10 membered bicyclic arylene, a 5-6 membered heteroarylene ring having 1-3 heteroatoms independently selected from nitrogen, oxygen, and sulfur, or an 8-10 membered bicyclic heteroarylene ring having 1-4 heteroatoms independently selected from nitrogen, oxygen, and sulfur, wherein:
Cy¹ is optionally substituted with one or two groups independently selected from halogen, —R^{c}, —CN, —NO₂, —OR^{c}, —N(R^{c})₂, and —SR^{c}, wherein each R^{c} is independently hydrogen or a C₁₋₂ alkyl group optionally substituted with 1-3 groups independently selected from halogen, —OH, —NH₂, —SH, and —CN;
Cy² is an optionally substituted group selected from phenyl, a 5-8 membered saturated or partially unsaturated carbocyclic ring, a 7-10 membered saturated or partially unsaturated bicyclic carbocyclic ring, a 5-8 membered saturated or partially unsaturated heterocyclic ring having 1-2 heteroatoms independently selected from nitrogen, oxygen, and sulfur, a 7-10 membered saturated or partially unsaturated bicyclic heterocyclic ring having 1-3 heteroatoms independently selected from nitrogen, oxygen, and sulfur, an 8-10 membered bicyclic aryl ring, a 5-6 membered heteroaryl ring having 1-3 heteroatoms independently selected from nitrogen, oxygen, and sulfur, or an 8-10 membered bicyclic heteroaryl ring having 1-4 heteroatoms independently selected from nitrogen, oxygen, and sulfur;
L¹ is an optionally substituted, straight or branched bivalent C₁₋₆ alkylene chain;
L² is —NR¹— or —C(O)NR¹—;
R and R¹ are independently hydrogen or an optionally substituted C₁₋₆ aliphatic group; and
Ring A is an aromatic ring selected from the group consisting of Ring A¹, Ring A², Ring A³, Ring A⁴, and Ring A⁵, wherein:
(a) Ring A¹ is: 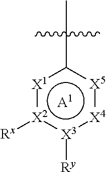
wherein:
X¹, X⁴ and X⁵ are independently CR⁴ or N;
X² is C or N, provided that when X² is N, R^{x} and R^{y} are taken together with their intervening atoms to form a fused heteroaromatic ring;
X³ is C;
R^{x} and R^{y} are independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂; or
R^{x} and R^{y} are taken together with their intervening atoms to form a 5-7 membered partially unsaturated or aromatic fused ring having 0-3 ring heteroatoms independently selected from nitrogen, oxygen, and sulfur; wherein:
any substitutable carbon on the ring formed by R^{x} and R^{y} is optionally substituted with —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —C═NN(R³)₂, —C═NOR², —N(R³)C(O)NR³)₂, —N(R³)SO₂N(R³)₂, —N(R³)SO₂R², or —OC(O)N(R³)₂, and
any substitutable nitrogen on the ring formed by R^{x} and R^{y} is optionally substituted with —R², —C(O)R², —CO₂R², —C(O)C(O)R², —C(O)CH₂—C(O)R², —S(O)R², —S(O)₂R², —C(O)N(R³)₂, —SO₂N(R³)₂, —OC(O)R², or —OC(O)N(R³)₂;
each R² is independently hydrogen or an optionally substituted group selected from C₁₋₆ aliphatic, phenyl, a 3-8 membered saturated or partially unsaturated carbocyclic ring, a 4-8 membered saturated or partially unsaturated heterocyclic ring having 1-2 heteroatoms independently selected from nitrogen, oxygen, and sulfur, a 7-10 membered saturated or partially unsaturated bicyclic heterocyclic ring having 1-4 heteroatoms independently selected from nitrogen, oxygen, and sulfur, an 8-10 membered bicyclic aryl ring, a 5-6 membered heteroaryl ring having 1-3 heteroatoms independently selected from nitrogen, oxygen, and sulfur, or an 8-10 membered bicyclic heteroaryl ring having 1-4 heteroatoms independently selected from nitrogen, oxygen, and sulfur;
each R³ is independently —R², or two R³ on the same nitrogen are taken together with the nitrogen to form an optionally substituted 5-8 membered saturated or partially unsaturated ring having 1-4 heteroatoms independently selected from nitrogen, oxygen, and sulfur; and
each R⁴ is independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂;
(b) Ring A² is: 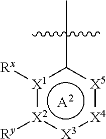
wherein:
X¹ and X² are independently C or N, provided that when X¹ or X² is N, R^{x} and R^{y} are taken together with their intervening atoms to form a fused heteroaromatic ring;
X³, X⁴, and X⁵ are independently CR⁴ or N;
R^{x} and R^{y} are independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂; or
R^{x} and R^{y} are taken together with their intervening atoms to form a 5-7 membered partially unsaturated or aromatic fused ring having 0-3 ring heteroatoms independently selected from nitrogen, oxygen, and sulfur; wherein:
any substitutable carbon on the ring formed by R^{x} and R^{y} is optionally substituted with —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —C═NN(R³)₂, —C═NOR², —N(R³)C(O)NR³)₂, —N(R³)SO₂N(R³)₂, —N(R³)SO₂R², or —OC(O)N(R³)₂, and
any substitutable nitrogen on the ring formed by R^{x} and R^{y} is optionally substituted with —R², —C(O)R², —CO₂R², —C(O)C(O)R², —C(O)CH₂—C(O)R², —S(O)R², —S(O)₂R², —C(O)N(R³)₂, —SO₂N(R³)₂, —OC(O)R², or —OC(O)N(R³)₂;
each R² is independently hydrogen or an optionally substituted group selected from C₁₋₆ aliphatic, phenyl, a 3-8 membered saturated or partially unsaturated carbocyclic ring, a 4-8 membered saturated or partially unsaturated heterocyclic ring having 1-2 heteroatoms independently selected from nitrogen, oxygen, and sulfur, a 7-10 membered saturated or partially unsaturated bicyclic heterocyclic ring having 1-4 heteroatoms independently selected from nitrogen, oxygen, and sulfur, an 8-10 membered bicyclic aryl ring, a 5-6 membered heteroaryl ring having 1-3 heteroatoms independently selected from nitrogen, oxygen, and sulfur, or an 8-10 membered bicyclic heteroaryl ring having 1-4 heteroatoms independently selected from nitrogen, oxygen, and sulfur;
each R³ is independently —R², or two R³ on the same nitrogen are taken together with the nitrogen to form an optionally substituted 5-8 membered saturated or partially unsaturated having 1-4 heteroatoms independently selected from nitrogen, oxygen, and sulfur; and
each R⁴ is independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂;
(c) Ring A³ is: 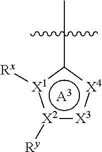
wherein:
X¹ and X² are independently C or N;
X³ and X⁴ are independently CR⁴, NR⁵, N, O, or S, as valency permits;
R^{x} and R^{y} are independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂; or
R^{x} and R^{y} are taken together with their intervening atoms to form a 5-7 membered partially unsaturated or aromatic fused ring having 0-3 ring heteroatoms independently selected from nitrogen, oxygen, and sulfur; wherein:
any substitutable carbon on the ring formed by R^{x} and R^{y} is optionally substituted with —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —C═NN(R³)₂, —C═NOR², —N(R³)C(O)N(R³)₂, —N(R³)SO₂N(R³)₂, —N(R³)SO₂R², or —OC(O)N(R³)₂, and
any substitutable nitrogen on the ring formed by R^{x} and R^{y} is optionally substituted with —R², —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², or —OC(O)N(R³)₂;
each R² is independently hydrogen or an optionally substituted group selected from C₁₋₆ aliphatic, phenyl, a 3-8 membered saturated or partially unsaturated carbocyclic ring, a 4-8 membered saturated or partially unsaturated heterocyclic ring having 1-2 heteroatoms independently selected from nitrogen, oxygen, and sulfur, a 7-10 membered saturated or partially unsaturated bicyclic heterocyclic ring having 1-4 heteroatoms independently selected from nitrogen, oxygen, and sulfur, an 8-10 membered bicyclic aryl ring, a 5-6 membered heteroaryl ring having 1-3 heteroatoms independently selected from nitrogen, oxygen, and sulfur, or an 8-10 membered bicyclic heteroaryl ring having 1-4 heteroatoms independently selected from nitrogen, oxygen, and sulfur;
each R³ is independently —R², or two R³ on the same nitrogen are taken together with the nitrogen to form an optionally substituted 5-8 membered saturated or partially unsaturated ring having 1-4 heteroatoms independently selected from nitrogen, oxygen, or sulfur;
each R⁴ is independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂; and
each R⁵ is independently —R², halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂;
(d) Ring A⁴ is: 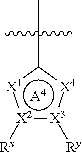
wherein:
X¹ and X⁴ are independently CR⁴, NR⁵, N, O, or S, as valency permits;
X² and X³ are independently C or N;
R^{x} and R^{y} are independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂; or
R^{x} and R^{y} are taken together with their intervening atoms to form a 5-7 membered partially unsaturated or aromatic fused ring having 0-3 ring heteroatoms independently selected from nitrogen, oxygen, and sulfur; wherein:
any substitutable carbon on the ring formed by R^{x} and R^{y} is optionally substituted with —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —C═NN(R³)₂, —C═NOR², —N(R³)C(O)N(R³)₂, —N(R³)SO₂N(R³)₂, —N(R³)SO₂R², or —OC(O)N(R³)₂, and
any substitutable nitrogen on the ring formed by R^{x} and R^{y} is optionally substituted with —R², —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², or —OC(O)N(R³)₂;
each R² is independently hydrogen or an optionally substituted group selected from C₁₋₆ aliphatic, phenyl, a 3-8 membered saturated or partially unsaturated carbocyclic ring, a 4-8 membered saturated or partially unsaturated heterocyclic ring having 1-2 heteroatoms independently selected from nitrogen, oxygen, and sulfur, a 7-10 membered saturated or partially unsaturated bicyclic heterocyclic ring having 1-4 heteroatoms independently selected from nitrogen, oxygen, and sulfur, an 8-10 membered bicyclic aryl ring, a 5-6 membered heteroaryl ring having 1-3 heteroatoms independently selected from nitrogen, oxygen, and sulfur, or an 8-10 membered bicyclic heteroaryl ring having 1-4 heteroatoms independently selected from nitrogen, oxygen, and sulfur;
each R³ is independently —R², or two R³ on the same nitrogen are taken together with the nitrogen to form an optionally substituted 5-8 membered saturated or partially unsaturated ring having 1-4 heteroatoms independently selected from nitrogen, oxygen, and sulfur;
each R⁴ is independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂; and
each R⁵ is independently —R², halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂;
(e) Ring A⁵ is: 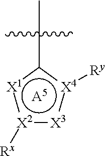
wherein:
X¹ and X³ are independently CR⁴, NR⁵, N, O, or S, as valency permits;
X² and X⁴ are independently C or N;
R^{x} and R^{y} are independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂;
each R² is independently hydrogen or an optionally substituted group selected from C₁₋₆ aliphatic, phenyl, a 3-8 membered saturated or partially unsaturated carbocyclic ring, a 4-8 membered saturated or partially unsaturated heterocyclic ring having 1-2 heteroatoms independently selected from nitrogen, oxygen, and sulfur, a 7-10 membered saturated or partially unsaturated bicyclic heterocyclic ring having 1-4 heteroatoms independently selected from nitrogen, oxygen, and sulfur, an 8-10 membered bicyclic aryl ring, a 5-6 membered heteroaryl ring having 1-3 heteroatoms independently selected from nitrogen, oxygen, and sulfur, or an 8-10 membered bicyclic heteroaryl ring having 1-4 heteroatoms independently selected from nitrogen, oxygen, and sulfur;
each R³ is independently —R², or two R³ on the same nitrogen are taken together with the nitrogen to form an optionally substituted 5-8 membered saturated or partially unsaturated ring having 1-4 heteroatoms independently selected from nitrogen, oxygen, and sulfur;
each R⁴ is independently —R², oxo, halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂; and
each R⁵ is independently —R², halo, —NO₂, —CN, —OR², —SR², —N(R³)₂, —C(O)R², —CO₂R², —C(O)C(O)R², —C(O)CH₂C(O)R², —S(O)R², —S(O)₂R², —C(O)N(R³)₂, —SO₂N(R³)₂, —OC(O)R², —N(R³)C(O)R², —N(R³)N(R³)₂, —N(R³)C(═NR³)N(R³)₂, —C(═NR³)N(R³)₂, —C═NOR², —N(R³)C(O)N(R³)₂, —N(R³)SO₂N(R³)₂, —N(R³)SO₂R², or —OC(O)N(R³)₂.

2. The compound according to claim 1, wherein Ring A is Ring A¹, and Ring A¹ is:

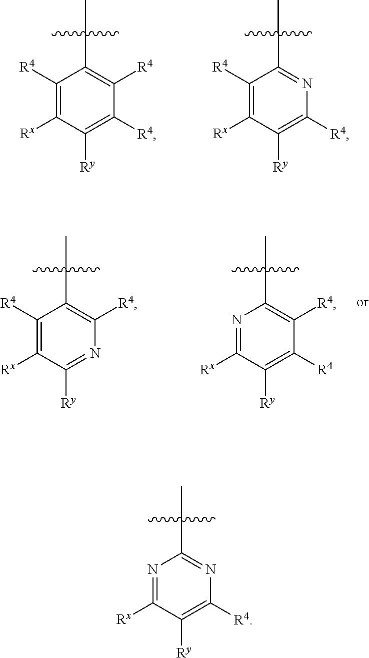

3. The compound according to claim 1, wherein Ring A is Ring A¹, and Ring A¹ is: 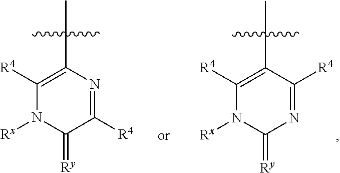
wherein R^{x} and R^{y} are taken together to form a fused heteroaromatic ring.

4. The compound according to claim 1, wherein Ring A is Ring A², and Ring A² is:

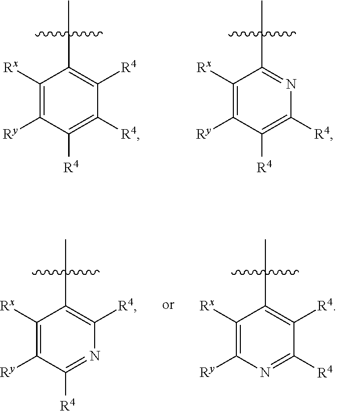

5. The compound according to claim 1, wherein Ring A is Ring A², and Ring A² is: 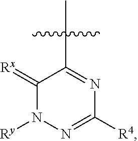
wherein R^{x} and R^{y} are taken together to form a fused heteroaromatic ring.

6. The compound according to claim 1, wherein Ring A is Ring A³, and Ring A³ is:

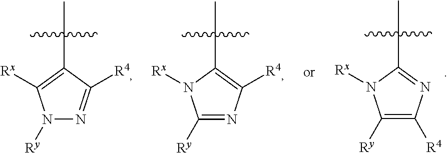

7. The compound according to claim 1, wherein Ring A is Ring A⁴, and Ring A⁴ is:

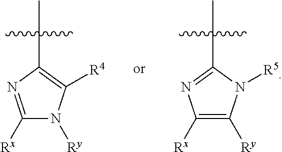

8. The compound according to claim 1, wherein Ring A is Ring A⁵, and Ring A⁵ is:

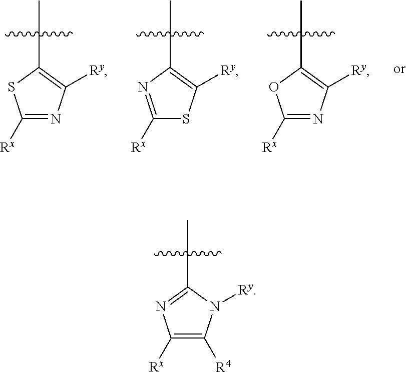

9. The compound according to claim 2, wherein Ring A is 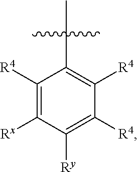
and at least one of R^{x}, R^{y}, and R⁴ is —OH, —OCH₃, or —NH₂.

10. The compound according to claim 1, wherein R^{x} and R^{y} are independently —R², halo, —CN, —OR², —N(R³)₂, or —N(R³)C(O)R².

11. The compound according to claim 1, wherein at least one of R^{x} and R^{y} is optionally substituted C₁₋₆ aliphatic, halo, —CN, —OCH₃, —NH₂, —NHC(O)CH₃, —NH(C₁₋₆ alkyl), or —N(C₁₋₆ alkyl)₂.

12. The compound according to claim 1, wherein at least one of R^{x} and R^{y} is hydrogen.

13. The compound according to claim 1, wherein one of R^{x} and R^{y} is selected from the group consisting of:
(a) an optionally substituted 5-6 membered saturated heterocyclic ring having 1-3 heteroatoms independently selected from nitrogen, oxygen, or sulfur;
(b) an optionally substituted 5-6 membered heteroaryl ring having 1-3 heteroatoms independently selected from nitrogen, oxygen, or sulfur;
(c) an optionally substituted 8-10 membered saturated or partially unsaturated bicyclic carbocyclic ring;
(d) an optionally substituted 8-10 membered bicyclic aryl ring;
(e) an optionally substituted 8-10 membered saturated or partially unsaturated bicyclic heterocyclic ring having 1-4 heteroatoms independently selected from nitrogen, oxygen, and sulfur; and
(f) an optionally substituted 8-10 membered bicyclic heteroaryl ring having 1-4 heteroatoms independently selected from nitrogen, oxygen, and sulfur.

14. The compound according to claim 13, wherein one of R^{x} and R^{y} is an optionally substituted group selected from phenyl, imidazolyl, pyridyl, morpholinyl, pyrimidinyl, piperidinyl, piperazinyl, pyrazinyl, pyrrolidinyl, pyrrolyl, pyrazolyl, triazolyl, tetrazolyl, thienyl, furyl, thiazolyl, isothiazolyl, thiadiazolyl, oxazolyl, isoxazolyl, oxadiaziolyl, pyridazinyl, triazinyl, benzofuranyl, indolyl, quinolinyl, isoquinolinyl, benzimidazolyl, imidazopyridyl, purinyl, indazolyl, pyrrolopyridyl, quinazolinyl, and quinoxalinyl.

15. The compound according to claim 1, wherein R^{x} and R^{y} are taken together with their intervening atoms to form a 5-membered partially unsaturated or aromatic fused ring having 1-3 heteroatoms independently selected from nitrogen, oxygen, and sulfur.

16. The compound according to claim 15, wherein R^{x} and R^{y} are taken together with their intervening atoms to form a pyrrolidino-, imidazolidino-, imidazolidono-, pyrrolo-, pyrazolo-, imidazolo-, triazolo-, thieno-, furo-, thiazolo-, isothiazolo-, thiadiazolo-, oxazolo-, isoxazolo-, or oxadiaziolo-fused ring.

17. The compound according to claim 1, wherein R^{x} and R^{y} are taken together with their intervening atoms to form a 6-membered partially unsaturated or aromatic fused ring having 1-3 heteroatoms independently selected from nitrogen, oxygen, and sulfur.

18. The compound according to claim 17, wherein R^{x} and R^{y} are taken together with their intervening atoms to form a dioxano-, morpholino-, morpholinono-, tetrahydropyrimidino-, piperazino-, piperidino-, pyrazino-, pyrido-, pyrimidino-, or pyridazino-fused ring.

19. The compound according to claim 1, wherein R^{x} and R^{y} are taken together with their intervening atoms to form a fused benzene ring.

20. The compound according to claim 1, wherein R^{x} and R^{y} are taken together with their intervening atoms to form a 7-membered partially unsaturated fused ring having 1-3 heteroatoms independently selected from nitrogen, oxygen, and sulfur.

21. The compound according to claim 20, wherein R^{x} and R^{y} are taken together with their intervening atoms to form an azepino-, diazepino-, azepinono-, or diazepinono-fused ring.

22. The compound according to claim 15, wherein the ring formed by R^{x} and R^{y} is substituted with —NH₂, —CH₃, —OH, —CF₃, or —SH.

23. The compound according to claim 1, wherein Ring A is any one of the groups shown in Table 1.

24. The compound according to claim 23, wherein Ring A is one of the following groups shown in Table 1: vi, vii, x, xxi, xxii, xxvii, xxviii, xxxii, xxxiii, xxxiv, xxxv, xliii, xliv, xlv, xlvii, xlviii, l, li, liv, lv, lxviii, lxxi, lxxii, lxiii, lxxv, lxxxi, lxxxiii, lxxxiv, lxxxvii, lxxxviii, xc, xciii, xcix, c, cxii, cxvi, cxxv, cxxvii, cxxx, cxxxvii, clx, clxvii, clxviii, or clxxxv.

25. The compound according to claim 1, wherein R is hydrogen.

26. The compound according to claim 1, wherein R is hydrogen and L¹ is an optionally substituted, straight or branched C₁₋₄ alkylene chain.

27. The compound according to claim 1, wherein Cy¹ is a 5-6 membered heteroarylene having 1-3 heteroatoms independently selected from nitrogen, oxygen, and sulfur.

28. The compound according claim 39, wherein Cy¹ is thiazolylene or pyrazinylene.

29. The compound according to claim 1, wherein Cy¹ is phenylene.

30. The compound according to claim 1, wherein L² is —NH—.

31. The compound according to claim 1, wherein L² is —C(O)NH—.

32. The compound according to claim 1, wherein Cy¹ is phenylene and L² is —C(O)NR¹—.

33. The compound according to claim 1, wherein Cy² is selected from the group consisting of:
(a) an optionally substituted 5-membered heteroaryl ring having 1-3 heteroatoms independently selected from nitrogen, oxygen, and sulfur;
(b) optionally substituted phenyl;
(c) an optionally substituted 6-membered heteroaryl ring having 1-3 heteroatoms independently selected from nitrogen, oxygen, and sulfur;
(d) an optionally substituted 8-10 membered bicyclic aryl ring; and
(e) an optionally substituted 8-10 membered bicyclic heteroaryl ring having 1-4 heteroatoms independently selected from nitrogen, oxygen, and sulfur.

34. The compound according to claim 33, wherein Cy² is an optionally substituted group selected from phenyl, pyridyl, pyrazinyl and pyrimidinyl.

35. The compound according to claim 1, wherein Cy² any one of the groups shown in Table 2.

36. The compound according to claim 1, wherein said compound is of formula II: 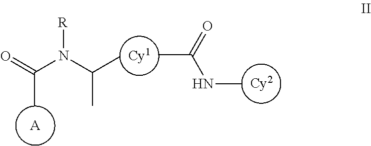
or a pharmaceutically acceptable salt thereof, wherein:
Cy¹ is phenylene or a 5-6 membered heteroarylene having 1-3 heteroatoms independently selected from nitrogen, oxygen, and sulfur, wherein Cy¹ is optionally substituted with 1-2 groups selected from halogen, C₁₋₂ alkyl, C₁₋₂ haloalkyl, —CN, —NO₂, —OH, —O(C₁₋₂ alkyl), —NH₂, —NH(C₁₋₂ alkyl), —N(C₁₋₂ alkyl)₂, —SH, or —S(C₁₋₂ alkyl); and
Cy² is optionally substituted phenyl or an optionally substituted 6-membered heteroaryl ring having 1-3 nitrogens.

37. The compound according to claim 36, wherein said compound is of formula II-a or II-b:

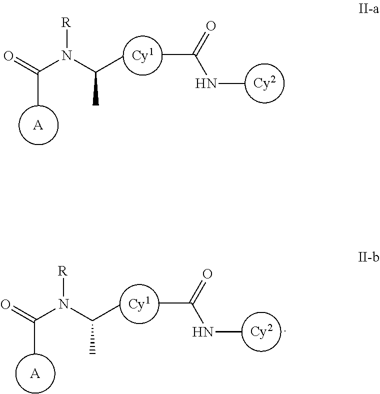

38. The compound according to claim 37, wherein said compound has one of the following formulae:

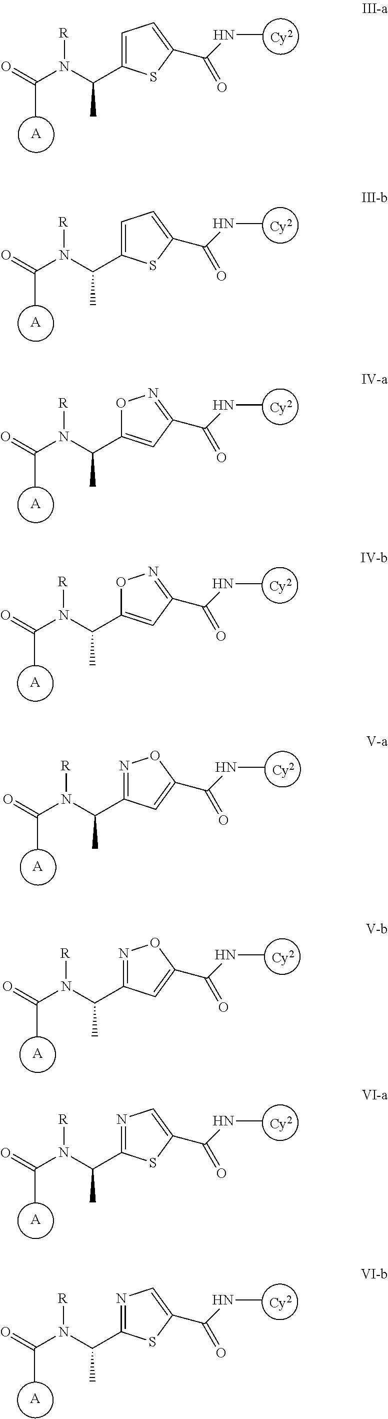

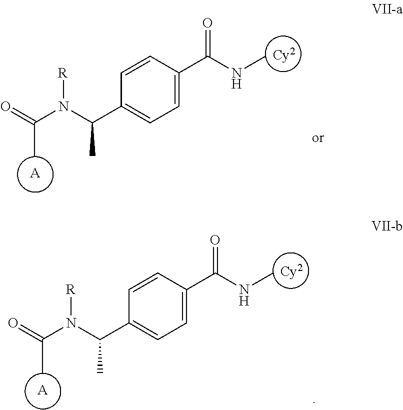

39. The compound according to claim 1, wherein said compound is of formula VIII: 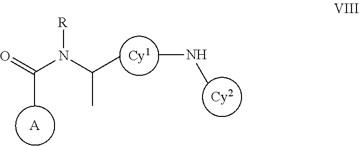
or a pharmaceutically acceptable salt thereof, wherein:
Cy¹ is phenylene, a 5-6 membered saturated or partially unsaturated heterocyclylene having 1-2 heteroatoms independently selected from nitrogen, oxygen, and sulfur, or a 5-6 membered heteroarylene having 1-3 heteroatoms independently selected from nitrogen, oxygen, and sulfur, wherein Cy¹ is optionally substituted with 1-2 groups selected from halogen, C₁₋₂ alkyl, C₁₋₂ haloalkyl, —CN, —NO₂, —OH, —O(C₁₋₂ alkyl), —NH₂, —NH(C₁₋₂ alkyl), —N(C₁₋₂ alkyl)₂, —SH, or —S(C₁₋₂ alkyl); and
Cy² is optionally substituted phenyl or an optionally substituted 6-membered heteroaryl ring having 1-3 nitrogens.

40. The compound according to claim 39, wherein said compound is of formula VIII-a or VIII-b:

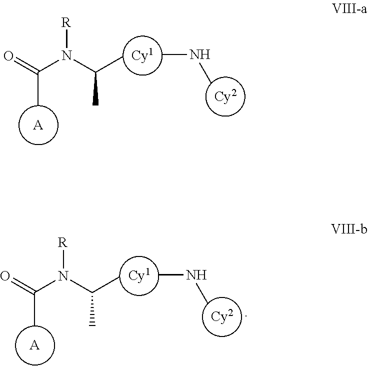

41. The compound according to claim 40, wherein said compound is of formula IX-a, IX-b, X-a, or X-b:

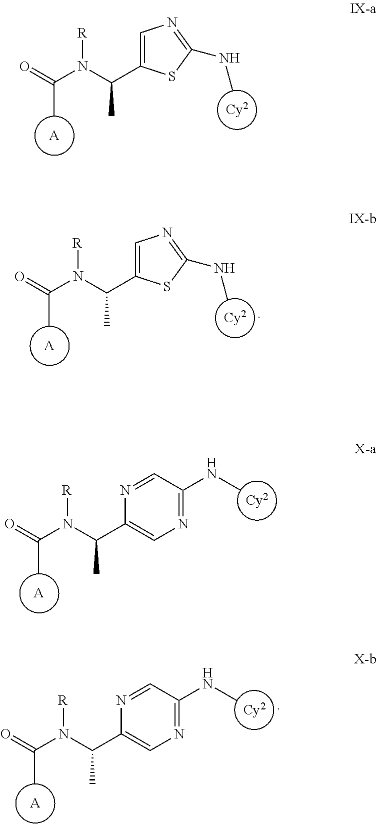

42. The compound according to claim 1, wherein said compound selected from the compounds depicted in Table 3.

43. The compound according to claim 42, wherein said compound is one of the following compounds depicted in Table 3: 2, 4, 6, 9, 12, 13, 14, 15, 19, 20, 28, 30, 35, 37, 38, 40, 42, 199, 203, 205, 208, 224, 232, 236, 240, 241, 243, 244, 245, 269, 274, 297, 268, 274, 297, 174, 176, 180, 183, 188, 201, 292, 267, 265a, 265b, 345, 346, 348, 298, or 287.

44. A pharmaceutical composition comprising a compound according to claim 1 and a pharmaceutically acceptable carrier, adjuvant, or vehicle.

45. The composition of claim 44, in combination with a therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating destructive bone disorders, an agent for treating liver disease, an anti-viral agent, an agent for treating blood disorders, an agent for treating diabetes, or an agent for treating immunodeficiency disorders.

46. A method of inhibiting Raf kinase activity in a patient; or a biological sample, which method comprises administering to said patient, or contacting said biological sample with a compound according to claim 1, or a pharmaceutical composition thereof.

47. A method of treating or lessening the severity of a Raf-mediated disorder in a mammal suffering such disorder, wherein the disorder is selected from a proliferative disorder, a cardiac disorder, a neurodegenerative disorder, an autoimmune disorder, a condition associated with organ transplant, an inflammatory disorder, an immunologically-mediated disorder, a viral disease, or a bone disorder, the method comprising the step of administering to said patient a compound according to claim 1, or a pharmaceutical composition thereof.

48. The method according to claim 47, wherein the disorder is selected from melanoma, leukemia, colon cancer, breast cancer, gastric cancer, ovarian cancer, lung cancer, brain cancer, laryngeal cancer, cervical cancer, renal cancer, cancer of the lymphatic system, cancer of the genitourinary tract (including bladder cancer and prostate cancer), stomach cancer, bone cancer, lymphoma, glioma, papillary thyroid cancer, neuroblastoma, and pancreatic cancer.

49. The method according to claim 47, comprising the additional step of administering to said patient an additional therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating destructive bone disorders, an agent for treating liver disease, an anti-viral agent, an agent for treating blood disorders, an agent for treating diabetes, or an agent for treating immunodeficiency disorders, wherein:
said additional therapeutic agent is appropriate for the disease being treated; and
said additional therapeutic agent is administered together with said composition as a single dosage form or separately from said composition as part of a multiple dosage form.
